SEQUENCE LISTING (56 sequences)

SEQ ID NO: 1 - DNA, 4860 bp, Homo sapiens
  misc_feature (4804)..(4804): n is a, c, g, or t
ggcagtcggcgaactgtctgggcgggaggagccgtgagcagtagctgcactcagctgcccgcgcggcaaagaggaaggcaagccaaacagagtgcgcagagtggcagtgccagcggcgacacaggcagcacaggcagcccgggctgcctgaatagcctcagaaacaacctcagcgactccggctgctctgcggactgcgagctgtggcggtagagcccgctacagcagtcgcagtctccgtggagcgggcggaagccttttttctccctttcgtttacctcttcattctactctaaaggcatcgttattagagggtgcttaaaaagtacagatcaactggatggatgaatggatggaagaggatggaatatcttaacaaaacacattttccttaagtaaattcatgcatactccaaataaaatacagaatgtgaagtatctctgaactgtgctgttgaatatggtagctactagctacatgaaaatcctgttgtgaataagaaggattccacagatcacataccagagcggttttgcctcagctgctctcaactttgtaatcttgtgaagaagctgacaagcttggctgattgcagtgcactatgaggactgaatgacagtgggttttaattcagatatttcaagtgttgtgcgggttaatacaacaaactgtcacaagtgtttgttgtccgggacgtacattttcgcggtcctgctagtatgcgtggtgttccactctggcgcccaggagaaaaactacaccatccgagaagaaattccagaaaacgtcctgataggcaacttgttgaaagaccttaacttgtcgctgattccaaacaagtccttgacaactactatgcagttcaagctagtgtacaagaccggagatgtgccactgattcgaattgaagaggatactggtgagatcttcactaccggcgctcgcattgatcgtgagaaattatgtgctggtatcccaagggatgagcattgcttttatgaagtggaggttgccattttgccggatgaaatatttagactggttaagatacgttttctgatagaagatataaatgataatgcaccattgttcccagcaacagttatcaacatatcaattccagagaactcggctataaactctaaatatactctcccagcggctgttgatcctgacgtaggcataaacggagttcaaaactacgaactaattaagagtcaaaacatttttggcctcgatgtcattgaaacaccagaaggagacaagatgccacaactgattgttcaaaaggagttagatagggaagagaaggatacctatgtgatgaaagtaaaggttgaagatggtggctttcctcaaagatccagtactgctattttgcaagtaagtgttactgatacaaatgacaaccacccagtctttaaggagacagagattgaagtcagtataccagaaaatgctcctgtaggcacttcagtgacacagctccatgccacagatgctgacataggtgaaaatgccaagatccacttctctttcagcaatctagtctccaacattgccaggagattatttcacctcaatgccaccactggacttatcacaatcaaagaaccactggatagggaagaaacaccaaaccacaagttactggttttggcaagtgatggtggattgatgccagcaagagcaatggtgctggtaaatgttacagatgtcaatgataatgtcccatccattgacataagatacatcgtcaatcctgtcaatgacacagttgttctttcagaaaatattccactcaacaccaaaattgctctcataactgtgacggataaggatgcggaccataatggcagggtgacatgcttcacagatcatgaaattcctttcagattaaggccagtattcagtaatcagttcctcctggagaatgcagcatatcttgactatgagtccacaaaagaatatgccattaaattactggctgcagatgctggcaaacctcctttgaatcagtcagcaatgctcttcatcaaagtgaaagatgaaaatgacaatgctccagttttcacccagtctttcgtaactgtttctattcctgagaataactctcctggcatccagttgatgaaagtaagtgcaacggatgcagacagtgggcctaatgctgagatcaattacctgctaggccctgatgctccacctgaattcagcctggatcgtcgtacaggcatgctgactgtagtgaagaaactagatagagaaaaagaggataaatatttattcacaattctggcaaaagataatggggtaccacccttaaccagcaatgtcacagtctttgtaagcattattgatcagaatgacaatagcccagttttcactcacaatgaatacaaattctatgtcccagaaaaccttccaaggcatggtacagtaggactaatcactgtaactgatcctgattatggagacaattctgcagttacgctctccattttagatgagaatgatgacttcaccattgattcacaaactggtgtcatccgaccaaatatttcatttgatagagaaaaacaagaatcttacactttctatgtaaaggctgaggatggtggtagagtatcacgttcttcaagtgccaaagtaaccataaatgtggttgatgtcaatgacaacaaaccagttttcattgtccctccttacaactattcttatgaattggttctaccgtccactaatccaggcacagtggtctttcaggtaattgctgttgacaatgacactggcatgaatgcagaggttcgttacagcattgtaggaggaaacacaagagatctgtttgcaatcgaccaagaaacaggcaacataacattgatggagaaatgtgatgttacagaccttggtttacacagagtgttggtcaaagctaatgacttaggacagcctgattctctcttcagtgttgtaattgtcaatctgttcgtgaatgagtcagtgaccaatgctacactgattaatgaactggtgcgcaaaagcattgaagcaccagtgaccccaaatactgagatagctgatgtatcctcaccaactagtgactatgtcaagatcctggttgcagctgttgctggcaccataactgtcgttgtagttattttcatcactgctgtagtaagatgtcgccaggcaccacaccttaaggctgctcagaaaaacatgcagaattctgaatgggctaccccaaacccagaaaacaggcagatgataatgatgaagaaaaagaaaaagaagaagaagcattcccctaagaacctgctgcttaattttgtcactattgaagaaactaaggcagatgatgttgacagtgatggaaacagagtcacactagaccttcctattgatctagaagagcaaacaatgggaaagtacaattgggtaactacacctactactttcaagcctgacagccctgatttggcccgacactacaaatctgcctctccacagcctgccttccaaattcagcctgaaactcccctgaatttgaagcaccacatcatccaagaactgcctctcgataacacctttgtggcctgtgactctatctccaagtgttcctcaagcagttcagatccctacagcgtttctgactgtggctatccagtgacaaccttcgaggtacctgtgtccgtacacaccagaccgactgattccaggacatgaactattgaaatctgcagtgagatgtaactttctaggaacaacaaaattccattccccttccaaaaaatttcaatggattgtgatttcaaaattaggctaagatcattaattttgtaatctagatttcccattataaaagcaagcaaaaatcatcttaaaaatgatgtcctagtgaaccttgtgctttctttagctgtaatctggcaatggaaatttaaaatttatggaagagacagtgcagcacaataacagagtactctcatgctgtttctctgtttgctctgaatcaacagccatgatgtaatataaggctgtcttggtgtatacacttatggttaatatatcagtcatgaaacatgcaattacttgccctgtctgattgttgaataattaaaacattatcttccaggagtttggaagtgagctgaactagccaaactactctctgaaaggtatccagggcaagagacatttttaagaccccaaacaaacaaaaaacaaaaccaaaacactctggttcagtgttttgaaaatattcactaacataatattgctgagaaaatcatttttattacccaccactctgcttaaaagttgagtgggccgggcgcggtggctcacgcctgtaatcccagcactttgggaggccgaggcgggtggatcacgaggtcaggagattgagaccatcctggctaacacggtgaaaccccatctccactaaaaatacaaaaaattagcctggcgtggtggcgggcgcctgtagtcccagctactcgggaggctgaggcaggagaatagcgtgaacccgggaggcggagcttgcagtgagccgagatggcgccactctgcactccagcctgggtgacagagcaagactctgtctcaaaaagaaaaaaatgttcaatgatagaaaataattttactaggtttttatgttgattgtactcatggtgttccactccttttaattattaaaaagttatttttggggtgggtgtggtggctcacaccgtaatcccagcactttgggaggccgaggtgggtggatcacctgaggtcaggagttcaagaccagtntggccaacatggcgaaaccccgttttaaaaaaaaaaaaaaaaaaaaaaaagaaaaa

SEQ ID NO: 2 - protein, 1037 aa, Homo sapiens [sequence omitted]

SEQ ID NO: 3 - DNA, 21 bp, Artificial
  Synthetic Oligonucleotide
aagcactgaagcaccagtgac

SEQ ID NO: 4 - DNA, 21 bp, Artificial
  Synthetic Oligonucleotide
aagcattgaagcaccagtgac

SEQ ID NO: 5 - DNA, 21 bp, Artificial
  Synthetic Oligonucleotide
aaacaagcagaattctgaatg

SEQ ID NO: 6 - DNA, 21 bp, Artificial
  Synthetic Oligonucleotide
aaacatgcagaattctgaatg

SEQ ID NO: 7 - DNA, 21 bp, Artificial
  Synthetic Oligonucleotide
aagaaactaaggcagatgatg

SEQ ID NO: 8 - DNA, 20 bp, Artificial
  Synthetic Primer
ctcctggcaaaaggtcagag

SEQ ID NO: 9 - DNA, 20 bp, Artificial
  Synthetic Primer
agcttttgctcctctgcttg

SEQ ID NO: 10 - DNA, 21 bp, Artificial
  Synthetic Primer
gagggtagatcatctctgcct

SEQ ID NO: 11 - DNA, 23 bp, Artificial
  Synthetic Primer
cctgattcaaatgagattgtgga

SEQ ID NO: 12 - DNA, 20 bp, Artificial
  Synthetic Primer
tgcctgcaggtcctagaagt

SEQ ID NO: 13 - DNA, 20 bp, Artificial
  Synthetic Primer
aatcttggctcattgcaacc

SEQ ID NO: 14 - DNA, 20 bp, Artificial
  Synthetic Primer
taggaggaaacacaagagat

SEQ ID NO: 15 - DNA, 21 bp, Artificial
  Synthetic Primer
agaaagttacatctcactgca

SEQ ID NO: 16 - DNA, 20 bp, Artificial
  Synthetic Primer
atggatgatgatatcgccgc

SEQ ID NO: 17 - DNA, 20 bp, Artificial
  Synthetic Primer
aagcatttgcggtggacgat

SEQ ID NO: 18 - DNA, 22 bp, Artificial
  Synthetic Primer
aattgggtaactacacctacta

SEQ ID NO: 19 - DNA, 21 bp, Artificial
  Synthetic Primer
ctcgaaggttgtcactggata

SEQ ID NO: 20 - protein, 15 aa, Artificial
  Synthetic Peptide
SIPENSAINSKYTNP

SEQ ID NO: 21 - protein, 15 aa, Artificial
  Synthetic Peptide
NMQNSEWATPNPENR

SEQ ID NO: 22 - protein, 15 aa, Artificial
  Synthetic Peptide
ETKADDVDSDGNRVT

SEQ ID NO: 23 - DNA, 20 bp, Artificial
  Synthetic Primer
ttagattgggctttggaacc

SEQ ID NO: 24 - DNA, 21 bp, Artificial
  Synthetic Primer
gcttcctgaatagctcctgct

SEQ ID NO: 25 - DNA, 20 bp, Artificial
  Synthetic Primer
caaaattgagcgcctatgtg

SEQ ID NO: 26 - DNA, 20 bp, Artificial
  Synthetic Primer
ttgctctaggaaccctcagc

SEQ ID NO: 27 - DNA, 20 bp, Artificial
  Synthetic Primer
ggcaaaaatctcggaatgac

SEQ ID NO: 28 - DNA, 20 bp, Artificial
  Synthetic Primer
aaaggtggagatgcaagtgg

SEQ ID NO: 29 - DNA, 20 bp, Artificial
  Synthetic Primer
atccagtcttccttgccttt

SEQ ID NO: 30 - DNA, 20 bp, Artificial
  Synthetic Primer
ttctgggaggctctctgttc

SEQ ID NO: 31 - DNA, 20 bp, Artificial
  Synthetic Primer
caggtgaaagggtcttcagg

SEQ ID NO: 32 - DNA, 26 bp, Artificial
  Synthetic Primer
aggacataatttgttctatgttccac

SEQ ID NO: 33 - DNA, 21 bp, Artificial
  Synthetic Primer
tttttcaggcctctttgtgtc

SEQ ID NO: 34 - DNA, 26 bp, Artificial
  Synthetic Primer
tgtgtctacacactaacagtgaagga

SEQ ID NO: 35 - DNA, 21 bp, Artificial
  Synthetic Primer
tggtgatgtggaagcaacata

SEQ ID NO: 36 - DNA, 26 bp, Artificial
  Synthetic Primer
aaggtgagaaataatgctctgaagtt

SEQ ID NO: 37 - DNA, 21 bp, Artificial
  Synthetic Primer
gctctccacttgcccctttta

SEQ ID NO: 38 - DNA, 18 bp, Artificial
  Synthetic Primer
gttcccaatttctcagcc

SEQ ID NO: 39 - DNA, 20 bp, Artificial
  Synthetic Primer
gggaggaattcaccctgaaa

SEQ ID NO: 40 - DNA, 20 bp, Artificial
  Synthetic Primer
cctgccccaaattaagaaaa

SEQ ID NO: 41 - DNA, 27 bp, Artificial
  Synthetic Primer
gcgaagcttgtggcattgtgccatttg

SEQ ID NO: 42 - DNA, 35 bp, Artificial
  Synthetic Primer
gcgctcgagtcaaaatccaaataaagtatatggcc

SEQ ID NO: 43 - DNA, 31 bp, Artificial
  Synthetic Primer
gcgctcgagagcccactcaattcctattgag

SEQ ID NO: 44 - DNA, 31 bp, Artificial
  Synthetic Primer
ctcgagaccttctttggtcaaggtaagtaaa

SEQ ID NO: 45 - DNA, 31 bp, Artificial; identical to SEQ ID NO: 44
  Synthetic Primer

SEQ ID NO: 46 - DNA, 30 bp, Artificial
  Synthetic Primer
ctcgagccttggatagttccagttgtaaag

SEQ ID NO: 47 - DNA, 20 bp, Artificial
  Synthetic Primer
ggacttcaccgcacctgatg

SEQ ID NO: 48 - DNA, 20 bp, Artificial
  Synthetic Primer
ctggcagtctccaaacgcat

SEQ ID NO: 49 - DNA, 21 bp, Artificial
  Synthetic Primer
ggatttggtcgtattgggcgc

SEQ ID NO: 50 - DNA, 20 bp, Artificial
  Synthetic Primer
gttctcagccttgacggtgc

SEQ ID NO: 51 - DNA, 20 bp, Artificial
  Synthetic Primer
cggaagctgaagaaacttgg

SEQ ID NO: 52 - DNA, 20 bp, Artificial
  Synthetic Primer
cgtgtccagcacacactaca

SEQ ID NO: 53 - DNA, 20 bp, Artificial
  Synthetic Primer
atggatgatgatatcgccgc

SEQ ID NO: 54 - DNA, 20 bp, Artificial
  Synthetic Primer
aagcatttgcggtggacgat

SEQ ID NO: 55 - DNA, 10 bp, Homo sapiens
acaacaagct

SEQ ID NO: 56 - DNA, 11 bp, Homo sapiens
acaacagagct

(19) United States
(12) Patent Application Publication
(10) Pub. No.: US 2007/0248535 A1
(43) Pub. Date: Oct. 25, 2007

(54) Methods to treat or prevent hormone-resistant prostate cancer using siRNA specific for protocadherin-PC, or other inhibitors of protocadherin-PC expression or activity

(75) Inventors: Ralph Buttyan, New York, NY (US); Mitchell C. Benson, New York, NY (US); Min-Wei Chen, Flushing, NY (US); Francis Vacherot, Creteil (FR); Luis Carlos Soares Queires, Salvador (BR); Stephane Terry, Creteil (FR); Alexandre De La Taille, Paris (FR); Sixtina Gil Diez De Medina, Paris (FR)

Correspondence Address: WilmerHale/Columbia University, 399 PARK AVENUE, NEW YORK, NY, 10022, US

(73) Assignees: THE TRUSTEES OF COLUMBIA UNIVERSITY IN THE CITY OF NEW YORK, New York, NY (US); INSERM, Paris (FR)

(21) Appl. No.: 11/349,473

(22) Filed: Feb. 7, 2006

Related U.S. Application Data
(60) Provisional application No. 60/650,628, filed on Feb. 7, 2005.
(60) Provisional application No. 60/690,232, filed on Jun. 13, 2005.

(51) Int. Cl. A61K 51/00; A01K 67/00; A61K 31/7052; C07H 21/00; C12N 1/00; C12Q 1/68; G01N 33/53; C12N 5/00; C07K 16/00; A61K 38/00; A61K 39/395

(52) U.S. Cl. 424/1.49; 424/1.11; 424/1.69; 424/1.73; 424/130.1; 424/9.1; 435/243; 435/325; 435/410; 435/6; 435/7.1; 514/1; 514/2; 514/44; 530/387.9; 530/388.1; 536/23.1; 800/13; 800/14; 800/15; 800/17; 800/18; 800/3

(57) ABSTRACT

The invention is directed to compounds and methods for treating or preventing hormone-resistant prostate cancer using siRNA specific for protocadherin-PC, or other inhibitors of protocadherin-PC expression or activity, including antisense oligonucleotides and antibodies. The invention also provides for the use of protocadherin-PC as an in vivo prostate cancer biomarker, and includes a kit for detecting prostate cancer in biological samples. Also covered by the invention is a transgenic non-human mammal engineered to overexpress protocadherin-PC specifically in the prostate.

This application claims priority to U.S. Application No. 60/650,628, which was filed Feb. 7, 2005 and U.S. Application No. 60/690,232, which was filed Jun. 13, 2005, both of which are hereby incorporated by reference in their entireties.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND OF THE INVENTION

According to recent estimates by The American Cancer Society, over 30,000 men will die of prostate cancer this year; this number is not significantly different from their projections in previous years. Virtually all of these deaths from prostate cancer will occur in men with hormone-resistant (androgen-independent) disease.

Prostate cancer is a malignancy that develops and progresses under the influence of androgenic steroids. This influence is consistent with the use of various forms of androgen depletion therapies to treat patients diagnosed with metastatic prostate cancer for which surgery is no longer an effective treatment option. Androgen depletion provides rapid palliative relief to patients suffering pain as a consequence of bone metastatic prostate cancer and clinical study has proven that it extends the life span of the advanced prostate cancer patient even though the extension is only a matter of months. The transient effectiveness of androgen depletion therapy for prostate cancer patients is based upon its apparent ability to suppress proliferation of the tumor cells and, in the in vivo setting of the patient, induce apoptosis of, at least, a fraction of these cells. Inevitably, however, residual prostate tumor cells that survive androgen depletion therapy progress to a state where they are considered to be androgen-insensitive because their growth and survival is no longer suppressed in the androgen depleted environment of the treated patient, and it is these androgen-insensitive tumor cells that are associated with the relatively high morbidity and mortality of advanced disease.

To make more significant progress towards reducing overall deaths from this disease while preserving the quality of life for men that have it, it is important to identify better, less toxic means for targeting the androgen-independent prostate cancer cell for elimination from the body of the hormone-resistant prostate cancer patient.

### SUMMARY OF THE INVENTION

The invention provides for a nucleic acid comprising from about 7 to about 30 nucleotides that specifically binds to a region from about nucleotide 3023 to about nucleotide 3727 of SEQ ID NO:1, wherein the nucleic acid is capable of inhibiting expression of protocadherin-PC. SEQ ID NO:1 (FIGS. 26A-26D) is the complete mRNA sequence encoding human protocadherin-PC, comprising nucleotides 1 through 4860, where the protein coding sequence is represented by nucleotides 614 through 3727 (Accession No. AF277053; Chen et al., Oncogene 21:7861-7871 (2002)). In one embodiment, the nucleic acid comprises RNA, antisense RNA, small interfering RNA (siRNA), double stranded RNA (dsRNA), short hairpin RNA (shRNA), cDNA or DNA. In another embodiment, the nucleic acid comprises a sequence within the region of from about nucleotide 3023 to about nucleotide 3727 of SEQ ID NO:1. In an additional embodiment, the nucleic acid comprises a sequence about 70% identical to the complement of a portion of the sequence from about nucleotide 3023 to about nucleotide 3727 of SEQ ID NO:1. In a specific embodiment, the nucleic acid comprises at least one of SEQ ID NO:3, 4, 5, 6, or 7.

The invention provides for a nucleic acid comprising the sequence of SEQ ID NO:3. The invention also provides for a nucleic acid comprising the sequence of SEQ ID NO:4. The invention provides for a nucleic acid comprising the sequence of SEQ ID NO:5. The invention further provides for a nucleic acid comprising the sequence of SEQ ID NO:6. The invention also provides for a nucleic acid comprising the sequence of SEQ ID NO:7.

The invention provides for nucleic acids useful for inhibiting expression or function of protocadherin-PC, which has been shown to be upregulated in hormone-resistant prostate tumors from patients and in hormone-resistant variants of cultured human prostate cancer cells. These nucleic acids, for example siRNAs and shRNAs, are useful to reduce expression of protocadherin-pc in hormone-resistant prostate cancer cells, and subsequently block the wnt signaling pathway leading to death of hormone-independent tumor cells. These nucleic acids represent useful therapeutic agents for hormone-resistant prostate cancer patients. The nucleic acids may also be useful for treating other advanced male cancers and other cancers in which protocadherin-PC is expressed.

In an additional embodiment, the nucleic acid comprises a UU overhang or a TT overhang. In yet another embodiment, the nucleic acid comprises at least one chemically modified nucleotide or at least one modified internucleotide linkage to render it resistant to enzymatic degradation. In a further embodiment, the modified nucleotide comprises a 2′-O-methoxy-residue. In another embodiment, the modified nucleotide linkage is a phosphorothioate linkage.

One aspect of the invention provides for a nucleic acid comprising a nucleic acid expression vector encoding a short hairpin RNA (shRNA), wherein the shRNA comprises the small interfering RNA (siRNA) nucleotide sequence of SEQ ID NO: 3, 4, 5, 6, or 7. In one embodiment, the shRNA comprises SEQ ID NO: 3, 4, 5, 6, or 7 in an expression vector.

The invention also provides for a host organism comprising a nucleic acid of the invention. In one embodiment, the host is a prokaryote or a eukaryote. In another aspect, the invention is directed to a cell comprising a nucleic acid of the invention. The invention also encompasses a mammal comprising a cell of the invention. For example, a xenograft model for prostate cancer in which a tumor comprising human prostate cancer cells expressing anti-protocadherin-PC siRNA is grafted into a mouse to assess the influence of protocadherin-PC on tumor growth.

Provided for by the present invention is an antibody or antigen-binding fragment thereof that specifically binds to the Y-chromosome-encoded homologue of protocadherin-PC comprising the polypeptide amino acid sequence of SEQ ID NO:2 (FIG. 27), wherein the antibody or antigen-binding fragment thereof does not bind to the X-chromosome-encoded homologue of protocadherin-PC. Also provided for by the invention is an antibody or antigen-binding fragment thereof that binds to the Y-chromosome encoded homologue of protocadherin-PC and binds to the X-chromosome encoded homologue of protocadherin-PC.

The invention is directed to a hybridoma cell line designated HB 0337 LIU and deposited at the CNCM under No. I-3560. The invention is directed to another hybridoma cell line designated HB 0337 SSA and deposited with the CNCM under No. I-3561. Both hybridoma cell lines were deposited on Jan. 24, 2006 with the Collection Nationale de Cultures de Microorganismes (CNCM), Institut Pasteur, 25 rue de Docteur Roux, F-75724 Paris Cedex 15, under the provisions of the Budapest Treaty on the International Recognition of the Deposit of Microorganisms for the Purposes of a Patent Procedure. The invention also provides for a monoclonal antibody produced by hybridoma cells deposited with the CNCM under No. I-3560. The invention further provides for a monoclonal antibody produced by hybridoma cells deposited with the CNCM under No. I-3561.

The invention provides for a method for comprises treating cancer in a subject, the method comprising administering to the subject an effective amount of an inhibitor of protocadherin-PC. In certain embodiments, the cancer comprises prostate, breast, melanoma, oral, colon, ovarian, endometrial, hepatocellular carcinoma, or head and neck tumors or any combination thereof.

The invention also provides a method for treating hormone-resistant prostate cancer in a subject, the method comprising administering to the subject an effective amount of an inhibitor of protocadherin-PC. In one embodiment, the hormone-resistant prostate cancer is also resistant to chemotherapy and/or radiation therapy.

The invention provides for a method for treating prostate cancer in a subject, the method comprising administering to the subject a combination of one or more androgen-withdrawal therapies and an effective amount of an inhibitor of protocadherin-PC. In one embodiment, the androgen-withdrawal therapy comprises surgical orchiectomy. In another embodiment, the androgen-withdrawal therapy comprises medical hormone therapies including but not limited to anti-androgens and luteinizing hormone-releasing hormone agonists.

According to the methods of the invention, the inhibitor comprises a small interfering RNA (siRNA) that specifically binds a nucleic acid encoding protocadherin-PC, an antisense oligonucleotide that specifically binds a nucleic acid encoding protocadherin-PC, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof. In additional embodiments, the inhibitor comprises a protein interaction inhibitor that disrupts protocadherin-PC binding domains, FHL-2 binding domains, or β-catenin binding domains. In accord with the methods of the invention, an effective amount comprises an amount of inhibitor effective to arrest, delay or reverse the progression of the cancer.

The invention provides for a method for treating prostate cancer in a subject, the method comprising administering to a subject an effective amount of a radiolabeled compound capable of specifically binding to protocadherin-PC. In certain embodiments, the compound comprises a small interfering RNA (siRNA) that specifically binds a nucleic acid encoding protocadherin-PC, an antisense oligonucleotide that specifically binds a nucleic acid encoding protocadherin-PC, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof. The invention provides for an antibody that specifically binds the Y-chromosome encoded homologue of protocadherin-PC or specifically binds the X-chromosome encoded homologue of protocadherin-PC. The invention also provides for an antibody that binds to both the Y-chromosome encoded homologue and the X-encoded homologue of protocadherin-PC. In another embodiment, the compound comprises a nucleic acid that is capable of specifically binding to another nucleic acid, or fragment thereof, encoding protocadherin-PC.

In another aspect, the invention provides for a method for in vivo imaging of cancer in a subject, the method comprising (a) administering to the subject a radiolabeled compound capable of specifically binding to protocadherin-PC or FHL-2; and (b) detecting the presence of the radiolabeled compound in the subject, thereby imaging cancer in the subject. In specific embodiments, the cancer comprises prostate cancer or breast cancer. In other embodiments, the compound comprises a small interfering RNA (siRNA) that specifically binds a nucleic acid encoding protocadherin-PC, an antisense oligonucleotide that specifically binds a nucleic acid encoding protocadherin-PC, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof. In further embodiments, the compound comprises a nucleic acid specific for a nucleic acid, or a fragment thereof, encoding protocadherin-PC or FHL-2. In additional embodiments, the compound is detected by MRI, SPECT, CT, or ultrasound.

The invention also provides for a method for identifying whether a test compound is capable of inhibiting protocadherin-PC protein activity, the method comprising (a) contacting a protocadherin-PC protein with (i) a test compound and (ii) a β-catenin or an FHL-2 or both; and (b) determining whether activity of the protocadherin-PC protein of step (a) is inhibited as compared to the activity of a protocadherin-PC protein in the absence of the test compound, so as to identify whether the test compound is capable of inhibiting protocadherin-PC protein activity. In various embodiments, the determining comprises (a) determining binding of the protocadherin-PC protein to the β-catenin and/or to the FHL-2, (b) determining whether the protocadherin-PC is capable of translocating β-catenin to the cytoplasm, (c) determining whether protocadherin-PC is activating the wnt signaling pathway or increasing the expression of LEF-1/TCF target genes in the cancer cell, (d) determining whether protocadherin-PC is modulating the expression of the androgen receptor protein, or (e) any combination thereof. In another embodiment, the contacting is achieved by applying the test compound to cells expressing the protocadherin-PC, the β-catenin, and the FHL-2.

Provided for by this invention is a method for identifying whether a test compound is capable of inhibiting protocadherin-PC binding to β-catenin or FHL-2, the method comprising (a) contacting a protocadherin-PC protein with (i) a test compound and (ii) a β-catenin or an FHL-2 or both; and (b) determining whether binding of the protocadherin-PC protein to the β-catenin and/or the FHL-2 is inhibited compared to binding of the protocadherin-PC protein to the β-catenin and/or the FHL-2 in the absence of the test compound, so as to identify whether the test compound is capable of inhibiting the protocadherin-PC binding to the β-catenin or the FHL-2. In certain embodiments of this method, the test compound comprises a nucleic acid, a small molecule, a peptide, a PNA, a peptidomimetic, or an antibody. In one embodiment, the method is carried out for more than one hundred compounds. In another embodiment, the method is carried out in a high-throughput manner.

In yet another aspect, the invention provides for a method for identifying whether a test compound is capable of inhibiting gene expression of protocadherin-PC, the method comprising (a) contacting a nucleic acid encoding a protocadherin-PC protein with a test compound; and (b) determining whether the protocadherin-PC gene expression is inhibited compared to protocadherin-PC gene expression in the absence of the test compound. In an embodiment of the method, the determining comprises measuring transcription of the protocadherin-PC gene. In another embodiment, the determining comprises measuring protocadherin-PC mRNA. In yet another embodiment, the determining comprises measuring translation of protocadherin-PC RNA into protein. In an additional embodiment, the determining comprises quantifying protocadherin-PC protein.

Another aspect of this invention provides for a kit for determining whether or not a subject has or may develop prostate cancer, the kit comprising (a) an antibody or an antigen-binding fragment thereof, that specifically binds to a protocadherin-PC or an FHL-2; and (b) at least one negative control sample that does not contain a protocadherin-PC antigen or an FHL-2 antigen. In an embodiment, the kit further comprises a positive control sample that contains a protocadherin-PC antigen in an amount characteristic of a human prostate cancer cell. In a further embodiment, the antibody or antigen-binding fragment is labeled with a detectable signal. In another embodiment, the antibody comprises monoclonal antibodies produced by hybridoma cells designated HB 0337 LIU and deposited with the CNCM under No. I-3560. In an additional embodiment, the antibody comprises monoclonal antibodies produced by hybridoma cells designated HB 0337 SSA and deposited with the CNCM under N. I-3561.

The present invention provides for a transgenic non-human mammal whose genome comprises a transgene comprising a nucleic acid encoding a protocadherin-PC operably linked to a tissue-specific promoter. In certain embodiments, the mammal is a mouse, a primate, a bovine, or a porcine. In a specific embodiment, the tissue-specific promoter is a prostate-specific probasin gene promoter element.

The invention also provides for an F1 transgenic mouse produced from a cross between a transgenic mouse of this invention and a transgenic mouse of the TRAMP line (strain: C57BL/6-Tg(TRAMP)8247Ng/J; Jackson Lab No. 003135) or any other mouse that develops prostate cancer.

Provided for in another aspect is a method for determining whether a test compound is capable of treating prostate cancer, the method comprising (a) administering an effective amount of a test compound to a transgenic non-human mammal whose genome comprises a transgene comprising a nucleic acid encoding a protocadherin-PC operably linked to a tissue-specific promoter, wherein the transgenic non-human mammal has prostate cancer; (b) measuring progression of prostate cancer in the transgenic non-human mammal of (a); (c) comparing the measurement of progression of prostate cancer of step (b) to that of a sibling of the transgenic non-human mammal, wherein the sibling was not administered the test compound, and wherein an arrest, delay or reversal in progression of prostate cancer in the transgenic non-human mammal of (a) indicates that the test compound is capable of treating prostate cancer.

This invention provides for an isolated prostate cancer cell that does not express a protocadherin-PC gene, wherein the naturally occurring prostate cancer cell does express the protocadherin-PC gene.

The invention encompasses compositions comprising one or more of the nucleic acids of the invention and a pharmaceutically acceptable carrier.

The subject on which the method is employed may be any mammal, e.g. a human, mouse, cow, pig, dog, cat, rat, rabbit, or monkey.

The administration of the agent may be effected by intralesional, intraperitoneal, intramuscular, intratumoral or intravenous injection; by infusion; or may involve liposome- or vector-mediated delivery; or topical, nasal, oral, anal, ocular or otic delivery, or any combination thereof.

In the practice of the method, administration of the inhibitor may comprise daily, weekly, monthly or hourly administration, the precise frequency being subject to various variables such as age and condition of the subject, amount to be administered, half-life of the agent in the subject, area of the subject to which administration is desired and the like.

In connection with the method of this invention, a therapeutically effective amount of the inhibitor may include dosages which take into account the size and weight of the subject, the age of the subject, the severity of the prostate cancer, the method of delivery of the agent and the history of the symptoms in the subject.

### BRIEF DESCRIPTION OF THE FIGURES

FIGS. 1A-1C. PCDH-PC expression increases wnt-mediated signaling in prostate and other cancer cells. FIG. 1A. Comparative Western blot analysis of β-catenin protein in nuclear extracts from control (untransfected) and pCMV-myc (empty vector) transfected LNCaP cells or from LNCaP cells maintained for 7 days in androgen-free medium or transfected for 48 hrs with a PCDH-PC expression vector (above) or for lamin A/C (below) loading control shows that nuclear β-catenin is only detected in cells that express PCDH-PC. FIG. 1B. β-galactosidase-normalized luciferase activity in LNCaP cells subsequent to 48 hrs transfection with the Tcf-sensitive pTOP reporter vector; (Left Panel) cells maintained for 7 days in normal medium (FBS) or 7 days in androgen-free medium (CS-FBS) (Right Panel) cells transfected for 48 hrs with empty vector (pcDNA3) or PCDH-PC expression vector (pPCDH-PC) as indicated. FIG. 1C. β-galactosidase normalized luciferase activity in pTOP transfected DU145 (Left Panel), CWR22rv-1 (Middle Panel) or HCT116 cells (Right Panel) co-transfected with empty vector (pcDNA3) or pPCDH-PC, as indicated. Bars indicate standard error of means from 3 different experiments.

FIG. 2. RT-PCR confirms upregulated expression of wnt7b, cox-2 and c-myc mRNA in LNCaP cells transfected PCDH-PC expression vector. cDNA from LNCaP cells transfected for 48 hrs with PCDH-PC expression vector or control (pCMV-myc) empty vector were amplified with primers specific for human wnt 7b, cox-2, c-myc or β-actin for 24, 28 or 32 cycles and the PCR products were electrophoresed on agarose gels and visualized under UV light. Results shown are for 28-cycle amplification.

FIGS. 3A-3D. PCDH-PC expression is associated with neuroendocrine transdifferentiation of prostate cancer cells. FIG. 3A. LNCaP cells were grown in normal medium (control), androgen-free medium (CS-FBS) or in normal medium supplemented with db-cAMP, IL-6 or NS-398. Western blot of protein extracts were probed with antibody against human NSE (Top Panel), human chromogranin-A (Middle Panel) or human β-actin (Bottom Panel). FIG. 3B. Same cells were extracted for RNAs that were converted to cDNA and subject to PCR for 32 cycles with primers for human β-actin (Upper Band) or for PCDH-PC (Lower Band). PCR products from each reaction were mixed together and electrophoresed on an agarose gel that was stained with ethidium bromide and visualized under UV light. FIG. 4C. LNCAP cells grown in normal medium (control LNCaP) or in androgen free medium (CS-FBS LNCaP) or transfected for 48 hrs with empty vector (pCMV-myc), PCDH-PC expression vector or mutant, stabilized β-catenin expression vector were extracted for protein. A Western blot made from these extracts was probed for expression of NSE (Upper Panel), chromogranin A (Middle Panel) or β-actin (Lower Panel). FIG. 3D. PC-3 cells were transfected for 48 hrs with empty vector (pCMV-myc) or with PCDH-PC expression vector and a Western blot made from protein extracts of these cells were probed for expression of NSE (Top Panel) or β-actin (Bottom Panel).

FIGS. 4A-4B. siRNAs against PCDH-PC suppress PCDH-PC expression and NE transdifferentiation of LNCaP cells. FIG. 4A. LNCaP cells were transfected with the pPCDH-PC-myc expression vector and were co-transfected with siRNA against human lamin or siRNAs 181, 190 or 208 designed to suppress PCDH-PC expression. A Western blot made against protein extracts from these cells were probed with anti-myc (Top Panel) to identify expression of the 110 kd PCDH myc-tagged protein, anti-α-actin (Middle Panel) or anti-human E-cadherin (Bottom Panel). FIG. 4B. A repeated experiment that includes control LNCaP cells (no transfection) or pCMV-PCDH-myc transfected LNCaP cells co-transfected with siRNAs against lamin or against PCDH-PC (181, 190 or 208). The Western blot was probed for NSE expression (Top Panel) or for β-actin expression (Bottom Panel).

FIGS. 5A-5C. siRNAs against PCDH-PC suppress PCDH-PC expression, TCF-mediated transcription and NE transdifferentiation in LNCaP cells grown in androgen-free medium. FIG. 5A. RNAs extracted from LNCaP cells grown in normal medium (Control) or in androgen-free medium (CS-FBS, None) were compared toRNAs from LNCaP cells grown in androgen-free medium transfected with siRNAs against PCDH-PC (181, 190 and 208) or siRNA against lamin by RT-PCR using primers specific for PCDH-PC (Top Panel) or β-actin (Bottom Panel). PCR reaction products were electrophoresed on agarose gels and were visualized after ethidium bromide staining under UV light. FIG. 5B. LNCaP cells cultured in normal medium (Control) were compared to LNCaP cells grown in androgen-free medium for 7 days without or with transfection with siRNA 181 against PCDH-PC or siRNA against lamin for expression of luciferase from the TCF-sensitive reporter pTOP for normalized luciferase activity. FIG. 5C. Protein extracts from LNCaP cultures grown under the same conditions as A, above, were compared by Western blot analysis for expression of NSE (Top Panel) or expression of β-actin (Bottom Panel).

FIG. 6. Dominant negative TCF suppresses the ability of PCDH-PC to induce neuroendocrine transdifferentiation. LNCaP cells were co-transfected with pCMV-myc (empty vector), pPCDH-PC-myc, pβ-catenin or pDN-TCF, as indicated for 48 hrs. Protein extracts were analyzed by comparative Western blotting for expression of NSE (Top panel) or expression of β-actin (Bottom panel).

FIGS. 7A-7C. siRNA against β-catenin suppresses the ability of PCDH-PC expression to induce neuroendocrine transdifferentiation of LNCaP cells. FIG. 7A. Untransfected LNCaP cells (Control LNCaP) or LNCaP cells transfected for 48 hrs with siRNAs against β-catenin or lamin. Protein extracts of these cells were compared by Western blotting for expression of β-catenin (Top Panel) or β-actin (Bottom Panel). FIG. 7B. Comparative Western blot analysis of untransfected (Control LNCaP) cells or LNCaP cells transfected with pPCDH-PC-myc and no siRNA or siRNA against β-catenin or lamin for expression of NSE (Top Panel) or expression of β-actin (Bottom Panel). FIG. 7C. Comparative Western blot analysis of LNCaP cells grown in normal medium (Control LNCaP) or in androgen-free medium (CS-FBS) for 7 days without (no siRNA) or with transfection with siRNA against β-catenin or lamin for expression of NSE (Top Panel) or β-actin (Bottom Panel).

FIGS. 8A-8B. Expression of PCDH-PC mRNA in human prostatic cell cultures and in xenograft of LNCaP cells. FIG. 8A. Expression of PCDH-PC mRNA was investigated on prostatic cell cultures. cDNA from tumor cell lines (LNCaP, -TR and SSR) and from different separated primary cultures of (benign) human prostatic cells were examined for PCDH-PC by semi-quantitative RT-PCR. PCDH-PC mRNA expression was not detected in epithelial and stromal cells from different separated primary cultures. However, its mRNA was present in different LNCaP cell lines and was higher in apoptosis resistant lines LNCaP-TR and LNCaP-SSR compared to LNCaP parental cell line. Number indicated different culture preparations. FIG. 8B. To evaluate the relative expression of PCDH-PC mRNA in xenograft tumor cells, LNCAP cells were injected subcutaneously into male nude mice. Castration was performed when the tumor size was approximately 0.3 cm³. Mice were sacrificed 4 weeks after castration and their tumors were removed and fixed in formalin and embedded in paraffin. Tissue sections obtained from xenograft tumor, before (left panel) and 4 weeks (right panel) after castration of the host, were used to detect PCDH-PC mRNA by using in situ hybridization technique. This procedure was based on the use of digoxigenin-labeled PCDH-PC antisense probes. Increase of protocadherin-PC mRNA in LNCAP xenograft tumors was induced by castration. Negative control was obtained with PCDH-PC sense probe applied on tissue section of xenograft tumor after 4 weeks of the castration (insert of right panel). Magnification: ×200.

FIGS. 9A-9B. Tumorigenicity of protocadherin-PC overexpressed LNCaP cells in castrated nude mice. FIG. 9A. 2×10⁶ of either control LNCaP cells or PCDH-PC transformed LNCaP (LNCaP-pcdh-PC-myc) cells were injected into 8 nude mice castrated 1 week before injection. After 7 weeks, mice injected with control cells had no visible or palpable tumor (0/8) whereas 100% of castrated mice xenografted with LNCaP-pcdh-PC-myc cells formed tumors (8/8). Tumor volume was determined as described in Materials and Methods. FIG. 9B. Hematoxylin and eosin staining showed these tumors were highly vascularized. Magnification: ×200.

FIG. 10. Protocadherin-PC mRNA expression in human prostatic tissues. Relative expression of protocadherin-PC mRNA in prostatic tissues was determined by semi quantitative RT-PCR and by comparison with an internal control, the TBP mRNA. Values corresponded to the mean of protocadherin-PC expression levels in RNAs extract from different groups: the peripheral (n=7), central (n=9) and transitional (n=6) zones of the normal prostate, the benign hyperplastic prostate (n=15), untreated (n=10) and treated (n=8) prostate tumors and hormonal refractory patients (n=9).

FIG. 11. In situ localization of protocadherin-PC mRNA in prostatic tissues. In situ hybridization technique was performed on formalin fixed paraffin embedded tissue using digoxigenin-labeled protocadherin-PC antisense probes. Panels a and b, tissues from normal prostate. Note the staining corresponding to protocadherin-PC mRNA was mainly localized in the basal epithelial cells. Differentiated glandular cells were faint or negative staining. No staining was obtained with protocadherin-PC sense probe applied on normal tissue section (insert of panel b). Representative results of ISH performed on primary (untreated) cancers were presented in panel d. Tumor cells (indicated by arrows) were strongly positive for protocadherin-PC staining compared to adjacent normal epithelial cells. In tissues obtained from patients treated by hormonal therapy (panel d) and from hormone-refractory human prostate cancers (panels e-f). Strong staining corresponding to protocadherin-PC mRNA was localized in all tumor cells (indicated by arrows) and in normal (atrophic) epithelial cells. Magnification: panels a-f and insert of panel b×200; panel b×1000.

FIG. 12. Protocadherin-PC mRNA is expressed in some human normal tissues. Relative expression of Pcdh-PC mRNA in different human normal tissues (brain, duodenum, kidney, liver, lung, placenta, prostate, skeletal muscle, spleen and urothelium) was determined by semi quantitative RT-PCR.

FIG. 13. Comparison of Protocadherin-PC and AR transcripts expressed in human hormonal refractory prostate cancer tissues. The relative expression of AR and protocadherin-PC were analyzed in 4 normal prostate tissues (NP) and 9 hormone-refractory prostate tumors (HRCaP). The expression levels of the protocadherin-PC and the androgen receptor mRNA were determined by comparison respectively with TBP and GADPH mRNA levels. Note that except the HRCaP-3 sample which displayed both overexpression of AR and Pcdh-PC, there was no correlation between high level expression of these two molecules (p>0.5).

FIG. 14. Proteins were extracted from untransfected LNCaP cells (Control) or from LNCAP cells that were transfected for 48 hrs with mutated β-catenin (pbeta-Cat) or PCDH-PC (pPCDH-PC-myc) expression vectors or with an empty expression vector (pCMV-myc). Equal aliquots of protein were electrophoresed on a polyacrylamide gel and then blotted onto a PVDF filter to produce a Western blot. The same blot was probed with an antibody against Akt protein (top panel) or against phosphorylated Akt (ser 473) (second panel) or with an antibody against MDM2 protein (third panel) or phosphorylated MDM2 protein (bottom panel). Results show that transfection with PCDH-PC or β-catenin highly upregulate phosphorylation of Akt and its downstream target MDM2.

FIG. 15. Proteins were extracted from untransfected LNCaP cells maintained in androgen free medium for 7 days (CS-FBS Control) or from 7-day androgen-free LNCaP cells that were transfected for 48 hrs with β-catenin siRNA (CS-FBS+beta-Cat siRNA) or dominant negative Tcf (CS-FBS+DN-Tcf) or PCDH-PC siRNA 181 (CS-FBS+PCDH-PC siRNA) or lamin siRNA (CS-FBS+lamin siRNA). Equal aliquots of protein were electrophoresed on a polyacrylamide gel and then blotted onto a PVDF filter to produce a Western blot. The same blot was probed with an antibody against Akt protein (top panel) or against phosphorylated Akt (ser 473) (second panel) or with an antibody against MDM2 protein (third panel) or phosphorylated MDM2 protein (bottom panel). Results show that suppression of PCDH-PC expression or β-catenin expression/activity block the upregulation of Akt phosphorylation that is found when prostate cancer cells are cultured in androgen-free conditions. The blockade of Akt phosphorylation by PCDH-PC siRNA could be involved in the process through which this molecule induces the death of prostate cancer cells under androgen-free conditions.

FIGS. 16A-16B. A CHIP Assay identifies functional LEF-1/TCF binding sites within the proximal promoter of the hAR gene. FIG. 16A. Scheme identifies relative sites of potential LEF-1/TCF binding sites within the first 2000 bp 5′ upstream of the start of transcription (TSS) of the hAR gene and sites of primer amplification products used to analyze DNA extracted from immunoprecipitated chromatin from cell specimens. FIG. 16B. Ethidium bromide-stained agarose gel profiles of PCR reaction products from input control DNA (In), β-catenin antibody immunoprecipitated control transfected (empty vector) LNCaP cell chromatin DNA (Con), β-catenin transfected LNCaP cell DNA (Cat) or PCDH-PC transfected LNCaP cell DNA (PCDH).

FIGS. 17A-17B. FIG. 17A. Northern blot analysis of t6 (PCDH-PC) expression in parental LNCaP, hormone-resistant LNCaP (-TR or -SSR) or in LNCaP cells cultured for 5 or 10 days in androgen-free (CSS) medium. PCDH-PC is not expressed in parental LNCaP cells but highly expressed in hormone-resistant and cells grown in androgen-free medium. FIG. 17B. Rnase protection assay shows upregulation of PCDH-PC transcript (protected fragment 249 bp) in LNCaP xenograft at 2 weeks following castration of the host mouse when tumor is regrowing once again.

FIG. 18. Selective killing of LNCaP cells grown in androgen-free medium by PCDH-PC siRNA. LNCaP cells grown in normal medium or in androgen-free medium (phenol red-free RPMI supplemented with CS-FBS) for 5 days were transfected with PCDH-PC siRNA (#181) or with lamin siRNA, as indicated for a further 48 hrs. Cells were collected, fixed and stained with propidium iodide and were analyzed by flow cytometry. Bars represent the % population of cells in the sub-GO peak considered to be apoptotic. The bars are averages based on two measurements under each condition. No siRNAs were untransfected cells.

FIG. 19. Graphic summary describing the putative relationship between prostate adenocarcinoma and NE-transdifferentiated prostate cancer. Environmental stimuli such as hormone withdrawal can induce the NE trans-differentiation process and the trans-differentiated NE-like cancer cells gain the ability to feed prostate cancer, even at a distant site, a number of peptide hormones that increase proliferative activity and protect from apoptosis-inducing therapies.

FIGS. 20A-20C. FIG. 20A. Northern blot analysis of pro-PC expression in LNCaP variants or in parental LNCaP cells maintained in charcoal-stripped serum (CS-FBS) shows expression of pro-PC mRNA in apoptosis-resistant variants (-TR & -SSR) and in hormone-deprived parental LNCaP. FIG. 20B. Evaluation of pro-PC protein on Western blot shows similar expression pattern. FIG. 20C. LNCAP-TR or stably transfected with pro-PC (-T6-2, -4) cDNA are resistant to phorbol ester induced apoptosis compared to parental LNCaP or -T6-5 that does not express pro-PC.

FIG. 21. RT-PCR reactions products from cDNAs of normal prostate regions or microdissected prostate cancers (from untreated or hormonally-treated patients as indicated). Primer pairs amplify common region of pro-PC and PCDHX gene product but pro-PC related cDNA is 13 bp shorter in this region due to deletion of small region. 500 ng cDNA were amplified for 35 cycles and electrophoresed on agarose gels. Bands are visualized by ethidium bromide staining under UV light. Y-specific cDNA sequence is increased in hormone-resistant tumors.

FIGS. 22A-22B. FIG. 22A. In situ hybridization of thin section of human prostate containing untreated prostate cancer (identified by arrows) using a digoxigenin-labeled RNA probe from a region common to Pro-PC/PCDHX. Prostate cancer cells are positive, as are rare basal cells within the normal epithelium that may represent neuroendocrine cells. FIG. 22B. In situ hybridization of thin section of human prostate containing hormone-resistant prostate cancer using a digoxigenin-labeled RNA probe from a region common to pro-PC/PCDHX shows strong staining of tumor regions but lack of staining of non-tumor area.

FIGS. 23A-23D. FIG. 23A. Immunoprecipitates using anti-pro-PC or control pre-immune serum were screened for co-precipitation of β-catenin on Western blots. Left lane contains recombinant β-catenin control. FIG. 23B. Luciferase levels of LNCaP variants 48 hrs after transfection with pTOP (Tcf-sensitive reporter vector). Apoptosis-resistant variants (-TR and -SSR make significantly more luciferase (normalized to β-gal co-transfection). * indicates p-values compared to parental LNCaP. FIG. 23C. Western blots of nuclear fractions from control LNCaP (untransfected or transfected with empty plasmid for 48 hrs) or from pPro-PC transfected cells were probed with anti-β-catenin (above) or lamin (below). FIG. 23D. LNCaP or HT119 cells were transfected with empty vector (pCMV-myc) or pPro-PC+pTOP/pβ-gal for 48 hrs. Bars show mean normalized luciferase expression. * indicates P values compared to pCMV/pTOP/pβ-gal control.

FIG. 24. Agar Plate (with X-gal substrate) streaked with yeast “negative control” (non-reactive combination of prey/bait cDNAs) that lacks green staining; yeast “positive controls” (provided in the yeast-2-hybrid kit or yeast co-transfected with human E-cadherin bait and beta-catenin prey (known to bind together) stains green; or yeast co-transfected with PCDH-PC cDNA (Proto-PC) and human FHL-2 recombinant cDNA. (stains green). These results support the idea that PCDH-PC and FHL-2 are protein binding partners.

FIG. 25. In vitro “pulldown” assay confirms binding between pro-PC protein and FHL-2 protein. Expression plasmids for pro-PC (tagged with myc) or FHL-2 (tagged with HA) were in vitro transcribed and in vitro translated in the presence of ³⁵S-met. Proteins were immunoprecipitated, as indicated, electrophoresed and exposed to film for autoradiography. Left 2 lanes show pro-PC can be immunoprecipitated by anti-myc and FHL-2 can be precipitated by anti-HA whereas these proteins cannot be precipitated by the opposing antibody (middle 2 lanes). In right 2 lanes, combined extracts, FHL-2 is co-precipitated with anti-myc and pro-PC is co-precipitated with anti-HA (arrows) demonstrating in vitro direct binding of these 2 molecules.

FIGS. 26A-26D. Complete mRNA sequence encoding Y-chromosome encoded human protocadherin-PC, comprising nucleotides 1 through 4860, where the protein coding sequence is represented by nucleotides 614 through 3727 (Accession No. AF277053; Chen et al., Oncogene 21:7861-7871 (2002)).

FIG. 27. Amino acid sequence for human protocadherin-PC encoded for by nucleotides 614 through 3727 of SEQ ID NO:1

FIG. 28. Nucleotide sequence of siRNA 181 targeting the X-chromosome-encoded homologue of protocadherin-PC (SEQ ID NO:3)

FIG. 29. Nucleotide sequence of siRNA 181 targeting Y-chromosome-encoded protocadherin-PC (SEQ ID NO:4). Note the cytosine to thymine point mutation at position six compared to SEQ ID NO:3

FIG. 30. Nucleotide sequence of siRNA 190 targeting the X-chromosome-encoded homologue of protocadherin-PC (SEQ ID NO:5)

FIG. 31. Nucleotide sequence of siRNA 190 targeting Y-chromosome-encoded protocadherin-PC (SEQ ID NO:6). Note the adenine to thymine point mutation at position six compared to SEQ ID NO:5

FIG. 32. Nucleotide sequence of siRNA 208 targeting both Y-chromosome-encoded protocadherin-PC and the X-chromosome-encoded homologue (SEQ ID NO:7).

FIG. 33. Specificity of monoclonal antibodies has been evaluated by ELISA. 100 ng of rPCDH-PC were coated in each well of the microtiter plates. The purified monoclonal antibody was tested at different concentrations. The negative control was performed with proteins extracted from BL21(DE3)RIPL cells transformed with an empty vector pET3a.

FIG. 34. Specificity of monoclonal antibodies has been evaluated by western-blotting. Eukaryotic rPCDH-PC was expressed in vitro using the TNT T7-Quick coupled Transcription/translation system. 1 μg of pcDNA3-PCDH-PC vector was added to 50 μl of reaction mixture. The negative control was performed by using an empty pcDNA3 vector. After the transcription/translation reaction, 5 μl aliquot of each reaction were analyzed by western blot. Monoclonal antibody LIU detected a 110 kDa protein corresponding to PCDH-PC.

FIG. 35. Specificity of monoclonal antibodies has been evaluated by immunohistochemistry performed on prostate cell lines. PCDH-PC-expressed cell line (PC3/PCDH-PC, stably transfected with pcDNA3-PCDH-PC vector) and control PC3 cells (cells transfected with an empty pcDNA3 vector) were cultured on 4-well Lab-Tek chambered cover. Cells were fixed in 4% paraformaldehyde and permeabilized with 0.2% Triton X-100. Cells were then stained for PCDH-PC. Monoclonal antibody SSA specifically bound to PC3/PCDH-PC cells and not to control cells.

FIGS. 36A-36B. Specificity of antibodies has been evaluated by immunohistochemistry performed on human tumor prostate specimens. FIG. 36A. Monoclonal antibody LIU strongly detected PCDH-PC in formalin fixed paraffin-embedded hormone refractory tumor cells. FIG. 36B. This staining was competed by excess of recombinant PCDH-PC demonstrating the specificity of the antibody. FIG. 36C. Positive immunostaining of cells in human prostate cancer containing tissues is indicated by a brown coloration (peroxidase-detection) selectively found in the prostate cancer cells of this specimen.

FIGS. 37A-37D. Localization of PCDH-PC protein in prostatic tissues. Immunohistochemistry technique was performed on formalin fixed paraffin embedded tissue using antibody SSA. FIG. 37A. Tissues from normal prostate. Note the staining corresponding to PCDH-PC protein was mainly localized in the basal epithelial cells. FIG. 37B. Similar results were obtained with benign prostatic hyperplasia (BPH) specimens. FIG. 37C. Tumor cells from untreated CaP were positive for PCDH-PC staining. FIG. 37D. In tissues obtained from hormone-refractory human prostate cancers strong staining corresponding to PCDH-PC protein was localized in all tumor cells. Magnification: FIGS. 37A-37D×200.

FIG. 38. Sandwich ELISA using antibodies SSA and LIU detected a circulating form of PCDH-PC protein in serum of certain hormone-refractory prostate cancer patients. Number indicated different samples.

FIG. 41. Ethidium bromide-stained agarose gel profiles of PCR reactions products from input control LNCaP DNA (In), b-catenin antibody immunoprecipitated chromatin from 48 h Ad-lac Z transduced LNCaP cells (Con) or from 48 h Ad-Wnt 1 transduced LNCaP cells (Wnt-1). Results show that sheared chromatin within three regions of the hAR promoter were immunoprecipitated by the antibody in b-catenin and PCDH-PC transfected cells as well as the known LEF-1/TCF binding elements within the promoters of the cyclin D1 and c-myc gene but these regions were not immunoprecipitated in control transfected cells.

FIGS. 42A-42C. The promoter of the human androgen receptor gene contains b-catenin sensitive elements that upregulate luciferase expression in chimeric reporter vectors. FIG. 42A. Chimeric hAR promoter/luciferase reporter vectors with varying amounts of upstream hAR promoter (left) were co-transfected into LNCaP cells along with empty vector (pcDNA3) or b-catenin and normalized luciferase activity was measured after 48 h (right). Results show progressive increase in luciferase as promoter element length is increased. FIG. 42B. Comparison of normalized luciferase expression from vector 5, above with wildtype, deleted (A at −1162) or mutated (G instead of A at −1162) LEF-1/TCF binding site (−1158 to −1164) when co-transfected with empty vector or β-catenin, as indicated. FIG. 42C. Semiquantitative RT-PCR analysis of hAR (Top) or G3PDH (Bottom) mRNA expression in LNCaP cells or Wnt-activated LNCaP cells (grown in androgen-free medium for 3, 6 or 9 days or transfected with PCDH-PC or b-catenin) or in LNCaP-E-T6 cells (stably transfected with ecdysterone-inducible PCDH-PC expression vector) with or without ponasterone (Pon).

FIGS. 43A-43C. Expression of hAR protein is downregulated in Wnt-activated LNCaP cells by a proteasomal degradation pathway. FIG. 43A. Western blot shows relative expression of hAR or actin in control LNCaP cells (Control) or in LNCaP cells transfected with β-catenin or PCDH-PC or LNCaP cells grown in androgen-free medium for 7 days. FIG. 43B. Western blot shows hAR protein is likewise downregulated in LNCaP cells transduced for 48 h with Ad-Wnt-1 but not from cells transduced with Ad-Lac Z. FIG. 43C. Expression of hAR protein in Wnt-activated cells (β-catenin transfected or cultured in androgen-free medium for 7 days) is restored to levels commensurate with elevated hAR mRNA levels when Wnt-stimulated cells were treated with proteasome inhibitors, MG132 or lactacystin.

FIGS. 44A-44B. Suppression of MDM2 expression or direct Akt activity relieves Wnt-mediated suppression of hAR protein expression. FIG. 44A. Western blot (top) shows that MDM2 protein expression is suppressed by greater than 88% by an siRNA that targets the gene and this siRNA relieves the Wnt-mediated suppression of hAR expression induced by transfection with β-catenin or PCDH-PC. (middle). Actin control (bottom). FIG. 44B. Western blot shows that direct suppression of Akt signaling by inhibitor 5233705 but not by PI3-kinase inhibitor LY294002 relieves Wnt-mediated suppression of hAR protein (top) and Wnt-mediated upregulation in phosphorylation of MDM2 (middle) in β-catenin transfected LNCaP cells. Actin control (bottom).

FIG. 45. Proteasome inhibitors block the suppression of MDM2 phosphorylation and suppress degradation of PP2A B subunit protein in Wnt-activated LNCAP cells. Western blots show that Wnt-activation (by β-catenin transfection or culture of LNCaP cells for 7 days in androgen-free medium) upregulates phosphorylation of MDM2 (top) that is blocked by proteasome inhibitors MG132 or lactacystin and this activity corresponds with loss of the regulatory subunit B of PP2A that is blocked by proteasome inhibitors (middle). There was no change in the PP2A catalytic C subunit levels in Wnt-activated or proteasome-inhibitor treated cells (bottom).

### DETAILED DESCRIPTION OF THE INVENTION

The patent and scientific literature referred to herein provides knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

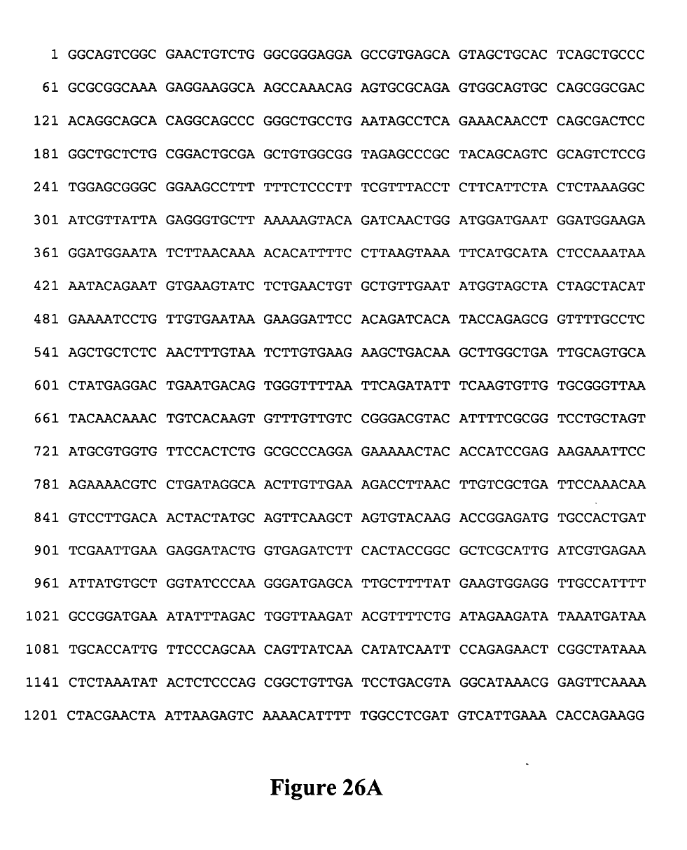

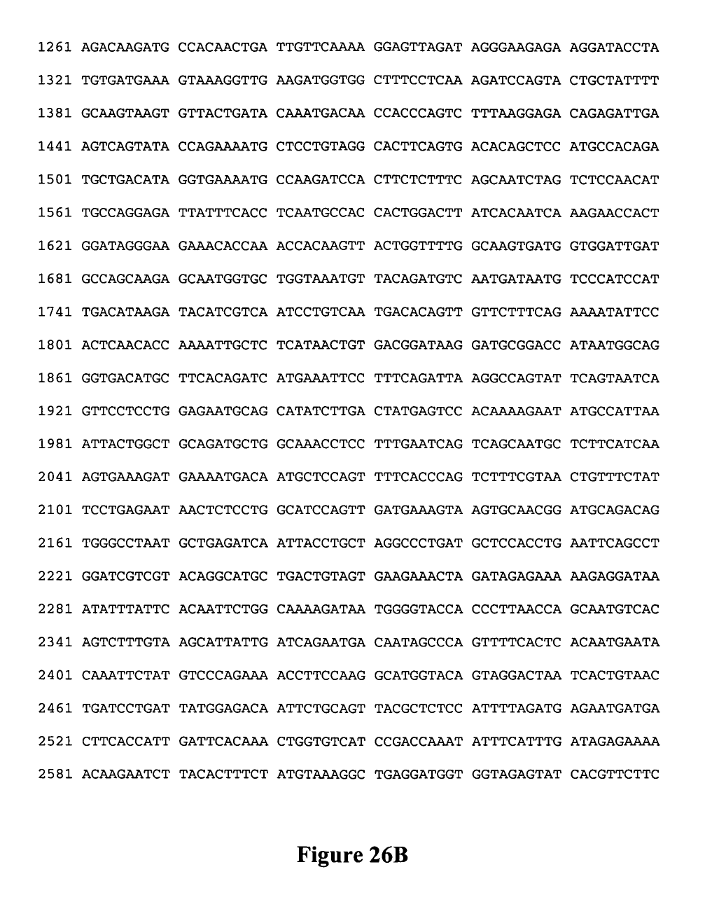

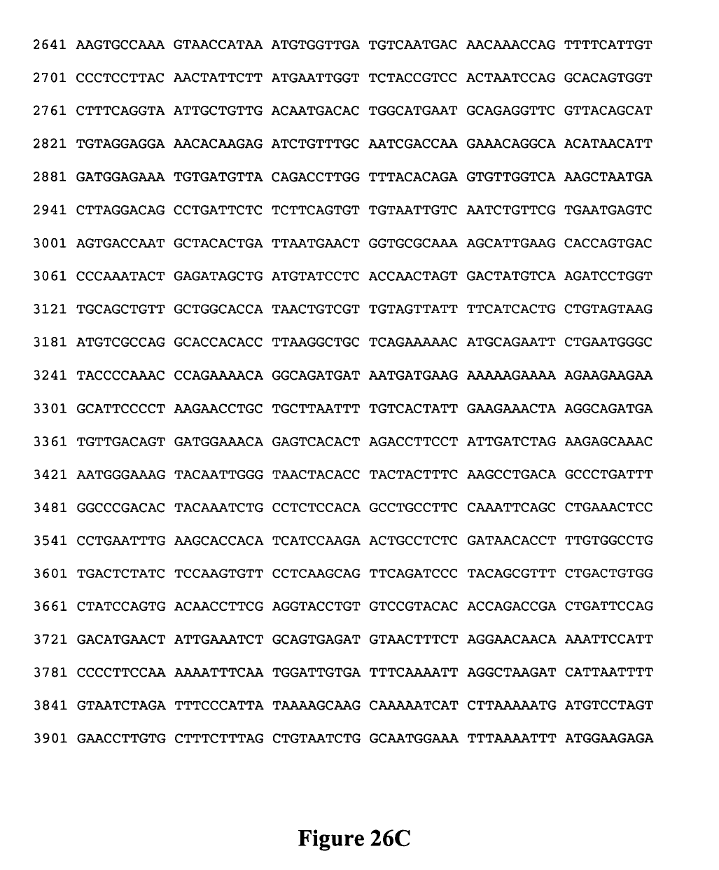

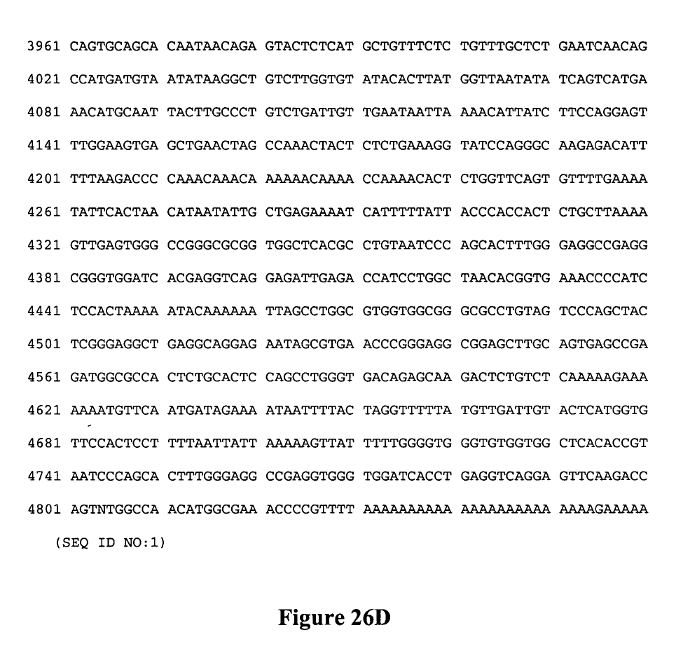

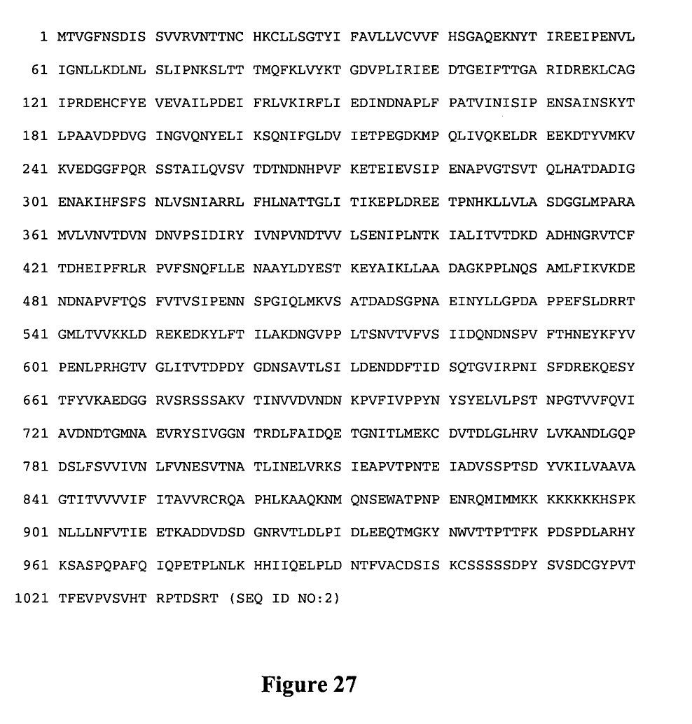

Protocadherin-PC (also referred to herein as PCDH-Y or pro-PC) is expressed from an orphan gene, meaning that there is only one copy of the gene that is localized on the human Y-chromosome. Thus the protocadherin-PC gene product is only expressed in male tissues. Protocadherin-PC is also a “human only” gene product, having evolved from another protocadherin orphan gene homologue present on the primate X-chromosome. The X-chromosome encoded homologue of protocadherin-PC (designated PCDH-X) is also expressed in humans. SEQ ID NO:1 shown in FIGS. 26A-26D represents the complete mRNA sequence encoding Y-chromosome encoded human protocadherin-PC, comprising nucleotides 1 through 4860, where the protein coding sequence is represented by nucleotides 614 through 3727 (Accession No. AF277053; Chen et al., Oncogene 21:7861-7871 (2002)). The human protocadherin-PC amino acid sequence (SEQ ID NO:2, FIG. 27) is encoded for by nucleotides 614 through 3727 of SEQ ID NO:1. Protocadherin-PC has been shown to induce the wnt signaling pathway in prostate cancer cells by inhibiting the translocation of β-catenin from the nucleus, thereby enhancing the accumulation of β-catenin in the nucleus and increasing transcription. The nucleotide sequence of from about 3601 to about 3635 of SEQ ID NO:1 represents a binding domain which can mediate the interaction between protocadherin-PC and β-catenin. Protocadherin-PC has also been shown to interact with FHL-2, although the binding domains responsible for this interaction have not yet been elucidated.

It is a discovery of the present invention that there is a connection between the expression and function of protocadherin-PC in prostate cancer cells and the resistance of prostate cancer to androgen withdrawal therapies. Protocadherin-PC is encoded on the human Y chromosome and is also referred to as protocadherin-Y (PCDH-Y) to distinguish it from the X-encoded homologue, protocadherin-X (PCDH-X; Accession No. AC004388). The expression of this unusual male-specific member of the cadherin gene family is selectively upregulated in cultured human prostate cancer cells when they are selected for apoptosis-resistance or when they are exposed to androgen-free conditions. Ablation of PCDH-PC expression or activity is a unique target for clinical therapy for hormone-resistant prostate cancer because it is a male-specific gene product and obviously, women survive just fine without it; and it is expressed mainly in (male) brain and in scattered basal cells of the normal prostate, so complications in other tissues can be avoided by using compounds that do not cross the brain-barrier.

The present invention provides that protocadherin-PC plays a role in the transition of androgen-sensitive prostate cancer cells to androgen-resistant prostate cancer cells, thereby influencing the onset or progression of hormone-resistant disease. Protocadherin-PC is highly overexpressed in hormone-resistant prostate tumors from patients and in hormone-resistant variants of the prostate cancer cell line, LNCaP. When androgen-sensitive LNCaP cells are transfected with protocadherin-PC, hormone resistance is conferred to them with respect to their ability to form tumors in castrated male nude mice. Upregulation of protocadherin-PC in prostate cancer cells upon androgen-deprivation induces the activity of the wnt signaling pathway; a pathway that is known to become highly active during the development of aggressive colon, oral, and skin (melanoma) cancers in humans. Activation of the wnt pathway by protocadherin-PC in prostate cancer cells drives the cells to acquire neuroendocrine cell-like properties associated with the synthesis and release of neuroendocrine hormones that help prostate cancer cells grow in an androgen-independent state.

The invention provides for induction of wnt signaling in prostate cancer cells by protocadherin-PC, thereby enhancing β-catenin accumulation in the nucleus and increasing DNA transcription from TCF/LEF-1 binding elements. In one aspect, protocadherin-PC binds to β-catenin. As demonstrated by immunoprecipitation studies, protocadherin-PC co-precipitates with β-catenin from androgen-insensitive LNCaP cells. These cells also have abnormalities in their intracellular β-catenin distribution pattern, consistent with the ability to demonstrate enhanced luciferase production using a TCF-promoted luciferase reporter vector (Chen et al., Oncogene 21:7861-7871 (2002); de la Taille et al., Clin Can Res 9:1801-1807 (2003)). In another aspect of the present invention, protocadherin-PC binds to the human four and a half LIM domain protein, FHL-2. A yeast-2-hybrid screen of a LNCAP cDNA library identified FHL-2 as a protocadherin-PC binding protein (See Example 6). The invention provides for FHL-2 mediation of the interaction between protocadherin-PC and β-catenin, thereby mediating the effects of protocadherin-PC on wnt signaling in prostate cancer cells. To further elucidate the biological effects of protocadherin-PC interactions with β-catenin, FHL-2, or other proteins, the invention provides for mutated versions of protocadherin-PC in which one or more binding domains has been disrupted or deleted. This would allow one to determine whether the protein-protein interactions play a role in protocadherin-PC-mediated prostate cell killing.

Studies on the androgen receptor (AR) gene and gene products have shown that some androgen-insensitive prostate cancers from patients contain tumor cells with hyperactive androgen signaling associated with the presence of mutations in the AR gene (that make AR promiscuous with regards to its ability to accept alternate steroid ligands) or in association with amplification of the AR gene (that increases basal expression of AR protein) (Craft et al., Cancer Mets Rev 17:421-427 (1999); Buchanan et al., Cancer Mets Rev 20:207-223 (2001); Culig et al., J Urol 170:1363-1369 (2003); Taplin et al., J Cell Biochem 91:483-490 (2004); Cornauer et al., Int J Oncol 23:1095-1102 (2003)). The present invention provides for regulation of the expression of the human gene by protocadherin-PC (See Examples 4 and 10).

The activity of Akt, or protein kinase B, is critical for cell survival. Induction of Akt phosphorylation and activation can be induced by wnt signaling in neuronal cell lines and the prostate cancer cell line, PC-3 (Fukumoto et al., J Biol Chem 276:17479-17483 (2001); Ohigashi et al., Prostate 62: 61-68 (2005)). As provided for by this invention, inhibition of protocadherin-PC gene expression suppresses phosphorylation of Akt in LNCAP cells (See Example 3).

Compounds

The invention provides for embodiments where the inhibitor of protocadherin-PC comprises nucleic acid compounds that inhibit protocadherin-PC; such as a protocadherin-PC small interfering RNA (siRNA), an antisense oligonucleotide, or a peptide nucleic acid (PNA), that specifically binds a nucleic acid encoding protocadherin-PC; a ribozyme that specifically cleaves a nucleic acid encoding protocadherin-PC; a small molecule; an antibody or antigen binding fragment thereof; a peptide; or a peptidomimetic.

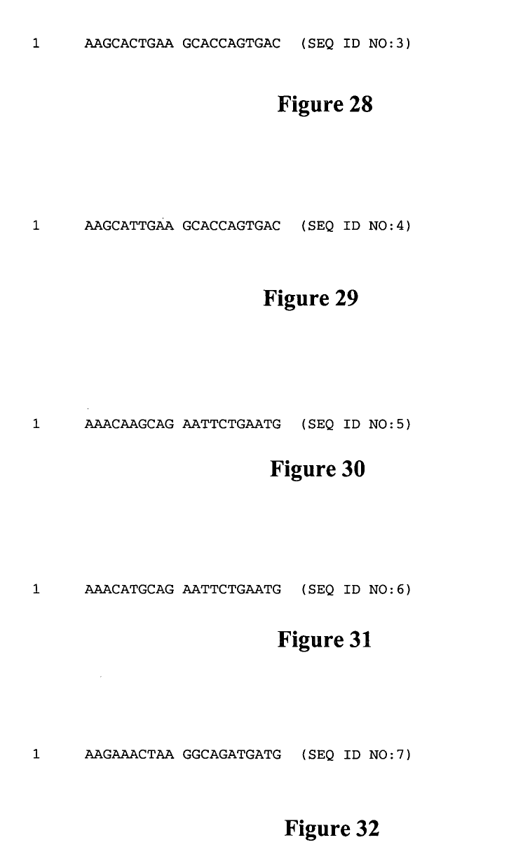

The invention provides for a nucleic acid comprising from about 7 to about 30 nucleotides that specifically binds to a region from about 3023 to about 3727 of SEQ ID NO:1, wherein the nucleic acid is capable of inhibiting expression of protocadherin-PC. The invention also provides for one or more nucleic acids from about 7 to about 29 nucleotides, from about 7 to about 28 nucleotides, from about 7 to about 27 nucleotides, from about 7 to about 26 nucleotides, from about 8 to about 30 nucleotides, from about 8 to about 29 nucleotides, from about 8 to about 28 nucleotides, from about 8 to about 27 nucleotides, from about 9 to about 30 nucleotides, from about 9 to about 29 nucleotides, from about 9 to about 28 nucleotides, from about 10 to about 30 nucleotides, from about 10 to about 29 nucleotides, or from about 11 to about 30 nucleotides that specifically binds to a region from about 3023 to about 3727 of SEQ ID NO:1, wherein the nucleic acid is capable of inhibiting expression of protocadherin-PC. In one embodiment, the nucleic acid comprises RNA, antisense RNA, small interfering RNA (siRNA), double stranded RNA (ds RNA), short hairpin RNA (shRNA), cDNA or DNA. In another embodiment, the nucleic acid comprises a sequence within the region of from about nucleotide 3023 to about nucleotide 3727 of SEQ ID NO:1. In an additional embodiment, the nucleic acid comprises a sequence about 70% identical to the complement of a portion of the sequence from about nucleotide 3023 to about nucleotide 3727 of SEQ ID NO:1. In a preferred embodiment, the nucleic acid comprises SEQ ID NO:3, 4, 5, 6 or 7 (FIGS. 28, 29, 30, 31 and 32, respectively). The invention also provides for an embodiment wherein the nucleic acid comprises a UU overhang or a TT overhang. In yet another embodiment provided for by the invention, the nucleic acid comprises at least one modified internucleotide linkage or at least one chemically modified nucleotide to render it resistant to enzymatic degradation. In a specific embodiment, the modified internucleotide linkage is a phosphorothioate linkage. In another embodiment, the modified nucleotide comprises a 2′-O-methoxy-residue.

The present invention encompasses a composition comprising one or more nucleic acids provided for by the invention and a pharmaceutically acceptable carrier.

One aspect of this invention provides for an isolated prostate cancer cell that does not express a protocadherin-PC gene, wherein the naturally occurring prostate cancer cell does express the protocadherin-PC gene.

siRNA

RNA interference (RNAi) is a method of gene-specific silencing which employs sequence-specific small interfering RNA (siRNA) to target and degrade the gene-specific mRNA prior to translation. Methods for designing specific siRNAs based on an mRNA sequence are well known in the art and design algorithms are available on the websites of many commercial vendors that synthesize siRNAs, including Dharmacon, Ambion, Qiagen, GenScript and Clontech.

In the context of the present invention, three different siRNAs targeting PCDH-PC were designed using the siRNA Target Finder software program available through Ambion, Inc. The anti-PCDH-PC siRNAs targeted the PCDH-PC mRNA sequence at position 3043-3062 (#181; SEQ ID NO: 4, FIG. 29), 3098-3117 (#190; SEQ ID NO:6, FIG. 31) or 3345-3364 (#208; SEQ ID NO: 7, FIG. 32) on the PCDH-PC mRNA. The 21 bp siRNAs were constructed using the 19 bp core sequences described above with 2 nucleotide UU overhangs and these siRNAs were produced and provided by Ambion, Inc.

PCDH-PC-specific siRNA selectively induces cell death of androgen-deprived LNCAP cells (See Example 1). The results show that culture of LNCaP cells in androgen-free medium for 7 days is associated with a slight increase in apoptosis compared to control medium, however the PCDH-PC siRNA induces greater than 4× more cell death (58% dead cells) than comparable untransfected cells or cells transfected with lamin siRNA. Also note that the ability of PCDH-PC siRNA to induce cell death is specific to cells grown in androgen free medium, not in normal medium.

Antisense

Antisense oligonucleotides (ASOs) are small deoxy-oligonucleotides with a sequence complementary to the mRNA of the target gene (Crooke, (1993) Curr. Opin. Invest. Drugs, 2: 1045-1048; Stein and Cheng, (1993) Science, 261: 1004-10012; Hawley and Gibson (1996) Antisense & Nucleic Drug Dev., 6: 185-195; Crooke, S. T. (2003) Ann. Rev. Med., 55: 61-95; Kalota, et al., (2004) Cancer Biol. & Therapy, 3: 4-12; Orr, et al., (2005) Meth. Mol. Med., 106: 85-111). They bind to the target mRNA through complementary base-pairing and attract the binding of RNase H, an enzyme that degrades double strand RNA, thus destroying the target mRNA (18-25). While unmodified ASOs can be as sensitive to degradation as RNA, chemical modification of the phosphodiester backbones can make them resistant to degradative action of nucleases in in vivo situations (nonlimiting examples include phosphorothioate- or 2′-O-[2-methoxyethyl]-backbone modifications) (Monia, et al. (1996) J. Biol. Chem., 271: 14533-1440; also see U.S. Pat. Nos. 5,652,355 and 5,652,356).

ASOs offer many unique aspects that make them likely to be rapidly translated into clinical trials in humans with prostate cancer: 1) they are simple defined chemical agents can be synthesized in bulk under highly controlled (good clinical practice) conditions; 2) they can be delivered to patients systemically in controlled doses, making it more likely that they can even reach distal metastases; 3) they are not known to have potential for genetic damage, as with other biological agents (viruses) that are being developed and tested for gene therapy strategies and; 4) gene-targeting ASO agents are already in clinical trials for several different cancers, thus there already is a body of literature regarding their use in humans. For example, see U.S. Pat. No. 6,066,500 which describes antisense compounds, including oligonucleotides, and methods of use for modulating the expression of β-catenin and for treatment of diseases associated with expression of β-catenin, especially colorectal cancer and melanomas.

The present invention provides for phosphothio-modified antisense oligonucleotides that are capable of inhibiting the expression of protocadherin-PC. SEQ ID NOS:3, 4, 5, 6, and 7 comprise non-limiting examples of anti-protocadherin-PC phosphothio-modified antisense oligonucleotides provided for by this invention (See Example 9).

shRNA

The invention also provides for a nucleic acid comprising a nucleic acid expression vector encoding a short hairpin RNA (shRNA), wherein the shRNA comprises the siRNA nucleotide sequence of SEQ ID NO:3, 4, 5, 6, or 7. In one embodiment, the shRNA comprises SEQ ID NO:3, 4, 5, 6, or 7 in an expression vector. In one aspect of the invention, a host organism comprises a nucleic acid of the invention. In an additional embodiment, the host is a prokaryote or a eukaryote. In another embodiment, a cell comprises a nucleic acid of the invention. In yet another embodiment, a non-human mammal comprises one or more cells provided for by the invention.

Small interfering RNAs can be expressed in vivo in the form of short, fold-back, hairpin loop structures known as short hairpin RNAs (shRNAs) comprising the siRNA sequence of interest. When expressed in a cell, shRNA is rapidly processed by intracellular machinery into siRNA. Expression of shRNAs is accomplished by ligating the shRNA into an expression cassette of a double stranded RNA (dsRNA) expression vector. Expression may be driven by RNA polymerase III promoters (See U.S. Pat. No. 6,852,535). Plasmid vectors for expression of shRNAs are commercially available from vendors such as Gene Therapy Systems, Ambion and Stratagene. U.S. Publication No. 2005/0019918A1 describes the use of a lentiviral vector for in vivo siRNA expression. Methods for DNA and RNA manipulations, including ligation and purification, are well known to those skilled in the art. Vectors comprising shRNA expression cassettes may be introduced into prokaryotic or eukaryotic cells using methods known to one skilled in the art.

Xenograft tumor models are widely used to study human diseases in non-human mammals. To study the impact of protein expression on tumor growth, cells harboring vectors expressing siRNA that specifically inhibits expression of the can be implanted into an immunodeficient mouse under conditions which promote the formation of a tumor consisting of the implanted cells. As described in U.S. Publication No. 2005/0019918A1, malignant melanoma cells infected with a lentiviral vector expressing siRNA targeting mutated BRAF mRNA were implanted subcutaneously into immunodeficient mice and tumor volume was measured chronologically to determine the impact of BRAF on tumor growth. A xenograft mouse model was used to demonstrate that cervical and lung cancer cells transfected with plasmids expressing shRNAs targeted to PLK1 resulted in reduced tumor growth (Spankuch et al., J Natl Cancer Inst 96:862-872 (2004)).

Short hairpin RNAs are available through commercial vendors, many vendors also have online algorithms useful for designing shRNAs (i.e., Clontech, ExpressOn, Gene Link and BD Biosciences).

PNA

Peptide nucleic acids (PNAs) comprise naturally-occurring DNA bases (i.e., adenine, thymine, cytosine, guanine) or artificial bases (i.e., bromothymine, azaadenines, azaguanines) attached to a peptide backbone through a suitable linker. Nonlimiting examples of PNA backbone linking moieties include amide, thioamide, sulfinamide or sulfonamide linkages. Preferably, the linking moieties in the PNA backbone comprise N-ethylaminoglycine units, and the bases are covalently bound to the PNA backbone by methylene-carbonyl groups. PNAs bind complementary DNA or RNA strands more strongly than a corresponding DNA. They can be utilized in a manner similar to antisense oligonucleotides to block the translation of specific mRNA transcripts. PNA oligomers can be prepared according to the method provided by U.S. Pat. No. 6,713,602. U.S. Pat. No. 6,723,560 describes methods for modulating transcription and translation using sense and antisense PNA oligomers, respectively. Also included in this patent are methods for administration of PNAs to a subject such that the oligomers cross biological barriers and engender a sequence specific response. The PNA can be attached to a targeting moiety, such as an internalization peptide, facilitate uptake of the PNA by cells or tissues.

Within the scope of the present invention are PNAs specific for protocadherin-PC, and methods of administration of PNAs to a subject.

Peptides and Peptidomimetics

Protocadherin-PC inhibitors such as peptides or peptidomimetics are also provided for by the invention. Peptides may be synthesized by methods well known in the art, including chemical synthesis and recombinant DNA methods. A peptidomimetic is a compound that is structurally similar to a peptide, such that the peptidomimetic retains the functional characteristics of the peptide. Peptidomimetics include organic compounds and modified peptides that mimic the three-dimensional shape of a peptide. As described in U.S. Pat. No. 5,331,573, the shape of the peptidomimetic may be designed and evaluated using techniques such as NMR or computational techniques. Protocadherin-PC inhibitors can be designed based on the structural characteristics of protocadherin-PC, FHL-2 and β-catenin. Mutational analyses known in the art may be used to define amino acids or amino acid sequences required for protein-protein interactions. Simcha et al. demonstrate mapping of the minimal β-catenin-interacting region of DE-cadherin and determination of critical amino acids for the β-catenin/DE-cadherin interaction (Simcha et al., Mol Biol Cell 12:1177-1188 (2001)). WO9942481A2 describes peptides or analogous molecules derived from the interaction domains of β-catenin and LEF-1/TCF, APC, conductin and E-cadherin which inhibit the protein-protein interactions in order to influence the activity of the proteins.

Within the scope of the present invention are peptide or peptidomimetic inhibitors sharing sufficient homology with and binding to the interaction domains, or portions thereof, which may be used, for example, to block complex formation between protocadherin-PC and β-catenin, or protocadherin-PC and FHL-2, thereby creating a lesion in the signaling pathway and inhibiting downstream events, such as gene transcription.

The invention encompasses a composition comprising one or more peptides provided for by the invention and a pharmaceutically acceptable carrier. The invention also encompasses a composition comprising one or morepeptidomimetics provided for by the invention and a pharmaceutically acceptable carrier.

Antibodies

In one aspect of the invention, antibodies or fragments thereof are used as inhibitors of protocadherin-PC activity. FHL-2-specific antibodies are commercially available from vendors such as Bethyl Laboratories, Abnova Corp. and Abcam. β-catenin-specific antibodies are commercially available from vendors such as Novus Biologicals, R & D Systems and Abcam. Anti-protocadherin-PC antibodies are described in Chen et al., Oncogene 21:7861-7871 (2002).

The invention provides for an antibody, or antigen-binding fragment thereof, that specifically binds to the Y-chromosome encoded homologue of protocadherin-PC comprising the polypeptide amino acid sequence of SEQ ID NO:2 (FIG. 27), wherein the antibody or antigen-binding fragment thereof does not bind to the X-chromosome encoded homologue of protocadherin-PC. Also provided for by this invention is an antibody, or fragment thereof, that binds to the Y-chromosome-encoded protocadherin-PC and binds to the X-chromosome-encoded homologue of protocadherin-PC. The invention provides for nucleic acid sequences that encode antibodies, or fragments thereof, that bind to protocadherin-PC. Within the context of the invention, the antibody, or fragment thereof, can be monoclonal, polyclonal, chimeric or humanized.

The invention also provides for a hybridoma cell which produces antibodies that bind to protocadherin-PC. For example, three hybridoma cell lines have been established which produce anti-protocadherin-PC antibodies (See Example 8). The hybridoma cell lines are designated as SSA, LIU and C32. The SSA and LIU cell lines were deposited on Jan. 24, 2006 with the Collection Nationale de Cultures de Microorganismes (CNCM), Institut Pasteur, 25 rue de Docteur Roux, F-75724 Paris Cedex 15, under the provisions of the Budapest Treaty on the International Recognition of the Deposit of Microorganisms for the Purposes of a Patent Procedure. The deposited hybridoma cell line SSA is assigned as HB 0337 SSA and is designated as number CNCM I-3560. The deposited hybridoma cell line LIU is assigned as HB 0337 LIU and is designated as number CNCM I-3561.

The invention also encompasses use of the antibodies provided by the invention for diagnostic or therapeutic purposes. For example, the antibodies may be used for staining human prostate cancer specimens to diagnose hormone-refractory prostate cancer. The antibodies may also be used, for example, for discriminating between hormone-refractory prostate cancer and hormone-responsive prostate cancer. Additional exemplary uses of the antibodies include use as a tumor marker for early detection of prostate cancer, use in the pre-treatment staging of prostate cancer, use in the post-treatment monitoring of prostate cancer, use as a marker to distinguish between indolent verses aggressive prostate cancer, and use as a research tool to elucidate the molecular mechanisms involved in prostate cancer initiation and progression. Use of the inventive antibodies in serum-based tests to detect aggressive prostate cancer in humans is also within the scope of the invention (see Example 8).

The invention encompasses a composition comprising one or more anitbodies provided for by the invention and a pharmaceutically acceptable carrier. The invention also encompasses a composition comprising one or more hybridoma cells provided for by the invention and a pharmaceutically acceptable carrier.

Small Molecules

In another aspect of the invention, protocadherin-PC inhibitors comprise small molecules capable of blocking protocadherin-PC expression or binding. Within the scope of the invention, the small molecule comprises an organic molecule. Also within the scope of the invention, the small molecule comprises an inorganic molecule. Protein-protein interaction inhibitors may act directly via inhibition at the protein-protein interface, or indirectly via binding to a site not at the interface and inducing a conformational change in the protein such that the protein is prohibited from engaging in the protein-protein interaction (Pagliaro et al., Curr Opin Chem Biol 8:442-449 (2004)). U.S. Publication No. 2005/0032245A1 describes methods for determining such inhibitors and evaluating potential inhibitors that prevent or inhibit protein-protein interactions. U.S. Publication No. 2004/0204477A1 describes an interaction inhibitor that binds to a binding domain on β-catenin, thereby disrupting the interaction between β-catenin and TCF-4.

Additional examples for determining inhibitors of protocadherin-PC use the protein crystal structure of protocadherin-PC. The crystal structure of protocadherin-PC may be used to screen for protocadherin-PC inhibitors or to design protocadherin-PC inhibitors. One of ordinary skill in the art can solve the crystal structure of protocadherin-PC and determine sites which confer protocadherin-PC function. Based on the crystal structure, in silico screens of compound databases may be performed to discover compounds that would be predicted to inhibit protocadherin-PC. These compounds can then be evaluated in assays to determine if they inhibit protocadherin-PC function. Additionally, the crystal structure can be used to design compounds (i.e., rational drug design) that would be predicted to inhibit protocadherin-PC function based on the structure of the compound, then the compound can be tested in assays to determine if they inhibit protocadherin-PC function.

Methods for Treating Cancer

Similar to the normal prostate gland that develops, matures and functions under the hormonal influence of androgenic steroids, prostate cancer also requires androgenic steroids for its development and progression. This need for androgen is consistent with the common treatment for advanced disease, androgen withdrawal therapies. Unfortunately, these types of therapies are only transiently suppressive of the disease, and hormonally-treated prostate cancer eventually relapses into an androgen-independent or hormone-resistant state. Once in this hormone-resistant state, prostate cancer can be highly resistant to other common forms of cancer therapeutics such as chemotherapy and radiation.

The invention provides for a method for treating cancer in a subject, the method comprising administering to the subject an effective amount of an inhibitor of protocadherin-PC. In certain embodiments, the cancer comprises at least one of prostate, breast, melanoma, oral, ovarian, endometrial, hepatocellular carcinoma or head and neck tumors. The invention also provides for a method for treating hormone-resistant prostate cancer in a subject, the method comprising administering to the subject an effective amount of an inhibitor of protocadherin-PC. The invention provides for an embodiment where the hormone-resistant prostate cancer is also resistant to chemotherapy and/or radiation therapy. In another aspect, the invention provides for a method for treating prostate cancer in a subject, the method comprising administering to the subject one or more androgen-withdrawal therapies and an effective amount of an inhibitor of protocadherin-PC. The invention provides for embodiments where the androgen-withdrawal therapy comprises surgical orchiectomy (removal of one or both testicles) or medical hormone therapies, including but not limited to antiandrogens and luteinizing hormone-releasing hormone agonists.

In certain embodiments of the methods of the invention, the inhibitor comprises a protein interaction inhibitor that disrupts protocadherin-PC binding domains, FHL-2 binding domains, or β-catenin binding domains. In other embodiments, the subject is a human, mouse, rabbit, monkey, rat, bovine, pig or dog. In other various embodiments, the administering comprises intralesional, intraperitoneal, intramuscular, intratumoral or intravenous injection; infusion; liposome- or vector-mediated delivery; or topical, nasal, oral, ocular, otic delivery, or any combination thereof. Other embodiments encompass an effective amount of inhibitor comprising an amount effective to arrest, delay or reverse the progression of the cancer.

This invention encompasses a method for treating prostate cancer in a subject, the method comprising administering to a subject an effective amount of a radiolabeled compound capable of specifically binding to protocadherin-PC. In an embodiment, the compound comprises an antibody, antibody fragment, peptide, or peptidomimetic specific for protocadherin-PC. In another embodiment, the compound comprises a nucleic acid that is capable of specifically binding to another nucleic acid, or fragment thereof, encoding protocadherin-PC.

Protocadherin-PC is an intracellular target in prostate cancer cells, thus in a preferred embodiment, the compounds provided for by this invention can cross the cell membrane and inhibit the expression or activity of protocadherin-PC. Nonlimiting examples known in the art of methods by which compounds may enter a cell include transduction peptides, transmembrane carrier peptides, internalization factors and liposomes. U.S. Pat. Nos. 5,652,122, 5,670,617, 6,589,503 and 6,841,535 describe membrane-permeable peptides that are useful as transfection agents to facilitate the efficient cellular internalization of a broad range and size of compounds including nucleic acids, oligonucleotides, proteins, antibodies, inorganic molecules and PNAs. U.S. Pat. No. 5,922,859 describes a method for facilitating endocytosis of therapeutically active nucleic acids (i.e., antisense oligonucleotides, ribozymes or plasmid DNA) into cells using an internalizing factor such as transferrin. As described in U.S. Pat. Nos. 5,135,736 and 5,169,933, covalently linked complexes (CLCs) comprising a targeting moiety, a therapeutically active compound (i.e., toxins, radionuclides or peptides) and a peptide facilitating translocation/internalization of the complex across the cell membrane and into the cytoplasm. Also see U.S. Publication No. 20050008617A1, describing compositions and methods for delivery of siRNAs and shRNAs and U.S. Pat. No. 5,593,974 covering localized oligonucleotide therapy.

The invention provides for the discovery that compounds specifically binding to protocadherin-PC may be used to target radioisotopes directly to prostate cancer cells, thereby specifically treating prostate cancer. Ilustratively, U.S. Publication No. 20040052727A1 discloses a method for prostate cancer therapy using radiolabeled organic molecules targeted to the androgen receptor. U.S. Pat. No. 6,274,118 describes a method for treating non-prostatic endocrine cancers using entities that have been constructed to specifically target PSA expressed in breast tumors. As described in U.S. Pat. No. 6,787,335, labeled antibodies that specifically bind mammary gland cancer specific gene products can be injected into patients with mammary gland cancer for the purpose of treating the mammary gland cancer. For prostate cancer therapy, the monoclonal antibody J591, which targets the extracellular domain of prostate specific membrane antigen (PSMA) expressed on prostate cancer cells, has been evaluated in clinical trials and found have antitumor activity in patients (Nanus et al., J Urol 170 (6 Pt. 2):S84-88 (2003); Bander et al., Semin Oncol 30:667-677 (2003); J Clin Oncol 22:2522-2531 (2004)). U.S. Pat. Nos. 6,107,090 and 6,767,771 are directed toward antibodies and other biological agents that may be used for targeted radioisotope treatment of prostate cancer.

Methods For Cancer Imaging and Detection

In Vivo Cancer Imaging

The present invention provides for a method for in vivo imaging of cancer in a subject, the method comprising (a) administering to the subject a radiolabeled compound capable of binding to protocadherin-PC or FHL-2; and (b) detecting the presence of the radiolabeled compound in the subject, thereby imaging cancer in the subject. In one embodiment, the cancer comprises prostate cancer or breast cancer. In another embodiment, the compound comprises an antibody, antibody fragment, peptide, or peptidomimetic. In another embodiment, the compound comprises a nucleic acid specific for a nucleic acid, or fragment thereof, encoding protocadherin-PC or FHL-2. In yet another embodiment, the compound is detected by MRI, SPECT, CT, or ultrasound.

The invention provides for the discovery that protocadherin-PC and FHL-2 can be used as cancer biomarkers. Protocadherin-PC and FHL-2 expression is measurable and correlates with prostate cancer prognosis and outcome. Additionally, measurable biomarkers can indicate the efficacy of drug treatment. Expression of biomarkers can be measured using in vivo imaging techniques, for example, detecting a radiolabel on a compound specifically bound to a target protein or a target nucleic acid. Compounds that have been employed for imaging include antibodies, antibody fragments, peptides, peptidomimetics, nucleic acids and small molecules. For example, U.S. Publication No. 20040052727A1 discloses a method for prostate cancer imaging using radiolabeled organic molecules targeted to the androgen receptor. U.S. Pat. No. 6,274,118 describes a method for localizing non-prostatic endocrine cancers in vivo using entities that have been constructed to target PSA and that can be detected by an imaging procedure. As described in U.S. Pat. No. 6,787,335, labeled antibodies that specifically bind mammary gland cancer specific gene products can be injected into patients suspected of having mammary gland cancer for the purpose of diagnosing or staging the disease status of the patient.

Labeled antibodies and antibody fragments have been used in combination with various imaging techniques, such as immunoscintography, single-photon emission computed tomography (SPECT), magnetic resonance imaging (MRI), positron emission tomography (PET), computer tomography (CT) and ultrasound, to target tumors and metastases in patients with various types of cancer (See Furster et al., Q J Nucl Med 47:109-115 (2003); Simms et al., BJU Int 88:686-691 (2001); Hu et al., World J Gastroenterol 4:303-306 (1998); Buist et el, Int J Gynecol Cancer 2:23-34 (1992); Miraillie et al., J Clin Endocrinol Metab 90:779-788 (2005)).

A radiolabeled peptidomimetic targeting the vitronectin receptor has been used to image tumors in a mouse model of mammary adenocarcinoma (Harris et al., Cancer Biother Radiopharm 18:627-641 (2003)). For prostate cancer imaging, the 7E11-C5.3 monoclonal antibody (capromab pendetide or ProstaScint) is used to target the intracellular domain of prostate specific membrane antigen (PSMA) (Troyer et al, Urol Oncol 1:29-37 (1995)). Radiolabeled derivatives of this antibody are used for imaging of prostate cancer in patients (Lamb and Faulds, Drugs Ageing 12:293-304 (1998); Rosenthal et al., Tech Urol 7:27-37 (2001)). Other antibodies targeting PSMA have also been developed and used for imaging prostate cancer in patients (Fenely et al., Prostate Cancer Prostatic Dis 3:47-52 (2000)). U.S. Pat. Nos. 6,107,090 and 6,767,771 are directed toward antibodies and other biological agents that may be used for imaging prostate cancer.

Peptides labeled with positron emitters have been developed to localize neuroendocrine tumors expressing a somatostatin receptors, the melanocortin 1 receptor and the bombesin receptor (Maecke et al., J Nucl Med 46(Supp 1): 172S-178S (2005). In preclinical trials, the same study demonstrated some success with bombesin-specific peptides in patients with prostate cancer. Internalization of a radiolabeled peptide into prostate cancer cells in culture and in a rat xenograft model of prostate tumors has also been demonstrated (Zitzmann et al., Clin Can Res 11:139-146 (2005)).

To determine if a nucleic acid can be used for MRI imaging of gene expression in prostate cancer, a peptide nucleic acid (PNA) specific for c-myc mRNA was labeled with an MRI contrast agent, then conjugated to a transmembrane carrier peptide and transfected into a prostate adenocarcinoma cell line (Heckl et al., Cancer Res 63:4766-4772 (2003)). The labeled PNA bound to the upregulated c-myc mRNA in the prostate tumor cells and the MRI contrast agent was retained inside the cells, thereby specifically increasing the MRI signal intensity in the tumor cells.

Targeted in vivo imaging of offers the possibility of defining the extent of localized and metastatic disease. Imaging studies can be used to define targets, such as protocadherin-PC and FHL-2, useful for developing specific anticancer agents, particularly agents that specifically target prostate carcinoma.

Detecting Cancer in a Sample

The present invention provides for a kit for determining whether or not a subject has or may develop prostate cancer, the kit comprising (a) an antibody or an antigen-binding fragment thereof, that specifically binds to a protocadherin-PC or an FHL-2; and (b) at least one negative control sample that does not contain a protocadherin-PC antigen or an FHL-2 antigen. In one embodiment, the kit further comprises a positive control sample that contains a protocadherin-PC antigen in an amount characteristic of a human prostate cancer cell. In another embodiment, the antibody or antigen-binding fragment is labeled with a detectable signal.

According to the invention, kits can be assembled which are useful for detecting the expression protocadherin-PC or FHL-2 protein in samples from patients who have, or who are suspected of having, prostate cancer. For diagnosis of prostate cancer, biopsy specimens, such as from prostate tissue or prostate tumors, are the most likely source of samples for analysis. The kit may comprise materials for collecting and preserving the biopsy sample. For example, the sample may be preserved by techniques known to those skilled in the art, such as formalin fixing, dehydration, cryopreservation, paraffin embedding. Sections of preserved tissue can be mounted on microscope slides for analysis. For non-preserved samples, cells from the sample can be directly fixed onto a microscope slide.

To detect protocadherin-PC or FHL-2 protein in the sample, conventional immunohistochemistry techniques may be used. Briefly, in the context of the resent invention, a prostate biopsy sample is contacted with antibodies specifically binding to protocadherin-PC or FHL-2. The antibody may be directly labeled with a detectable signaling molecule, such as a detectable fluorescent compound, a radioactive isotope, a chemiluminescent compound or a bioluminescent compound. Alternatively, if the antibodies specifically binding to protocadherin-PC or FHL-2 are not directly labeled, the bound antibodies may be indirectly detected using labeled secondary antibodies or other molecules, such as protein A, that bind to the first antibody. One skilled in the art would recognize signaling molecules that can be useful for directly or indirectly labeling antibodies. The kits may include control samples, i.e. samples that contain protocadherin-PC or FHL-2 protein in an amount characteristic of a human prostate cancer cell and samples that do not contain protocadherin-PC or FHL-2 protein. Illustrative examples of kits useful for detecting cancer in a sample include U.S. Pat. Nos. 5,719,032 (melanoma and prostate cancer), 5,928,873 (colorectal cancer) and 6,482,599 (benign prostatic hyperplasia).

Drug Screening Assays

This invention provides for the discovery that protocadherin-PC can be used as a target in a drug screening assay to identify drugs that are capable of inhibiting protocadherin-PC expression or activity, thereby treating prostate cancer.

The present invention provides for a method for identifying whether a test compound is capable of inhibiting protocadherin-PC protein activity, the method comprising (a) contacting a protocadherin-PC protein with (i) a test compound and (ii) β-catenin or FHL-2 or both; and (b) determining whether the activity of the protocadherin-PC protein of step (a) is inhibited as compared to the activity of a protocadherin-PC protein in the absence of the test compound, so as to identify whether the test compound is capable of inhibiting protocadherin-PC activity. In various embodiments, the determining comprises (a) determining binding of the protocadherin-PC protein to the β-catenin and/or to the FHL-2, (b) determining whether the protocadherin-PC is capable of translocating β-catenin to the cytoplasm, (c) determining whether protocadherin-PC is activating the wnt signaling pathway or increasing the expression of LEF-1/TCF target genes in the cancer cell, (d) determining whether protocadherin-PC is modulating the expression of the androgen receptor protein, or (e) any combination thereof. In another embodiment, the contacting is achieved by applying the test compound to cells expressing the protocadherin-PC, the β-catenin and the FHL-2.

Methods for assessing the extent of binding interactions between proteins are well known in the art. Nonlimiting examples include ELISA assays, western blot analyses, radioimmunoassay, immunoprecipitation analyses, two-dimensional gel electrophoresis and mass spectrometry. Illustratively, to determine the extent of interaction between protocadherin-PC and β-catenin using an ELISA assay, antibodies specific for β-catenin are immobilized on a solid support, such as a polystyrene well. The sample to be analyzed is then incubated in the well. In this case, the sample to be analyzed may contain a test compound, protocadherin-PC protein and β-catenin protein. Beta-catenin binds specifically to the antibody immobilized in the well. If the test compound does not disrupt the interaction between protocadherin-PC and β-catenin, then protocadherin-PC will become bound in the well via the protein-protein interaction. If the test compound is successful in disrupting the interaction, then unbound protocadherin-PC will be washed out of the well, along with unbound test compound and unbound β-catenin, by a series of washes. A reporter antibody specifically directed to protocadherin-PC is then added to the well. The antibody may be linked to an enzyme that catalyzes the conversion of a colorless substrate to a colored product. If protocadherin-PC is engaged in an interaction with β-catenin, the reporter antibody will bind specifically to the complex via protocadherin-PC and a colored reaction product will result. If the test compound inhibited the interaction, the reporter antibodies will be washed out of the well by a series of washes and a color change will not be detected. The exemplary assays listed here can be carried out on purified proteins, samples derived from cells or tissue extracts.

In a specific embodiment, the test compound may be applied to cells expressing protocadherin-PC, β-catenin and FHL-2. Intracellular protein-protein interactions may be visualized by techniques known in the art. Nonlimiting examples of such techniques include immunocytochemistry with antibodies specific for protocadherin-PC, β-catenin and FHL-2, and fluorescence resonance energy transfer (FRET) between proteins of interest engineered to express fluorescent tags. Alternatively, following application of the test compound, cell lysates may be prepared and protein-protein interactions may be assessed by the in vitro methods listed above.

The present invention encompasses a method for identifying whether a test compound is capable of inhibiting protocadherin-PC binding to β-catenin or FHL-2, the method comprising (a) contacting a protocadherin-PC protein with (i) a test compound and (ii) a catenin or an FHL-2 or both; and (b) determining whether binding of the protocadherin-PC protein to the β-catenin and/or the FHL-2 is inhibited compared to binding of the protocadherin-PC protein to the β-catenin and/or the FHL-2 in the absence of the test compound, so as to identify whether the test compound is capable of inhibiting the protocadherin-PC binding to the β-catenin or the FHL-2. In one embodiment, the test compound comprises a nucleic acid, an small molecule, a peptide, a PNA, a peptidomimetic, or an antibody. In another embodiment, the method is carried out for more than one hundred compounds. In yet another embodiment, the method is carried out in a high-throughput manner.

An exemplary binding site useful as a target in the screening methods of this invention is a protocadherin-PC amino acid sequence that mediates an interaction between protocadherin-PC and β-catenin. This amino acid sequence is encoded by the nucleotide sequence of from about 3601 to about 3635 of SEQ ID NO:1 (FIGS. 26A-26D).

This invention further encompasses a method for identifying whether a test compound is capable of inhibiting gene expression of protocadherin-PC, the method comprising (a) contacting a nucleic acid encoding a protocadherin-PC protein with a test compound; and (b) determining whether the protocadherin-PC gene expression is inhibited compared to protocadherin-PC gene expression in the absence of the test compound. In one embodiment, the determining comprises measuring transcription of the protocadherin-PC gene. In another embodiment, the determining comprises measuring protocadherin-PC mRNA. In another embodiment, the determining comprises measuring translation of the protocadherin-PC RNA into protein. In yet another embodiment, the determining comprises quantifying protocadherin-PC protein.

Methods that can be used to measure transcription (i.e., mRNA levels) and translation (i.e., protein levels) are well known to those skilled in the art. Such methods include, without limitation, reverse transcriptase PCR (RT-PCR), in situ hybridization, Northern blot, immunohistochemistry, radioimmunochemistry, western blot, ELISA, two-dimensional gel electrophoresis, and mass spectrometry. For example, using all or a portion of a nucleic acid encoding protocadherin-PC as a hybridization probe, the expression of protocadherin-PC mRNA can be measured. Binding of the hybridization probe to the protocadherin-PC mRNA may be quantitated by various means, including but not limited to radioactive labeling or fluorescent labeling. To illustrate one method for quantitation of protocadherin-PC protein, western blotting can be carried out by first separating proteins in a sample by polyacrylamide gel electrophoresis, then transferring the proteins to a membrane such as nitrocellulose by a method such as electroelution. Proteins of interest can be detected with specific antibodies labeled with measurable readout signals such as radioactive elements or fluorescent compounds, or enzymes that catalyze colorimetric or chemiluminescent substrates.

Transgenic Non-Human Mammals

This invention provides for a transgenic non-human mammal whose genome comprises a transgene comprising a nucleic acid encoding a protocadherin-PC operably linked to a tissue specific promoter. In one embodiment, the non-human mammal is a mouse, a primate, a bovine, or a porcine. In another embodiment, the tissue-specific promoter is the prostate-specific probasin gene promoter element. In one aspect, the invention encompasses an F1 transgenic mouse produced from a cross between the transgenic mouse of this invention and a transgenic mouse of the TRAMP line (strain C57BU6-Tg(TRAMP)8247Ng/J; Jackson Lab No. 003135) or any other mouse that develops prostate cancer.

U.S. Pat. No. 5,952,488 describes a DNA sequence cloned from the rat probasin gene promoter region which confers prostate-specific gene expression in transgenic non-human mammals. Using the prostate-specific rat probasin gene promoter sequence, expression of the oncoprotein SV40 T antigen (Tag) specifically in the prostate of transgenic mice provided a mouse model for the development and progression of prostate cancer (Greenberg et al., Mol Endocrinol 8:230-239 (1994); Greenberg et al., Proc Natl Acad Sci USA 92:3439-3443 (1995)). Methods for construction of the rat probasin-SV40 Tag transgene, and for production and screening of transgenic mice expressing the transgene are provided in U.S. Pat. No. 5,907,078. This transgenic mouse model for prostate cancer is known as the TRAMP (transgenic adenocarcinoma mouse prostate) model and is used to study primary and metastatic prostate cancer (Gingrich et al., Cancer Res 56:4096-4102 (1996)). The TRAMP model has been used to assess the efficacy of chemotherapeutic and chemopreventive agents in the treatment of prostate cancer (Kolluri et al., Proc Natl Acad Sci 102:2525-2530 (2005); Raghow et al., Cancer Res 62:1370-1376 (2002); Gupta et al., Cancer Res 60:5125-5133 (2000)). To study the effect of gene therapy to replace oncogenic p53 molecules with tumor suppressor p53 mutants, transgenic mice were generated using the rat probasin promoter for prostate-specific expression of mutated p53, the mice were then bred to the TRAMP mice, resulting in F1 mice with reduced tumor growth and increased survival (Hernandez et al., Mol Cancer Res 1:1036-1047 (2003)).

In addition to the TRAMP mouse model of prostate cancer, another series of transgenic mice have been developed as a model for prostate cancer. Transgenic mice of the LADY line differ from the TRAMP model by targeting only the large T antigen to the prostate via the probasin promoter, as opposed to the TRAMP model which targets the large and small T antigens to the prostate (Kasper et al., Lab Invest 78(6):i-xv (1998); Masumori et al., 61:2239-2249 (2001)). Transgenic mice from the LADY line have been used to study the efficacy of chemopreventive agents against prostate cancer (Venkateswaran et al., Cancer Res 64:5891-5896 (2004)). Rat probasin promoter-directed overexpression of the protease hepsin in a LADY mouse allowed the assessment of the impact of hepsin expression on the progression and metastasis of primary prostate tumors (Klezovitch et al., Cancer Cell 6:185-195 (2004)).

The rat probasin gene promoter has been used in multiple studies to generate transgenic mouse lines expressing a prostate-specific transgene (See Yan et al., Prostate 32:129-139 (1997) (transgenic mouse expressing prostate-specific chloramphenicol acetyl transferase gene); Kindblom et al., Endocrinology 144:2269-2278 (2003) (transgenic mouse expressing prostate-specific prolactin gene); Hernandez et al., Mol Cancer Res 1: 1036-1047 (2003) (transgenic mouse expressing prostate-specific p53 mutant); Elgavish et al., Prostate 61:26-34 (2004) (transgenic mouse expressing prostate-specific p53 mutant); Konno-Takahashi et al., J Endocrinol 177:389-398 (2003) (transgenic mouse expressing prostate-specific IGF-1).

In the context of the present invention, transgenic mouse lines may be constructed in which protocadherin-PC expression is targeted to the mouse prostate through the rat probasin gene promoter sequence. Transgenic mice expressing prostate-specific protocadherin-PC can be used to study chronic upregulation of wnt signaling, increases in the neuroendocrine-like characteristics and enhanced potential to acquire pro-malignant characteristics by the epithelial cell population in the prostates of the transgenic mice. The mice will also be useful to study changes in gene expression patterns and expression of gene products in the wnt signaling pathway and neuroendocrine differentiation.

Transgenic mice expressing prostate-specific protocadherin-PC may display phenotypic alterations such as bladder abnormalities, abnormalities in prostate nuclei, or both. The mice may not display overt cancer or outright signs of cancer. One explanation for this type of outcome is that protocadherin-PC may not cause cancer, rather expression of protocadherin-PC may increase the aggressiveness of already established tumors. Thus, if overt cancer is not observed in the transgenic mice expressing protocadherin-PC in the prostate, the mice can be bred to other transgenic mice which have been shown to develop prostate cancer (for example the TRAMP or LADY transgenic models of prostate cancer) to determine if protocadherin-PC can make the tumors more aggressive.

Methods for producing transgenic mouse lines are used routinely in the art and would be known to one skilled in the art. For example, in the present invention, the prostate-specific expression of protocadherin-PC can be accomplished using a replication-deficient adenovirus carrying the cDNA of SEQ ID NO:1 linked to the probasin promoter, such as the pPB-AAR2 expression vector (Andriani et al., J Natl Cancer Inst 93:1314-1324 (2001); Kakinuma et al., Cancer Res 63:7840-7844 (2003)). Founder mice can be identified by detection of transgene expression in tail DNA. Founder mice are bred into non-transgenic mice to expand each founder line. Prostate-specific expression of protocadherin-PC in progeny can be determined by immunohistochemical methods known in the art.

An aspect of the present invention provides for an F1 transgenic mouse produced from a cross between a transgenic mouse expressing prostate-specific protocadherin-PC and a mouse of the TRAMP or LADY models to assess the effect of protocadherin-PC expression on the aggressiveness of prostate cancer, i.e, neuroendocrine differentiation. In a nonlimiting example, a protocadherin-PC transgenic mouse can be crossed with a transgenic mouse of a LADY subline (12-T7) known not to give rise to aggressive neuroendocrine-like tumors. The F1 mouse will demonstrate whether expression of protocadherin-PC will make the LADY 12-T7 tumor model more aggressive and more likely to give rise to adenocarcinomas with a neuroendocrine phenotype (mediated by activation of the wnt signaling pathway). Assessment of neuroendocrine tumor development in the F1 mice can be assessed by immunohistochemical analysis of prostates for markers of neuroendocrine differentiation (i.e., increased expression of chromo-A, synaptophysin, and other neuropeptide hormones).

The present invention further provides for a method for determining whether a test compound is capable of treating prostate cancer, the method comprising (a) administering an effective amount of a test compound to a transgenic non-human mammal whose genome comprises a transgene comprising a nucleic acid encoding a protocadherin-PC operably linked to a tissue-specific promoter, wherein the transgenic non-human mammal has prostate cancer; (b) measuring progression of prostate cancer in the transgenic non-human mammal of (a); (c) comparing the measurement of progression of prostate cancer of step (b) to that of a sibling of the transgenic non-human mammal, wherein the sibling was not administered the test compound, and wherein an arrest, delay or reversal in progression of prostate cancer in the transgenic non-human mammal of (a) indicates that the test compound is capable of treating prostate cancer.

An arrest, delay or reversal in the progression of prostate cancer in mice can be assessed by physically measuring the weight and volume of the prostate or the volume of palpable tumors. Serum levels of IGF-I and IGFBP-3 can also be indicative of prostate cancer progression.

Terms

In one aspect of the invention, the compound can be combined with a carrier. The term “carrier” is used herein to refer to a pharmaceutically acceptable vehicle for a pharmacologically active agent. The carrier facilitates delivery of the active agent to the target site without terminating the function of the agent. Non-limiting examples of suitable forms of the carrier include solutions, creams, gels, gel emulsions, jellies, pastes, lotions, salves, sprays, ointments, powders, solid admixtures, aerosols, emulsions (e.g., water in oil or oil in water), gel aqueous solutions, aqueous solutions, suspensions, liniments, tinctures, and patches suitable for topical administration.

In one non-limiting embodiment of the invention, “specifically binds” in the context of binding of a nucleic acid to a target, means the nucleic acid binds to the target under moderate to high stringency, or where the target is at least about 70% identical to the nucleic acid. Computer-based algorithms known in the art can be used to design oligonucleotides that will target unique sequences within a nucleic acid encoding a protocadherin-PC, so as to minimize binding of the oligonucleotide to nucleic acids that do not encode a protocadherin-PC.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of <20%.

The term “effective” is used herein to indicate that the inhibitor is administered in an amount and at an interval that results in the desired treatment or improvement in the disorder or condition being treated (e.g., an amount effective to arrest, delay or reverse the progression of prostate cancer).

In some embodiments, nonlimiting examples of the subject include: human, mouse, rabbit, monkey, rat, bovine, pig or dog.

Pharmaceutical formulations include those suitable for oral or parenteral (including intramuscular, subcutaneous and intravenous) administration. Forms suitable for parenteral administration also include forms suitable for administration by inhalation or insufflation or for nasal, or topical (including buccal, rectal, vaginal and sublingual) administration. The formulations may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

The following examples illustrate the present invention, and are set forth to aid in the understanding of the invention, and should not be construed to limit in any way the scope of the invention as defined in the claims which follow thereafter.

### EXAMPLES

### Example 1

### A Human- and Male-Specific Protocadherin That Acts Through the Wnt Signaling Pathway to Induce Neuroendocrine Transdifferentiation of Prostate Cancer Cells

Protocadherin-PC(PCDH-PC, pro-PC or PCDH-Y) is a gene product that is selectively expressed in apoptosis- and hormone-resistant human prostate cancer cells. The gene encoding PCDH-PC is on the human Y-chromosome in a region that was translocated from the X-chromosome during the evolutionary transition from primates to humans. Compared to its X-homologue, PCDH-PC has a small deletion in its coding sequence that removes the signal sequence and the protein encoded by this gene is cytoplasmically localized. PCDH-PC also has a small serine-rich domain in its C-terminal region that is homologous to the β-catenin binding site of classical cadherins and hormone-resistant variants of prostate cancer cells that express PCDH-PC have high levels of β-catenin protein in their nuclear fractions consistent with evidence that these cells have increased wnt-signaling. Transfection of human prostate cancer, LNCaP, cells with PCDH-PC expression vectors or culture of LNCaP cells in androgen-free medium, an experimental condition that induces expression of PCDH-PC, activates wnt signaling in these cells as assessed by nuclear accumulation of -catenin protein, increased expression of luciferase from a reporter vector promoted by Tcf binding elements and increased expression of wnt target genes such as c-myc, cyclin D and Cox-2. Moreover LNCaP cells transfected with the PCDH-PC expression vector or grown in androgen-free medium transdifferentiate to neuroendocrine- (NE-) like cells marked by elevated expression of neuron specific enolase and chromogranin-A. NE transdifferentiation is also observed when LNCaP cells are transfected by a stabilized β-catenin expression vector. Increased wnt signaling and NE transdifferentiation of LNCAP cells induced by culture in androgen-free medium was suppressed by siRNAs that target PCDH-PC as well as by dominant-negative Tcf or siRNA against β-catenin supporting the hypothesis that increased expression of PCDH-PC is driving NE transdifferentiation by activating wnt signaling. These findings enhance the understanding of the process through which prostate cancers progress to aggressive and hormone-resistant states in humans.

Prostate cancer is a malignancy that develops and progresses under the influence of androgenic steroids. This influence is consistent with the use of various forms of androgen depletion therapies to treat patients diagnosed with metastatic prostate cancer for which surgery is no longer an effective treatment option. Androgen depletion provides rapid palliative relief to patients suffering pain as a consequence of bone metastatic prostate cancer and clinical study has proven that it extends the life span of the advanced prostate cancer patient even though the extension is only a matter of months (Klotz, 2000; Debryne, 2002) The transient effectiveness of androgen depletion therapy for prostate cancer patients is based upon its apparent ability to suppress proliferation of the tumor cells and, in the in vivo setting of the patient, induce apoptosis of, at least, a fraction of these cells (Isaacs et al., 1994; Denmeade et al., 1996). Inevitably, however, residual prostate tumor cells that survive androgen depletion therapy progress to a state where they are considered to be androgen-insensitive because their growth and survival is no longer suppressed in the androgen depleted environment of the treated patient, and it is these androgen-insensitive tumor cells that are associated with the relatively high morbidity and mortality of advanced disease.

Studies to identify the molecular basis for the development of androgen insensitivity of prostate cancer cells often focus on the androgen receptor (AR) gene and gene products (Craft and Sawyer, 1999; Buchanan et al., 2001; Culig et al., 2003; Taplin and Balk, 2004; Cornauer et al., 2003). These studies show that some androgen-insensitive prostate cancers from patients contain tumor cells with hyperactive androgen signaling associated with the presence of mutations in the AR gene (that makes AR promiscuous with regards to its ability to accept alternate steroid ligands) or in association with amplification of the AR gene (that increases basal expression of AR protein). Other experimental evidences that prostate cancer cells with increased expression of AR co-activators increases the ability of AR to function in low androgen levels (Sampson et al., 2001) or that activation of AR through mitogen-activated cell signaling pathways leads to ligand-independent transcriptional activity of the AR protein (Yeh et al., 1999; Chen et al., 2000; Lin et al., 2001; Ueda et al., 2002) have not been sufficiently translated to the human situation to identify the frequency with which these perturbations might be found in hormone-insensitive human tumors.

Alternatively, given the belief that hormone therapies for prostate cancer act by inducing apoptosis of prostate cancer cells, hormone-insensitive prostate cancer cells may have perturbations in their ability to mount an apoptotic response in an androgen-depleted environment. Bcl-2 expression is frequently upregulated in hormone insensitive prostate cancers retrieved from patients and elevated bcl-2 expression has been shown to confer an androgen-insensitive phenotype on a prostate cancer cell line that is normally androgen-sensitive (Catz and Johnson, 2003; Furumurthy et al., 2001; Raffo et al., 1995). Other perturbations of apoptotic pathway regulators reportedly found in hormone insensitive prostate cancer cells in patients include upregulated NFκB- and Akt-signaling (Lessard et al., 2002; Malik et al., 2002), either of which can contribute to an apoptosis resistant state under experimental conditions.

To identify other gene products associated with the acquisition of apoptosis- and hormone-resistance by prostate cancer cells, a model cell system was established by transiently exposing a prototypic human androgen-sensitive cell line, LNCaP, to stimuli (phorbol ester or serum starvation) that induced apoptosis of a majority of these cells during a 24 hr period (Chen et al., 2002). By expanding the surviving populations and repeating the exposure/expansion of survivor paradigm several more times, two variant cell lines were created, LNCaP-TR and LNCaP-SSR, that were resistant to the stimuli used to select them as well as to the alternate apoptotic stimuli that was not used in their selection. These variant cell lines were androgen-insensitive when tested for their ability to form tumor xenografts in castrated male immunodeficient mice (Chen et al., 2002). Use of a comparative genetic screening technique then allowed identification of a gene product that was selectively expressed in the apoptosis-resistant and androgen-insensitive variant lines but not in the parental LNCaP cell line (Chen et al., 2002). Analysis of the sequence of the major transcript (4.5 kb) of the gene product selectively expressed in the variant prostate cancer cell lines revealed that it is a unique member of the protocadherin gene family encoded by a gene localized on the Y-chromosome of humans (at Yp 11.2) (Blanco et al., 2000) and because of its association with human prostate cancer, the gene is named protocadherin-PC(PCDH-PC) (Chen et al., 2002). Growth of parental LNCaP cells in a medium free of androgens or castration of male mice bearing LNCaP xenograft tumors also induces expression of PCDH-PC (Chen et al., 2002).

PCDH-PC was evolutionarily derived from a homologous gene present on the human X-chromosome (PCDHX) that lies within a region of the chromosome (at Xq21.3) that was duplicated and translocated to the Y-chromosome during the transition from higher primates to humans (Blanco et al., 2000). The coding region of the PCDH-PC gene (also referred to as PCDHY) shares 98.1% sequence homology with the PCDHX gene. Aside from occasional nucleotide differences scattered throughout the coding region, the Y-linked gene has a deletion of a contiguous 13 bp sequence (present in exon 4 of the X-linked gene) as well as complete deletion of 3 potential exons (#7, 8 and 8A as defined in Blanco-Arias et al., 2004) that are present in some splice variants of PCDHX mRNA. The 13 bp deletion in the PCDH-PC gene has important consequences for the polypeptide(s) encoded by this gene. This deletion results in a major transcript with an AUG codon embedded within a strong Kozak consensus sequence that preferentially translates to a protocadherin polypeptide lacking a signal sequence (Chen et al., 2002; Blanco et al., 2000). This is consistent with our finding that a polyclonal antibody raised against a polypeptide sequence within the C-terminal domain of PCDH-PC recognizes a protein of the appropriate molecular weight that fractionates with the cytoplasm of LNCaP-TR and -SSR cells (Chen et al., 2002). Thus, the major protein encoded by the PCDH-PC transcript is predominantly localized in the cytoplasm rather than membrane bound, as with most other members of the cadherin gene family.

Another important property of PCDH-PC is the presence of a small serine-rich domain within the C-terminal region of the polypeptide that is homologous to the β-catenin binding domains found in classical cadherins (E-, P- and N-cadherin) (Chen et al., 2002). Immunoprecipitation of PCDH-PC from LNCaP-TR and -SSR cell extracts co-precipitated β-catenin (Chen et al., 2002), supporting the functional interaction of these two molecules within the apoptosis-resistant cells. Moreover, the apoptosis-resistant LNCaP variants that express PCDH-PC had anomalies in their intracellular β-catenin distribution pattern (LNCaP-SSR and -TR have β-catenin in the cytoplasmic and nuclear fractions whereas parental LNCaP cells have β-catenin strictly localized to the membrane fraction) and this was consistent with the ability to demonstrate enhanced luciferase production in the apoptosis-resistant LNCaP variants using a Tcf-promoted luciferase reporter vector (Chen et al., 2002; de la Taille et al., 2003). Collectively, these preliminary data show that PCDH-PC encodes a cytoplasmic protein that interacts with β-catenin and induces cell signaling through the wnt pathway mediated by nuclear accumulation of β-catenin and enhanced transcription from Tcf/LEF-1 binding elements on DNA. This also shows that the apoptosis-resistant phenotype present in the LNCaP variants that express PCDH-PC might be related to its ability to stimulate wnt signaling, especially since it was shown that wnt signaling can induce apoptosis-resistance in other tumor cell systems (Chen et al., 2001; Queires et al., 2005).

Most studies were based on the experimentally-derived LNCaP cell variants that express PCDH-PC, but some studies also show that transfection of parental LNCaP cells with a PCDH-PC expression vector increased the relative apoptosis-resistance of these cells and conferred a hormone-resistant phenotype on them as evidenced by their ability to form tumors in castrated male immunodeficient mice (Chen et al., 2002; Queires et al., 2005). Studies of clinical specimens of human prostate cancer also show that PCDH-PC expression is frequently upregulated in hormone-resistant prostate tumor cells (Queires et al., 2005), supporting that PCDH-PC expression is associated with the development of hormone-resistant prostate cancer in humans. This invention shows that PCDH-PC expression stimulates wnt signaling in prostate cancer cells as shown by examining for biomarkers of wnt signaling activation in LNCaP and other human cancer cells that are transiently transfected with PCDH-PC. An unexpected change was noted in the differentiation pattern of PCDH-PC transfected prostate cancer cells that has led us to study whether this gene product and its actions on the wnt signaling pathway might also be involved in a well recognized transdifferentiation process in which prostate cancer cells acquire phenotypic characteristics of neuroendocrine- (NE-) like cells. The invention provides for methods to inhibit progression of human prostate cancer to the advanced or hormone-insensitive stage.

Cell Lines. The human prostate cancer cell lines, LNCaP, DU145, CWR22rv-1 and PC-3 were obtained from the ATCC (Manassas, Va.) as was the human colon cancer cell line, HCT116. LNCaP and DU145 cells were maintained in RPMI-1640 medium. PC-3 cells are maintained in F12K medium. HCT116 cells were maintained in DMEM. All media are supplemented with 10% fetal bovine serum (FBS) and penicillin/streptomycin unless noted. For androgen-free conditions, LNCaP cells were cultured in phenol-red free RPMI medium containing 10% charcoal-stripped fetal bovine serum (CS-FBS) as previously described (Shen et al., 1997). This culture condition was previously shown to induce PCDH-PC expression in LNCAP cells (Chen et al., 2002) as well as to initiate a transdifferentiation process in which the LNCaP cells acquire morphological and biochemical features of neuroendocrine-like cells (Shen et al., 1997). Other medium additives include dibutyrl cyclic AMP (db-cAMP, 1 mM, Sigma Chemical Company, St. Louis, Mo.), interleukin-6 (IL-6, 50 ng/ml, Upstate Biotechnology, Inc., Lake Placid, N.Y.) or NS-398 (5 μM, Cayman Chemical Co., Ann Arbor, Mich.) as noted.

Expression Vectors, Transfection Protocols and Luciferase/β-Glactosidase Assays. PCDH-PC cDNA was inserted into the mammalian expression vectors pcDNA3 (Invitrogen Life Technologies, Inc., Carlsbad, Calif.) or into pCMV-myc (BD Biosciences, Clontech, Inc., Palo Alto, Calif.) so that the PCDH-PC product generated from this vector (pPCDH-PC-myc) has a C-terminal myc tag. An expression vector containing cDNA encoding a mutated (stabilized) form of human β-catenin (Tetsu and McCormick, 1999) was used. A dominant-negative Tcf expression vector used was previously described (Chen et al., 2001). The Tcf-sensitive reporter vector, pTOP and a CMV-promoted β-galactosidase expression vector were obtained from Upstate Biotechnology, Inc. Transfections used for protein or RNA extractions were done in 35 cm² dishes with cells plated at 50% density 12-16 hrs prior. Aliquots of expression plasmid DNA (totaling 6 ugs) were mixed with Lipofectamine 2000 (Invitrogen Life Technologies, Inc.) in antiobiotic-free, serum-free medium as described by the manufacturer and were applied to the cultures. Transfections used for measurement of luciferase and β-galactosidase activities were done in 12-well plates and equal aliquots of the pTOP reporter vector (900 ng) were mixed with 100 ng of a CMV-promoted β-galactosidase expression vector so that all wells received 1 ug of DNA mixed with lipofectamine-2000, as above. Medium was changed after 4 hrs to a serum-containing medium without antibiotics for the remainder of the 48 hr transfection period. Luciferase activity in cell extracts was measured using the Luciferase Assay System of Promega, Inc. (Madison, Wis.). β-galactosidase activity was also measured in the same cell extracts using the β-galactosidase Enzyme Assay System of Promega, Inc. All experiments involving luciferase reporter vectors were done in triplicate for each point.

siRNAs and Transfection of Cultured Human Prostate Cancer Cells. Commercial siRNAs targeting human β-catenin and lamin A were purchased from Dharmacon, Inc. (Chicago, Ill.). Three different siRNAs targeting PCDH-PC were designed using the siRNA Target Finder software program available through Ambion, Inc. (Austin, Tex.). The anti-PCDH-PC siRNAs targeted sequences at position 3043-3062 (#181; SEQ ID NO:4; FIG. 29), 3098-3117 (#190; SEQ ID NO:6; FIG. 31) or 3345-3364 (#208; SEQ ID NO:7; FIG. 32) on the PCDH-PC mRNA. The 21 bp siRNAs were constructed using the 19 bp core sequences described above with 2 nucleotide UU overhangs and these siRNAs were produced and provided by Ambion, Inc. siRNAs were transfected or co-transfected (with other expression vectors as described) into LNCaP cells at 100 nM final concentrations using Lipofectamine 2000 transfection reagent (Invitrogen Life Technologies, Inc.) in serum-free medium as instructed by the manufacturer. 48 hrs after transfection, cells were harvested and extracted for protein or RNA as described below. Protein Extraction from Cultured Cells and Nuclear Isolation Procedures. Monolayer cultures were washed once in cold phosphate-buffered saline (PBS) and then cells were scraped into PBS and pelleted by low-speed centrifugation. Cell pellets were extracted in RIPA buffer as previously described (Raffo et al., 1995). RIPA extracts were centrifuged at 10,000×g to remove debris prior to protein assay and analysis. For nuclear isolation from cultured cells, monolayers containing 5×10⁶ cells were washed twice in cold PBS and were scraped into a buffer containing 10 mM HEPES, pH 7.9, 10 mM KCl, 1 mM DTT, 10 mM EDTA and 0.4% polyoxyethylene nonyl phenol (IGEPAL) with a 1× protease inhibitor cocktail (Sigma, Inc., St. Louis, Mo.). The cell suspensions were maintained on ice on a rocking platform for 10 min and were centrifuged at 15,000×g for 3 min at 4° C. The pellets were suspended in 150 μl of 20 mM HEPES, pH 7.9, 0.4 M NaCl, 1 mM EDTA, 1 mM DTT with 1× protease inhibitor cocktail and vortexed for 15 sec. The suspensions were maintained on ice on a rocking platform for 2 hrs then insoluble debris was removed by centrifugation at 15,000×g for 5 min. Aliquots of whole cell and nuclear extracts were assayed for protein using the BioRad DC Protein Assay (BioRad, Inc., Hercules, Calif.).

Western Blot Analysis of Proteins. Aliquots of cell extracts containing equal amounts of protein were electrophoresed on 10% polyacrylamide gels and the proteins in the gel were electrotransferred to PVD filters as previously described (Raffo et al., 1995). Antibodies used in Western blot analyses were mouse monoclonal antibodies obtained from Dako, Inc. (Anti-neuron specific enolase and anti-chromogranin A antibodies, Carpenteria, Calif.), Santa Cruz Biotechnology, Inc. (anti-β-catenin and anti-lamin A/C, Santa Cruz, Calif.) or Sigma Chemical Co, Inc (anti-human β-actin). Primary antibody dilutions were prepared according to manufacturer's recommendations and detection of primary antibody binding to the Western blots was done using a horseradish peroxidase-labeled secondary goat anti-mouse antibody (Santa Cruz Biotechnology, Inc.). Chemiluminescent detection of secondary antibody binding to the filters was done using Luminol reagent (Santa Cruz Biotechnology, Inc.) and exposing the filters to film (Kodak XAR5). Bands on the film were compared to pre-stained molecular weight markers that were co-electrophoresed on each gel to ascertain that the band recognized by any given antibody was of the appropriate mass.

Targeted cDNA Microarray Expression Analysis. RNAs were extracted from LNCAP cells maintained for 10 days in CS-FBS medium or from LNCaP cells transfected with empty (pCMV-myc) vector, pCMV-PCDH-PC-myc vector or an expression vector for stabilized (mutant) β-catenin for 48 hrs using the Superarray mRNA purification kit (Superarray Biosciences, Inc., Frederick, Md.). The mRNAs were converted to biotin-16-dUTP-labeled cDNA using the GE Array Ampo Labeling Kit (Superarray Biosciences, Inc.). Labeled cDNAs were hybridized to individual human wnt-target gene cDNA microarrays (GE array Q series) from Superarray Biosciences, Inc. overnight and hybridization was detected using the Genearray Chemiluminescent Detection kit (Superarray Biosciences, Inc.) followed by exposure to Kodak XAR-5 film. All microarrays were processed in batch and exposed on the same film. The films were scanned and were analyzed using the Gene Array Analysis Software, Scanalyze and results from different experimental paradigms were compared to a control array that was hybridized to LNCaP cDNA using the software program Gene Array Analyzer of Superarray Biosciences. Confirmation of increased expression of c-myc, Cox-2 and wnt 7b mRNAs in PCDH-PC transfected LNCAP cells was done by multi-cycle RT-PCR using the following primers for c-myc: forward-5′-CTCCTGGCAAAAGGTCAGAG-3′ (SEQ ID NO:8), reverse-5′ AGCTTTTGCTCCTCTGCTTG-3′ (SEQ ID NO:9); Cox-2: forward-5′GAGGGTAGATCATCTCTGCCT-3′ (SEQ ID NO:10), reverse-5′-CCTGATTCAAATGAGATTGTGGA-3′ (SEQ ID NO:11); and wnt 7b-5′ TGCCTGCAGGTCCTAGAAGT-3′ (SEQ ID NO:12), reverse-5′-AATCTTGGCTCATTGCAACC-3′ (SEQ ID NO:13) at 24, 28 and 32 cycles. Equal aliquots of PCR product were electrophoresed on agarose gels and were visualized under UV light after staining with ethidium bromide. PCR product size was ascertained by comparison to a molecular weight marker run on an adjacent lane.

RNA Extraction and RT-PCR Analysis. Cell monolayers were rinsed and scraped into cold PBS for RNA extraction using the Rneasy Mini Kit from Qiagen, Inc. (Valencia, Calif.). The RNA was converted to cDNA using the Superscript Reverse Transcriptase Kit of Invitrogen Life Technologies, Inc. RNA was quantified by spectrometry at 260 nm and 2.0 μg aliquots were PCR amplified using Taq polymerase (Invitrogen Life Technologies, Inc) using primer sets designed to amplify a 938 bp region of PCDH-PC (5′ primer: 5′ TAGGAGGAAACACAAGAGAT-3′ (SEQ ID NO:14); 3′ primer: 5′-AGAAAGTTACATCTCACTGCA-3′ (SEQ ID NO:15); cycled at 94° for 30 sec; 51° for 30 sec; 72° for 4 min for 25 cycles) or using a primer set designed to amplify a 1,134 bp fragment of human β-actin cDNA (5′primer: 5′-ATGGATGATGATATCGCCGC-3′ (SEQ ID NO:16); 3′ primer: 5′-AAGCATTTGCGGTGGACGAT-3′ (SEQ ID NO:17) cycled at 94° for 30 sec; 53° for 30 sec; 72° for 4 min for 28 cycles). Equal aliquots of PCR reaction products were electrophoresed on a 0.8% agarose gels adjacent to molecular weight markers and were visualized following ethidium bromide staining under UV light.

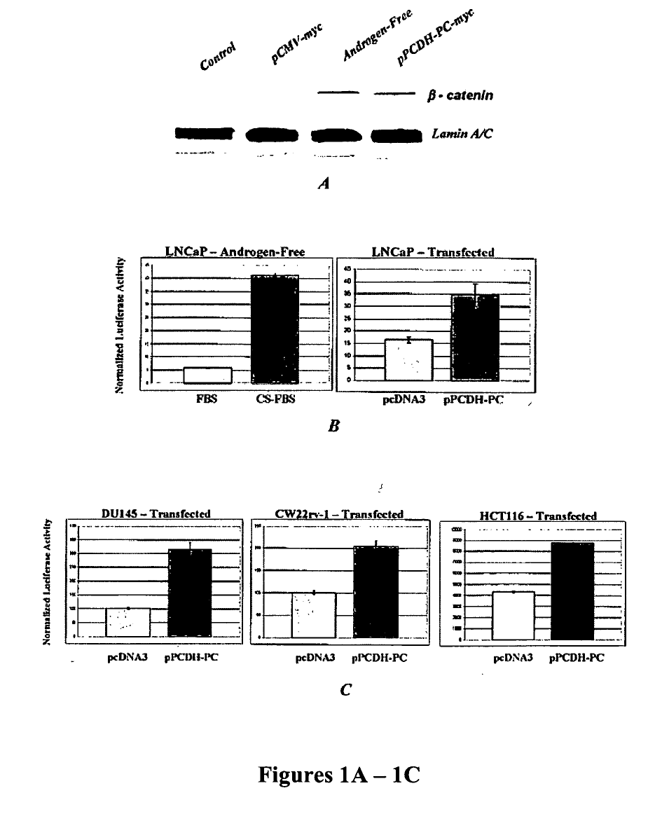

Protocadherin-PC Expression Upregulates Wnt Signaling in Prostate and Other Cancer Cell Lines. The end point of the canonical wnt signaling pathway is marked by increased nuclear accumulation of β-catenin protein and increased expression of gene products that are transcriptionally regulated by the Tcf family of transcription factors (Lustig and Behrens, 2003). Wnt signaling was upregulated in PCDH-PC expressing variants of LNCaP cells that were selected in vitro for resistance to apoptotic agents (Chen et al., 2002). To show that wnt signaling is modulated by expression of PCDH-PC, LNCaP cells were grown in androgen-free medium, a condition that induces expression of PCDH-PC, or LNCaP cells were directly transfected with a PCDH-PC expression vector to determine conditions which increased nuclear levels of β-catenin protein in these cells. Isolated nuclear fractions of parental LNCaP cells or LNCaP cells transfected with an empty expression plasmid (pCMV-myc) did not have detectable β-catenin protein as assessed by Western blotting analysis (FIG. 1A). However, both PCDH-PC transfected LNCaP cells and LNCaP cells maintained for 10 days in androgen-free medium, had high levels of β-catenin protein in their nuclear fractions (FIG. 1A). The ability of PCDH-PC transfection to induce nuclear accumulation of β-catenin in LNCaP cells was also consistent with analysis of luciferase activity in these and other human prostate and colon cancer cells co-transfected with a luciferase reporter vector (pTOP) that is promoted by a DNA sequence containing multiple Tcf binding elements. LNCaP cells express significantly more luciferase from this reporter vector when co-transfected with a PCDH-PC expression vector than when co-transfected with an empty vector (FIG. 1B). Likewise, LNCAP cells cultured for 8 days in androgen-free medium expressed significantly more luciferase when transiently transfected with the pTOP reporter vector when compared to LNCaP cells cultured in normal medium (FIG. 1B). As well other human prostate (DU145 and CWR22rv-1) and colon cancer (HCT116) cells expressed significantly more luciferase from pTOP when co-transfected with a PCDH-PC expression vector than when co-transfected with an empty expression vector (FIG. 1C).

A commercially prepared, targeted human wnt-pathway cDNA microarray analytical procedure was used to assess whether wnt target genes were upregulated by transfection with PCDH-PC or culture of LNCaP cells in androgen-free medium. The targeted microarray utilized contains spots for 37 different cDNAs of known canonical wnt-targets (genes regulated by the Tcf/LEF-1 transcription factor), four gene products referred to as non-canonical wnt targets (upregulated in association with a change in cellular Ca⁺⁺ ion metabolism induced by wnt signaling) as well as 65 other gene products representing molecules potentially involved in the wnt signaling process. Individual arrays were hybridized to labeled cDNA prepared from control LNCaP cells or from LNCaP cells cultured in androgen-free medium for 10 days as well as to cDNA from LNCaP cells transfected with the PCDH-PC expression vector or a stabilized β-catenin expression vector for 48 hrs. Expression patterns on the test arrays were then compared to the control array (hybridized to LNCaP cDNA) to identify differences in gene expression associated with the experimental conditions.

TABLE 1
Summary of changes in human wnt-target gene expression (increased
2-Fold or greater) measured in LNCaP cells transfected for 48 hrs with
a PCDH-PC or stabilized β-catenin expression vector or in LNCaP cells
grown for 7 days in androgen-free medium (CS-FBS). Changes in
expression of individual gene products under each test condition were
determined by comparison to the gene expression profile of untransfected
LNCaP cells.

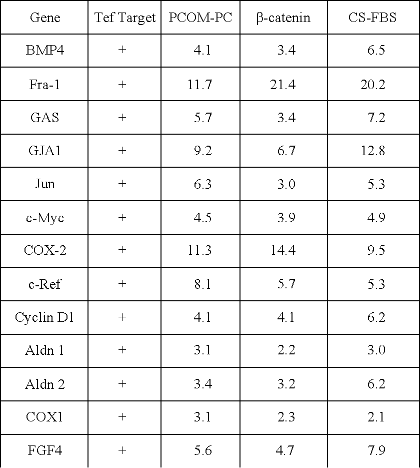

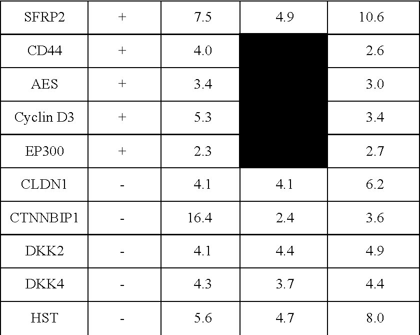

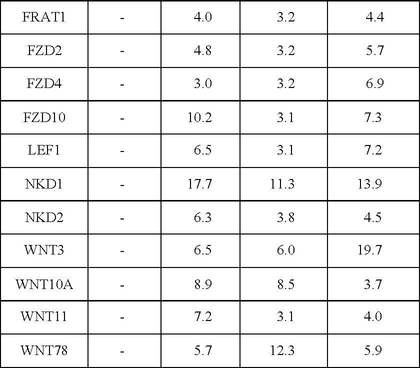

The results of these analyses (Table 1) showed that 18 of the 37 known canonical wnt target genes spotted on the array were upregulated by at least 2-fold or greater under both test conditions involving increased expression of PCDH-PC (cultured in androgen-free medium or transfected with PCDH-PC). Most of these genes (with the exception of 4 as indicated in Table 1) were also upregulated to a similar extent by transfection with stabilized β-catenin. The remaining 19 canonical Tcf-regulated gene cDNAs on the array were not significantly upregulated under any of the test conditions (culture in androgen-free medium or transfection with PCDH-PC or with β-catenin. Two of the non-canonical wnt target genes (iNOS and COL1A1) present on the array were also upregulated 2-fold or greater under all 3 test conditions. The three tested experimental conditions also induced numerous gene products that are involved in the wnt signaling process (exemplified by several different wnts and frizzled receptors) (Table 1), supporting that wnt signaling might have a feed-back loop (Leung et al., 2002) in prostate cancer cells that further influences the wnt signaling action in these cells.

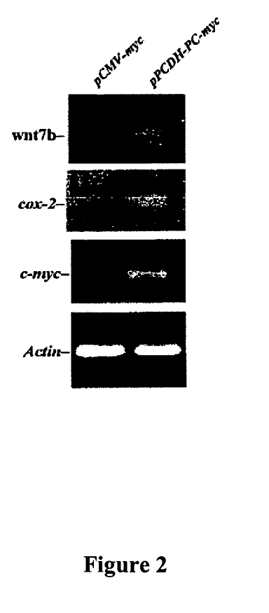

Semi-quantitative RT-PCR analysis was conducted on cDNA prepared from parental LNCaP cells or LNCaP cells transiently transfected with PCDH-PC using primers specific for small regions of the human c-myc, Cox-2 and wnt 7b transcripts (FIG. 2). This assay was performed with 3 different cycles (24, 28 and 32 cycles) for each primer set and the results were similar for each condition, showing increased levels of PCR product in the PCDH-PC transfected cells.

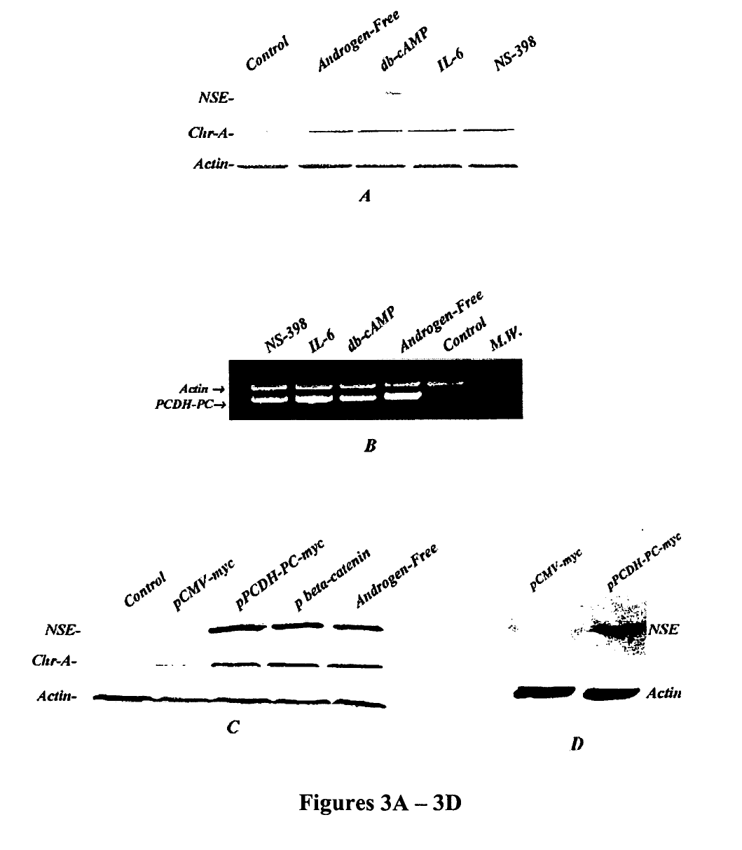

Protocadherin-PC Expression is Also Associated with Transdifferentiation of Prostate Cancer Cells to a Neuroendocrine Cell-Like Phenotype. Chronic culture of LNCaP cells in a medium depleted of androgens upregulates the expression of PCDH-PC (Chen et al., 2002) and this condition is also associated with a unique transdifferentiation process in which these cells gradually acquire morphological and other phenotypic characteristics of a neuroendocrine- (NE-) like cell type (Shen et al., 1997). Aside from androgen-depleted conditions, others have reported that culture of LNCaP cells in medium supplemented with dibutyral cyclic AMP (db-cAMP), IL-6 or NS-398, a selective cox-2 inhibitor, also induce NE transdifferentiation (Bang et al., 1994; Deeble et al., 2001; Meyer-Seigler, 2001). Since PCDH-PC expression was found to be highly upregulated in LNCaP cells maintained in androgen-free medium, we assessed whether these other NE transdifferentiation inducer agents might also upregulate PCDH-PC expression. Results of a Western blot survey of protein extracts made from control LNCaP cells or cells chronically cultured in androgen-free medium (10 days) or in normal medium supplemented with 1 mM db-cAMP, 50 ng/ml IL-6 or 5 nM NS-398 (5 days) showed that the expression of neuron specific enolase (NSE) and chromogranin-A proteins, two prominent biomarkers of NE transdifferentiation were highly upregulated in each of these conditions (FIG. 3A). When a second set of cultures treated with these same conditions were extracted for RNA and the RNAs were analyzed by RT-PCR for expression of PCDH-PC, all NE transdifferentiated cells had highly upregulated expression of PCDH-PC mRNA (FIG. 3B) in contrast to control LNCaP cells that do not express PCDH-PC. Thus, upregulated expression of PCDH-PC appears to accompany NE transdifferentiation of LNCaP cells induced by a wide variety of stimuli. More significantly, direct transient transfection of LNCaP cells with a PCDH-PC expression vector also induced NSE and chromogranin-A protein expression (FIG. 3C) indicating that this molecule is likely causative of NE transdifferentiation rather than just a correlative biomarker. Similar results were found when a different prostate cancer cell line, PC-3 was transiently transfected with PCDH-PC (FIG. 3D). Finally, since PCDH-PC expression is associated with increased wnt signaling experiments were also carried out to determine whether transfection of parental LNCaP cells with a stabilized mutant of β-catenin was sufficient to induce NE transdifferentiation. Results of transient transfection of LNCaP cells shown in FIG. 3C confirms that β-catenin transfection is also an efficient inducer of NSE and chromogranin-A expression and supports the idea that increased wnt signaling associated with PCDH-PC expression is involved in the NE transdifferentiation process of LNCaP cells. This is supported as well by findings of increased nuclear accumulation of β-catenin protein and increased expression from a TCF-promoted reporter vector in LNCAP cells chronically maintained in androgen-free medium (FIGS. 1B and 1C), a condition in which PCDH-PC expression is highly upregulated.

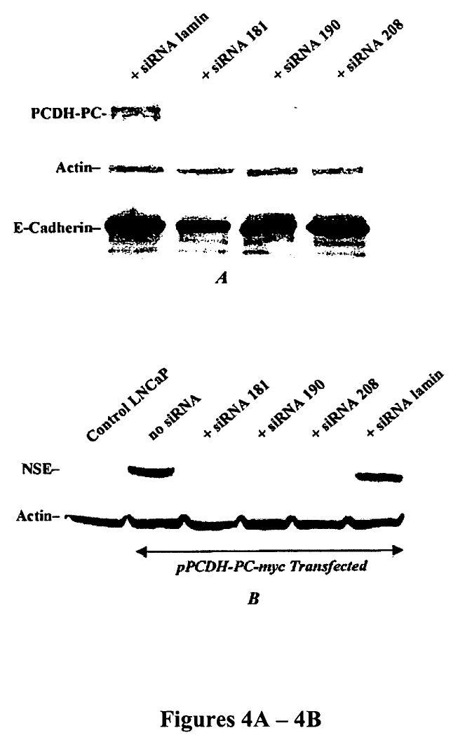

Suppression of Protocadherin-PC Expression Blocks/Suppresses the Induction of Wnt Signaling and Neuroendocrine Transdifferentiation of Prostate Cancer Cells Grown in Androgen-Free Medium. To show the relationship between PCDH-PC expression and NE differentiation of prostate cancer cells, three different siRNAs were designed and tested that target unique sequence regions of the PCDH-PC transcript. The design of the siRNAs avoided any potential regions of homology with cadherin box sequences or transmembrane domain sequences. When any of these 3 siRNAs were co-transfected into LNCaP cells along with the myc-tagged PCDH-PC expression vector, they strongly suppressed expression of myc-tagged PCDH-PC polypeptide whereas co-transfection of the PCDH-PC expression vector along with siRNA targeting the lamin gene product did not suppress expression of the PCDH-PC polypeptide (FIG. 4A). Expression of another cadherin family gene, E-cadherin, was unaffected by any of the PCDH-PC-specific siRNAs (FIG. 4A). Repetition of this experiment using a different set of LNCaP cells and evaluation of the effects of these PCDH-PC specific siRNAs showed that they blocked the ability of PCDH-PC transfection to induce NSE (FIG. 4B), consistent the suppression of PCDH-PC expression preventing NE transdifferentiation. The siRNAs were also tested to determine if they would suppress the ability of exposure to androgen-free medium to induce wnt signaling in LNCaP cells and, as shown in FIG. 4B, the PCDH-PC-specific siRNA 181 (SEQ ID NO:4; FIG. 29) completely suppressed the ability of 7 days culture in androgen free medium to induce wnt signaling in these cells as indicated by the suppression of induced luciferase expression from the pTOP reporter vector that was transfected into them during the last two days of culture. Additionally, all 3 of the PCDH-PC siRNAs strongly suppressed the induction of NSE protein expression in LNCaP cells cultured for 7 days in androgen free medium, whereas the siRNA against human lamin did not affect the ability of PCDH-PC transfection to induce NSE expression (FIG. 4C).

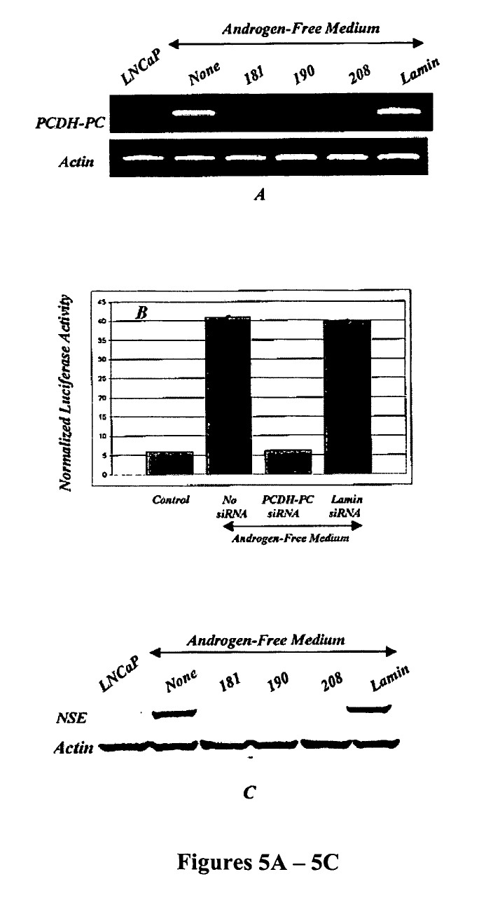

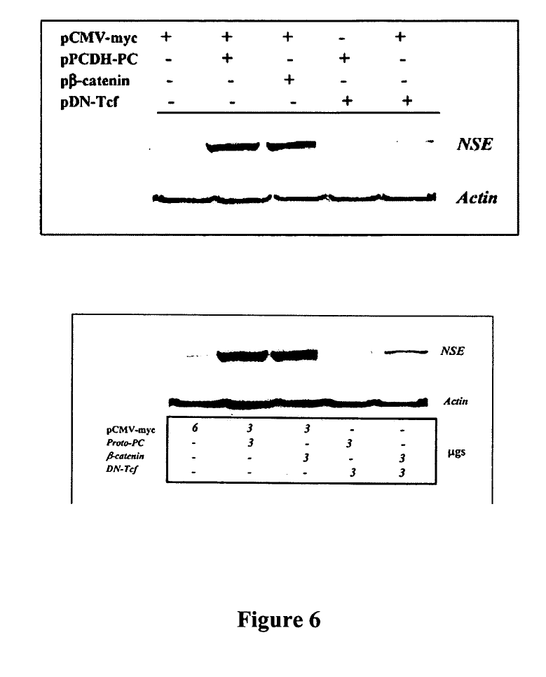

Suppression of Wnt-Signaling Blocks Neuroendocrine Transdifferentiation of Prostate Cancer Cells Induced by Protocadherin-PC Expression. NE transdifferentiation of LNCaP cells could be induced by transfection with a PCDH-PC expression vector, a condition that upregulates wnt signaling, or by transfection with a stabilized β-catenin expression vector. In further tests to prove that activation of the wnt signaling pathway is involved in the action of PCDH-PC in inducing NE transdifferentiation, suppression of wnt signaling was evaluated to determine if it is sufficient to suppress NE transdifferentiation induced by PCDH-PC in transfected or androgen-free LNCAP cells. A dominant negative (DN-) Tcf was analyzed for its ability to suppress NE transdifferentiation induced by transfection with PCDH-PC or stabilized β-catenin. As is shown in FIG. 5, co-transfection of LNCaP cells with PCDH-PC and DN-Tcf or β-catenin and DN-Tcf strongly suppressed the upregulation of NSE expression induced by PCDH-PC or β-catenin when they were co-transfected with an empty vector control. This was also tested with the use of a commercially-supplied siRNA that targets human β-catenin. As shown in FIG. 6A, the β-catenin siRNA was able to reduce β-catenin protein expression in LNCaP cells by 95% (as evaluated by densitometry of the Western blot shown in FIG. 5A) following a 48 hr transfection period, compared to control untransfected LNCaP cells or LNCaP cells that were transfected with an siRNA against lamin. When the β-catenin siRNA was co-transfected with the PCDH-PC expression vector, induced NSE expression was significantly reduced whereas siRNA against lamin did not affect the ability of PCDH-PC to induce NSE expression in LNCaP cells (FIG. 6B). Likewise, transfection of LNCaP cells with siRNA against β-catenin strongly suppressed the ability of culture in androgen-free conditions (CS-FBS) to induce NSE expression in these cells (FIG. 6C). Collectively, these results show that β-catenin/Tcf-mediated transcription is critical for NE transdifferentiation of LNCaP cells induced by PCDH-PC or by culture under androgen-free conditions and implicates wnt signaling as the common mediating factor in NE transdifferentiation of prostate cancer cells associated with PCDH-PC expression.

The data shows, using naturally selected prostate cancer cell lines (Chen et al., 2002), that expression of the PCDH-PC protein is associated with upregulation of wnt signaling in prostate and other human cancer cells. This is shown by the finding that PCDH-PC expression in commonly utilized human prostate cancer cell lines (either subsequent to transient transfection with a PCDH-PC expression vector or subsequent to growth of an androgen-sensitive prostate cancer cell line in medium depleted of androgens) leads to nuclear accumulation of β-catenin, increased expression of a luciferase reporter from a Tcf-sensitive promoter element and increased expression of wnt-target genes such as c-myc, cyclin D, c-ret and cox-2. PCDH-PC may enable β-catenin, one of the end molecules of the wnt signaling pathway, to escape the degradative process that regulates its access to the nucleus. A region of homology is described within the C-terminal region of PCDH-PC and the β-catenin binding sites of classical cadherins and data show that PCDH-PC co-immunoprecipitates with β-catenin, indicating that there is a functional interaction of these two molecules. PCDH-PC has a nuclear localization consensus sequence even though significant levels of PCDH-PC protein have only been detected in cytoplasmic fractions of prostate cancer cells to date. Yeast-2-hybrid studies have been conducted to identify other binding partners of PCDH-PC and have shown that FHL-2 protein, a co-activator of β-catenin/Tcf transcriptional activity (Wei et al., 2003) also binds PCDH-PC (see Example 7) and may also indicate that PCDH-PC protein provides a scaffolding to bring FHL-2 and β-catenin into juxtaposition and facilitates activation of Tcf-mediated transcription. For prostate cancer, the studies indicate that PCDH-PC expression and its downstream effects on the wnt signaling pathway are linked to a unique process in which prostate cancer cells transdifferentiate to a NE-like state. Whereas the finding that PCDH-PC expression can induce wnt signaling has many implications for the process through which prostate cancers might progress to a hormone- and apoptosis-insensitive state, the finding that this gene product is linked to NE transdifferentiation adds support for the role of this gene in prostate cancer.

Like other visceral tissues in the human body, the normal human adult prostate gland contains a small fraction of neuroendocrine cells, widely dispersed throughout the epithelial cell layer (Cohen et al., 1993). These cells have also been referred to as endocrine-paracrine cells or amine precursor uptake and decarboxylation (APUD) cells, but their identifying characteristics include a unique morphology of long cellular processes that extend into the planes of the epithelial cell layers and the presence of a large number of dense intracellular secretory granules within the cytoplasm that store a diverse collection of neurosecretory peptides (exemplified by bombesin, calcitonin, parathyroid-like hormone, serotonin and proadrenomedullin) that have the potential to influence the growth and survival of the other cells types within the epithelial layer in which they are interspersed (Cohen et al., 1993). These NE cells have a role in the biology of human prostate cancer development and progression, especially in the process through which advanced prostate cancer progresses to hormone independence following hormonal therapy. There is a relatively rare subset of prostate cancer patients that present initially with homogenous NE cell tumors (referred to as Small Cell Carcinoma of the Prostate) that arise from the prostate gland (Randolph et al., 1997). While this type of prostate cancer is relatively rare (estimated to be approximately 60 new patients a year in the United States), the prognosis for these patients is poor as these tumors are highly metastatic and generally poorly responsive to therapies. However, even the far more common form of prostate cancer, adenocarcinoma of the prostate, shows clinical evidence for the potential influence of NE cells on this disease. Like the normal epithelium of the prostate gland, prostate adenocarcinomas often have NE-like cells interspersed amongst the malignant epithelial cells (di Sant'Agnese, 1992). Attempts to quantify the presence of NE cells within surgically ressected prostate tumors and to correlate NE cell populations with clinical parameters of these tumors such as stage, grade or disease-free survival are controversial; there have been several studies that have found such associations (Weinstein et al., 1996; McWilliam et al., 1997; Bollito et al., 2001), but just as many, if not more, that have not (Krupski et al., 2000; Ahlegren et al., 2000; Segawa et al., 2001). However, NE cells tend to be clustered within foci of primary and metastatic tumors as was revealed in analysis of smaller collections of prostate tumors or multiple metastases from individual patients (di Sant'Agnese, 1992; Roudier et al., 2003) as well as in a more large-scale assay of prostate tissues done using human prostate tissue microarrays (Mucci et al., 2000). Therefore, the task or correlating prostate tumor characteristics with NE cell populations is likely complicated by the irregular distribution of NE cells and tumor sampling limitations may be one reason that the results of these kinds of studies have been so conflicted. There have also been attempts to correlate prostate tumor or patient characteristics with NE biomarkers (chromogranin-A, neuron specific enolase or bombesin) in serum samples obtained from patients. Here again, several studies have found these serum biomarkers to be useful correlative factors (Kadmon et al., 1991; Tarle et al., 1994; Berruti et al., 2000), while others have not.

With regard to distinction of hormone-refractory prostate cancer with the use of tumor and serum NE biomarkers, however, there is much more agreement in the various studies that assessed NE cells in hormonally-treated tumors and NE biomarkers in patient serums. These studies consistently show that NE tumor and serum biomarkers are upregulated following hormonal therapy of advanced prostate cancer patients (Ito et al., 2001; Isshiki et al., 2002; Ismail et al., 2002; Tarle et al., 200; Hirano et al., 2004), strongly suggesting either that NE cells in the tumor are increased by these kinds of treatments or that the tumor cells are increasingly taking on characteristics of NE cells. Indeed, the latter conclusion is consistent with basic research showing that cultured human prostate cancer cell lines or tumor xenografts can directly undergo the NE transdifferentiation process in response to specific stimuli (characterized by the development of long cellular extensions similar to cultured neuronal cells in addition to increased expression of NE gene products such as chromogranin-A, NSE, synaptophysin and peptide hormones including bombesin and parathyroid like hormone) (Shen et al., 1997; Leung et al., Bang et al., 1994; Deeble et al., 2001). A recent study showing that NE differentiated prostate cancer cells xenografted into one flank of a mouse enables the development of tumors from androgen-dependent prostate cancer cells that are xenografted into the opposing flank implies that NE-differentiated prostate cancer cells might be able to release systemic factors (likely neuropeptide hormones) that support growth of androgen-dependent tumor cells at a distant site (Jin et al., 2004).

This Example shows that siRNAs that silence PCDH-PC expression in hormonally deprived LNCaP cells suppress the ability of these cells to undergo NE transdifferentiation and directly identifies a potential role for PCDH-PC expression in the NE differentiation process experienced by these and other prostate cancer cell lines. Other results show that suppression of the wnt signaling pathway (by dominant negative Tcf or siRNA against β-catenin) effectively blocks NE transdifferentiation of LNCAP cells maintained in androgen free medium or transfected by PCDH-PC also supports that the NE transdifferentiation pathway of prostate cancer cells driven by PCDH-PC expression is dependent upon the ability of PCDH-PC to increase wnt signaling. Aberrant wnt signaling may be considered to be associated with the development of several prominent human cancers such as colon and breast cancer as well as melanoma, and the wnt signaling pathway is also important for many normal differentiation processes including those of the neural crest derivative cells and tissues, bone, muscle and kidney (Lustig et al., 2003; Moon et al., 2002; Hendriks et al., 2003; van Es et al., 2003). The results shown in this Example show that the activation of the wnt signaling pathway via increased PCDH-PC expression in hormonally-deprived prostate cancer cells may significantly alter the biological properties of these cells in a manner that increases their potential for aggressiveness in a treated prostate cancer patient. Analyses of human prostate tumors have already identified the presence of (wnt) activating mutations in β-catenin that are present in a relatively small proportion of the tumors analyzed (Voeller et al., 1998; Chesire et al., 2000). However, clinical studies citing evidence of nuclear β-catenin and increased wnt signaling in aggressive and hormone refractory prostate cancers in humans (de la Taille et al., 2003; Chesire et al., 2002; Chen et al., 2004) also indicate that increased wnt signaling is an important factor in the progression of prostate cancer to end stage disease to an extent that is not accounted for by the small proportion of prostate tumors with mutated β-catenin. Evidence for wnt signaling in advanced prostate cancer is associated with increased PCDH-PC expression in the tumor cells following hormonal therapies. Therapeutic agents that can specifically suppress PCDH-PC expression or wnt signaling activation in prostate cancer cells, as provided for by the invention, may have considerable value in treatment of advanced prostate cancer in humans.

### Example 2

### Overexpression of Protocadherin-PC mRNA in Hormone-Resistant Human Prostate Cancer

The characterization of a novel gene product, protocadherin-PC(PCDH-PC), shows that it is expressed by apoptosis-resistant variants of the human prostate cancer cell line, LNCaP. This Example analyzes whether transfection of the parental LNCaP cells with PCDH-PC induces a state of hormone-resistance. LNCAP cells transfected with PCDH-PC were tested for their ability to form tumor xenografts in castrated male nude mice. The Example also provides characterization of PCDH-PC mRNA expression level and localisation in human prostate and prostate cancer (CaP) tissues. PCDH-PC mRNA expression and its localisation were studied by semi-quantitative RT-PCR and by in situ hybridization (ISH) performed on normal prostate, BPH, untreated CaP, hormone-treated CaP and hormone-resistant CaP.

In contrast to control-transfected cells, PCDH-PC transfected LNCaP cells were able to form tumors in castrated male nude mice. Semi-quantitative RT-PCR procedure demonstrated that normal human prostate cells and tissues expressed little or no PCDH-PC-related mRNA and that this low level of expression was maintained in untreated CaP cells. ISH showed that expression of PCDH-PC-homologous transcripts was restricted to some epithelial cells in normal tissue and to CaP cells in tumors. In contrast, hormone-resistant CaP cells were found to express significantly higher levels of PCDH-PC-related mRNA, by both RT-PCR and ISH analysis. Comparison of PCDH-PC mRNA and androgen receptor mRNA levels in hormone refractory CaP did not show correlation between the overexpression of these two molecules.

Through factors as diverse as increased aging of populations and improved methods of diagnosis, prostate cancer has become a major source of cancer-related morbidity and mortality for men in Western nations (Gittes, 1991; Landis et al., 1999). When detected in the advanced stages, patients with the disease are almost invariably treated by some form of hormonal therapy in an attempt to deplete the levels of endogenous androgenic steroids or to block the ability of these steroids to activate transcription through the androgen receptor (AR) protein (Schultze et al., 1987; Grayhack et al., 1987; Carter and Isaacs, 1990). Androgen-ablation therapy successfully shrinks primary and metastatic lesions of prostate cancer by inducing apoptosis of androgen-dependent prostate cancer cells (Gittes, 1991; Grayhack et al., 1987; Kyprianou et al., 1990; Westin et al., 1995). This therapy, however, is not known to be curative. Rather, a subset of prostate tumor cells are inevitably able to survive in an androgen-deprived environment and these cells provide a repository for the eventual relapse of the tumor in a hormone-resistant form that often shows resistance to more traditional forms of therapy (radiation or chemotherapy) as well.

The molecular mechanisms through which prostate cancer cells acquire resistance to hormonal therapies appear to be complex and diverse. Evidence supports the concept that changes in the androgen-signaling pathway play some role in this process. AR gene mutation and amplification in hormone-resistant prostate cancers suggest that androgen-mediated signaling may be hyperactive in these tumor cells, while cross talk between growth factor receptor and AR signaling pathways and excessive recruitment of AR transcriptional co-activator also have been postulated as mechanisms for its aberrant function (Feldman and Feldman, 2001). Studies of cultured prostate cancer cells and animal tumor xenograft models have also provided evidence that the activation of other cellular signaling pathways, e.g increased mitogen activated protein kinase signaling and receptor tyrosine kinase activation (Craft et al., 1999), can stimulate androgen receptor activity in the absence of ligand in some prostate cancer cells. Alterations in apoptosis-signaling molecules found in hormone resistant prostate cancers suggest that other molecular mechanisms related to apoptosis control might also participate in the transition to androgen independence. Overexpression of the apoptosis-suppressing protein, bcl-2 (Colombel et al., 1992; Miyake et al., 1999; Raffo et al., 1995), increased Akt activation and signaling (Paweletz et al., 2001; Malik et al., 2002), and inactivation of tumor suppressor genes like p53 (Navone et al., 1993; Heidenberg et al., 1995) and ANX7 (Srivastava et al., 2001) have also been shown to increase resistance of prostate cancer cells to hormonal deprivation.

More recently, a comparative genetic analysis of some apoptosis-resistant prostate cancer cell lines has led to the description of a new potential mechanism through which prostate cancer cells might acquire resistance to hormones and other therapeutic agents. Naturally selected derivatives of the human LNCaP cell line that are apoptosis-resistant in vitro and hormone-resistant in vivo were shown to overexpress a novel member of the protocadherin gene family, protocadherin-PC(PCDH-PC) (Chen et al., 2002). PCDH-PC has complete homology with a gene product encoded on the human Y chromosome (previously referred to as PCDHY, at Yp11-2) and has close homology (98.1%) with a gene product (PCDHX) encoded by the human X chromosome (at Xq21-3) (Blanco et al., 2000; Yoshida and Sugano, 1999). Since the area of the Y chromosome containing the PCDHY/PCDH-PC gene lies within a region of the Y chromosome that was acquired by duplication and translocation of a portion of the X-chromosome during human evolution, the PCDH-PC gene product is also distinctly human-specific (Blanco et al., 2000; Yoshida and Sugano, 1999). Aside from occasional nucleotide differences within the coding region, PCDHY/PCDH-PC is also distinguished from PCDHX in that it lacks a small 13 bp continuous sequence that is present in the PCDHX encoded gene (Chen et al., 2002; Blanco et al., 2000; Yoshida and Sugano, 1999). This distinction is important in that the 13 bp region lost from the PCDHY/PCDH-PC gene includes a potential AUG start site. Further analysis of the PCDH-PC transcript expressed in the resistant prostate cancer cells revealed that it would preferentially translate to a protein that lacks a signal sequence as an apparent consequence of the missing 13 bp domain and cellular fractionation of LNCaP cells that express PCDH-PC showed that the protein was cytoplasmic localized, consistent with the lack of a signal sequence (Chen et al., 2002).

While PCDH-PC expression was discovered in experimentally selected apoptosis-resistant prostate cancer cell lines, this gene product confers resistance to apoptosis on prostate cancer cells as shown by a demonstration that LNCaP cells stably transformed with PCDH-PC cDNA were able to better survive an acute exposure to phorbol ester, a condition that induces apoptosis in LNCaP parental cells (Chen et al., 2002). The PCDH-PC peptide sequence also contains a β-catenin binding site localized within its COOH terminus (Chen et al., 2002) expression of PCDH-PC in the apoptosis-resistant variants of LNCaP cells has been shown to be associated with a change in the intracellular localization of β-catenin protein (from the outer membrane of the apoptosis-sensitive parental cell line to the cytoplasm and nucleus of apoptosis-resistant cell lines) as well as increased endogenous transcriptional activity from an LEF-1/TCF promoter element in the apoptosis-resistant variant lines (de la Taille et al., 2003). Based on studies of these LNCaP derivative cell lines, expression of PCDH-PC was shown to induce apoptosis and hormone resistance in prostate cancer cells through the upregulation of β-catenin mediated transcriptional activity similar to effects found when β-catenin activity is modulated during the progression of colon cancer.

To assess whether PCDH-PC expression plays a role in the natural progression of human prostate cancers to the hormone resistant state, This Example provides a survey of primary human tissues, including normal and cancerous specimens of human prostate, to evaluate these parameters. The results of semi-quantitative analysis of PCDH-PC mRNA expression in these tissues are presented and show that the expression of mRNA homologous to the PCDH-PC gene product is closely linked to the acquisition of hormone resistance in human prostate cancer cells. A comparison of expression of PCDH-PC and AR in these same tissues was used to determine whether there is correlation between overexpression and progression to hormone refractory prostate cancer. The results show that PCDH-PC and AR induce prostate cancer progression through two independent mechanisms.

Human Tissues Collection. Human tissues from normal, benign hyperplasic and malignant prostate were obtained from radical prostatectomy specimens or transurethral resections. A representative sample was taken from each tissue for histopathological and immunohistochemical assessment and an adjacent piece was placed in liquid nitrogen for RNA extraction. Five groups of patients were included in this study: Group 1 were patients with normal prostate (obtained from donors, n=15); Group 2 were patients with benign prostate hypertrophy, (BPH; n=15); Group 3 were hormone-naive (untreated) prostate cancer patients (n=13); Group 4 were prostate cancer patients who received 6-month adjuvant hormonal therapy prior to radical prostatectomy (androgen deprivation by luteinizing hormone-releasing hormone (LH-RH) analog or by orchidectomy) (n=9) and; Group 5 were hormone refractory prostate cancer patients (cancer progression despite hormone therapy; n=11). Whole normal prostates were sampled according to McNeal's zonal anatomy (McNeal, 1981). Normal human tissues (brain, kidney, liver, placenta, duodenum, lung, spleen, urothelium and skeletal muscle) were obtained from donors. For in situ hybridization (ISH) and immunohistochemistry (1HC) studies, prostate tissue samples were fixed for 24 hours in formalin and embedded in paraffin. Five ttm sections were collected on Super Frost Plus slides (Knittel Glaser, Germany) and processed for ISH or IHC immediately.

LNCaP Sublines and Xenograft Tumor Tissues. LNCaP parental and apoptosis-resistant LNCaP derivative cells (LNCaP-TR or -SSR) and LNCaP xenograft tumor tissues were prepared as previously described (Chen et al., 2002). Stable transfection of parental LNCaP cells using the 4.8 kbp PCDH-PC cDNA cloned into the pCMV-myc vector (Clontech, Inc., Palo Alto, Calif., USA) or the pCMV-myc (empty vector) alone was accomplished using lipofectin as previously described (Chen et al., 2002). Stable transfectants were selected under G418 and were cloned using a cloning ring strategy. Expression of the 110 kd myc-tagged PCDH-PC protein in the transformed cells was identified by Western blot analysis of protein extracted from pCMV-PCDH-PC-myc transformed cells using a mouse monoclonal anti-myc tag antibody (Clontech, Inc., Palo Alto, Calif.).

Tumor xenografts in Castrated Nude Mouse. 7-week-old nude mice (Harlan Bioproducts for Science, Inc., Indianapolis, Ind.) were castrated via scrotal incision and one week later, groups of these mice (n=8) were subcutaneously implanted with 2×10⁶ control LNCaP transformed with pCMV-myc empty plasmid or with 2×10⁶ PCDH-PC overexpressing LNCaP cells transformed with pCMV-PCDH-PC-myc vector, both mixed with 100 μl of Matrigel. Tumor size was measured weekly and tumor volume was calculated using the formula as previously reported (Taguchi et al., 2000): V=π×H(H²+3a²)/6 where a=(L+W)/4, H=height of tumor determined by caliper measurement, L=length of tumor and W=width of tumor.

Establishment of Primary Cultures. BPH tissue was obtained from men undergoing suprapubic prostatectomy. The histological status of the tissue was checked by an independent pathologist. Prostate tissue washed with phosphate-buffered saline to remove all trace of blood before being into approximately 1 mm³ pieces using forceps and scissors. The diced tissue was then incubated for 20 h at 37° C. in a collagenase solution (300 U/ml). After digestion, epithelial acinar and stromal cells were separated by centrifugation. The epithelial cells were resuspended in KSM medium (Invitrogen, France) supplemented with 2% FCS, 5 ng/ml of EGF and 50 μg/ml of BPE. Stromal cells were resuspended in RPMI 1640 containing 10% FCS. Separated cells were then incubated at 37° C. in 5% CO2. Identity and purity of the separated cultures were confirmed by immunohistochemistry and phase contrast microscopy.

RT-PCR Quantification of PCDH-PC and AR Expression in Cell Cultures and in Human Tissues. RNA was extracted from frozen tissue or cells according to Chirgwin et al. (1979) using 4 M guanidinium thiocyanate and was collected on a cesium chloride cushion. The amount of PCDH-PC-homologous mRNA was determined by semi-quantitative RT-PCR by comparison with an internal control, an ubiquitous transcription factor TBP as previously reported (Gil-Diaz de Medina et al., 1998). The primers sequences for AR, TBP and GADPH are as described by Gil-Diez de Medina et al. (1998). The primers sequences for PCDH-PC were: 5′-AATTGGGTAACTACACCTACTA-3′ (SEQ ID NO:18) (sense primer) and 5′-CTCGAAGGTTGTCACTGGATA-3′ (SEQ ID NO:19) (antisense primer). Twenty-six cycles were used for the co-amplification of PCDH-PC and TBP. After gel electrophoresis, the PCR-amplified products were quantified with a Molecular Dynamics 300 Phosphorlmager (Sunnyvale, Calif., USA). Each measure was repeated in three independent PCR reactions and found to be identical within 15%. No amplification was observed when reverse transcriptase was omitted from the reverse transcription reaction.

Probes and Labeling. A 249 bp PCDH-PC cDNA (Chen et al., 2002) was used as a template to generate by unidirectional PCR a single strand cDNA probe. The sense and antisense probes were obtained by using respectively either PCDH-PC forward or reverse primer. The PCR reaction mix contained a final concentration of 100 ng cDNA, 67 mM KCl, 10 mM Tris-HCl pH 8.8, 10 mM (NH₄)₂SO₄ 0.01% Tween 20, 1.5 mM MgCl₂, 0.1 mM each of dATP, dCTP, dGTP, 0.065 mM dTTP, 0.035 mM 11-digoxigenin dUTP and 1 μM of either forward or reverse primer. Five units of DNA polymerase (Eurobio, France) were added to a final reaction volume of 100 μl and the amplification process was 5 min at 94° C. before 35 cycles with 1 min denaturation at 94° C., 1 min annealing at 55° C. and 1 min extension at 72° C. Digoxygenin labeled probes were purified by 0.1 M NaCl/EtOH precipitation and their specific activity was quantified by dot-blot using anti-digoxigenin as primary antibody and adjusted to a concentration of 0.5 μg/ml.

In situ Hybridization. Five μm paraffinized sections were heated for 30 minutes at 60° C. and deparaffinized by three washes in xylene and rehydrated in increasing ethanol. Sections were incubated for 20 min in 0.2 N HCl at room temperature. After washing with 5 mM MgCl₂/PBS, sections were incubated for 15 min with 0.3% Triton X-100/PBS. Tissues were then digested with 10 μg/ml of proteinase K for 30 min at 37° C. in 20 mM Tris pH 7.4 containing 5 mM EDTA. Inactivation of enzyme was performed with 0.2% glycine/PBS for 10 min. After washing with PBS, tissues were refixed with 4% formaldehyde/PBS for 5 min at room temperature. After 2 washes with PBS, sections were incubated for 15 min at 45° C. with 10 mM DTT/PBS and acetylated for 10 min in 0.25% acetic anhydride diluted in 0.1 M triethanolamine. Slides were rinsed in 2×SSC and prehybridized for 3 hours at 60° C. with hybridization buffer containing 4×SSC, 1× Denhart, Dextran sulfate 10%, 100 μg/ml of salmon sperm DNA, 100 μg/ml tRNA and 50% formamide. Hybridization was carried out by incubation at 60° C. overnight in hybridization buffer supplemented with 5 μg/ml of sense or antisense digoxigenin probe. Slides were washed 30 min at 2×SSC with 50% formamide and 45 min at 42° C. in 20 mM β-mercaptoethanol diluted in 0.1×SSC, respectively. After saturation of non specific binding sites with saturation buffer containing 1% blocking buffer, 2% normal sheep serum diluted in 0.15 M NaCl, 0.1 M maleic acid, pH 7.5, the alkaline phosphatase-labeled antidigoxigenin conjugated antibody (Roche, France) was added, diluted in saturation buffer. After 4 washes, antibody complex was revealed by alkaline phosphatase substrate (nitroblue tetrazolium and 5-bromo-4-chloro-3-indolyl-phosphate in 0.1 M Tris-HCl, 0.1 M NaCl, 0.05 M MgCl2; pH 9.5) containing 1 mM levamisol. Color precipitation development was monitored at room temperature.

Statistical Analysis. The data obtained by RT-PCR was analyzed for statistical significance by using Mann-Whitney U-test. A p value below 0.05 was considered to denote statistical significance.

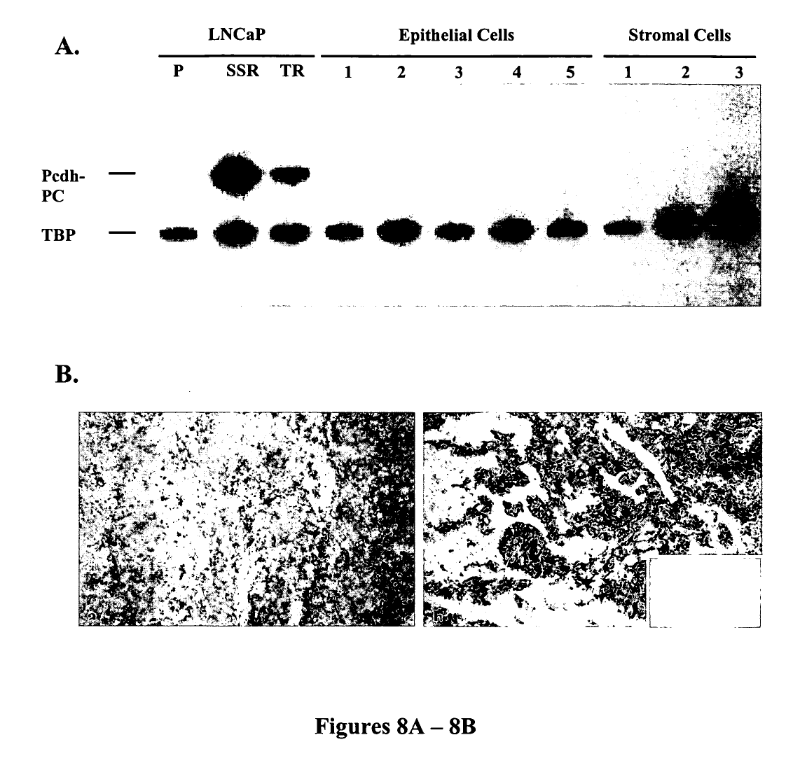

Expression of PCDH-PC mRNA in LNCaP Cell Variants and in Primary Human Prostate-Derived Cell Lines. PCDH-PC was described in apoptosis-resistant variants of the human prostate cancer cell line, LNCaP (Chen et al., 2002). Using a semi-quantitative RT-PCR technique with an internal expression control (TBP mRNA, a ubiquitously expressed transcription factor), relative PCDH-PC mRNA expression was measured in a variety of cultured human prostate cells. As shown in FIG. 8A, results of the assay show that PCDH-PC mRNA levels were much lower in the parental (apoptosis-sensitive) LNCaP cells than in the apoptosis-resistant-TR and -SSR derivatives, confirming results previously obtained by Northern blot analysis of RNAs from these cell types. PCDH-PC mRNA was not detected in any primary cultures of (benign) human prostate cells in the assay, regardless as to whether they were stromal or epithelial in origin.

An in situ hybridization procedure was also used to evaluate the relative expression of PCDH-PC-homologous mRNA in primary xenografts of LNCaP cells (in intact or castrated male immunodeficient mice). Previously an RNase protection assay had shown evidence that PCDH-PC mRNA was dramatically upregulated in LNCaP xenografts during the acquisition of hormone resistance following castration of the host (Chen et al., 2002). Thin sections from individual LNCaP xenograft tumors obtained from intact males or from males at 4 weeks after castration were hybridized, in situ, to digoxygenin-labeled sense or antisense PCDH-PC cDNA probe and hybridization of the probe was detected by an immunohistochemical procedure to detect digoxygenin. As shown in FIG. 8B, the antisense probe was found to hybridize only to human tumor cells in the xenograft and the intensity and distribution of hybridization was found to be significantly greater in the hormone resistant tumors growing at 4 weeks after castration.

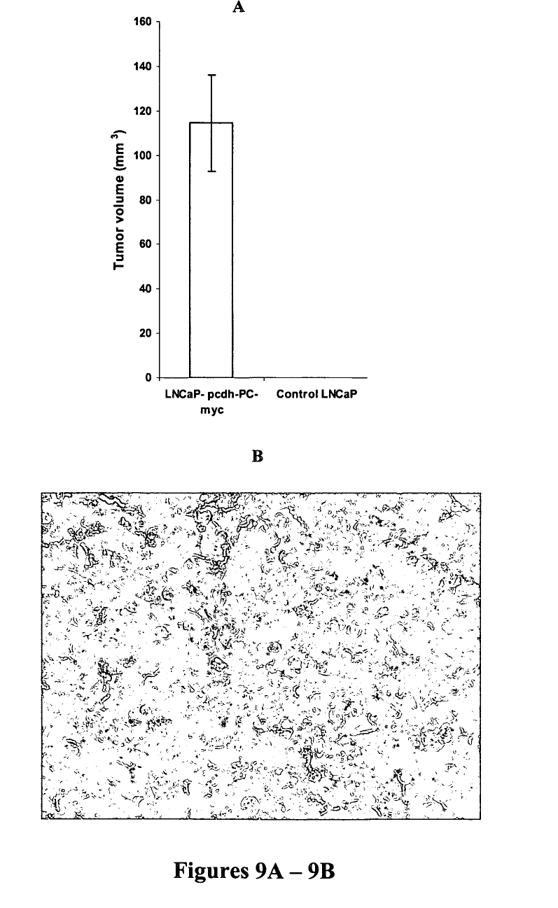

Expression of PCDH-PC enables LNCaP cells to form tumors in castrated male nude mice. A previous report (Chen et al., 2002) showed that LNCaP-TR and LNCaP-SSR cell lines, apoptosis-resistant variants of the parental LNCaP cells that express high levels of PCDH-PC, were hormone resistant based upon their ability to form large tumors in castrated male nude mice, whereas parental LNCaP cells were not able to form tumors in similarly castrated male mice. In order to investigate whether PCDH-PC expression might directly convert parental LNCaP cells to a hormone-resistant state, castrated nude male mice (1 week) were implanted with either control LNCaP cells (transformed with pCMV-myc empty vector) or PCDH-PC-transformed LNCaP cells (transfected with pCMV-PCDH-PC-myc vector; LNCaP-PCDH-PC-myc cells). After 7 weeks, mice injected with control cells had no visible or palpable tumor (0/8) whereas mice receiving PCDH-PC transformed cells all had tumor (8/8) and their average size was 114.6±21.8 (mean ±SEM) mm³. These tumors were extremely vascularized and a photomicrograph of a thin section from one of these tumors is shown in FIG. 9.

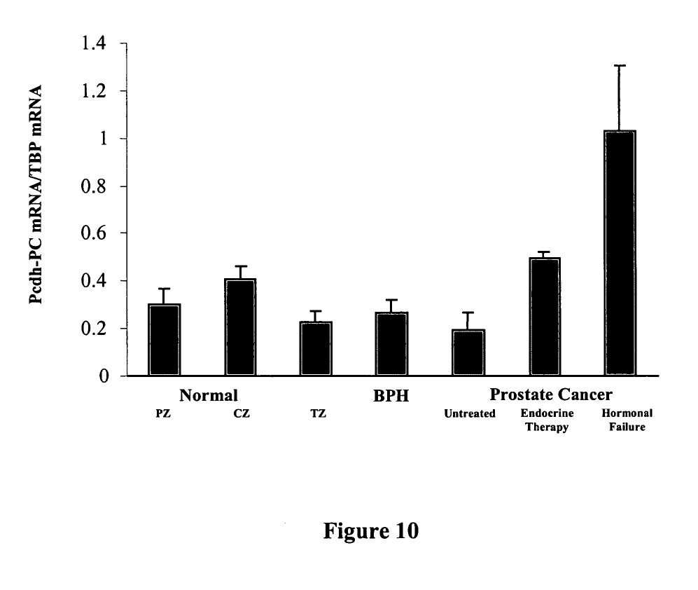

Expression of PCDH-PC-Homologous mRNA in Primary Human Prostate Tissues. The semi-quantitative RT-PCR assay was used to examine expression levels of protocadherin-PC in RNAs extracted from 63 different specimens consisting of normal or diseased (benign and malignant) human prostates. The results of this survey (FIG. 10) showed a low-level expression of PCDH-PC-related mRNA in all normal prostate tissues, regardless as to whether they were derived from the peripheral, central or transitional zones of the prostate (mean relative expression of 0.302±0.169; 0.411±0.119 and 0.231±0.134, respectively). This low level of expression was maintained in several specimens of diseased prostate tissues consisting of BPH or untreated (localized) prostate cancers (0.287±0.131, BPH; 0.196±0.204, untreated cancers). A small number (8) of primary localized prostate tumors obtained from patients who had received 6 months of hormonal therapy prior to their surgery also demonstrated this low mean level expression of PCDH-PC mRNA (0.495±0.656). In contrast, tumors obtained from patients that were experiencing hormonal failure had a mean expression of PCDH-PC mRNA that was significantly greater than any of the other types of tissue or tumor (mean relative expression levels in hormonal failure patients=1.031±0.896 vs 0.307±0.507 for all other tumor specimens; p=0.017).

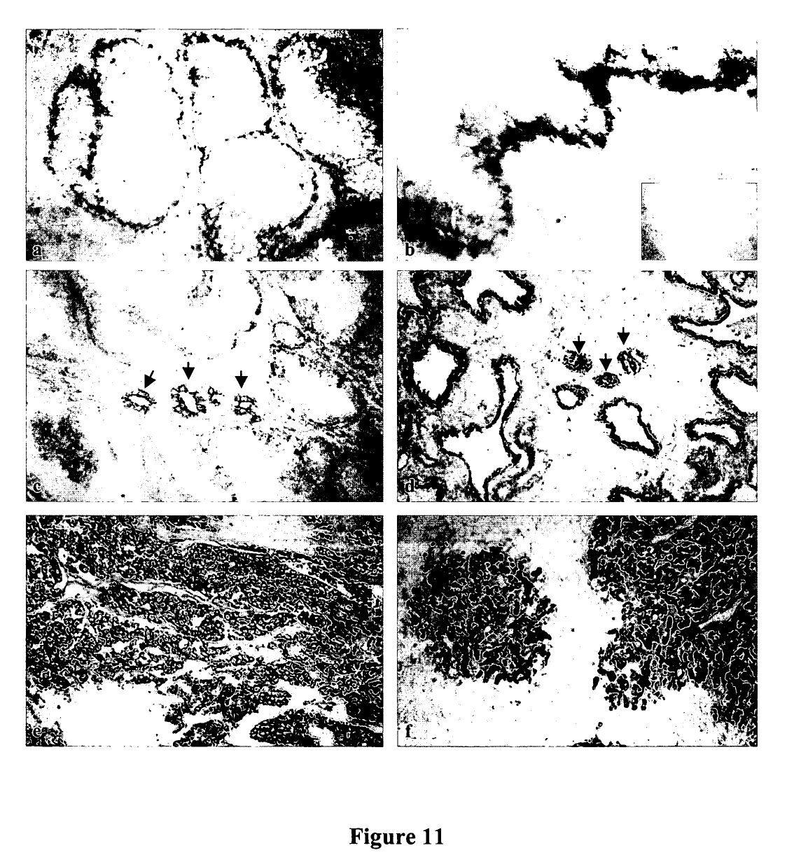

This difference in PCDH-PC mRNA expression was also found when tissue sections from similar groups of patients were analyzed by in situ hybridization procedures to evaluate PCDH-PC expression (FIG. 11). In all prostate tissues analyzed, hybridization of the PCDH-PC antisense probe was mainly localized to epithelial cells, although occasionally endothelial cells and smooth muscle cells appeared to be weakly stained. In the normal prostate tissues, PCDH-PC expression was predominantly found in the basal epithelium with less than 5% of ductal or acinar epithelial cells showing weak hybridization (FIGS. 11A-11B). In regions derived from the central zone of normal prostates, there did appear to be more extensive hybridization with the non-basal epithelium and in some regions up to 48% of the epithelial cells were weakly labeled. For specimens containing BPH, the hybridization pattern was very similar to that found in normal transitional zone epithelium with labeling of basal cells and rare and weak labeling of acinar epithelial cells. In the specimens containing prostate tumors from untreated patients, all tumor cells were found to be positive for hybridization to the PCDH-PC antisense probe and these cells generally had a more intensity of staining when compared with cells in the benign regions of these specimens (FIG. 11C). No difference in staining level was observed in a comparison of the staining of epithelial cells in benign regions directly adjacent to tumors with normal peripheral or transition zone tissues. However, significantly more intense hybridization was observed in the cells of all (localized) tumors obtained from patients with 6 months or more of hormonal therapy prior to surgery as well as in the epithelial cells present in the benign but atrophic glands present in these specimens (FIGS. 11D-11F). These data support the results of the semi-quantitative RT-PCR assay and show that hormonal deprivation induces PCDH-PC-related mRNA expression in both normal (but atrophic) and cancerous prostate epithelial cells, similar to results in cultured and xenograft prostate cancer cells.

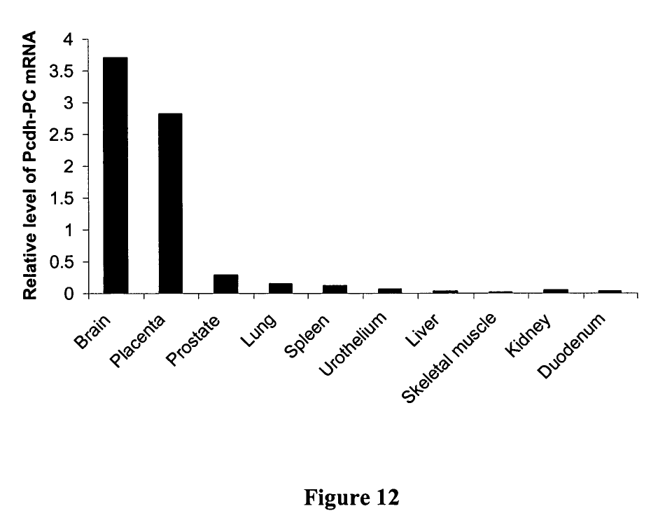

Expression of PCDH-PC-Homologous mRNA in Other Normal Human Tissues. RNAs from a variety of other normal human tissues (brain, liver, lung, spleen, skeletal muscle, duodenum, prostate, urothelium, kidney and placenta) were also evaluated for PCDH-PC expression using the semi-quantitative RT-PCR assay and this was compared to the levels expressed in normal human prostates (FIG. 12). The results of the surveys show that some form of PCDH-PC mRNA is expressed in normal prostate (at a low level) and in human placenta and brain (at much higher levels). All other tissues examined lacked expression of PCDH-PC-related transcripts. Based upon previous finding that the sequence of the PCDH-PC cDNA, cloned from apoptosis-resistant prostate cancer cells, displayed extensive homology (98.1%) with the PCDHX gene product, but that it differed significantly in lacking a contiguous stretch of 13 basepairs (bp) near the translation start site of the potentially encoded polypeptide, RT-PCR was performed on mRNAs isolated from the various human tissues that expressed PCDH-PC in order to more specifically identify whether the expression was from the X— (PCDHX) or Y-linked (PCDH-PC) gene in normal or malignant prostate. Using a set of PCR primers that allow amplification of a small (130 bp) region from within putative exon 4 of the PCDH-PC transcript (containing the site of the 13 bp deletion as defined from the genomic sequence of X-chromosome gene), RT-PCR was used to amplify mRNA extracted from 2 normal human prostate, 2 untreated human prostate tumors, 2 hormone-resistant human prostate tumors and normal human brain and placenta. The PCR amplification product obtained from each of these procedures was directly sequenced and the sequence demonstrated that the brain and placenta expressed a form of PCDHX mRNA that contained the 13 base pair sequence. In contrast, the sequence of the PCR product amplified from the hormone resistant prostate cancer lacked the 13 base pair sequence corresponding to the PCDH-PC-encoded homologue. However, in normal prostate and in untreated prostate tumor, no definite sequence was obtained because of the presence of several PCR amplified products. These results were consistent with observations in apoptosis resistant cell lines showing that the expression of the PCDH-PC homologue (as opposed to the PCDHX homologue) was preferentially expressed in hormone resistant prostate cancer.

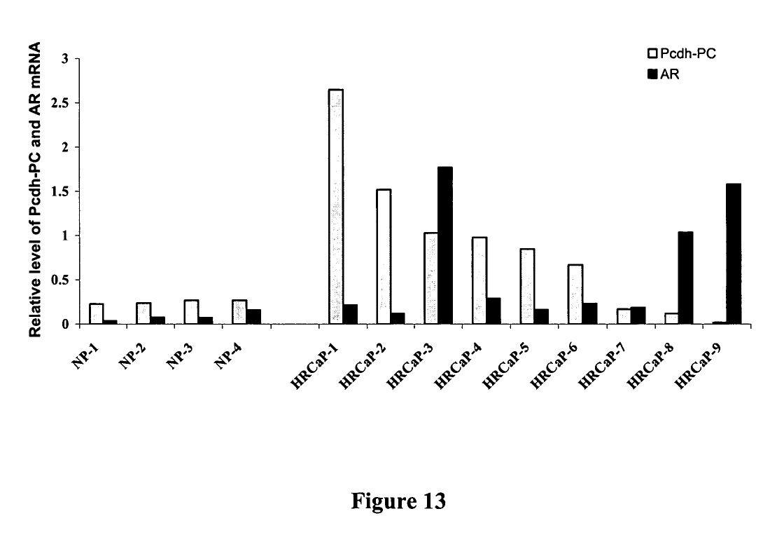

Comparison of PCDH-PC mRNA versus AR mRNA in Hormone-Refractory Human Prostate Cancer. Previous in vitro and xenograft data as well as this Example show that the expression of PCDH-PC emerges during the acquisition of resistance to androgen withdrawal. Numerous reports have described the crucial role of the AR in the development of resistance to hormone therapy of prostate cancer (Feldman and Feldman, 2001). Sometimes resistance to hormonal therapy is associated with increased expression of AR (Visakorpi et al., 1995; Linja et al., 2001; Latil et al., 2001). To evaluate any potential relationship between PCDH-PC and AR expression, semi-quantitative RT-PCR was carried out to compare the relative level of mRNA corresponding to these two molecules in specimens of hormone refractory prostate cancer (HRCaP, n=9). AR mRNA was detected in all HRCaP, however 3 of them (3/9) showed a relative higher mRNA level compared to normal prostate (FIG. 13). PCDH-PC mRNA was also detected in all HRCaP and five of them showed significantly higher mRNA levels compared to normal prostatic samples. There was no correlation between AR and PCDH-PC mRNA expression (p>0.5), except one sample (HRCaP-3) which both displayed high level of AR and PCDH-PC mRNA. The specimens with high expression of PCDH-PC were primarily in specimens expressing low level of AR. Similarly, the apoptosis-resistant variants LNCaP cells which overexpressed PCDH-PC were shown to have less AR mRNA expression than parental LNCaP cells (Chen et al., 2002).

Hormone treatment for advanced prostate cancer, although initially effective, is invariably complicated by the development of hormone resistance. There is experimental evidence to support the concept that some hormone resistant prostate cancer cells might be present in prostate tumors even before therapy is applied and that hormonal therapies might simply select these hormone resistant cells, allowing their eventual expansion (Craft et al., 1999; Isaacs et al., 1987). There is other evidence that suggests that the application of hormonal therapy may enable some prostate cancer cells to acquire hormone resistance through specific genetic changes that occur during adaptation to the low androgen environment of the hormonally-treated patient (Isaacs et.al., 1994; Nupponen et al., 1998; Stubbs et al., 1999). Regardless of whether either one or both of these paradigms are correct, it is likely that the androgen-resistant prostate cancer cell is genetically different from the androgen-sensitive prostate cancer cell and the ability to identify the genetic differences that confer hormone resistance to prostate cancer cells is a prelude to the development of better and more effective therapies for the disease.

The results of the studies presented in this Example support a unique genetic change in prostate cancer cell lines that had acquired resistance to apoptosis following repeated exposure to apoptotic agents (Chen et al., 2002). The loss of apoptosis-sensitivity was attributed to the induced expression of one particular gene product in the apoptosis-resistant cell lines, PCDH-PC, that was not found to be expressed in the apoptosis-sensitive parental cell line (LNCAP) from which they were selected. The sequence of the cDNA encoding PCDH-PC showed one long open reading frame and analysis of the polypeptide that would be encoded by this reading frame showed that it was an unusual member of the cadherin gene family, having features of both proto- and classical cadherins subtypes (Chen et al., 2002). Moreover, both structural and experimental analysis showed that the PCDH-PC protein expressed in the apoptosis-resistant prostate cancer cells lacks potential membrane attachment (due to the lack of a signal sequence within the translated protein) and it was abundantly expressed in the cytoplasm of apoptosis-/hormone-resistant LNCaP cells. PCDH-PC had been previously described as a unique gene product encoded by the human Y chromosome (PCDHY) and it is believed to have arisen as a result of a duplication and translocation of a gene (PCDHX) from the X chromosome (at Xq21-3). Whereas the PCDHY/PCDH-PC encoded protein lacks a signal sequence, the protein encoded by the X-chromosome homologue has a small but critical extra 13 bp sequence in its coding region that would translate to a protein with a functional signal sequence, thus the homologous gene product on the X chromosome would likely be membrane-localized as other protocadherins. Expression of PCDH-PC was associated with a redistribution of (wild-type) α-catenin protein from the membrane to the cytoplasm and nucleus of LNCaP cells as well as with a significant increase in the endogenous transcriptional activity from a β-catenin-specific promoter element (de la Taille et al., 2003). The coincidence of cytoplasmic PCDH-PC expression in conjunction with dysregulation of β-catenin activity may explain the basis for acquired apoptosis- (and hormone-) resistance in these variant LNCaP cell lines.

Studies carried out in this Example tested whether transfection of parental LNCaP cells, long known to have an androgen-sensitive phenotype with regards to their inability to form tumor xenografts in castrated male nude mice, might gain a hormone-resistant phenotype following transfection with PCDH-PC. Indeed, PCDH-PC transformed LNCaP cells readily formed tumors in castrated male nude mice in contrast to LNCaP cells transfected with an empty expression vector, thus directly demonstrating that PCDH-PC transfection not only confers an apoptosis-resistant phenotype (Chen et al., 2002) but also a hormone-resistant phenotype. Additional presented in this Example is a survey of normal and cancerous human prostate tissues to determine whether PCDH-PC expression is associated with hormone-resistance and also to determine where PCDH-PC might be expressed in these tumors. Semi-quantitative comparative analyses of prostate tissues suggest that PCDH-PC expression was low in normal prostate and in nontreated and early-treatment cancers whereas it was significantly higher in hormone-resistant prostate cancers. In situ hybridization showed that expression of the PCDH-PC-homologous transcripts was restricted to basal cells and occasional acinar epithelial cells in normal prostate tissue and to prostate cancer cells in tumor. Significantly more intense hybridization was observed in tumor cells derived from hormone-treated patients and in tumors from patients failing hormone therapy. These results show that PCDH-PC mRNA expression is acquired by tumor cells after hormonal deprivation and this is consistent with observations that LNCaP cells cultured in androgen-free medium upregulate PCDH-PC expression. Because of the extensive homology between the PCDH-PC and PCDHX gene products, most of the assays for PCDH-PC expression applied to the tissues and tumors would likely detect expression of either homologue. However, sequence analysis of PCR-amplified transcripts from hormone-resistant prostate tumors definitively showed that it was the PCDH-PC-specific transcript that was amplified from these tissues, whereas amplification of homologous transcripts from normal brain selectively detected the PCDHX homologue.

The hormone-resistant prostate tumors that were used in this study were also immunohistochemically surveyed for β-catenin. These tumors showed evidence for abnormal distribution of β-catenin within the cytoplasm and/or nucleus of the tumor cells (de la Taille et al., 2003). This abnormal distribution was rare in the small number of untreated prostate cancers examined. Immunohistochemical analysis of these specimens showed that β-catenin was almost always restricted to the membranes of the untreated cancer cells. The inability to detect any mutations in the β-catenin molecule expressed in the hormone-resistant cancer cells, suggests that β-catenin dysregulation found in the hormone-resistant prostate cancer cells might be the consequence of increasing expression of protocadherin-PC. Abnormal localization of β-catenin had been suggested to contribute to T cell factor (TCF) and androgen receptor signaling activation in prostate cancer (Cheshire and Isaacs, 2003). It is well established that the formation of nuclear β-catenin/TCF plays a pivotal role in the activation of Wnt target genes such as c-myc and cyclin D1. Moreover, β-catenin can interact with the androgen receptor and activate transcription in a ligand-dependent fashion (Truica et al., 2000). The androgen receptor was also shown to compete with TCF for β-catenin (Cheshire and Isaacs, 2003; Mulholland et al., 2003; Yang et al., 2002; Song et al., 2003). Significant correlation was not detected between the overexpression of the AR mRNA and PCDH-PC mRNA in hormone resistant prostate cancer. However, high level of PCDH-PC mRNA was mainly found in patients expressing markedly low level of AR mRNA. Several mechanisms have been postulated to explain the resistance of prostate cancer cells to hormone therapy including mutation/amplification of AR; alterations in the balance between coactivators and corepressors resulting in its activation and mechanisms independent of AR pathways (Feldman and Feldman, 2001). Here, the results point out a potential role of the PCDH-PC during prostate cancer progression without AR upregulated expression. Based on the data presented here, PCDH-PC could participate in the β-catenin cross talk between AR and TCF. Then, either the PCDH-PC could potentiate AR transcriptional activity via β-catenin regulation in presence of low basal level of AR or conversely it could be strictly linked to upregulate of the β-catenin-related transcription (CRT).

### Example 3

### Protocadherin-PC(PCDH-PC) Influences the Akt/Protein Kinase B Cell Signaling Pathway that Regulates Survival of Prostate Cancer Cells

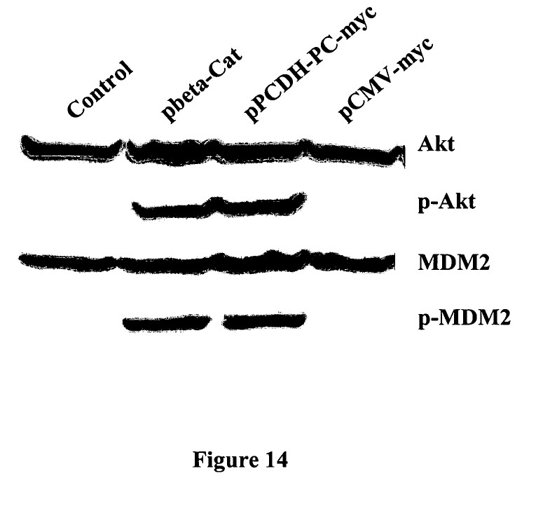

Akt/Protein Kinase B is a serine/threonine kinase protein that lies within the Phosphotidyl-Inositol 3-Kinase (PI3-Kinase) cellular signaling pathway that is responsive to insulin-like growth factor stimulation. Stimulation of PI3-Kinase results in phosphorylation of Akt, activating its ability to phosphorylate several other proteins downstream in this signaling pathway (such as MDM2, Forkhead transcription factor, caspase 9 and bad) that are important regulators of cellular responsiveness to apoptotic stimuli. Highly phosphorylated Akt often corresponds with a cell that is resistant to apoptosis and more likely to undergo proliferation. Indeed, there is increasing evidence that increased Akt phosphorylation is a biomarker of the most aggressive forms of human prostate cancer (Paweletz et al., 2001; Malik et al., 2002; Ayala et al., 2004; Assikis et al., 2004; Kreisberg et al., 2004) and there are ongoing efforts to develop inhibitors of Akt phosphorylation or inhibitors of phosphorylated Akt action to treat advanced (hormone-resistant) prostate cancers. FIG. 14 shows that the expression of protocadherin-PC (PCDH-PC) is associated increased aggressiveness of prostate cancer. As shown in FIG. 14, transfection of a human prostate cancer cell line (LNCaP) with a PCDH-PC expression vector increases phosphorylation of Akt protein as well as a critical downstream target of activated Akt, MDM2. PCDH-PC may stimulate cellular wnt signaling mediated by increased transcription from the beta-catenin/TCF heterodimeric transcription factor. Wnt signaling can be increased either by transfection with PCDH-PC or by transfection with a mutated beta-catenin. Also shown in FIG. 14 is that transfection of LNCaP cells with mutated beta-catenin also upregulates Akt and MDM2 phosphorylation and this supports that the action pathway of PCDH-PC is as follows:
PCDH-PC→wnt(β-catenin/TCF transcription)→Akt phosphorylation→MDM2 phosphorylation

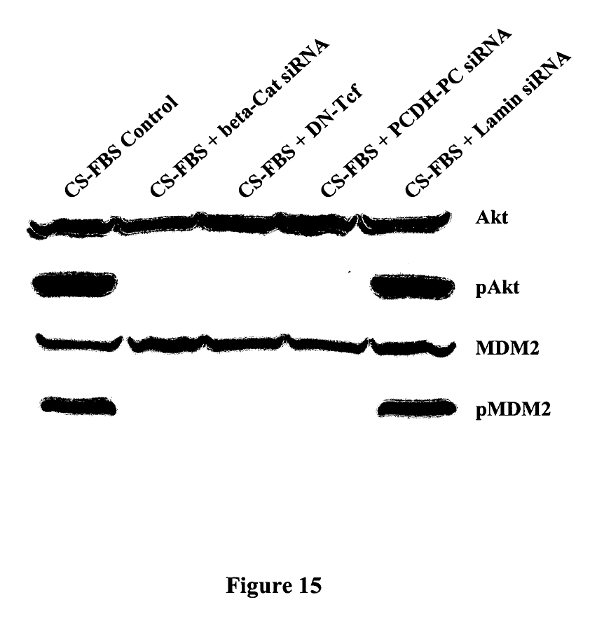

An additional way to evaluate the effects of PCDH-PC on Akt phosphorylation is to analyze prostate cancer cells grown in androgen-free medium, a condition that upregulates expression of PCDH-PC (Chen et al., 2002) and upregulates Akt phosphorylation. In a second experiment (FIG. 15), LNCaP cells were cultured in androgen-free medium (CS-FBS) for 5 days and then transfected these cells for an additional 2 days with siRNA that targets PCDH-PC (#181; SEQ ID NO:4; FIG. 29). The Western blot results of FIG. 15 shows that the levels of pAkt are reduced by the PCDH-PC siRNA, as well as by an siRNA against beta-catenin or by a dominant negative Tcf vector. The latter results show that PCDH-PC is acting through the wnt signaling pathway to induce Akt phosphorylation.

### Example 4

### Protocadherin-PC(PCDH-PC) Regulates Androgen Receptor (AR) Expression in Prostate Cancer Cells through Activation of the Wnt Signaling Pathway

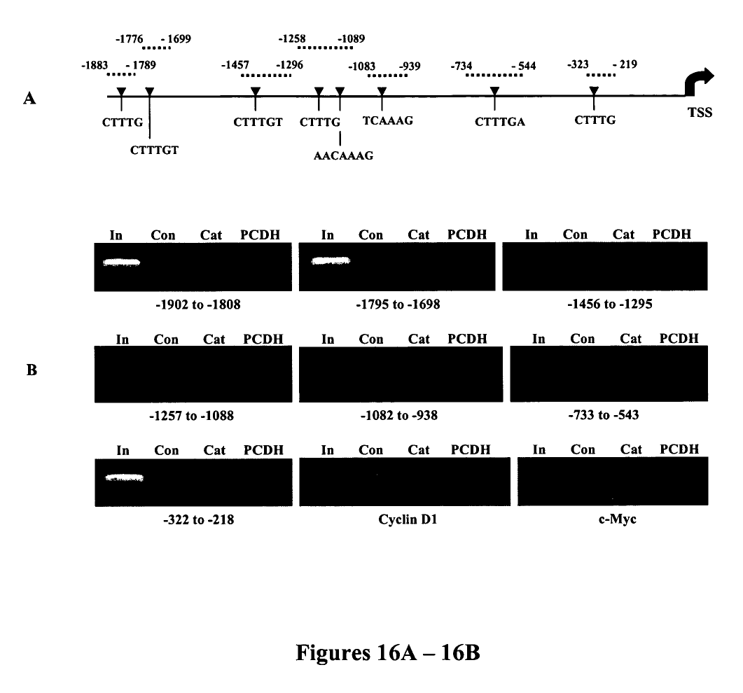

Androgenic steroids drive prostate cancer development and progression. These steroids act by means of a nuclear receptor protein referred to as the androgen receptor (AR). There is a great deal of interest in the role of the AR in prostate cancer, especially with regards to its involvement in the development of hormone refractory disease. Evidence suggests that AR expression is increased in hormone-resistant prostate cancers. PCDH-PC has been shown to regulate androgen receptor expression in prostate cancer cells. The evidence includes a detailed dissection of the promoter of the human androgen receptor gene in which three apparently functional Tcf binding sites were identified within the 2 kilobasepair region of DNA immediately upstream of the transcription start site of human AR. Data includes a completed chromatin immunoprecipitation assay in which show that antibodies to β-catenin protein were able to immunoprecipitate three small regions of the human AR promoter, each containing Tcf binding sites starting from fixed, fragmented chromatin extracted from PCDH-PC or β-catenin transfected human prostate cancer cells (LNCaP, FIG. 16).

Since it has been shown that PCDH-PC upregulates wnt signaling in human prostate cancer cells, the chromatin immunoprecipitation assay results show that PCDH-PC expression should correlate with higher levels of androgen receptor mRNA in prostate cancer cells. Human prostate cancer (LNCaP) cells transfected with an empty vector (negative control) or with an expression vector containing PCDH-PC cDNA for 48 hrs, then RNA was extracted from these cells and assayed for AR mRNA expression using a real-time PCR technique. The results of this experiment showed that AR mRNA was expressed approximately 20-fold higher in LNCaP cells transfected with PCDH-PC compared to LNCaP cells transfected with the empty vector. A comparison was made between AR mRNA expression in LNCaP cells grown in androgen-free medium (a condition that upregulates expression of PCDH-PC) and AR mRNA expression in LNCaP cells grown in normal medium (that do not express PCDH-PC). The comparison shows an approximate 16-fold upregulation of AR mRNA in the androgen-free LNCaP cells. These results show that PCDH-PC, by activating wnt signaling, leads to upregulation of AR mRNA expression. This finding suggests that PCDH-PC expression in hormone-resistant prostate cancers may lead to upregulation of AR and play a role in the pathogenesis of aggressive, late stage disease.

### Example 5

### Targeted Elimination of PCDH-PC Expression for Control of Hormone-Resistant Prostate Cancer

Androgen-sensitive prostate cancer cells become dependent upon the expression and activity of an unusual male gene product, protocadherin-PC(PCDH-PC) when they are deprived of androgens. The invention provides for a combination of androgen-deprivation therapy accompanied by a gene-specific PCDH-PC knockout therapy which would significantly increase the kill rate of prostate tumor cells (compared to androgen-deprivation therapy alone) and provide a means to control hormone-resistant prostate cancer in patients with this disease. The PCDH-PC gene product offers a unique target for gene suppression in clinical therapy of prostate cancer patients: 1) it is a male-specific gene product (encoded on the human Y-chromosome) and obviously, women survive just fine without it; 2) a preliminary survey (See Example 2) (using RT-PCR and in situ hybridization technologies) of human tissues indicates it is expressed mainly in (male) brain, placenta and in scattered basal cells (likely neuroendocrine cells) of the normal prostate; gene targeting agents that do not cross the brain-barrier are a therapeutic advantage because they avoid complications in other tissues.

Cultured human prostate cancer cells and animal (mouse-based) models of prostate cancer were used where the development and growth of hormone resistant human prostate tumor xenografts was prevented using a treatment strategy that combines castration with PCDH-PC suppression. Demonstrating efficacy of combined androgen-deprivation with PCDH-PC knockout therapy in these models would lead to subsequent development of effective means to suppress PCDH-PC expression in humans. This Example includes the development and testing of useful first-generation therapeutic agents, such as antisense oligonucleotides that target PCDH-PC. Antisense oligonucleotides (ASOs), while having some general drawbacks for gene-specific therapeutics, also offer many unique aspects that make them more likely to be rapidly translated into clinical trials in humans with prostate cancer: 1) they are simple defined chemical agents can be synthesized in bulk under highly controlled (good clinical practice) conditions; 2) they can be delivered to patients systemically in controlled doses, making it more likely that they can even reach distal metastases; 3) they are not known to have potential for genetic damage, as with other biological agents (viruses) that are being developed and tested for gene therapy strategies and; 4) gene-targeting ASO agents are already in clinical trials for several different cancers (including prostate cancer), thus there already is a body of literature regarding their use in humans. This Example includes testing of an experimental treatment paradigm that could be used in human prostate cancer patients to suppress hormone-resistant prostate cancer as well as development of potential first generation therapeutic reagents that could be used as therapeutics.

The American Cancer Society estimates of 2005 cancer trends for the U.S. were released Jan. 18, 2005 (American Cancer Society website www.cancer.org, Cancer Facts and Figures 2005). According to the estimates, over 30,000 men will die of prostate cancer this year and this number is not significantly different from their 2004 projection. Virtually all of these deaths from prostate cancer will occur in men with hormone-resistant (androgen-independent) disease. While a new combination chemotherapeutic drug regimen has been reported to extend survival of men with hormone-resistant prostate cancer (Petrylak et al., 2004), the survival advantage conferred by this new, toxic treatment regimen is only a matter of two months. Although this establishes a new standard for the treatment of the hormone-resistant prostate cancer patient, if significant progress is to be made towards reducing overall deaths from this disease while preserving the quality of life for men that have it, better, less toxic means must be identified for targeting the androgen-independent prostate cancer cell for elimination from the body of the hormone-resistant prostate cancer patient.

This invention provides for a gene product that is selectively expressed by androgen-independent prostate cancer, protocadherin-PC(PCDH-PC), and that might play a role in the development of a therapeutic protocol that targets androgen-independent prostate cancer cells for death and elimination. Studies that show that the expression of this unusual male-specific member of the cadherin gene family (encoded on the human Y-chromosome) is selectively upregulated in cultured human prostate cancer cells when they are selected for apoptosis-resistance or when they are exposed to androgen-free conditions (in vitro and in vivo) (Chen et al., 2002). Direct transfection of androgen-sensitive human prostate cancer cells (LNCaP) with PCDH-PC confers apoptosis- and hormone-resistance on them with respect to their ability to form tumors in castrated male nude mice (Chen et al., 2002; Quieres et al., 2005). A survey of human prostate tumor specimens shows that PCDH-PC is highly upregulated in androgen-resistant human prostate tumor cells (Quieres et al., 2005).

Studies show that the upregulation of PCDH-PC in prostate cancer cells induces the activity of a unique cell signaling pathway, wnt, that is also known to become highly active during the development of aggressive colon, oral, and skin (melanoma) cancers in humans (LoMuzio, 2001; Bright-Thomas and Haargest, 2003; Kikuchi, 2003; Brown, 2001; Polakis et al., 1999; Morin, 2003; Lustig and Behrens, 2003). Since the activity of the canonical wnt signaling pathway is associated with the development of apoptosis resistance (Chen et al., 2001; You et al., 2002), the effects of PCDH-PC on prostate cancer may be mediated through this signaling pathway. By activating wnt signaling in prostate cancer cells, PCDH-PC expression drives prostate cancer cells to acquire neuroendocrine- (NE-) cell-like properties (Yang et al., 2005) associated with the synthesis and release of NE hormones that help prostate cancer cells to grow in an androgen-independent state (Shen et al., 1997; Evangelou et al., 2004).

Reagents (for example, siRNAs) have been developed that selectively target and suppress PCDH-PC expression in cultured prostate cancer cells and studies show that these siRNAs strongly suppress the induction of wnt signaling in androgen-deprived prostate cancer cell as well as suppress their transdifferentiation to NE-like cells (Yang et al., 2005). These siRNA targeting agents selectively induce death of androgen-deprived prostate cancer cells. Androgen-deprivation switches prostate cancer cells from a state in which they were dependent upon androgen signaling for survival to a state in which they become dependent upon wnt signaling (via PCDH-PC expression) for survival. By blocking both of these signaling pathways at the same time (using androgen deprivation combined with suppression of PCDH-PC signaling), prostate cancer cells can be selectively targeted for death using a treatment paradigm (castration combined with antisense oligonucleotide therapy) that offers the potential for relatively low toxicity to the patient.

Animal models can be used by directly introducing recombinant DNA expression vectors (expressing shRNA targeting PCDH-PC) into cultured prostate cancer cells prior to their xenografting into mice. The invention provides for effective PCDH-PC targeting strategies based on Antisense Oligonucleotides (ASOs) or siRNA, for example, which could be rapidly developed and tested. ASOs are small (20-mer) deoxy-oligonucleotides with a sequence complementary to the mRNA of the target gene (Crooke, 1993; Stein and Cheng, 1993; Hawley and Gibson, 1996; Crooke, 2003; Kalota et al., 2004; Orr et al., 2005). While unmodified ASOs can be as sensitive to degradation as RNA, the invention provides for chemical modification of the phosphodiester backbones that can make them resistant to degradative action of nucleases in in vivo situations (Crooke, 1993; Stein and Cheng, 1993; Hawley and Gibson, 1996; Crooke, 2003; Kalota et al., 2004; Orr et al., 2005; Monia et al., 1996). There are already several ASO gene targeting strategies being tested for prostate cancers and new modification of the ASO backbone may improve their uptake into cells when injected into living animals (Shoji and Nakashima, 2004).

Androgen-ablation therapy (simple castration) combined with PCDH-PC gene knockout (via PCDH-PC shRNA expression or ASO therapy) has suppressive and regressive effects on androgen-sensitive human prostate cancer cells in an immunodeficient mouse xenograft system. This Example provides exemplary ASOs that strongly and selectively suppress PCDH-PC expression.

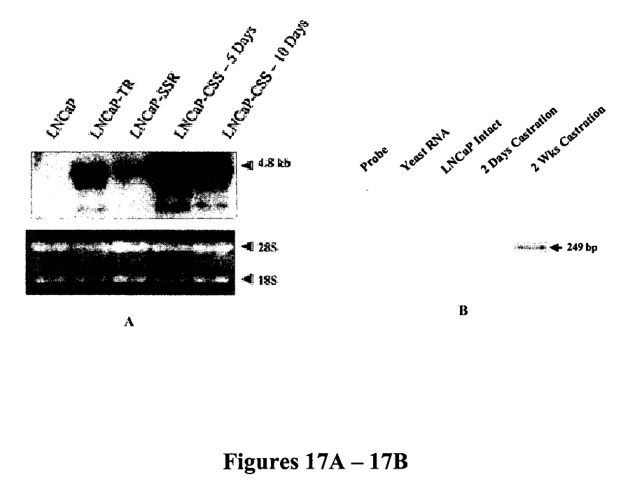

To identify novel gene products associated with the development of apoptosis-resistance by prostate cancer cells, subtractive hybridization-PCR technique was used to compare genes expressed in apoptosis- and hormone-sensitive LNCAP cells to apoptosis- and hormone-resistant variants developed in our lab (LNCaP-TR and LNCaP-SSR) (Chen et al., 2002). As a result of this comparison, one gene product, initially referred to as T6 (now referred to as PCDH-PC), was highly expressed in the resistant LNCaP cells compared to parental LNCaP cells (FIG. 17). Moreover, this gene product was highly expressed when parental LNCAP cells were cultured in androgen-deprived medium or when nude mouse hosts for LNCAP tumor xenografts were castrated (FIG. 17) (Chen et al., 2002).

The complete sequence of the 4.8 kb cDNA showed that it was a novel member of the protocadherin gene family, protocadherin-PC(PCDH-PC). The gene is unusual in several respects: 1) it is male-specific (encoded by the human Y-chromosome); 2) it is human-specific in that it was duplicated from a homologue on the X-chromosome that was translocated to the Y chromosome during evolution from higher primates to humans; 3) it differs from the X-homologue in that a small 13 bp region (present in the X-homologue) was deleted during the translocation and this deletion results in a transcript that preferentially translates to a protein lacking a signal sequence (Chen et al., 2002; Blanco et al., 2000), thus unlike other proto-cadherin gene family members, the protein encoded by PCDH-PC is cytoplasmic instead of membrane-bound; and 4) the protein encoded by this gene has a domain in its C-terminal region homologous to the β-catenin binding domains of classical cadherins (Chen et al., 2002). Studies show that β-catenin protein co-immunoprecipitates with PCDH-PC, indicating that these 2 proteins are binding partners (Chen et al., 2002). Cells that express PCDH-PC have abnormal nuclear accumulation of β-catenin (Chen et al., 2002). Since β-catenin is a molecule involved in the activation of wnt signaling (it complexes with TCF to enable TCF-dependent transcription of genes such as c-myc and cyclin D) (Van Noort and Clevers, 2002; Hecht and Kemler, 2000), and because of the unusual cytoplasmic nature of PCDH-PC, studies also determined whether LNCaP or other prostate cancer cells upregulate wnt signaling when they express PCDH-PC. Results showed that the hormone-resistant LNCaP derivatives that express PCDH-PC have abnormal accumulation of β-catenin protein in their cytoplasm and nucleus and that these cells have elevated expression of TCF/LEF-1 promoted genes (Chen et al., 2002; Lo Muzio, 2001). Studies were also carried out to determine whether transfection of prostate and other cancer cells with a PCDH-PC expression vector would induce wnt signaling (Bright-Thomas and Harrgest, 2003) and, as shown in FIG. 1, it strongly increases nuclear β-catenin accumulation and TCF-mediated gene expression in prostate and colon cancer cells. Additionally, culture of LNCaP cells in androgen-free medium, a condition that upregulates PCDH-PC expression is associated with upregulation of wnt-signaling (FIG. 2). Also, unlike control transfected (empty vector) LNCaP cells, which were unable to form tumors in castrated male nude mice (0/8 tumors formed in 6 wks), all castrated male nude mice developed tumors (8/8 in 6 wks) when they were subcutaneously injected with LNCaP-PCDH-PC cells (Quieres et al., 2005). Since wnt signaling is associated with increased resistance to apoptosis (Kikuchi, 2003; Brown, 2001), the ability of PCDH-PC expression to upregulate this signaling pathway may be a mechanism by which PCDH-PC exerts its effects.

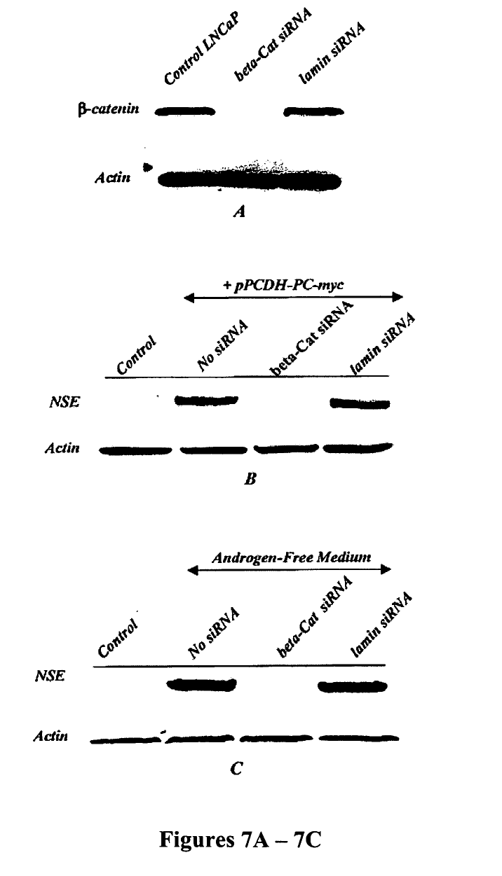

A wnt-signaling pathway targeted cDNA microarray assay was used to identify whether TCF-target genes (such as c-myc, cyclin D3 and COX-2) were upregulated by PCDH-PC transfection and results showed that the changes in gene expression in LNCaP cells induced by PCDH-PC expression were almost equivalent to those induced by transfection with stabilized β-catenin (Bright-Thomas and Haargest, 2003). Results additionally show that PCDH-PC expression is also associated with a unique transdifferentiation process in which prostate cancer cells acquire characteristics of neuroendocrine (NE) cells (Yang et al., 2005). This is highly relevant to the biology of advanced and hormone-resistant prostate cancer since the NE transdifferentiation process of prostate cancer cells is induced by hormone withdrawal (Shen et al., 1997; Evangelou et al., 2004) and it was also shown that growth of a NE-differentiated prostate cancer in one flank of a (immunodeficient) castrated male mouse enabled growth of an androgen-dependent prostate tumor xenograft in the opposing flank (Jin et al., 2004), suggesting that the numerous neuropeptide hormones secreted by NE transdifferentiated prostate cancers (such as bombesin, calcitonin and parathyroid hormone-related protein) (Abrahamsson, 1999; Hansson and Abrahamsson, 2001; Aprikian et al., 1998; Abrahamsson et al., 2000; Tovar-Sepulveda and Falzon, 2003) might be systemically over-riding androgen-regulated growth signaling in hormone-dependent prostate tumor cells. The data showing the link between PCDH-PC expression and NE transdifferentiation of PCa includes a study showing that several culture conditions (growth in androgen-free medium or in medium supplemented with dibutyral cyclic AMP, IL-6 or NS-398) that induce NE transdifferentiation (of LNCaP cells) are accompanied by upregulation of PCDH-PC (Yang et al., 2005) as well as direct evidence that transfection of LNCaP cells with PCDH-PC induces a NE-phenotype identified by upregulation of NE biomarkers (neuron specific enolase and chromogranin A expression) and morphological transition to a neuron-like cell in culture (Yang et al., 2005). The NE transdifferentiation process induced by PCDH-PC expression in prostate cancer cells is driven by activation of the wnt signaling process since it also can be blocked by dominant-negative TCF (Yang et al., 2005) or by an siRNA that selectively suppresses β-catenin expression (FIG. 7 and Yang et al., 2005).

Three different siRNAs have been designed and tested to silence PCDH-PC expression (Yang et al., 2005; and Example 1 above). Design of these siRNAs avoided the cadherin boxes and the transmembrane domain. When co-transfected into LNCaP cells with a plasmid that expresses a myc-tagged PCDH-PC, all 3 siRNAs strongly suppressed expression of PCDHPC-encoded protein, without affecting expression of β-actin or E-cadherin (FIG. 4). When these siRNAs were transfected into LNCaP cells that are grown in an androgen-free medium, they strongly suppress upregulation of PCDH-PC mRNA and activation of wnt signaling (FIG. 5). The data presented in FIGS. 4 and 5 also show that these siRNAs suppress NE transdifferentiation of LNCaP cells (shown by suppression of NSE expression), whether it was associated with direct transfection by PCDH-PC or by growth in androgen-free medium (FIGS. 4 and 5). Based upon these observations, the following sequence of events may be associated with androgen-deprivation of prostate cancer cells:

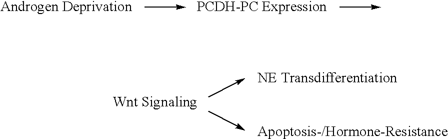

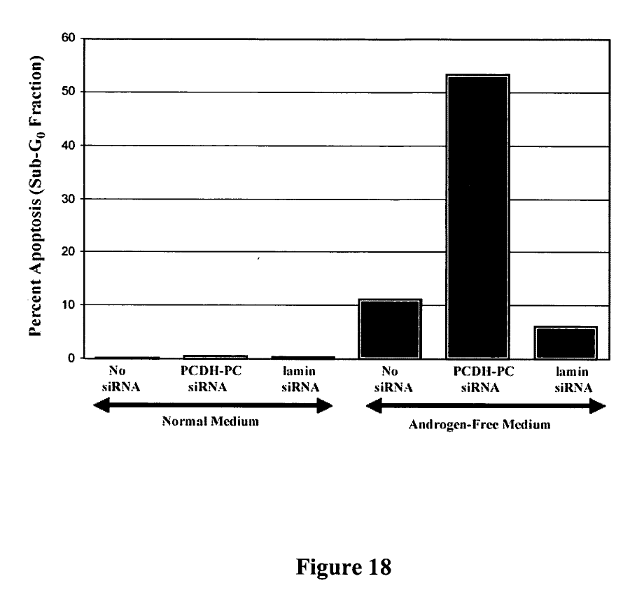

The PCDH-PC-mediated upregulation of wnt signaling in LNCaP cells in androgen-free medium may be substituting for androgen-signaling as a survival factor in these cells. If this is the case, then suppression of PCDH-PC expression in androgen-deprived LNCAP cells should kill these cells. Experimental results in FIG. 18 show (by flow cytometric measurement of the sub-Go peak) that a PCDH-PC-specific siRNA selectively induces cell death of androgen-deprived LNCaP cells.

These results show that culture of LNCaP cells in androgen-free medium for 7 days is associated with an increase in apoptosis compared to control medium, however the PCDH-PC siRNA induces greater than 4× more cell death (58% dead cells) than comparable untransfected cells or cells transfected with lamin siRNA. The ability of PCDH-PC siRNA to induce cell death is specific to cells grown in androgen free medium, not in normal medium.

Exposure of androgen-sensitive human prostate cancer cells to an androgen-deprived environment switches them from a state where they were dependent upon androgen signaling for their survival to a state in which they become dependent upon PCDH-PC-mediated wnt signaling for survival. The invention provides an experimental therapeutic strategy that combines androgen deprivation with suppression of PCDH-PC expression (for example, via shRNA or ASO targeting strategies) to suppress the development of androgen-independent tumor growth and induce tumor regression in immune-deficient mouse/human prostate cancer xenograft model systems. The invention provides for methods to specifically suppress PCDH-PC expression (such as shRNA expression vectors and ASOs) in androgen-sensitive human prostate cancer cells and to selectively induce death of the tumor cells under androgen-deprived conditions. These gene-targeting agents can be tested in preclinical animal prostate cancer models (human prostate tumor cell xenografts grown in immune deficient mice) to show the feasibility of combined PCDH-PC gene knockdown with castration therapy as an approach to newly diagnosed advanced (metastatic) prostate cancer or PCDH-PC knockdown for hormone-resistant prostate cancer.

Using the nucleotide sequence of our PCDH-PC siRNAs, the invention provides for shRNA expression plasmids that will be constitutively expressed in transfected LNCaP cells and that can be transfected into LNCAP cells and select and expand clones in which PCDH-PC expression is suppressed when the cells are grown in androgen-free medium. The invention provides PCDH-PC-specific phosphothio-modified Antisense Oligonucleotides (ASOs) that strongly suppress PCDH-PC expression in treated LNCAP cells transfected with a PCDH-PC expression vector or grown in androgen-free medium.

The invention provides for in vitro pre-clinical testing to show the extent that PCDH-PC-targeting shRNAs and ASOs induce death of LNCAP cells when they are cultured in androgen-free medium.

The invention provides for in vivo pre-clinical testing to demonstrate that suppression of PCDH-PC expression has clinical impact when combined with androgen-deprivation for the treatment of prostate cancer. This pre-clinical testing will consist of 3 types of experiments: 1) PCDH-PC shRNA transfected LNCaP tumors implanted into intact male nude mice will be tested to determine whether they experience a more profound tumor regression and prolonged response to castration when compared to control LNCaP tumors; 2) LNCaP (unmodified) tumors formed in intact male immunodeficient mice will be treated by combination anti-PCDH-PC ASOs and castration to identify ASOs that induce the most profound tumor regression and prolonged response period compared to castration alone or castration+non-targeting ASO; 3) The CWR22 human prostate tumor xenograft model will also be treated by combination anti-PCDH-PC ASO and castration to determine whether these tumors experience a significant regression and prolonged response when compared to castration or castration+non-targeting ASO.

siRNAs that deplete PCDH-PC expression in LNCaP cells selectively kill these cells when they are cultured in androgen-free medium. Note from FIG. 18, that the most potent PCDH-PC targeting siRNA (#181; SEQ ID NO:4; FIG. 29) so far kills approximately 58% of the cells (at least at 48 hrs). It may be possible to kill all of the LNCaP cells in androgen-free medium if PCDH-PC expression could be blocked in all of the cells. This can be tested by working with genetically pure populations of LNCaP cells in which PCDH-PC expression is severely impaired in all of the cells. Clones of the LNCaP cells can be developed that are severely impaired or totally blocked in their ability to upregulate PCDH-PC expression in androgen-free conditions because they express PCDH-PC-specific shRNA (Rye and Stigbrand, 2004; Berma and Dey, 2004) that targets and destroys PCDH-PC mRNA. These cells can be created by stable transfection with PCDH-PC shRNA targeting vectors and tested to show that they are much more profoundly susceptible to cell death under in vitro or in vivo conditions when they are deprived of androgens.

Using the same PCDH-PC cDNA sequences used in the design of PCDH-PC siRNAs, at least 3 different shRNA expression vectors can be created. LNCaP cells can be transfected with the individual vectors and stable transfectants can be cloned and tested to determine the extent to which the clones are blocked in their ability to express PCDH-PC mRNA and protein when they are cultured in androgen-free medium. The Gene Silencer PGshl-GFP vector by Gene Therapy Systems is a useful method because of: 1) the relative simplicity of the work needed to create a viable shRNA vector; 2) expression of the shRNA is driven by the human U6 RNA pol III promoter that drives high level expression of a GFP in LNCaP cells; 3) it contains a selectable G418-resistance marker and; 4) it co-expresses GFP which will enable rapid selection of transfected cells using a Flow Activated Cell Sorter. The vector is supplied as an open plasmid pre-digested with two different restriction endonucleases. The company provides a sequence template for the design of two (partially complementary) 63 base oligonucleotides that will anneal, leaving a double stranded insert with restriction endonuclease-compatible overhangs that can be directionally ligated into the vector. The three 19 bp PCDH-PC-complementary sequences (already tested in our siRNA) can be inserted into the oligo design so that the RNA expressed from this vector will form a double-strand hairpin that can be digested by Dicer to produce functional siRNAs. All vectors should be sequenced to confirm appropriate construction. Control vectors can also be constructed that: 1) do not have shRNA inserts or 2) that have scrambled PCDH-PC sequence inserts for control experiments. Purified vectors can be transfected into LNCaP cells using Lipofectamine 2000 and 48 hrs later, the cells can be run through a cell sorter to collect GFP-expressing cells. These cells are plated and subsequently selected in G418 to produce clones. Individual clones are expanded then exposed to androgen free medium for 3-5 days, RNA is extracted and converted into cDNA and then analyzed by semi-quantitative and Real-Time PCR to evaluate expression of PCDH-PC mRNA when compared to control LNCaP cells (transfected with empty or scrambled shRNA vectors). These cells can also be tested by transfection with a myc-tagged PCDH-PC expression vector and 48 hrs later, protein extracts are electrophoresed and Western blotted for evaluation of suppression of myc-tagged PCDH-PC (120 kd) compared to controls. The ability to detect suppression of PCDH-PC mRNA and myc-tagged protein expression is shown by our ability to do this in our preliminary experiments (14).

Antisense oligonucleotides (ASOs) can be designed and tested that target and suppress PCDH-PC expression so that they can be functionally tested in prostate cancer models. ASOs are short (20 nucleotide) deoxyribo-oligomers whose sequences are complementary to the target gene mRNA. They bind to the target mRNA through complementary base-pairing and attract the binding of RNase H, an enzyme that degrades double strand RNA, thus destroying the target mRNA (18-25). ASOs are rapidly becoming one of the preferred methods for gene targeting in the in vivo setting. They can be chemically modified to make them resistant to nucleases that abound in serum and cells (commonly phosphorothioate- or 2′-O-[2-methoxyethyl]-backbone modifications are used for this purpose), yet retain their ability to form double stranded bonds with mRNAs. They can be synthesized in mass batches suitable for pharmaceutical application, thus they represent an agent that, when proven to be effective gene suppressors, can be synthesized and mass produced like medicinal agents used for human health. Finally, they have low potential for immunological recognition nor are they known to be associated with genetic damage as with viral agents that are being considered for human gene therapies. As such, ASOs, at least, offer the potential of being a gene silencing agent that is most ready for rapid translation into human clinical trials. Moreover, contemporary chemical modifications of ASO backbones appear to make them more able to penetrate into cells of soft tissues, thus the technology driving this approach is advancing rapidly as well. There are already several different ASOs that are already undergoing clinical evaluation for prostate cancers (Gleave et al., 2002; Gleave et al., 2003; Retter et al., 2004; Chi and Gleave, 2004).

The invention provides for ASOs synthesized in batches with phosphoro thiorate-backbone modifications. Different ASOs may share partial homology. Poly-G or Poly-G-C stretches of more than 3 nts should be avoided, as these can lead to artifacts. Each of these ASOs can then be transfected individually into LNCaP cells that have been maintained in androgen-free medium for 5 days using a lipofectin reagent to increase intracellular uptake. Transfection continues for a further 48 hrs, at which time mRNA is extracted from the cell and subject to semi-quantitative RT-PCR analysis to assess the levels of PCDH-PC, actin or E-cadherin mRNA. Each ASO can be transfected into LNCaP cells together with an expression vector containing myc-tagged PCDH-PC cDNA (in this assay expression of the myc-tagged PCDH-PC protein will be measured after 48 hrs by Western blot). The ASO can be transfected into LNCaP cells that are stably transfected with the pTOP-FLASH luciferase reporter, maintained in androgen-free medium, to identify the extent to which luciferase expression is reduced by the ASO (monitors loss of functional effects of PCDH-PC expression). The ASOs can be tested for their activity in these assays, using scrambled sequence ASOs as negative controls. The invention also provides for combinations of the most effective ASOs (two or three together) which can also be used to reach a greater level of PCDH-PC expression suppression.

Reduction of PCDH-PC expression in LNCaP cells exposed to androgen-free medium can be an effective cell-death inducing paradigm in vitro. The siRNA against PCDH-PC kills 58% of LNCaP cells grown in androgen-free medium. One can expect that stable shRNA or ASOs will, at least match and preferably, exceed this level of cell death. Four different shRNA-expressing LNCaP clones that have the lowest PCDH-PC mRNA and protein expression in androgen-free medium can be split 1:5 to produce 20 plates of each clone, then 6 hrs later, the medium on 10 of the plates can be changed to androgen-free medium (phenol red free RPMI with 10% CS-FBS). At 24 hr intervals (up to 5 days), cells (adherent and floating) are collected from 2 plates each in normal medium or androgen-free medium and the cells are fixed and stained with PI for flow cytometric analysis. By counting 10,000 cells, the percent of the cell population in the sub-Go peak (dead cells) can be assessed using the CellQuest software program. The average from 2 plates (with 2 measurements each) can be compared to the sub-Go population of the same cells grown in normal medium from the same time point (again from 2 plates with 2 measurements) using a student T-test to determine whether there is a significant difference. The difference in the populations of dead cells with PCDH-PC shRNA (with androgen/without androgen) at each time point is also compared to the same measurements done on clones transfected with empty vector or clones transfected with scrambled-sequence shRNA vectors to show that effects are specific for cells that lack expression of PCDH-PC. Cells with knockout of PCDH-PC have differential death rates approaching 100% over the 5 day period following exposure to androgen free medium and these rates can be significantly greater than any rate observed in control clones. PCDH-PC ASOs can be transfected (using lipofectamine) into LNCaP cells grown in normal or androgen-deprived medium (for 5 days) to identify those that have the most potency in inducing death of LNCaP cells over the next 48 hrs. Plates of LNCaP cells (2 each) grown for 5 days in androgen-free medium can be exposed to increasing concentrations of a given ASO (10, 20 or 30 nM dissolved in androgen-free medium) and 48 hrs later, cells are collected for flow cytometric analysis. The sub-GO fraction of any given concentration can be compared to the sub-GO fraction of cells exposed to scrambled ASO to determine whether cell killing is specific for the PCDH-PC targeting ASO. This method allows for the identification of PCDH-PC ASOs with the most significant efficacy for specifically inducing cell death of androgen-deprived LNCAP cells.

Pre-Clinical Testing in Animal Models to Identify the Potential of PCDH-PC Knockdown Therapy with Androgen Deprivation.

PCDH-PC knockdown by stable shRNA expression enhances LNCaP tumor response to castration in a mouse xenograft model system. LNCaP with PCDH-PC expression stably reduced by shRNA vectors will be implanted into nude mice to show that tumors formed by these cells experience a much more profound response to castration than control LNCaP tumors (transfected with empty vectors or scrambled shRNA vectors). PCDH-PC shRNA clones and control clones can be tested. Individual clones (2×10⁶ cells) will be mixed with matrigel and injected s.c. into the flanks of male nude mice (to produce 2 groups of 10 mice/clone). Generally, 100% of male mice develop tumors within 1 month after implant. When the tumors reach the size of 250 mm² (by 3-4 weeks), one group/clone is surgically castrated. Tumor growth for both groups (castrated/uncastrated) is measured over another month (at least) at 2-3 day intervals. Tumor growth rates can be plotted as a function of time for each clone tested (3 different PCDH-PC shRNA clones and 3 different control clones). Statistical comparisons of growth rates between different groups can be done using the Kruskal-Wallis test. Past studies with this model system have shown that tumor growth halts for almost a 2 week period after castration and then resumes (LNCaP tumors generally don't regress after castration)—control clones will show this behavior whereas PCDH-PC shRNA clones will profoundly regress, and have an extended time until tumor growth is restored, or perhaps is never restored over the next 2 months.

PCDH-PC knockdown by ASOs enhances LNCaP tumor response to castration in a mouse xenograft model system. PCDH-PC targeting ASOs able to induce death of LNCaP cells in androgen-free medium are subjected to pre-clinical testing against parental LNCaP cells xenografted into male nude mice to determine if they enhance the response to castration. Parental LNCAP cells (2×10⁶) is mixed with matrigel and injected s.c. into mouse flanks (5 groups of 10 each). When tumor size reaches 250 mm₃, all mice are castrated. Group 1 is injected daily (intraperitoneally) with ASO-free vector only, Groups 2-4 are injected daily i.p with one of 3 effective PCDH-PC targeting phosphorothio-ASOs (10 mg/kg) and Group 5 will receive a scrarnbled, non-specific phosphoro-thio ASO at the same dose. Tumor volumes are measured at 2-3 day intervals using calipers and plotted as a function of time over the next month. Tumor growth rates can be compared between groups as above. The expected result is that Groups 1 and 5 will be growth-suppressed during the acute period following castration but continue to grow afterwards, whereas Groups 2, 3 and 4 will regress and be significantly suppressed in their ability to regrow. Using similar groups, the ASOs can be tested to determine their impact on the growth of already androgen-independent tumors (by initiating ASO therapy during the regrowing phase approximately 3 weeks after castration).

PCDH-PC knockdown by ASOs enhances CWR22 tumor response to castration in a mouse xenograft model system. To test whether PCDH-PC targeting by ASO (combined with castration) has a more general applicability for prostate cancer, experiments can be conducted in another androgen-sensitive human tumor xenograft system, CWR22. CWR22 tumors are passaged directly from prior xenografts, not from cultured cells. PCDH-PC mRNA may be upregulated in CWR22 tumors after castration of the host. Tumor mRNAs obtained before and at different times after castration can be assayed by RT-PCR for expression of PCDH-PC. Studies of human prostate cancers that show up-regulation of PCDH-PC expression in hormone-refractory tumors indicate that this model system is also likely to show upregulation of PCDH-PC.

### Example 6

### Protocadherin-PC and Prostate Cancer

Prostate cancer is an extremely common cancer in men and a prevalent source of cancer-related morbidity and mortality for men in Western countries. The etiological and genetic factors that influence the development of this disease in humans and the factors that drive the progression of early (indolent) prostate tumors to more aggressive states are poorly understood. However, it is clear that androgenic steroids are important for prostate cancer development and progression and this understanding is consistent with the use of various types of androgen-withdrawal strategies as therapeutics to treat prostate cancer patients, especially advanced disease. These therapies are believed to work by inducing apoptosis of a fraction of prostate cancer cells in the patient. But it is also clear that androgen-withdrawal therapies are only temporarily effective; most advanced prostate cancer patients will progress to hormone-refractory disease within a few years. Since it is this form of the disease that kills the patient, there has been an intense research effort to define the molecular basis for the development of hormone-refractory prostate cancer. While other studies have focused on evaluating the extent to which abnormal androgen-signaling might contribute to the origin of hormone-refractory prostate cancer, these studies focused on studying whether changes in the apoptotic-sensitivity of prostate cancer cells might have an important role in the development of therapeutic resistance. An unusual gene product associated with apoptosis- and hormone-resistant prostate cancer has been discovered and characterized. The gene product is termed protocadherin-PC (pro-PC). Amongst the more intriguing aspects of the pro-PC gene product is its human- and male-specific nature (the gene encoding pro-PC was acquired during a chromosomal transposition associated with the evolution from primates to humans and it is localized on the human Y-chromosome), the unique cytoplasmically-localized nature of its major translation product as well as its seeming ability to activate cell signaling through the wnt-signaling pathway in prostate cancer cells, a signaling pathway that is also known to be involved in oncogenesis of the colon, skin and other human tissues. Moreover, studies have revealed that pro-PC expression is associated with a transdifferentiation process wherein prostate cancer cells take on characteristics of neuroendocrine (NE)-like cells. Since this neuroendocrine transdifferentiation process is also associated with the transition of human prostate tumors to a hormone-resistant or aggressive state, the study of pro-PC product reveals information regarding the role of wnt signaling and neuroendocrine differentiation in prostate cancer biology and response to therapy.

The invention relates to expression of pro-PC in prostate cancer cells as well as the potential molecular mechanism(s) through which it might exert anti-apoptotic or pro-malignant effects. The invention includes: 1) pro-PC expression confers an apoptosis-resistant and neuroendocrine-like phenotype on prostate cancer cells through activation of the wnt-signaling pathway in these cells; 2) pro-PC's ability to activate wnt signaling in prostate cancer cells is mediated either through its ability to directly bind to β-catenin or by mediation of a heterodimeric transcription factor protein known as FHL-2; 3) expression/overexpression of pro-PC in the prostate glands of transgenic mice will induce wnt-mediated neoplasia associated with extensive NE transdifferentiation of prostate epithelial cells and drive indolent non-NE mouse prostate tumors to aggressiveness characterized by increased growth, metastatic ability and increased resistance to androgen withdrawal therapy.

Biological consequences associated with the expression of pro-PC in prostate/prostate cancer cells. The invention utilizes in vivo and in vitro models to show that pro-PC expression upregulates wnt signaling and induces a neuroendocrine-like phenotype in prostate/prostate cancer cells. As well, this work will identify pro-malignant effects of pro-PC expression in prostate tumor biology. In vivo models involving transgenic mouse generation with prostate-targeted pro-PC will be used to identify primary changes in prostate gene expression consistent with wnt signaling activation and neuroendocrine transdifferentation and changes in prostate epithelial cell morphology, growth behavior and differentiated phenotype will be assessed by a variety of analytical techniques. These transgenic models will be bred into one particular LADY transgenic model of prostate cancer (12T-7) that develops indolent, non-neuroendocrine pre-neoplastic lesions in the prostate to test whether prostate-specific pro-PC expression will drive this model to a more malignantly aggressive, neuroendocrine-like tumor model. Human prostate cancer cell line variants (LNCaP derivatives) with/without pro-PC expression will be compared for gene expression patterns using a microarray gene chip type of analysis to identify effects of pro-PC on wnt-target and neuroendocrine-specific genes in prostate cancer cells as well as to identify other potential signaling pathways that might be influenced by pro-PC expression. Finally, siRNA and short hairpin expression vectors, provided by the invention, that target and suppress pro-PC expression in prostate cancer cell lines will be used to functionally assess whether reduction of pro-PC expression suppresses wnt signaling and the neuroendocrine phenotype as well as to test whether this action suppresses the development of apoptosis- and hormone-resistance that is associated with pro-PC expressing prostate cancer cells.

Molecular mechanism through which pro-PC activates the wnt-signaling pathway in prostate cancer cells. Direct immunoprecipitation experiments suggest that the pro-PC protein binds to β-catenin protein (the end effector of wnt signaling), yet a yeast 2-hybrid analysis did not identify β-catenin as a direct pro-PC binding partner. Instead this type of analysis showed that FHL-2, a transcription factor that can form a heterodimer with β-catenin, was a direct binding partner of pro-PC and this interaction was confirmed by in vitro “pull-down” binding experiment involving these two proteins (pro-PC and FHL-2). The invention provides for methods to evaluate whether the homologous β-catenin binding site within the C-terminal domain of pro-PC is involved in wnt signaling activation or whether the interaction of pro-PC with β-catenin and wnt signaling activation is mediated by the FHL-2 protein. Small “in-frame” deletions within the 3′ domain of the pro-PC cDNA will be tested for loss of FHL-2 or β-catenin binding in vitro and for loss of wnt-signal activation potential in vivo. Knockout of FHL-2 in LNCaP cells with a siRNA procedure will be used to test the extent to which this protein is required for wnt signal activation by pro-PC.

Prostate cancer (PCa) is a major medical problem for men in developed countries. In the United States, the American Cancer Society (American Cancer Society website www.cancer.org, Cancer Facts and Figures 2004) predicts that there will be approximately 230,000 cases detected this year alone and that nearly 30,000 men will die of this disease. These statistics mean that PCa ranks second only to lung cancer as a cause of cancer deaths in U.S. men. Since PCa is so strongly associated with aging, our rapidly aging population is likely to be increasingly burdened by this disease. While faced with these overall grim statistics, there is reason to hope that progress is being made against PCa through intense screening programs using serum-based PSA measurements. Indeed, most clinicians treating this disease acknowledge a trend towards diagnosing prostate cancer at earlier stages when patients have a smaller tumor burden (Crawford, 2003). However, it has yet to be proven that these screening programs result in decreased mortality from this extremely common disease.

Like the normal prostate gland that develops, matures and functions under the influence of androgenic steroids, PCa also requires androgenic steroids for its development and progression. This need for androgen is consistent with the common treatment for advanced disease, androgen-withdrawal therapy (Denmeade and Isaacs, 2002). Androgen-withdrawal is believed to work, at least temporarily, because it induces apoptosis of some fraction of prostate cancer cells (Isaacs et al., 1994). Unfortunately, these types of therapies are only transiently suppressive of the disease and hormonally-treated PCa eventually relapses in a seemingly androgen-independent (or hormone-resistant) state (Debruyne, 2002). Once in this hormone-resistant state, PCa can be highly resistant to other common forms of cancer therapeutics such as chemotherapy and radiation. The simplicity of androgen-deprivation treatments and the general non-toxic nature of these therapies are an attractive incentive for their use. Therefore, there is a great interest in determining the epigenetic and genetic parameters that will lead to the development of hormonal-resistance in prostate tumor cells so as to be able to use this therapy more effectively or to increase its effectiveness for PCa control for a much longer period of time.

The androgen signaling pathway has been one of more obvious biochemical aspects of prostate cancer cell biology that research has focused on in attempting to identify mechanism(s) associated with hormone resistant disease. At this time there seems to be a degree of consensus among prostate cancer researchers that promiscuousness or hyper-activity of the androgen-signaling system in prostate cancer cells accompanies the progression to hormone resistance disease (Culig, 2003; Culig et al., 2003; Taplin and Balk, 2004). On the other hand, given the strong relationship between androgen withdrawal and the onset of apoptosis of PCa cells in vivo, there has also been some focus on determining whether aberrations in the apoptotic regulatory and execution machinery of prostate cancer cells might accompany progression to hormone resistance. Clinical evidence that the anti-apoptosis gene product, bcl-2, is overexpressed in advanced and hormone-resistant PCa cells combined with experimental research showing that bcl-2 overexpression can confer a hormone resistant characteristic on PCa cell lines supports the idea that defects in the apoptotic response mechanism plays some role in hormonal resistance of Pca (Colombel et al., 1993; Catz and Johnson, 2003; Apakam et al., 1996; Raffo et al., 1995). Likewise, p53 gene loss/mutations which are found most frequently in advanced and hormone-resistant PCa may be associated with a reduced apoptotic response of prostate cancer cells to androgen withdrawal as indicated in experimental research (Isaacs et al., 1994; Burchardt et al., 2001). More recently, hyperactivity of NF-Kappa-B signaling which can suppress apoptosis was also reported to be high in advanced human PCa (Lessard et al., 2003). This invention is directed to methods to change apoptotic machinery of PCa cells as involved in hormone resistance. The invention is directed to a very unusual gene product, a novel member of the cadherin gene family which is named protocadherin-PC (pro-PC), is upregulated in some apoptosis-resistant PCa cell lines (Chen et al., 2002). The studies reported below show this same gene product is also upregulated in naturally-occurring hormone resistant human prostate cancers in patients.

Protocadherin-PC and Prostate Cancer. Cadherins are a very large and diverse family of gene products that are related by distinct conserved regions of gene and protein sequences within their 5′/amino terminus referred to as cadherin boxes (Angst et al., 2001). Their diversity can be sorted into any one of 3 sub-families referred to as protocadherins, classical cadherins and desmosomal cadherins, mainly based upon the numbers of cadherin boxes present in any given family member (Angst et al., 2001; Suzuki, 1996). The most well characterized and functionally understood sub-family of cadherin genes are the classical cadherins that include E-, N- and P-cadherin which are well known to participate in intracellular adhesion through homophilic Ca⁺⁺-dependent interaction of their extracellular domains, and to participate in the regulation of certain important cellular signaling processes, especially wnt-signaling (Suzuki, 1996; Ivanov et al., 2001; Leckband and Sivasankar, 2000; Barth et al., 1997). However, the protocadherin subfamily, although the largest group of cadherin-related genes, is generally less well characterized than classical cadherins and, functionally, more poorly understood (Frank and Kemler, 2002). There is an especially large number of protocadherin genes on human chromosome 5 that lie within 3 distinct clusters (Suzuki, 2000). The protocadherins within these clusters are highly expressed within neuronal cells of the central and peripheral nervous system. To date, research on the function of the genes within these clusters suggests that they are important for formation of neural circuitry and especially for the formation and function of neuronal synapses (Hilschmann et al., 2001). It is remarkable that the pro-PC gene product that was identified is an orphan gene, meaning that there is only one copy localized on the human Y-chromosome (at Yp11.2), thus making it a unique gene product that can only be expressed in male tissues (Chen et al., 2002; Blanco et al., 2000). Moreover, pro-PC is a “human-only” gene product, having “evolved” from another protocadherin orphan gene homologue present on the primate (and remaining on the human) X-chromosome (at Xq21.3, named PCDHX) (Blanco et al., 2000). Apparently, a large region of the X-chromosome containing this region was duplicated onto the Y chromosome during the evolutionary transition from primates to humans and during this duplication and transposition, the Y-chromosome associated protocadherin gene lost a small (13 bp) but significant piece of an exon from the X chromosome gene. Additionally, the Y-chromosome gene has acquired a few single base pair changes during evolution so that it now shares 98.8% homology with the X-chromosome gene (Chen et al., 2002; Blanco et al., 2000). However, the cumulative nucleic acid sequence changes between the X- and Y-chromosomal genes drastically alters the potential translation products that can be derived from them. As discussed below, the preferred translation product of the Y-chromosome protocadherin gene lacks a signal sequence (Chen et al., 2002; Blanco et al., 2000), thus it differs significantly from the preferred translation product of the X-chromosome gene progenitor in that its translation product is cytoplasmic, rather than plasma membrane localized in cells that express it. Is it possible that this unique human-only, male-only pro-PC gene product that is expressed in the human prostate gland might have some relevance to the high frequency with which human males develop prostate cancer whereas males of most lower mammalian species (that lack the Y-homologue) are not plagued with this disease. The invention provides use of a transgenic model system to determine its oncogenic potential when abnormally expressed in the mouse prostate through transgenic technology.

As was mentioned above, another unusual aspect of pro-PC is the nature of the protein product that appears to be encoded by the translatable portion of the pro-PC mRNA. Evaluation of the primary sequence of the major transcript of pro-PC present in the apoptotic resistant LNCaP cell variants reveal that the pro-PC/PCDHY transcript has two potential AUG translation start sites within its 5′ region (Chen et al., 2002; Blanco et al., 2000) that would give rise to long-open reading frame peptides. Utilization of either of these start codons would give rise to two different, but homologous translation products that share common C-terminal domains but differ with respect to the N-terminal domains. This difference is critical, however; utilization of the more 5′ AUG translation start site in the pro-PC transcript would result in a cadherin protein with a signal sequence (and thus, likely to be membrane bound as with most other members of the cadherin-gene family) whereas utilization of the more 3′ AUG start would yield a cadherin protein that lacks the signal sequence, thus likely preventing its ability to be inserted properly into the cell membrane. Analysis of the “Kozak consensus sequence” in which the two AUG start sites lie shows that the 5′ AUG is embedded in TGAAUGA (SEQ ID NO:20), which conforms to the pattern YNNAUGA (SEQ ID NO:21), that was shown by Kozak to usually not serve as a translation start site (*) whereas the second AUG site is embedded in ACTAUGC (SEQ ID NO:22), which conforms to the pattern ANNAUGY (SEQ ID NO:23), which was found to serve as a strong translation start site (Kozak, 1983). This finding that the more downstream AUG is a more likely a translation start site conforms with our studies showing that an antibody made against a pro-PC-derived peptide sequence recognizes (on Western blots) an appropriate size protein that fractionates in the cytoplasm of apoptosis-resistant prostate cancer cell lines (Chen et al., 2002) whereas it does not recognize any proteins in the membrane fraction of these cells. An N-terminal “myc-tagged” pro-PC cDNA expression vector has been created and transfection of LNCaP cells with this vector results in an abundant cytoplasmic immunohistochemical staining pattern using anti-myc antibodies that differs significantly from the nuclear-specific staining pattern seen in untransfected LNCaP cells (identifying the presence of the normal nuclear c-myc protein). Thus, the pro-PC gene product is not only distinguished from other members of the protocadherin gene family by its human- and male-specific nature but also by its tendency to produce a (non-membrane bound) cytoplasmic protein upon translation.

The functional consequences of the expression of pro-PC in prostate cancer cells focuses on a region within the 3′ region of its translation product that encodes a small serine-rich domain with significant homology to the known β-catenin binding site of classical cadherins (Chen et al., 2002; Blanco et al., 2000; Stappert and Kemlar, 1994). β-catenin is the end molecule of the wnt signaling pathway and, when it is present in sufficient concentrations, can form a heterodimer with the TCF/LEF-1 transcription factor to mediate nuclear transcription of a number of different gene products that regulate differentiation, proliferation and apoptotic sensitivity of tissues and tumors (Gottardi and Gumbiner, 2001; Lustig and Behrens, 2003; Conacci-Sorrell et al., 2002; van Es et al., 2003; Aberle et al., 1997; Hajra and Fearon, 2002). In normal epithelial cells β-catenin is generally present on the plasma membrane where it is tightly bound to the cytoplasmic domain of cadherins. This appears to protect it from the degradative actions of GSK-3β, APC and the proteasome (Ivanov et al., 2001; Lustig and Behrens, 2003). Immunoprecipitation of pro-PC from extracts of the apoptosis-resistant prostate cancer cell lines resulted in co-immunoprecipitation of β-catenin (Chen et al., 2002), further supporting the idea that these two molecules may be binding partners (direct or indirect). Likewise, analysis of the apoptosis- and hormone-resistant PCa cells that were used in the discovery of pro-PC showed that these cell lines had abnormal accumulation of β-catenin in their cytoplasmic and nuclear fractions, whereas the parental LNCaP cells from which they were derived had β-catenin only in the membrane fraction (Chen et al., 2002). This abnormal cytoplasmic/nuclear accumulation of β-catenin in these cell lines was also consistent with elevated expression of a reporter (luciferase) from a catenin/TCF promoted vector (de la Taille et al., 2003), suggesting that the apoptosis- and hormone-resistant LNCaP variants (-TR and -SSR) had abnormally activated wnt signaling when compared to parental LNCaP cells (de la Taille et al., 2003). Since the apoptosis-resistant, pro-PC expressing LNCaP variants did not have mutations in β-catenin and since these cells had similar levels of APC protein when compared to LNCAP parental cells (Chen et al., 2002), these studies show that the wnt pathway might be activated in these cells through a mechanism showing an unexpected pathway for wnt signaling activation that is not used by other tumor cells. Transient transfection of LNCaP with a pro-PC expression vector induces high nuclear accumulation of β-catenin as well as increases expression of luciferase from the β-catenin/TCF promoted-luciferase reporter vector and directly links upregulation of pro-PC expression with upregulation of wnt signaling in these cells. Pathway focused (human wnt gene) cDNA microarray studies which analyzed the effects of pro-PC expression on gene expression of LNCaP cells confirms that many wnt-target genes are upregulated. These results led us to the aspect of the invention that pro-PC protects β-catenin from normal degradative processes through direct or indirect binding and, perhaps, shepherd it to the nucleus where it joins with the TCF/LEF-1 transcription factor to activate wnt signaling.

Such an activity has enormous implications for PCa progression to the hormone resistant state. The wnt signaling pathway is a powerful effector of carcinogenesis and progression in several common human tumor systems including colon and breast cancer, melanoma, oral cancers and head and neck tumors among others (Lustig and Behrens, 2003; van Es et al., 2003; Aberle et al., 1997; Hajra and Fearon, 2002; Bright-Thomas and Hargest, 2003; Lo Muzio, 2001; Kikuchi, 2003; Brown, 2001; Morin, 2003; Polakis et al., 1999; Morin, 1999). As best studied in colon cancer, wnt signaling often becomes dysregulated because of mutations or loss of the molecules that regulate the stability and half-life of the β-catenin protein product, including APC and GSK-3β (Polakis et al., 1999; Morin, 1999). These dysregulations lead to accumulation of β-catenin protein in the cytoplasmic and nuclear fractions of the cancer cells, increased transcription from β-catenin/TCF promoter elements and hyper-expression of some powerful proliferative control molecules including c-myc and cyclin D, both of which are known targets of wnt signaling and have also been mentioned as potential genetic factors in PCa development and progression (Karan et al., 2002; Drobnjak et al., 2000). As well, there is strong evidence that hyper-activation of wnt signaling (via increased expression and/or stability of β-catenin) can increase cellular resistance to apoptosis (including myc-mediated apoptosis) as well as anoikis (Chen et al., 2001; Orford et al., 1999; Longo et al., 2002; Ueda et al., 2002; You et al., 2002), although the mechanism associated with this particular effect is not yet clearly defined. The phenotypic effects of pro-PC expression in prostate cancer cells (i.e. apoptosis- and hormonal-resistance) are a direct result of its ability to activate the wnt signaling pathway in these cells.

The mechanism through which pro-PC activates wnt signaling in prostate cancer cells is useful in the methods of the invention. There is evidence for co-immunoprecipitation of pro-PC with β-catenin (Chen et al., 2002). A yeast 2-hybrid expression analysis was conducted and was expected to confirm the ability of pro-PC to form direct binding partners with β-catenin. A potent transcriptional co-activator of β-catenin, FHL2 (Wei et al., 2003; Martin et al., 2002), directly binds to pro-PC. The invention provides for a functional test for identifying whether the homologous β-catenin-like binding domain within the C-terminal region of pro-PC is critical to its ability to induce wnt signaling and to identify whether the interaction of FHL-2 protein with pro-PC is critical to wnt-signaling activation in PCa cells.

Neuroendocrine Cells, Neuroendocrine Transdifferentiation and Prostate Cancer. There is a propensity of PCa cells to undergo a “transdifferentiation” process in which they acquire characteristics of neuroendocrine- (NE-) like cells. NE cells are normally found in many tissue types, including the normal prostate, where they were believed to be derived from progenitor neural crest cells that migrated into these tissues during embryonic development. In normal adult tissues, these cells are generally rare and are widely interspersed amongst the epithelial cell population (Noordzij et al., 1995). Their most intriguing characteristic is their production and secretion of an abundance of neuropeptides (exemplified by bombesin, calcitonin, parathyroid-like hormone, serotonin and adrenomedullin) and other growth factors (including VEGF) that are believed to influence the surrounding epithelial cell populations (Abrahamsson and Di Sant'Agnese, 1993; Cohen et al., 1993; Gkonos et al., 1995; Chevalier et al., 2002). Indeed, there is a small proportion of PCa patients that present with overt prostate-derived NE tumors (referred to as small cell carcinoma of the prostate). While this type of prostate cancers is rare (estimated to be approximately 60 patients a year in the U.S.) (Randolph et al., 1997), it is extremely aggressive; patients with this form of prostate cancer have few treatment options and generally succumb to the disease in a very short time (Randolph et al., 1997; Papandreaou et al., 2002). However, a growing body of literature shows that this topic is highly relevant even to those patients with the overwhelmingly more common form of prostate cancer, adenocarcinoma of the prostate. There have long been reports in clinical literature showing that PCa progression, especially to the hormone-refractory state, is associated with the increased presence of overt NE-like cells in prostate tumors (di Sant'Agnese and Cockett, 1996; Abrahamsson, 1999; Ito et al., 2001; di Sant'Agnese, 2001; Monteunga et al., 2003) as well as increased levels of NE-derived peptides such as neuron-specific enolase (NSE) and chromogranin A (chromo-A) in the serum of advanced, hormone-refractory patients (Yu et al., 2001; Segawa et al., 2001; Kadmon et al., 1991; Tarle and Rados, 1991; Harding and Theodorsecu, 1999). Other clinical studies have found that high levels of these NE markers (in serum and tumors) are prognostic factors identifying reduced survival times in patients being treated for advanced disease (Hvamstad et al., 2003; Lilleby et al., 2001; Kamiya et al., 2003; Isshiki et al., 2002). The relevance of this topic for prostate cancer is amplified by the demonstration that cultured PCa cells can be directly induced to undergo a NE-transdifferentiation process in vitro by exposure to a diverse range of stimuli (Zelivianski et al., 2001). While this was first shown in experiments published in 1994 in which LNCaP and PC-3 cells were grown in medium supplemented with dibutyral cyclic AMP (db-cAMP) (Bang et al., 1994), in 1997, the observation was made that LNCaP cells, an androgen-sensitive human PCa cell line, would undergo NE transdifferentiation when chronically exposed to medium lacking androgens and that restoring androgens back to the medium suppressed this NE transdifferentiation state (Shen et al., 1997). Other laboratories have confirmed that chronic exposure of LNCaP cells to IL-6 or NS-398, a Cox-2 specific inhibitor, would also induce NE transdifferentiation (Murillo et al., 2001; Jimenez et al., 2001; Meyer-Siegler, 2001; Deeble et al., 2001). These kinds of observations suggest that the increased NE cells found in advanced, aggressive and hormone-refractory prostate tumors are likely transdifferentiated PCa cells and clinical observations showing increased numbers of NE cells in prostate tumors from patients following hormonal therapy strongly support this idea. Finally, there is increasing evidence from contemporary animal models of prostate cancer (human tumor xenografts and in transgenic mice [TRAMP and aggressive LADY mice] that tumor progression in these models is associated with the acquisition of NE characteristics by the tumor cells (Huss et al., 2004; Wang et al., 2004; Kaplan-Lefko et al., 2003; Masumori et al., 2004).

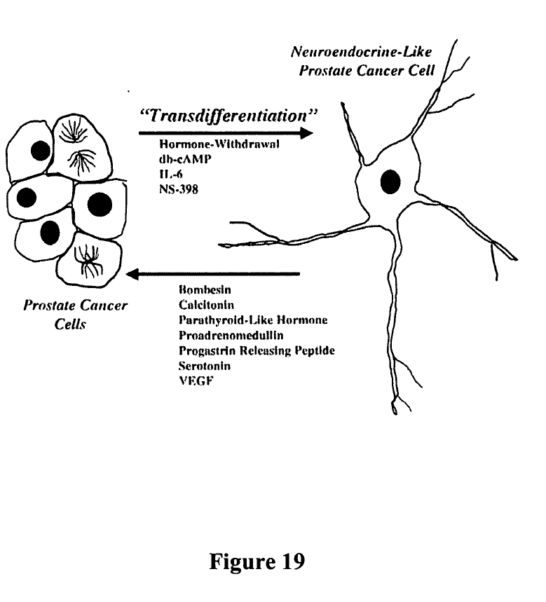

With regards to prostate cancer, the idea that PCa cells can directly undergo a transdifferentiation process that gives them properties of NE cells has a number of implications. First, as mentioned, transdifferentiated NE cells produce and secrete abundant amounts of numerous active neuropeptides. Evidence has been accumulating that non-NE human PCa cell lines have specific cell surface receptors for many of these peptides (Shah et al., 1994; Sun et al., 2000; Dizeya et al., 2004) and that these receptors promote cell division and apoptosis-resistance when engaged by the appropriate ligand. Thus, there is good reason to believe that the accumulation of NE-like cells within aggressive/hormone-refractory human prostate tumors may be “feeding” adjacent and even distant tumor cells with these peptide hormones, cumulatively increasing their growth rate and resistance to therapeutics. A recent study addresses this possibility using a xenograft model system and in elegant experiments, it was shown that implantation of a mouse NE-prostate tumor on one flank of a castrated immunodeficient mice was sufficient to enable growth of an androgen-dependent human prostate cancer cell line implanted in the opposing flank (Jin et al., 2004). Factors (most likely neuropeptides) shed from NE-differentiated prostate tumor cells support the growth of androgen-dependent tumor cells in a low androgen environment even when they are at a distant site (FIG. 19). The invention provides that PCa cells transformed by pro-PC acquire the characteristic that they can stimulate growth of androgen-dependent tumor cells at a distant site using a mouse xenograft model system.

The invention provides: 1) that pro-PC expression is highly upregulated in LNCaP cell lines exposed to androgen-free medium, a condition under which it was previously shown that these cells undergo NE transdifferentiation (Shen et al., 1997); 2) that pro-PC expression is associated with upregulation of wnt signaling mediated by increased β-catenin/Tcf transcription in LNCaP cells (de la Taille et al., 2003); 3) that increased wnt signaling in MMTV-induced mouse breast cancer is associated with transdifferentiation in breast cancer so that these cells give rise to cells with a myoepithelial phenotype (Li et al., 2003) and finally; 4) wnt signaling is important for differentiation of neural crest derivative cells (Yanfeng et al., 2003). Based on this collection of information, the potential relationship between pro-PC expression and NE transdifferentiation in prostate cancer cells was investigated and the data presented in this Example now shows: A) that 4 completely different stimuli that induce NE transdifferentiation of prostate cancer cells also induce upregulation of pro-PC expression; B) that transfection of LNCaP or PC-3 cells with a pro-PC expression vector directly induces NE transdifferentiation of these cells; C) that transfection of LNCaP cells with a stabilized (mutant) β-catenin expression vector also induces NE trans-differentiation, supporting the idea that wnt signaling is involved in the transdifferentiation process; and D) that NE transdifferentiation induced by pro-PC expression or culture in androgen-free medium can be blocked by suppression of β-catenin, the end point in the wnt signaling pathway, with an siRNA against β-catenin or by a dominant negative TCF.

Pro-PC expression induces wnt signaling that participates in the transdifferentiation process leading to the NE phenotype in prostate cancer cells. The invention provides uses of the molecular system(s) that drive NE transdifferentiation of prostate cancer cells in methods identify potential new molecular targets (found on NE cells) to attack the progression of prostate cancer and suppress the development of aggressive, hormone-independent tumors in patients with this disease.

Protocadherin-PC Expression and Apoptosis Resistance in Prostate Cancer (Chen, et al, 2002).

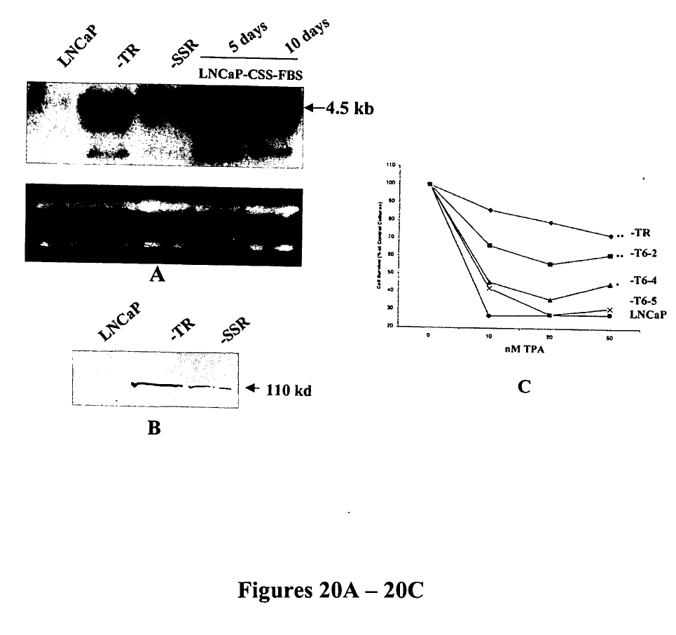

To identify new molecular mechanisms through which human prostate cancer cells might acquire resistance to apoptosis and thus to the therapeutic agents used to treat the disease, a prototypic human prostate cancer cell line, LNCaP, was subjected to repeated (acute) exposures to two different apoptotic agents. Expansion of surviving cell populations and repeated exposure to the particular apoptotic agent followed by further expansion and exposure paradigms resulted in the selection of two cell lines, LNCaP-TR (TPA-resistant) and LNCaP-SSR (serum starvation-resistant) that were found to be cross-resistant to the alternate apoptotic agent and, when implanted subcutaneously into castrated male nude mice, were readily able to form tumors in striking contrast to parental LNCaP cells which did not form tumors in castrated male nude mice. A subtractive-hybridization PCR technique was then used to identify gene products that were differentially expressed in the LNCaP-TR cells (when compared to parental LNCaP) and this technique allowed identification of a 259 bp “tag” sequence of a gene product that is highly overexpressed in -TR and -SSR cells in comparison to parental LNCaP cells (FIG. 20A). 5′ and 3′ RACE procedures were used to recover and characterize the entire gene product (4.8 kb cDNA) containing this tag sequence and, surprisingly, the gene product was a unique member of the cadherin gene family, based upon the presence of 7 cannonical cadherin box sequences in the 5′ domain. In fact, the number of cadherin boxes present in this gene product placed it in the sub-category of protocadherins. For this reason, the gene product was named, protocadherin-PC. Consistent with a potential relationship between the expression of this gene product and the hormone-resistant state of the prostate cancer cell, other experimentation showed that pro-PC (mRNA and protein) expression rises significantly when parental LNCaP cells were exposed to an androgen-free medium (FIGS. 20A and 20B) and in LNCaP xenograft tumors when their immunodeficient mouse hosts were castrated (Chen et al., 2002). Finally, LNCaP cells transfected with a pro-PC cDNA were found to be much more resistant to apoptotic stimuli than parental LNCaP cells, suggesting that this gene product might be sufficient for conferring the apoptotic-resistant phenotype that was detected in the -TR and -SSR variant cell lines (Chen et al., 2002; FIG. 20C).

With regards to the protein product encoded by pro-PC mRNA, it is highly unusual (for a member of the cadherin gene family) in that the major translation product lacks a signal sequence and, thus, is unlikely to be membrane bound as with most other cadherin-family gene products. An antibody (rabbit polyclonal) made against a unique peptide sequence of pro-PC detected an appropriate polypeptide synthesized in abundance in LNCaP-TR and -SSR cells but not in parental LNCaP cells and cell fractionation studies demonstrated that this protein is mainly present in the cytoplasmic fraction of the -TR and -SSR cells (Chen et al., 2002; Blanco et al., 2000). This cytoplasmic localization has subsequently been confirmed by the use of a myc-tagged pro-PC cDNA that induced intense cytoplasmic immunohistochemical staining with anti-myc antibodies following transfection to parental LNCaP cells.

While the initial 259 bp tag sequence for pro-PC that was isolated was not matched to other known human gene products in our genbank searches at the time, a search of genbank after complete sequencing of the RACE products then revealed perfect identity with a human gene sequence referred to as human protocadherin-Y (hPDCHY; Blanco et al., 2000). The work describing the hPDCHY gene was startling because it also showed that this specific gene product was a human-only gene product that is present on the human Y chromosome (Blanco et al., 2000). Apparently, protocadherin-PC/hPDCHY is derived from a homologous gene on the X-chromosome of primates and lower mammalian species (PDCHX), which is located within a cluster of genes on the X-chromosome that translocated to the human Y chromosome during evolution from primates. During this translocation, the pro-PC gene also apparently lost a contiguous 13 base pair sequence within the 5′ (translated) region of the gene and this loss explains the change in the translation start difference between the PDCHX and pro-PC gene product. Thus, the pro-PC/hPDCHY gene product is distinct from the PDCHX product not only in its preferential use of an alternate translation start that deletes the signal sequence but also in its presence on the human Y chromosome so this gene product can only be expressed in males.

Pro-PC in Human Prostate and Prostate Cancer Specimens

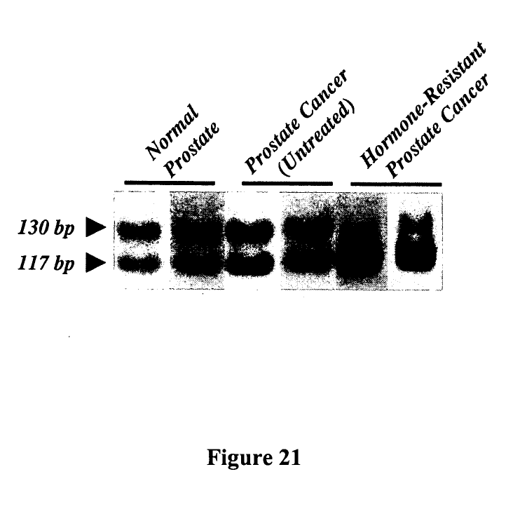

RT-PCR procedures on mRNA extracted from prostate cancer tissues or from microdissected human prostate tumors, by in situ hybridization procedures have been done and, more recently with an antibody against pro-PC. The RT-PCR procedure, at least, allows one to readily distinguish expression of the X-linked homologue (PCDHX) from the Y-linked homologue (pro-PC/PCDHY) with a set of primers that spans the 13 basepair deletion present in the Y-encoded gene product (FIG. 21). Analysis of some normal human tissues detected expression of the Y-encoded gene product in (non-pathological, male) human brain, prostate and (male-derived) placenta. Evaluation of the expression of pro-PC in human prostate/prostate cancer specimens was striking showed that expression of this gene product is related to the acquisition of hormonal resistance in human prostate cancers.

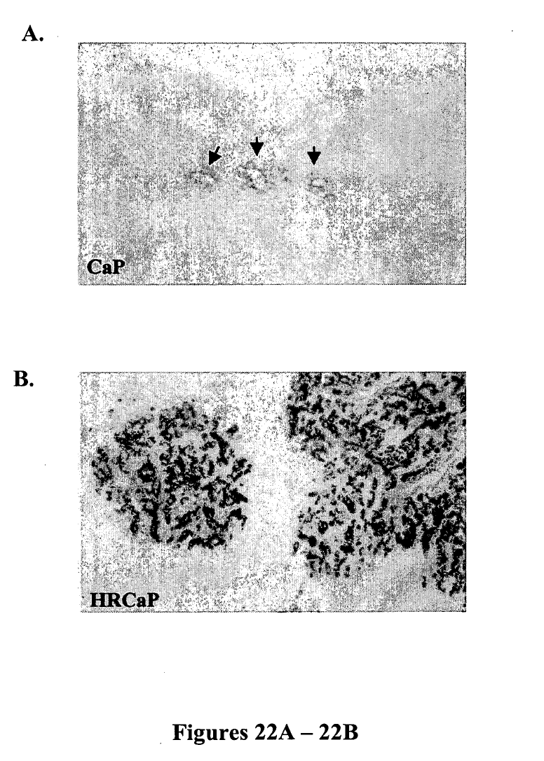

When this type of analysis was applied to multiple specimens of human prostate-derived specimens, there was a statistically significant increase in expression of pro-PC in hormonally-treated (3 months prior to radical prostatectomy) or hormone-resistant (regrowing after hormonal therapy) prostate cancers compared to normal human prostate or untreated prostate cancers. These data support the idea that expression of pro-PC is associated with survival of prostate cancer cells following hormonal therapy. In situ hybridization analysis of fixed human prostate cancers (using a probe that would recognize both the X- and Y-homologue) also demonstrates: 1) a significant upregulation in the expression of related gene product [presumably the Y-homologue] in hormone resistant prostate cancers; and 2) some cells within the basal layer of the normal human prostate are expressing a gene product homologous to pro-PC/PCDHX (as yet undefined since the probe was from a homologous region of the X-Y-encoded gene products) (FIGS. 22A and 22B).

Pro-PC expression is upregulated during the progression of prostate cancer to hormonal resistance. It appears that some scattered normal human prostate basal cells express gene products that are related to pro-PC/PCDHX. These selective basal cells may be neuroendocrine cells that are found scattered throughout the normal prostate basal epithelium.

Pro-PC and Wnt Signaling in PCa Cells (de la Taille, et al., 2003).

Wnt is a complex cellular signaling pathway that involves a cascading interaction of numerous molecules, the end result being increased transcription of target gene products having TCF-binding sites in their promoter region (exemplified by the human c-myc and cyclin D1 genes) (Lustig and Behrens, 2003). TCF is enabled to initiate transcription from TCF or LEF-1-responsive elements on DNA when it is heterodimerized to β-catenin protein, so most aspects of the wnt signaling pathway function to enable β-catenin protein to enter the nucleus and complex with TCF— or LEF-1 that is already present. In general, the cannonical wnt signaling pathway can be initiated by a wnt glycopeptide ligand binding to a frizzled receptor on the cell surface. This binding stimulates the frizzled receptor (through a cascade of molecular intermediates) to phosphorylate GSK-3β, inactivating this protein. Under non-wnt stimulating conditions, unphosphorylated GSK-3β phosphorylates free (unbound to cadherin) β-catenin protein, initiating a reaction involving APC, that rapidly ubiquitinates free β-catenin, targeting it for destruction by the proteasome. As with most cell signaling pathways, the molecular cascade associated with wnt signaling has many potential sites wherein mutations or dysregulation can lead to hyperactivity of the signaling process and these kinds of disturbances are found in several prominent animal and human tumor systems (Lustig and Behrens, 2003a and 2003b). However, the end point in wnt signaling is the accumulation of β-catenin in the nucleus and its interaction with Tcf or LEF-1 in transcriptional upregulation. In wnt-unstimulated cells, a store of β-catenin protein is stably retained at the cell membrane where it is protected from degradation due to its interaction with classical cadherins (as exemplified by E-, P- and N-cadherin) that have a distinct binding site for β-catenin within their C-terminal (cytoplasmic) domain.

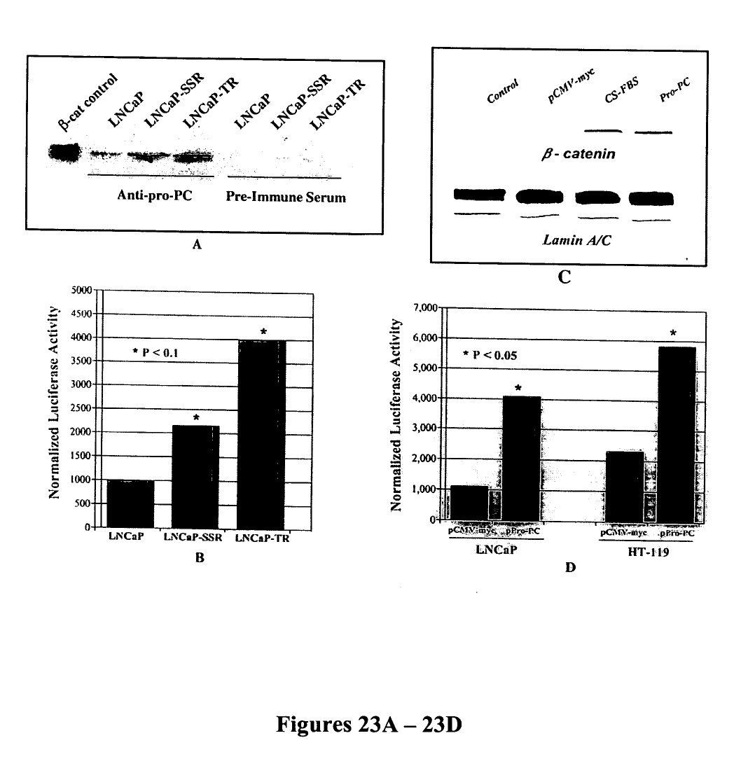

There is a short serine-rich domain within the C-terminal domain of pro-PC that resembles the β-catenin binding site of classical cadherins (Chen et al., 2002). Pro-PC was immunoprecipitated from apoptosis-resistant LNCaP sublines showed co-precipitation of a 92 kd peptide that was immunoreactive with anti-β-catenin antibody on Western blots (Chen et al., 2002) (FIG. 23A). Abnormalities of intracellular β-catenin localization or wnt-signaling in these resistant cell lines was studied and the results show that, in contrast to parental LNCaP cells in which β-catenin protein was strictly localized to the membrane fraction, apoptosis-resistant variants that express pro-PC had reduced β-catenin in membrane fractions and increased β-catenin sequestered in cytoplasmic and nuclear fractions. The altered β-catenin distribution pattern in these cells was also associated with increased signaling through the wnt-pathway as measured using a TCF-promoted luciferase reporter assay (de la Taille et al., 2003). In this assay, normalized luciferase activity is more than doubled in -SSR cells and quadrupled in -TR cells (FIG. 23B). These effects were not due to mutations in β-catenin since β-catenin cDNA amplified from all LNCaP cell lines was found to have the wildtype sequence. Likewise, no difference was detected in expression of APC protein between the LNCaP variants. The ability of transient transfection with a pro-PC expression vector to affect wnt signaling in LNCaP and other cells was assessed. Transient transfection of LNCaP cells induces nuclear accumulation of β-catenin (FIG. 23C) as well as significantly increased luciferase expression from a TCF-sensitive reporter vector (FIG. 23D) compared to cells transfected with empty vector. Finally, even human colon cancer cells (HT119) transiently transfected with pro-PC showed increased expression of normalized luciferase activity induced from the Tcf-sensitive reporter (FIG. 23D). These data indicate that even transient pro-PC expression increases nuclear β-catenin and transcriptional activity from a TCF-sensitive promoter, both strong indicators of wnt signaling activation.

A cDNA microarray analysis using the targeted cell signaling pathway microarrays of SuperArray, Inc. was done. These microarrays are spotted with a limited number of cDNAs (106 total for the GE array-Q Series human wnt-pathway microarray) and include an additional series of spots containing cDNAs for common housekeeping genes to allow relative quantification of expression levels. In these experiments, the following RNAs extracted from 4 different samples were compared: 1) control LNCaP cells transfected 48 hrs with empty vector (pCMV-myc); 2) LNCaP cells transfected 48 hrs with pro-PC vector; 3) LNCaP cells transfected 48 hrs with a stabilized (dominant-positive mutant) β-catenin; and 4) LNCaP cells maintained 10 days in phenol red free RPMI medium supplemented with 10% charcoal-stripped serum (CS-FBS, an androgen free condition known to induce NE transdifferentiation of LNCaP cells). mRNAs were extracted from the samples using the Superarray mRNA purification kit and the mRNAs were converted to biotin-16 dUTP labeled cDNA using the GE Array Ampo-Labeling kit. Labeled cDNAs were hybridized to individual microarrays overnight and hybridization was detected using the Genearray Chemi-luminescent Detection Kit followed by exposure to film. Scanned films were analyzed using Gene Array Analysis Software, Scanalyze. The program, Gene Array Analyzer was used to compare gene expression levels between control array and test array. The experiment was repeated with a new set of mRNAs.

Because this assay involves film-based detection and measurement, a cutoff of 3-fold change in mRNA level was set for the results. The results showed 3-fold or greater upregulation of 26 gene products under all 3 test conditions (Table 2) compared to control.

TABLE 2
Genes induced 3-fold+ in LNCaP cells transfected with Pro-PC,
β-catenin, or maintained 10 days in CS-FBS. Numbers indicate relative
increase in gene expression compared to control (empty vector
transfected) LNCaP Cells (wnt-pathway gene focused array).

| Gene | Tcf Target | Pro-PC | β-catenin | CS-FBS |
|---|---|---|---|---|
| BMP4 | + | 4.1 | 3.4 | 6.5 |
| Fra-1 | + | 11.7 | 21.4 | 20.2 |
| GAS | + | 5.7 | 3.4 | 7.2 |
| GJA1 | + | 9.2 | 6.7 | 12.8 |
| Jun | + | 6.3 | 3.0 | 5.3 |
| c-Myc | + | 4.5 | 3.9 | 4.9 |
| COX-2 | + | 11.3 | 14.4 | 9.5 |
| c-Ret | + | 8.1 | 5.7 | 5.3 |
| Cyclin D1 | + | 4.1 | 4.1 | 6.2 |
| Cyclin D3 | + | 5.3 | 4.2 | 3.4 |
| CLDN1 | − | 4.1 | 4.1 | 6.2 |
| CTNNBIP1 | − | 16.4 | 2.4 | 3.6 |
| DKK2 | − | 4.1 | 4.4 | 4.9 |
| DKK4 | − | 4.3 | 3.7 | 4.4 |
| HST | − | 5.6 | 4.7 | 8.0 |
| FRAT1 | − | 4.0 | 3.2 | 4.4 |
| FZD2 | − | 4.8 | 3.2 | 5.7 |
| F2D4 | − | 3.0 | 3.2 | 6.9 |
| FZD10 | − | 10.2 | 3.1 | 7.3 |
| LEF1 | − | 6.5 | 3.1 | 7.2 |
| NKD1 | − | 17.7 | 11.3 | 13.9 |
| NKD2 | − | 6.3 | 3.8 | 4.5 |
| WNT3 | − | 6.5 | 6.0 | 19.7 |
| WNT10A | − | 8.9 | 8.5 | 3.7 |
| WNT11 | − | 7.2 | 3.1 | 4.0 |
| WNT7B | − | 5.7 | 12.3 | 5.9 |

This list of gene products includes 10 that are primary Tcf transcriptional targets, including important cell regulatory genes (Jun, c-myc, cyclin D1, D3) as well as differentiation regulating gene products (BMP-4, Cox-2, c-Ret). Additionally, 16 gene products that play a role in wnt-signaling (but are not known targets of Tcf transcription) were upregulated including wnt pathway initiators (WNT3, 7B, 10A, 11), wnt receptors (FZD2, 4, 10) and even the LEF-1 transcription factor that is a Tcf family transcription factor. An RT-PCR procedure (FIG. 2, Cox-2) confirmed, at least, that Cox-2 mRNA is highly upregulated in pro-PC transfected cells supporting the microarray data. These data support the idea that pro-PC induces wnt-signaling as well as the idea that androgen-withdrawal is associated with upregulation of pro-PC expression and increased wnt signaling. Finally, several gene products are noted that were induced 3-fold or more in pro-PC transfected and CS-FBS treated cells but were not induced to this level in β-catenin transfected cells (Table 3). This finding indicates the possibility that pro-PC expression may have additional effects on PCa cells.

TABLE 3
Genes induced 3-fold+ in LNCaP cells transfected with Pro-PC
or maintained in CS-FBS but not when transfected with β-catenin.
Numbers indicate relative increase in expression.

| Gene | Tcf Target | Pro-PC | CS-FBS |
|---|---|---|---|
| NOS | + | 3.1 | 5.6 |
| AES | − | 3.4 | 3.1 |
| AXIN1 | − | 3.2 | 3.0 |
| AXIN2 | − | 3.4 | 6.3 |
| CDX1 | − | 3.1 | 2.1 |
| SFRP4 | − | 6.3 | 10.0 |
| WNT15 | − | 5.9 | 10.3 |
| WNT5B | − | 13.7 | 4.7 |
| WNT6 | − | 3.4 | 3.3 |

Stable lines of pro-PC transfected LNCaP cells have been established. These cells, which grow readily in culture, are also able to form tumors in castrated male nude mice (8/8 subcutaneous implants formed highly vascularized tumors at the site of implantation within 6 weeks after implantation). They also have an NE-like phenotype compared to parental LNCaP cells (see below) in that they express high levels of NSE and chromo-A. Finally, they have high nuclear levels of β-catenin and express 4.23 more normalized luciferase from a tcf-sensitive reporter vector than parental LNCaP cells.

Protocadherin-PC and Neuroendocrine Transdifferentiation of PCa Cells

The evidence above shows that pro-PC expression is accompanied by upregulation of wnt signaling in PCA cells. Whereas the wnt signaling pathway is highly investigated because of its involvement in the development of several human tumors, it is also a well studied because it a cellular signaling pathway that is required for morphogenesis and differentiation of many normal embryonic tissues, including the limb bud, kidney and neural crest cell derivatives (Yenfeng et al., 2003; Lustig and Behrens, 2003; Vainio, 2003; Yang, 2003). Considering the importance of wnt signaling for neural crest cell differentiation and our observations that chronic culture of LNCaP cells in medium depleted of androgens induces pro-PC expression (see FIG. 20), wnt signaling as well as transdifferentiation of these cells to the NE phenotype (de la Taille et al., 2003), the potential relationship between pro-PC, wnt signaling and NE transdifferentiation was explored in the prostate cancer cell model systems. To this end, studies were designed to evaluate whether pro-PC expression might be more extensively associated with NE transdifferentiation in PCa cells. In an initial experiment to further demonstrate the coincidental nature of these two events (pro-PC expression and NE transdifferentiation), LNCAP cells were exposed to a series of 4 different chronic culture conditions that are known to induce NE transdifferentiation of these cells (db-cAMP [1 mM], Il-6 [50 ng/ml] or NS-398 [5 μM] for 6 days or growth in phenol red-free medium with 10% charcoal-stripped serum (CSS-FBS for 10 days) (Bang et al., 1994; Shen et al., 1997; Murillo et al., 2001; Jimenez et al., 2001; Meyer-Siegler, 2001; Deeble et al., 2001). Western blot analysis of protein extracts from these chronically treated LNCaP cells for NE markers (NSE and chromo-A) shows that they were highly upregulated compared to control cells (FIG. 3A) and this was also evident by the altered morphology of the cells in which they acquired long cellular processes. When RNAs were extracted from the control or treated cells and analyzed by RT-PCR for expression of pro-PC, all treatments that induced NE differentiation in LNCaP cells also induced pro-PC expression (FIG. 3B). A more direct relationship between pro-PC expression and differentiation to the NE phenotype was found in an experiment in which pro-PC cDNA was transfected into LNCaP cells using a pro-PC expression vector (FIG. 3C). 48 hrs transfection with pro-PC highly upregulated NSE and chromo-A expression, similar to cells grown in CS-FBS. NE transdifferentiation was also induced in LNCAP cells by transient transfection with stabilized β-catenin, the end molecule in the wnt signaling pathway, showing that NE transdifferentiation is induced simply by activating wnt signaling (FIG. 3C) and coincidentally supporting the hypothesis that pro-PC expression activates wnt signaling that leads to NE transdifferentiation. Transfection of pro-PC into the PC-3 cell line highly induces upregulation of NSE and chromo-A, showing that this effect is not restricted to LNCaP cells.

Likewise, suppression of β-catenin expression (by an siRNA targeting β-catenin) (FIG. 7A) in LNCaP cells transiently transfected with pro-PC was sufficient to block NE transdifferentation (FIG. 7B). Finally, similar results (suppression of NE transdifferentiation) were obtained when pro-PC was co-transfected with a dominant negative Tcf (FIG. 6) which can also block wnt pathway signaling by suppressing of β-catenin/tcf-mediated transcription. These latter data strongly support the idea that the action of pro-PC in inducing NE transdifferentiation of PCa cells is dependent upon its ability to activate wnt signaling.

Protocadherin-PC Binding Partners

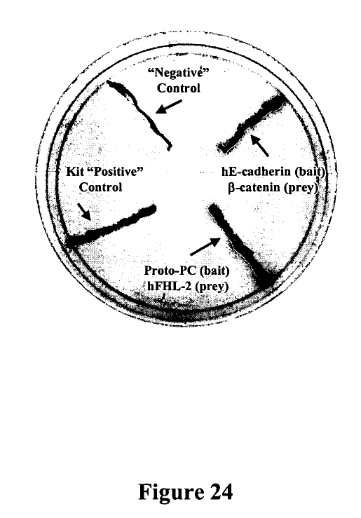

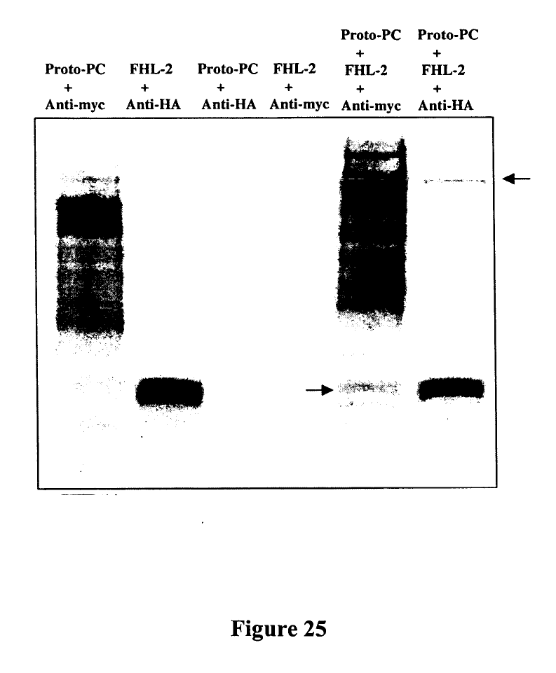

To better characterize the function of pro-PC and to ascertain the validity of the potential β-catenin binding site within the carboxy-terminal domain of the pro-PC protein, a yeast-2-hybrid screen was conducted in which pro-PC cDNA was used as the “bait” to identify binding partners (“prey”) that might be present in a cDNA library from LNCaP cells. These studies have resulted in the identification and confirmation of several strong pro-PC binding partners including human snapin, actinin alpha-4, ABCC4 (a transmembrane protein of the CFTR/MRP family), KIAA and the human four and half LIM domain protein, FHL2. Human metallothionine 2a, dihydrolipoamide-5-acetyltransferase and human filamin A alpha were found to be weaker binding partners. While many of these binding partners appear to be mainly cell structural proteins (and to reflect the potential for protocadherins to participate in structural aspects of a cell), one molecule that was not pulled out in this functional assay, β-catenin, which was in contrast to our expectations. A further effort was made to clone human β-catenin cDNA into the prey vector and to directly test for an interaction with pro-PC, using human E-cadherin as a positive “bait” to ensure that the yeast-2-hybrid screen could detect the interaction between cadherins and β-catenin, and, as shown in FIG. 24, a human E-cadherin bait was successful in detecting the interaction between these two molecules (E-cadherin and β-catenin). In FIG. 24, the strong positive interaction between FHL-2 and pro-PC in the yeast-2-hybrid assay is confirmed in an in vitro binding-immunoprecipitation (“pulldown”) assay (FIG. 25). It is possible that the pro-PC vector construct used in the yeast-2-hybrid screening assay is not suitable to detect a direct interaction between pro-PC and β-catenin and in vitro “pulldown” assays can be conducted to determine whether mixtures of recombinant pro-PC and β-catenin proteins might co-immunoprecipitate in this type of assay. Deletion or mutation of the homologous β-catenin binding site in the 3′ region of pro-PC cDNA may suppress the ability of this cDNA to induce wnt signaling in PCa cells. However, FHL-2 is a protein that is known to directly bind to β-catenin and to stimulate transcription from β-catenin/tcf sensitive reporter vectors, thus it is considered to be a co-activator of β-catenin-mediated transcription (Wei et al., 2003; Martin et al., 2002) as well as a co-activator of other transcription factors (Morlon and Sassone-Corsi, 2003; Muller, 2000). FHL-2 may be mediating the interaction between pro-PC and β-catenin. FHL-2 may be a critical mediator of the effects of pro-PC on wnt signaling in PCa cells. The domain(s) of pro-PC that directly binds to FHL-2 are useful in this invention. That activation of wnt signaling by pro-PC depends upon FHL2 binding, increases our understanding of the mechanism(s) through which pro-PC affects cell signaling in the PCa cell.

Transfection of LNCaP cells with pro-PC expression vectors induce a state of apoptosis-resistance and induce NE transdifferentiation of PCa cells. A means to specifically “knockout” pro-PC expression to affect acquisition of therapeutic resistance and NE transdifferentiation in PCa models is provided. siRNAs that are suitable for knocking out expression of pro-PC are provided by the invention. Using the siRNA design program on the Ambion website, 3 different siRNAs (FIG. 4A) have been designed and testes. Selection of these siRNAs was based upon the desire to avoid any portion of the pro-PC gene with highly conserved domains (i.e., the cadherin boxes as well as the signal sequence and transmembrane domain regions). Thus the 3 different 19 bp regions that have been targeted for creation of siRNAs lie significantly 3′ of the putative AUG start sites (at positions 3043-3062 [#181; SEQ ID NO:4, FIG. 29], 3098-3117 [#190; SEQ ID NO:6; FIG. 31] and 3345-3364 [#208; SEQ ID NO:7; FIG. 32] on the complete pro-PC cDNA) and they will also potentially silence any gene product arising from the X-chromosome gene. However, RT-PCR analyses of PCa cell line models generally show very low expression of the X-encoded homologue that does not change with progression to apoptosis- or hormone-resistance. A test of these siRNAs (FIG. 4A) shows that they each have suppressive effects against pro-PC protein expression in a transient transfection assay, although the one from the region closest to the 5′ of the cDNA [#181; SEQ ID NO:4] appears to be the most effective. The siRNAs of the invention should not influence the expression of other critical cadherin proteins such as E-cadherin.

Biological Consequences Associated with the Expression of Pro-PC in Prostate/Prostate Cancer Cells

Pro-PC was identified as a gene product upregulated in variants of human PCa cells (LNCaP) that had acquired apoptosis-resistance as a result of repeated exposure to apoptotic agents. These cells also acquired hormone resistance as shown by their ability to form tumors in castrated male nude mice. The pro-PC gene is a male- and human-specific member of the evolutionary “old” protocadherin gene family and the major translation product of this gene is atypical for the family because of its lack of a signal sequence and the presence of a small domain in its C-terminal region that shares extensive homology with β-catenin binding domain of evolutionarily more contemporary classical cadherin genes. Transfection of this gene back into apoptosis- and hormone-sensitive PCa cells directly confers apoptosis- and hormone-resistance and also induces a NE transdifferentiation process similar to that associated with the natural progression of human prostate cancers to the aggressive and hormone-resistant state. Pro-PC expression in PCa cells is associated with increased activity of the wnt signaling pathway, a cellular signaling pathway that is involved in oncogenesis of human colon, skin and other tissues and results have shown that blockade of wnt signaling (by an siRNA and dominant negative Tcf approach), at least, suppresses the ability of pro-PC to induce the NE transdifferentiation process in PCa cells. Upregulation of pro-PC activates wnt signaling, and, perhaps, other signaling pathways in PCa cells, contributing to a loss of apoptosis- and hormonal sensitivity as well as a NE transdifferentation process that facilitates hormone-independent growth. Moreover, given the relationship between activation of wnt signaling and the development/progression of other common human cancers, aberrant pro-PC expression in benign prostate epithelial cells might lead these cells to acquire pro-malignant characteristics. An in vivo model involving the generation and analysis of transgenic mice that express pro-PC in the prostate is provided. In vitro models involving cultured human prostate cancer cell systems are provided.

Construction and Analysis of Prostate-Targeted pro-PC Transgenic Mice. The construction of transgenic mouse lines in which pro-PC expression is targeted to the mouse prostate gland through the probasin gene promoter element is provided. Introduction of pro-PC gene expression into normal prostate epithelial cells of the mouse induce chronic upregulation of wnt signaling, an increase in NE-like characteristics and increased potential to acquire pro-malignant characteristics by the epithelial cell population in the prostate of these mice. Breeding a transgenic mouse with a “LOXed” β-catenin gene third exon (removal of this exon results in a “stabilized” β-catenin and chronic activation of wnt signaling) with the MMTV-Cre mouse produces a mouse in which a stabilized beta-catenin is expressed in the prostate gland. Heterozygotes of this breed have squamous differentiation of the breast where the stabilized β-catenin is also expressed, but the prostate develops hyperplasia, distinct PIN-like lesions and epithelia with squamous “transdifferentiation” that was uncharacterized for any gene expression pattern (Fournari et al., 2002). This squamous “transdifferentiation” of prostate epithelial cells in these mice may be an NE trans-differentiation phenotype. The mouse prostates from pro-PC transgenic mice are analyzed both with regards to changes in gene expression patterns (by mouse Affymetrix oligonucleotide microarray analysis) and with specific immunohistochemical staining techniques to identify changes in expression of gene products involved in the wnt signaling pathway and NE transdifferentiation. The microarray gene expression analyses will be used to determine whether and which particular wnt target genes are upregulated in the pro-PC expressing mouse prostates as well as to directly quantify changes in expression of gene products related to the NE phenotype. As well, this type of analysis will permit identification of other cell signaling systems might be altered by pro-PC expression, as the wnt-target specific microarray analysis of pro-PC transfected LNCaP cells has already shown some differences when compared to β-catenin transfected cells. The prostate glands from these mice will also be characterized by standard histology to identify potential pre- or frank-neoplastic/anaplastic changes similar or more aggressive than those found in the β-catenin prostate transgenic model described above and by immunohistochemistry to evaluate whether there might be evidence for increased wnt signaling (accumulation of cytoplasmic/nuclear β-catenin, upregulation of c-myc or cyclin D1 expression) or NE transdifferentiation (expression of chromo-A, synaptophysin and other NE-neuropeptides) as would be predicted based on experimental results.

Crossing pro-PC Transgenic Mice with LADY (12-T7) Transgenic Mice. One unique aspect of the LADY system (Masumori et al., 2001) is its tendency to have a longer latent period for adenocarcinoma development than the TRAMP model, and, more important for this project is the availability of specific LADY sublines that do not give rise to aggressive NE-differentiated tumors as is inevitably the consequence with the TRAMP model system (Kasper et al., 1998). Breeding the prostate-targeted pro-PC transgenic mice with 12-T7 LADY subline that exclusively develops high grade PIN without NE differentiation (Kasper et al., 1998) will be done to identify expression of pro-PC and frank adenocarcinomas with an NE phenotype (mediated by activation of the wnt signaling pathway) that resemble the TRAMP or more aggressive LADY model tumors in terms of their general progression pattern. This is a relatively straight-forward experiment that will involve extensive characterization of the pro-PC X 12-T7 crossed males using histological evaluation of prostates from these mice, immunohistochemical analysis of prostates (especially PIN and adenocarcinomas) for evidence of increased wnt signaling (accumulation of cytoplasmic/nuclear β-catenin, increased expression of c-myc and cyclin D1) and immuno-histochemical evaluation of these same prostate lesions for evidence of increased NE differentiation (increased expression of chromo-A, synaptophysin and other neuropeptide hormones). Moreover, these mice will be followed over an extended period to characterize malignant progression involving metastatic lesions, which will also be characterized for NE properties by immunohistochemistry.

Aside from these bi-transgenic breeding experiments, studies can be designed to address the seeming conundrum that aggressive mouse transgenic tumor systems (TRAMP or LADY 12T-10) progress to NE-like tumors (Greenberg et al., 1995; Masumori et al., 2001) whereas the pro-PC gene of this invention is a human-only gene product. Mice do have a homologue for the X-linked gene, PCDHX (Blanco et al., 2000) and it has been observed that this gene, at least in humans, has the potential of yielding over 100 different transcripts resulting from splice variations and alternate transcription start sites (Blanco-Arias et al., 2004). Mouse prostate tumor progression in these transgenic models may be accompanied by upregulation in expression of mouse PCDHX homologue splice variants that, like the gene product encoded by the human pro-PC gene, lack signal sequence or critical transmembrane domain regions. Mouse gene databases can be searched to identify the mouse homologue and obtain its sequence. Using this sequence, PCR primers can be designed to amplify different regions of the mouse PCDHX homologue transcript domain and use these primers to amplify cDNA prepared from RNA of the mouse NE-10 cell line (Jin et al., 2004). These experiments will assess whether the expression of the homologue is upregulated in the NE-10 cells compared to normal mouse prostate by real-time PCR techniques. Then an assessment can be made to determine whether variant cDNAs from NE-10 cells can be amplified using primer sets that span the cDNA region containing the signal sequence and trans-membrane domains. Variants will be identified by the presence of multiple bands on agarose gels following RT-PCR procedures. All variant bands will be cloned into plasmids for sequencing and this will allow identification of any variant bands that might correspond with splice variants lacking a signal sequence or transmembrane domains. Using this information, primer sets can be designed to amplify and characterize full transcripts of such variants and test their activity for promoting wnt-signaling activation and NE transdifferentiation in cell models. The ability to identify increased expression of mouse PCDHX homologue splice variants that are defective for membrane insertion in these cells might resolve the conundrum that aggressive transgenic mouse models of PCa develop NE-like tumors while lacking the pro-PC homologue.

Comparison of Gene Expression Patterns Associated with Pro-PC Expression in PCa Cell Lines to Gene Expression Patterns in Wnt-Activated and Control PCa Cell Lines. Some “targeted microarray” analyses have been conducted to query whether changes in gene expression in LNCaP cells elicited by transfection with pro-PC are similar to changes in gene expression associated with activation of wnt signaling in LNCAP cells (by transfection with stabilized β-catenin). There are many similarities in genes induced by these two actions and the results show wnt signaling is activated in pro-PC expressing LNCAP cells. Pro-PC may be acting through other cell signaling pathways, perhaps because of its interaction with cell structural components (identified in the yeast-2-hybrid assay of pro-PC interaction). An Affymetrix Human Gene Chip Assay will be used to assess whether expression of pro-PC in a PCa cell line (LNCaP) is associated with upregulation of the wnt-signaling pathway and NE trans-differentiation as well as to test whether there may be other signaling pathways that are stimulated by pro-PC that are independent of the wnt signaling pathway. Gene expression patterns in each of the “test” groups (pro-PC expression or stabilized β-catenin expression) will first be compared to the control group using a hierarchical clustering analytical procedure to identify those gene products that are changed as a result of: 1) pro-PC expression; or 2) wnt signaling activation by increased β-catenin activity. These initial data sets (changes in gene expression) will then be scanned to identify changes in gene expression (upregulation) associated with wnt signaling pathway activation to confirm the relationship between pro-PC and wnt signaling upregulation. The initial data sets will also be scanned for changes in gene expression (upregulation) of gene products known to be expressed in NE cells (as exemplified by NSE, chromo-A, synaptophysin, bombesin, PRTPH, calcitonin, pro-gastrin, etc) to get a general pattern confirming the acquisition of the NE phenotype in cells expression pro-PC or stabilized β-catenin. Finally, the processed data sets will be compared to each other to identify changes in gene expression that might be specific to pro-PC expressing cells (as a result of transfection or growth in CSS-FBS) but not to wnt-activated cells (transfected with stabilized β-catenin). These types of comparisons will be able to confirm the hypothesis that pro-PC expression leads to wnt signaling activation and NE transdifferentiation. As well, novel gene products may be identified that are specifically changed by pro-PC (but not by wnt activation) that would lead to the study of alternate effects of pro-PC action (based on activation of cellular signaling pathways independent of wnt). This study will also lead to data sets that can be scanned to identify potential changes in gene expression in PCa cells that might have the potential for significant influence on the malignant phenotype; for example, changes in gene expression of gene products generically associated with apoptosis-regulation (exemplified by gene products such as bcl-2, bax, bad, bcl-XL, etc); cell cycle progression (exemplified by cyclins and cyclin-dependent kinases, etc), or metastatic activity (exemplified by KAl 1, plasminogen activator, TIMPs, etc). Thus, this type of very controlled experimentation and analysis has the potential to yield striking data sets that will address the ideas set forth in this Example, plus the potential to yield new insights into prostate cancer progression associated with expression of pro-PC or wnt signaling activation.

Targeted Downregulation of Pro-PC Expression in PCa Cells and Its Effects on Wnt Signaling, NE Transdifferentiation and Apoptosis- and Hormonal-Resistance. A siRNA approach is being developed that specifically and effectively targets and reduces pro-PC expression in prostate cancer cells. These experiments will address by another means the relationship between pro-PC expression, wnt signaling, NE trans-differentiation and apoptosis- and hormonal-sensitivity by knocking down pro-PC express in our PCa cell models, then showing that pro-PC gene knockdown effects various downstream activities. Potential sequences have been identified within pro-PC that will be useful for this targeting and these sequences are sufficiently specific so they are not likely to influence expression of highly related gene products (such as classical cadherins). Once the specific activity of these siRNAs are identified, this information can be utilized to construct a short hairpin RNA (shRNA) expression vector that could be used to downregulate pro-PC expression in a more stable manner. However, with the availability of transient and more stable pro-PC silencing agents, the experimental plan straightforward and will include testing for reduction of wnt signaling and NE transdifferentiation using transient transfection of siRNAs into PCa cells that express pro-PC and testing for reduction of apoptosis- and hormonal sensitivity in these same cells using short hairpin (sh) stable transfection vectors.

Construction, Analysis and Breeding of Transzenic Mice. To produce the transgenic prostate-targeted-pro-PC mouse lines, the pro-PC cDNA (with a C-terminal myc tag) has been recombined into the pPB-ARR2 expression vector (Adriani et al., 2001). This vector has been sequenced to ascertain appropriate vector design. Founder mice (identified by transgene detection in tail DNA) will be bred into non-transgenic animals for expansion of each Founder line. Upon expansion of stocks, founder and younger progeny males will be sacrificed for dissection of individual prostate lobes and these will initially be processed for standard histology and immunostaining to confirm transgene expression (with anti-myc antibody) and to characterize any fundamental prostate abnormalities, especially of the epithelial layer. The expectation is that younger animals may develop a squamous appearing epithelium as described in the β-catenin prostate mice and older animals (3-6 months) may show evidence for epithelial hyperplasia or neoplasia as also described in the β-catenin prostate model. Sections will also be analyzed by various NE-product immunostains (chromo-A, synaptophysin, bombesin) to identify potential NE phenotypes of epithelial cells. Pro-PC may confer a more aggressive prostate phenotype than that seen in β-catenin prostate mice and prostate sections will be analyzed for signs of overt anaplasia. Continued breeding and expansion of founder lines will enable the collection of multiple prostate specimens from confirmed founder progeny at defined age periods: 3, 6, 8 and 12 months (at least 5 each), for extraction of mRNA and gene expression microarray analyses of the mRNA on Affymetrix Mouse Gene Chips (#430, version 2.0). Results of the gene expression array analysis will be compared to control (non-transgenic mouse) prostates and the data sets identifying changes in gene expression in pro-PC transgenics will be searched for gene products that evidence the activation of the wnt signaling pathway (37 known target genes including c-myc, cyclin D1 and Cox-2) and for gene products associated with the NE phenotype (exemplified by mouse synaptophysin, chromo-A and bombesin, etc) to confirm that pro-PC is, at least, associated with these changes.

Upon obtaining stable, breeding sublines of pro-PC transgenic mice, select males or females will be bred into the LADY 12T-7 subline to obtain bi-transgenic progeny. Tail clip DNA of progeny will be analyzed and progeny having both pro-PC and SV40 T-antigen transgenes will be selected for inbreeding to amplify and provide stocks for maintenance. Selected cross-bred males will be sacrificed at defined ages (6 wk, 3, 6 and 8 months) to provide prostate tissues (5 each) for histological analysis of prostate abnormalities as identified above and will be compared to purebred 12T-7 or pro-PC alone lines at matched ages for presence of prostate growth abnormalities, especially the appearance of frank anaplasia/invasive adenocarcinoma. Evidence for the development of invasive adenocarcinoma in mixed bred mice will be followed by analysis of age-matched males over a 8-12 month time period to identify the presence of prostate adenocarcinoma at metastatic sites by histological analysis of tissues obtained from sacrificed mice. Tumor-containing sections will be characterized by NE marker immunostaining as described to identify a NE phenotype.

Affymetrix Oligonucleotide Microarray Analysis of Gene Expression Patterns in Transgenic Mouse Prostates and in LNCaP Cells Expressing Pro-PC or Stabilized β-catenin. Expression microarray analysis will be carried out on two types of specimens: 1) dissected prostates obtained from pro-PC transgenic and control mice (using mouse-specific gene chips); and 2) LNCaP cells expressing pro-PC, stabilized β-catenin or control (transfected with empty vector) to evaluate expression patterns of wnt-signaling pathway and NE-specific genes as well as to identify differences in gene expression changes between pro-PC or β-catenin expressing cells (using human-specific gene chips). Briefly, after tissue (control or transgenic prostates) or cell (LNCaP cells; 1] transfected 48 hrs with empty vector; 2] transfected 48 hrs pro-PC; 3] cultured 10 days in androgen-free medium; 4] transfected 48 hrs with stabilized β-catenin) samples are initially homogenized, total RNA is isolated using the Qiagen RNeasy Kit and reagents and dissolved in RNase-free H₂O. Poly A+ RNA is reverse transcribed with T7-oligo(dT) primers in 1^{st} stand cDNA synthesis (Poly-A RNA control kit and One-Cycle cDNA Synthesis Kit of Affymetrix). cDNA is prepared using the Affymetrix Sample Cleanup Module and is used as a template for in vitro transcription amplification and biotin labeling using T7 RNA pol and biotinylated ribonucleotide analogues using the Affymetrix IVT Labeling Kit. The cRNA is fragmented into 35-200 base fragments by metal induced hydrolysis and the cRNA is provided to the facility for hybridized with Affymetrix GeneChip oligonucleotide microarrays (Mouse Genome 430 Version 2.0 or Human Genome U133 Plus 2.0, which contain over 45,000 probe sets representing 39,000 transcripts derived from “well-substantiated” human genes). For each specimen, two sets of chips will be used to compare the gene expression profiles of test specimens (pro-PC transgenic mouse prostate or pro-PC expressing LNCaP, androgen-free LNCaP or β-catenin expressing LNCaP) with controls (nontransgenic prostate or LNCaP transfected with empty vector.

Hybridized slides will be washed and scanned using the confocal laser scanner. Fluorescence intensities will be corrected for background noise, normalized, and then quantified. Hierarchical clustering analyses will be performed to group genes with similar patterns of expression (compared to control groups). For mouse or human studies, each test group data set will be observed for increased expression of 37 known wnt target genes as well as a collection of 67 genes involved in the wnt signaling pathway, as were present on the targeted microarray analysis already completed. Additionally, each test group data set will be observed for changes in expression of a large category of genes associated with the NE phenotype (as described throughout the application). For the LNCaP cell analysis, data sets from pro-PC transfected cells or androgen-free LNCaP cells will be compared and contrasted to stabilized β-catenin transfected cells to identify differences in expression patterns between these two sets (pro-PC expressing vs non-pro-PC expressing cells). A goal of these studies will be to identify the subset of gene products upregulated in pro-PC expressing cells that are not upregulated in non-pro-PC expressing cells as a means of identifying potential alternate signaling pathways affected by pro-PC expression but not by simple wnt signaling activation.

Silencing pro-PC Expression in PCa Cells to Show Direct Effect of Pro-PC on wnt Signaling, NE Transdifferentiation and Acquisition of Apoptosis- and Hormonal-Resistance

Effective siRNAs against pro-PC will be utilized in transient and stable transfection experiments to test the idea that suppression of pro-PC expression in LNCaP cells reduces wnt signaling, reduces NE transdifferentiation and suppresses development of apoptosis- and hormone-resistance. The first experiments will involve transient transfection (48, 72 hr analysis) and include samples of untransfected LNCaP cells (negative control), LNCAP cells transfected with pro-PC expression vector alone, pro-PC expression vector and scrambled siRNA or pro-PC and lamin siRNA (positive controls for wnt activation and NE transdifferentiation) and pro-PC expression vector LNCaP cells transiently co-transfected with the 3 pro-PC siRNAs (test specimens). Specimens will be analyzed for nuclear accumulation of β-catenin by cell fractionation and comparative Western blot procedures, induced expression of c-myc and cyclin D1 by real-time PCR and comparative Western blot procedures (markers of wnt activation) and for expression of NSE, chromo-a and synaptophysin (NE biomarkers). Reduction of wnt and NE markers by active pro-PC siRNAs but not by scrambled or lamin siRNA supports dependence of these actions (wnt signaling, NE transdifferentiation) on pro-PC expression. These siRNAs will be tested for their ability to suppress NE transdifferentiation of LNCaP cells induced by CS-FBS, db-cAMP, IL-6 or NS-398. Control and treated cells will be transiently transfected during the last 48 hrs of the treatment with the active siRNAs (or controls) and cell extracts will be evaluated for expression of NSE and chromo-A by Western blotting. Reduction of NE markers will indicate interference with NE transdifferentiaton. Similar experiments will be carried out in PC-3 cells that also undergo NE transdifferentiation in response to db-cAMP and 1′-6. A stable short hairpin expression vector will be designed using the sequence information of active siRNAs as well as a control vector with a scrambled sh sequence (negative control) within the Promega psiLENT vector and the U6 Hairpin Cloning System. These vectors will be used to transfect the -TR and -SSR variants of LNCaP as well as a stable transfected pro-PC expressing LNCaP variants. When reduction of pro-PC expression is confirmed in the active shRNA transfected variants (on Western blot), the transfected variants will be compared to control (parental LNCap) and variant untransfected -TR cells for sensitivity to apoptotic agents in vitro (TPA) and for ability to form tumors in castrated male nude mice using procedures already described. These experiments will assess whether pro-PC reduced cell variants lose resistance to TPA-induced apoptosis and whether these cells are less able to form tumors in castrated male nudes. Co-transfection with the wnt reporter vector (pTOP) will be used to assess downregulation of wnt signaling in these cells compared to controls.

Identification of the Molecular Mechanism Through which Pro-PC Activates the Wnt-Signaling Pathway in Prostate Cancer Cells.

Pro-PC expression is accompanied by changes in the subcellular localization of β-catenin and with activation of wnt-signaling in PCa cells. Hormone-resistant human prostate tumors upregulate pro-PC expression and also have aberrations in subcellular β-catenin localization suggesting that the wnt signaling pathway is frequently dysregulated (de la Taille et al., 2003). Pro-PC action induces wnt signaling in prostate cancer cells. Initially (Chen et al., 2002; de la Taille et al., 2003) a relationship was proposed between pro-PC expression and wnt signaling activation based on the ability of pro-PC protein to directly bind β-catenin, protect it from degradation and, ferry it to the nucleus where it could interact with Tcf/LEF-1. This was supported by data showing that pro-PC immunoprecipitation was accompanied by co-precipitation of β-catenin protein. However, yeast-2-hybrid studies do not support a direct interaction between pro-PC and β-catenin. Rather, the yeast-2-hybrid experiment identified a direct interaction between pro-PC and the FHL-2 protein. FHL-2, a member of the 2½ LIM domain gene family, is a known co-activator of β-catenin-promoted transcription, as well as a known direct binding partner of β-catenin (Wei et al., 2003; Martin et al., 2002). Whereas further experimentation will assess whether pro-PC might directly interact with β-catenin through in vitro “pulldown” assays, it is also a possibility that FHL-2 protein acts to mediate the binding of β-catenin with pro-PC (in a complex). FHL-2 co-immunoprecipitates with pro-PC/β-catenin complexes from prostate cancer cells. The invention provides a small deletion pro-PC expression vector that lacks FHL-2 or the putative β-catenin binding domain. siRNAs that target FHL-2 are provided.

Does FHL-2 co-precipitate with pro-PC/β-catenin from apoptosis- and hormone resistant prostate cancer cells? A recombinant FHL-2 with a C-terminal HA tag that is detectable on Western blot by anti-HA antibody is provided (see FIG. 25). This vector will be transfected into pro-PC expressing LNCaP cells (tagged with myc), immunoprecipitate pro-PC with anti-myc and evaluate the washed immunoprecipitates for β-catenin (using anti-β-catenin antibody) and FHL-2 (using anti-HA antibody) protein. Converse immunoprecipitates made using anti-β-catenin or anti-HA as the primary immunoprecipitating Ab will be probed for pro-PC (myc). In vitro “pulldown” studies using tagged proteins made in in vitro transcription translation reactions similar to experiments shown in FIG. 25 will be done to show that recombinant pro-PC, FHL-2 and β-catenin proteins can form immunoprecipitatable complexes when mixed together.

Identification of the FHL-2 binding domains on pro-PC. By identifying this (these) binding site(s), a recombinant pro-PC cDNA can be created that lacks this binding site and then tested to determine whether this molecule is able to activate wnt signaling or to confer apoptosis- or hormonal-resistance following transfection of parental LNCaP cells. This is a straightforward experiment that involves the selective generation of cDNAs that have small, but variable deletions, especially within the C-terminal domain that is homologous to the intracellular domain of the homologue PCDHX. This will be done using standard recombinant DNA procedures involving selective utilization of restriction endonuclease cut sites to remove small portions of DNA. Attention will be paid to creating deletions that do not induce any sort of frame-shift in the resulting protein product so that all other domains are maintained. This will be confirmed by sequencing all variants. The partially deleted cDNAs will be tested in the yeast-2-hybrid assay and pull down assays using the deleted pro-PC as the bait and FHL-2 cDNA as the prey. Using this method, pro-PC variants can be identified that fail to activate lacZ expression in the yeast cells. Upon finding such variants, the deleted regions that confer binding activity can be narrowed down by fine manipulation of the cDNA (for example, deletions and site-specific mutagenesis) and again, testing in the yeast-2-hybrid and pull down assays. All of the pro-PC deletion variants that lack activity in the yeast-2-hybrid assay will be tested for their ability to activate wnt signaling in LNCaP cells via a co-transfection study with the β-catenin/TCF sensitive reporter plasmid TOP or the inactive reporter plasmid FOP (all controlled with β-gal con-transfection vectors). It is expected that, if FHL-2 binding is an important mediator in the activation of wnt signaling by pro-PC, deletions of the FHL2 binding site will fail to activate wnt signaling. Likewise, cells transformed with these variants should lack apoptosis- and hormonal-resistance as tested in our in vitro and in vivo model systems. Since it is possible that the yeast-2-hybrid assay is not stringent enough to identify a direct interaction between pro-PC and β-catenin, the existence of such a direct interaction might be detected using a pull down type assay, and this type of assay should also be used. Other experiments will selectively delete the homologous β-catenin binding domain from pro-PC cDNA to test whether this action reduces the ability of the modified cDNA to induce wnt signaling or NE transdifferentiation in LNCaP cells.

Development and utilization of a siRNA strategy targeted against FHL-2 to suppress its expression in pro-PC transformed cells. Commercial sources will be used to design a suitable siRNA that targets FHL-2 expression in LNCaP cells and then test these (proven effective) siRNAs (and controls) for their ability to restore apoptosis-sensitivity to pro-PC transformed LNCaP cells and to suppress tumor formation of pro-PC transformed LNCaP cells in castrated male nude mice. This involves the construction of shRNA expression vectors utilizing sequence regions supported by siRNA experiments as described above. Detection of ability to reduce FHL-2 expression will be undertaken by evaluation of transfected LNCaP cells for FHL-2 mRNA reductions (by real-time PCR procedures) or by comparative Western blot procedures (compared to control scrambled siRNAs) using a commercial anti-FHL-2 antibody.

Another aspect of pro-PC that falls within the auspices of this Example is an evaluation and analysis of the regulatory elements that control expression of this gene in PCa cells and, experiments can be designed to dissect the promoter of the pro-PC gene to address this idea. Pro-PC effects wnt signaling in PCa cells and the β-catenin protein binds to and co-activate androgen receptor (AR) in PCa cells (Song et al., 2003; Pawlowski et al., 2002; Morlon et al., 2003). A novel aspect of wnt signaling involving regulation of AR expression by wnt-(β-catenin-) mediated signaling is provided. Active Tcf binding sites in the proximal human AR promoter are utilized when wnt signaling is activated (by pro-PC or β-catenin transfection, CHIP assay confirmed) and AR mRNA increases as a result of binding. However, a more intriguing aspect is that AR protein levels decline, even as AR mRNA levels significantly increase, giving a long-term effect of partial suppression of AR action with chronic wnt signal activation. The reduction of AR protein levels in prostate cancer cells by the wnt-signaling pathway is a function of increased proteolysis of AR.

### Example 7

### Interaction of PCDH-PC and FHL-2 Protein

Protocadherin-PC(PCDH-PC) expression activates the canonical wnt signaling pathway (identified by increased nuclear accumulation of the beta-catenin protein and increased transcription from the Tcf/LEF-1 transcription factor) in human prostate cancer cells and this action may be responsible for increasing the aggressive characteristics (including therapeutic resistance) of prostate cancer cells (Yang et al., 2005). To better understand how PCDH-PC expression affects wnt signaling, a yeast-2-hybrid assay was performed to identify other proteins that directly bind to PCDH-PC. In this assay, the PCDH-PC cDNA is fused to a portion of the Gal-4 transcription factor and this was used as a “bait” to screen a recombinant cDNA library from the human prostate cancer cell line, LNCaP, in which each cDNA was likewise fused to the other portion of the GAL-4 protein. When recombinant “bait” PCDH-PC protein directly binds to any other protein encoded by the prostate cancer cell library, the two portions of GAL-4 are brought into juxtaposition, activating its β-galactosidase enzymatic activity mediating metabolic breakdown of the artificial X-gal substrate that produces a blue-green color when metabolized. Here, 8 recombinant human cDNAs were isolated that encoded proteins that gave a “positive” reaction in the Yeast-2-hybrid assay. The individual “positive” cDNAs were sequenced and the gene products encoded by these cDNAs were identified as: 1) human actinin alpha-4; 2) human snapin, a SNARE-associated protein; 3) human ABCC4 sub-family C(CFTR/MRP, Member 4); 4) human KIAA; 5) human filamin A, alpha; 6) human Kelch-like ECH-associated proprotein 1; 7) human dihydrolipoamide S-acetyltransferase; and 8) human four and half lim domain protein (FHL-2). FIG. 24 shows an agar plate (containing the X-gal substrate) in which a yeast colony transfected with both the PCDH-PC bait and recombinant human FHL-2 cDNA has been streaked. Notice that this streaked colony has a blue-green coloration indicating the positive interaction between the gene products encoded by the two recombinant vectors.

Whereas most of the cDNAs found in this assay represent gene products that are considered to have a “structural” function within cells, the FHL-2 gene product was particularly interesting (with regards to the potential activation of the wnt signaling pathway by PCDH-PC) because FHL-2 was previously identified as a co-activator of β-catenin/LEF-1/Tcf-mediated transcription in human cells (Wei et al., 2003; Martin et al., 2002). As well, FHL-2 is known to be a co-activator of human androgen receptor-mediated transcription (Martin et al., 2002). Therefore, the potential interaction between PCDH-PC and FHL-2 protein might have functional consequences for the activation of wnt signaling in prostate cancer cells as well as functional consequences for androgen-receptor mediated transcription that is believed to participate in prostate cancer cell behavior.

To further substantiate the potential direct binding interaction of PCDH-PC with FHL-2, an in vitro transcription/translation procedure has been performed in the presence of radioactive (³⁵S)-methionine to produce ³⁵S-labeled recombinant human PCDH-PC protein (tagged with a portion of the human c-myc protein) and ³⁵S-labeled recombinant human FHL-2 protein (tagged with a portion of the hemaglutinin [HA] molecule). As shown in FIG. 25, a commercially-available antibody that recognizes the myc-tag can immunoprecipitate the PCDH-PC (Proto-PC) protein but not the FHL-2 protein. Likewise, an antibody that recognizes the HA antigen can immunoprecipitate the FHL-2 protein but not PCDH-PC (Proto-PC). When PCDH-PC (Proto-PC) and FHL-2 are mixed together, the antibody against the myc-tag co-precipitates FHL-2 protein along with PCDH-PC and the antibody against the HA tag co-precipitates PCDH-PC protein along with FHL-2. This further supports the idea that PCDH-PC and FHL-2 are functional binding partners. Based upon this data, PCDH-PC binding to FHL-2 may facilitate the activation of wnt signaling and the FHL-2 binding domain on the PCDH-PC protein may be a target for the suppression of wnt signaling in prostate cancer cells that express PCDH-PC and have a potential therapeutic action against hormone-resistant human prostate cancer cells that express PCDH-PC.

### Example 8

### Anti-Protocadherin-PC Antibodies for Use as Prostate Cancer Research and Diagnostic Tools

Recombinant human PCDH-PC, polyclonal and monoclonal antibodies against human PCDH-PC have been produced. Methods for detecting the presence of PCDH-PC in human prostate samples have been developed. These antibodies can be used, for example, 1) as a tumor marker for early detection of prostate cancer; 2) for pre-treatment staging of prostate cancer; 3) for post-treatment monitoring of prostate cancer; 4) as a marker to distinguish between indolent versus aggressive prostate cancer; and 5) as a research tool to elucidate the molecular mechanisms involved in prostate cancer initiation and progression.

Production of Rabbit Polyclonal Antibodies which Specifically Recognize the Protocadherin-PC

The peptides (SIPENSAINSKYTNP (SEQ ID NO:24), NMQNSEWATPNPENR (SEQ ID NO:25) and ETKADDVDSDGNRVT SEQ ID NO:26)) that correspond to three different regions of the protocadherin-PC have been synthesised and coupled with a carrier protein KLH (mollusk Megathura crenulata). A mixture of the 3 peptides was then used for rabbit's immunization. Rabbits were immunized as follows: The primary immunization is performed using a PBS solution containing the Freund adjuvant together with 100 μg of the immunogen. Injections have been monitored by employing a multi-sites strategy. Then animals were immunized later three times at 3-week intervals. The titration of the produced antibodies was evaluated by a standard ELISA technique. After 4 immunizations, the animals were sacrificed and antibodies were purified on affinity column. Each synthetic peptide is separately coupled to Sepharose beads. (NHS-activated Sepharose™ 4 Fast Flow, Amersham Biosciences). Serums were loaded onto the different columns allowing the specific purification of antibodies depending on their affinity with each peptide.

Production of Monoclonal Antibodies to PCDH-PC

Production and Purification of Human Recombinant PCDH-PC (rPCDH-PC)

The cDNA coding for human protocadherin-PC was isolated by Chen et al, (Oncogene, 2002 Nov. 7; 21:7861-71). It was cloned into pET3a vector, thereby placing the target cDNA under the control of the T7 promoter. pET3a-PCDH-PC was transformed into E. coli strain BL21(DE3)RIPL which expresses T7 polymerase upon induction with IPTG (isopropyl β-d-thiogalactoside). Appropriate transformants were identified by restriction analysis and sequencing. The expressed rPCDH-PC was verified by western blot analysis.

Large-scale isolation of PCDH-PC was performed as fellow. BL21(DE3)RIPL/pET3a-PCDH-PC culture was grown in 50 ml of Luria-Bertani (LB) broth at 37° C. with 100 μg/ml ampicillin in a shaking incubator overnight. A 5 ml sample of this culture was grown in 500 ml of prewarmed LB broth/ampicillin until the A₆₀₀ increased to about 0.7. IPTG was added to a final concentration of 0.1 mM to induce the synthesis of PCDH-PC. After 4 h of cultivation at 20° C., the cells were harvested by centrifugation (5000 g; 10 min). The cell pellet was then used to extract recombinant PCDH-PC.

The cell pellet washed with buffer A (100 mM Tris, pH 8.0, 100 mM NaCl and 1 mM EDTA). After centrifugation (5000 g, 5 min). The cell pellet was suspended in buffer A. Lysozyme was then added to final concentration of 1 mg/ml and incubated 20 min at room temperature. After centrifugation (5000 g for 10 min), the pellet was resuspended in buffer A containing additional 1% sodium deoxycholate. This was followed by 10 minutes incubation on ice. MgCl₂ and DNAse I were added to final concentrations of 8 mM and 50 μg/mL respectively. The suspension was conserved on ice during 1 hour and subjected to centrifugation at 12500 g for 15 min at 4° C. The pellet washed two times with buffer A containing 1% NP-40 and once with phosphate-buffered saline (PBS). Of note, each wash was followed by centrifugation at 12500 g for 15 min. Resulted inclusion bodies corresponding to the recombinant protocadherin-PC were solubilized in 50mM Tris, pH 8, 6, 6 M guanidine and 1 mM DTT for overnight at 4° C. The solution was clarified by centrifugation at 12500 g for 30 minutes. The supernatant was loaded onto a size-exclusion chromatography column (Sephacryl S-300, Amersham Biosciences) monitored with an in-line UV monitor. Elution was performed using Tris buffer 50 mM containing 6M guanidine, 1 mM DTT, pH 8,6. Fractions of 1 mL were collected. The presence of PCDH-PC in these fractions was tested by Enzyme-linked Immunosorbent Assay (ELISA) employing rabbit polyclonal antibodies anti-PCDH-PC. Positive fractions were pooled and subjected to dialysis against PBS. The solution was subjected to centrifugation at 12500 g for 10 min at 4° C. The supernatant corresponding to soluble recombinant PCDH-PC was separate to the insoluble PCDH-PC (precipitated form). These two fractions were stored at −20° C.

Immunization of Mice

Four-week old female Balb/c mice were injected intraperitoneally (IP) with 200 μg of recombinant human PCDH-PC with complete Freund's adjuvant (Sigma). This was followed after 2 weeks by three further IP immunizations at 2 weeks intervals. In this process each mouse was administrated 200 μg of PCDH-PC in incomplete Freund's adjuvant. Following the third boost, the mice were bled and serum antibody titers against PCDH-PC checked by ELISA using the rabbit polyclonal antibodies anti-PCDH-PC. Three days before fusion, mice with the highest titer were given a final intravenous injection of 50 μg of soluble PCDH-PC.

Fusion and Cloning

The mice immunized with PCDH-PC were sacrificed by cervical dislocation and the spleens were removed into a 60 mm. petri dish containing 5 ml of sterile DMEM. After rinsing, the spleens were transferred to a second dish and perfused. The spleen cells were pipetted into a 50 ml centrifuge tube. Centrifugation was carried out at 1000 rpm for 10 min. The pellet was suspended in serum free DMEM and cell number was counted. Spleen cells were mixed with myeloma cells (P3X63AG8/653, ATCC CRL1580) at a ratio of 5:1 (1×10⁸ splenocytes: 2×10⁷ myeloma cells) and centrifuged at 1000 rpm at 10 min. The cells were then washed once with DMEM medium and centrifuged again at 1000 rpm in a 50 ml conical tube. The supernatant is discarded, the cell sediment is gently loosened by tapping, 1 ml of 45% (v/v) of polyethylene glycol 1000 (Sigma) was dropwise added to the mixture, followed by incubation at 37° C. for 2 minutes. 5 ml of DMEM was added dropwise at room temperature within a period of 3-4 min. Afterwards 5 ml of DMEM containing 10% FCS was added dropwise within 1 min, mixed thoroughly, filled to 50 ml with DMEM containing 10% FCS and subsequently centrifuged for 5 min at 1000 rpm. The sedimented cells were resuspended in hypoxanthine-azaserine selection medium (100 nmol/1 hypoxanthine, 1 μg/ml azaserine in DMEM+10% FCS). Cells were seeded in 96 wells of microtiter plates at 5×10⁴ cells per well. Every 2 days, ½ of the medium was replaced by fresh selection medium. Growth of clones was monitored by viewing under an inverted microscope. After approximately ten days, small colonies of hybridoma cells appeared and were present in nearly all wells. In order to identify hybridoma colonies which synthesized and secreted antibodies having the specificity for PCDH-PC, the supernatants from wells showing growth were tested by ELISA. Cells identified as capable of producing anti-PCDH-PC were subjected to cloning by the limiting dilution method in the following manner. The culture of those hybridomas were counted by staining with Trypan Blue and diluted with DMEM containing 10% FCS to give a concentration of 3 cells/ml. 100 μl of cell suspension were added per well to a 96 well plate (calculated to provide about 0.3 cell per well). After 2 weeks, visible hybridomas were tested for antibodies production. Furthermore, a number of additional screening techniques (i.e., immunohistochemistry, western blot) were utilized to characterize the antibodies. The screening and stability test steps were repeated several times with the PCDH-PC specific antibody-producing hybridomas showing the highest stability and antibody specificity. A final selection of the best hybridomas was made and the hybridomas designated as follows: SSA, LIU and C32. SSA and LIU cell lines were deposited (in accordance with the requirement of the Budapest Treaty for patent purposes) on Jan. 24, 2006 with the Collection Nationale de Cultures de Microorganismes (CNCM), Institut Pasteur, 25 rue du Docteur Roux, F-75724 Paris Cedex 15. These cell lines are assigned as HB 0337 SSA (CNCM I-3561) and HB 0337 LIU (CNCM I-3560).

Antibody Purification

The monoclonal antibodies secreted by the selected hybridoma cells are suitably purified from cell culture medium or ascites fluid by conventional immunoglobulin purification procedures such as, for example, ammonium sulfate precipitation, protein A-Sepharose chromatography, dialysis, or affinity chromatography.

Techniques Used to Characterize Antibodies Anti PCDH-PC

Enzyme-Linked Immunosorbent Assay (ELISA)

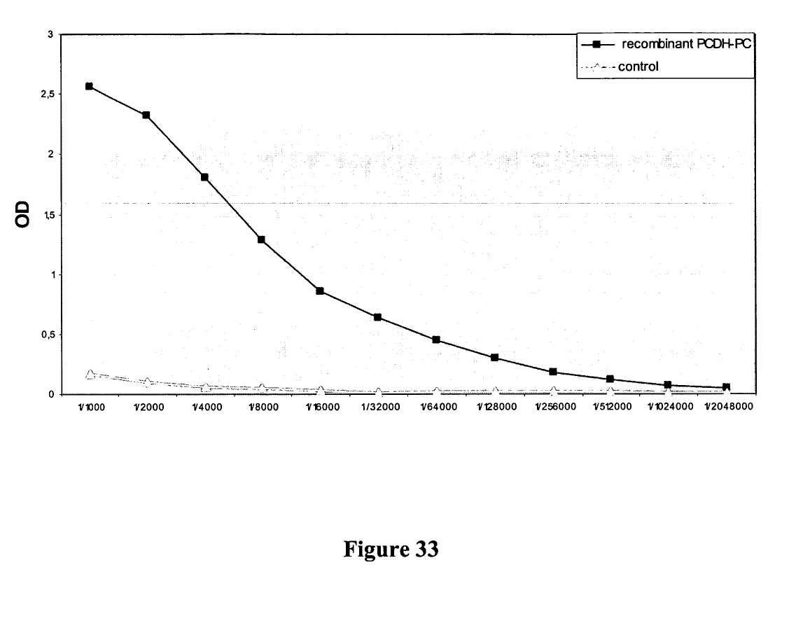

Wells of a 96 well microtiter plate (Immulon, Dynatech Laboratories.) were coated overnight with 100 ng/well of recombinant PCDH-PC in a 0.01 M carbonate coating buffer (pH 9.6). Plates were washed with phosphate buffered saline (PBS, pH 7.4) containing 0.1% Tween 20 (PBST). Plates were blocked with PBS containing 2% (w/v) bovine serum albumin (BSA) for 60 min at 37° C. After addition of culture supernatants or purified antibody (diluted in PBST+2% BSA) for 60 min, plates were washed with PBST and incubated with 2^{nd} step antibody (depending on the isotype of monoclonal anti-PCDH-PC, either peroxidase-conjugated goat anti-mouse IgG or anti-mouse Ig M was used, Jackson Immuno Research Laboratories). After an additional 60 min, plates were washed with PBST and incubated with peroxidase substrate solution (ABTS) (Sigma). Plates were read with a Microplate Reader (Dynatech) and results are shown in FIG. 33.

Sandwich ELISA

A capture monoclonal antibody anti-PCDH-PC diluted at 10 μg/ml was first diluted in 0.1 M bicarbonate buffer, pH 9.2 and then 100 μl was added to each well of the microtiter plates. The antibody coated plate was incubated at 37° C. for 2 hours, followed by overnight at 4° C. The plates were emptied and washed with PBS containing 0.1% tween20 (PBST). The unoccupied sites are blocked with 125 μl of blocking buffer containing PBS and 2% BSA for 1 hour at 37° C. The plate is emptied and washed three times with PBST. The solution containing PCDH-PC (i.e. biologic fluid) is added to the plate in a volume of 125 μl per well. After 1 h30 at 37° C., the plate is washed three times with PBST. 100 μl of a second antibody anti-PCDH-PC labelled with biotin (diluted in PBS containing 1% BSA) is added to the wells. The labeling of antibodies with biotin is performed by using the Biotin Protein labeling Kit (Roche Applied Science) according to the recommendations of the manufacturer. After 1 hour of incubation at 37° C., plates were washed three times with PBST. Streptavidin-europium (Perkin Elmer Life Sciences) at 1/1000 in europium assay (Tris-buffered saline, 15 μg/ml diethylenetriamineN,N,N(1),N(2),N(2)-pentaacetic acid, 0.1% Tween 20, 0.5% BSA) was at to each well and incubated for 20 min at 37° C., followed by wash as above. Enhancer solution (Perkin Elmer Life Sciences) was added and europium florescence was measured using a Wallac Victor plate reader. The positive control of experiment was performed with eukaryotic soluble rPCDH-PC. Eukaryotic recombinant proteins were expressed in vitro using the TNT T7-Quick coupled Transcription/translation system (Promega) according to the recommendations of the manufacturer.

Western Blot

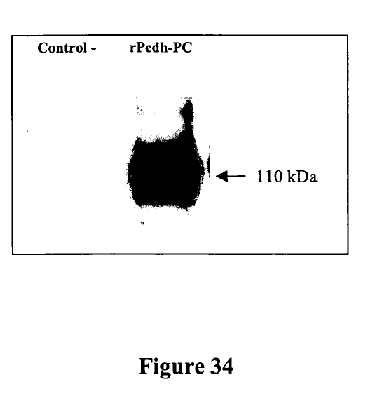

PCDH-PC was electrophoresed in a 7.5% SDS polyacrylamide gel. The protein was then transferred to PVDF membrane (Millipore Immobilon-P) in a transfer buffer (25 mM Tris, 192 mM glycine, pH 8.9; with 20% methanol). After 2 hours transfer in a Bio-Rad transfer apparatus, the blotted membrane was rinsed with PBS and blocked with PBS containing 5% (w/v) non-fat dry milk. The membrane was incubated with monoclonal antibodies containing supernatants or purified antibodies diluted in PBS containing 0.1% tween20 and 5% non-fat milk for 60 min at room temperature. After washing, the membrane was incubated with the 2nd step antibody (depending on the isotype of monoclonal anti-PCDH-PC, either peroxidase-conjugated goat anti-mouse IgG or anti-mouse Ig M was used) for an additional 60 min. After extensive washing with PBS containing 0.1% Tween 20, the presence of antibody was visualized by using the ECL Western blotting detection reagents (Amersham Biosciences). Results are shown in FIG. 34.

Immunohistochemistry

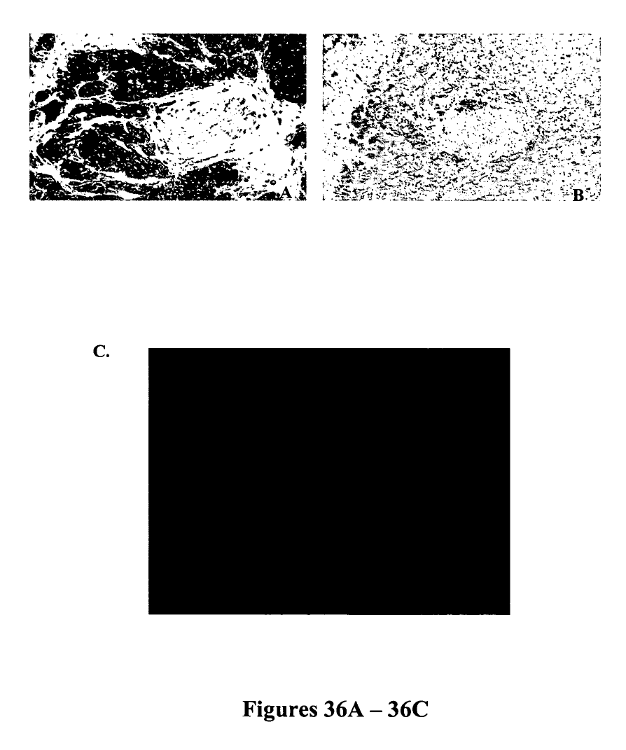

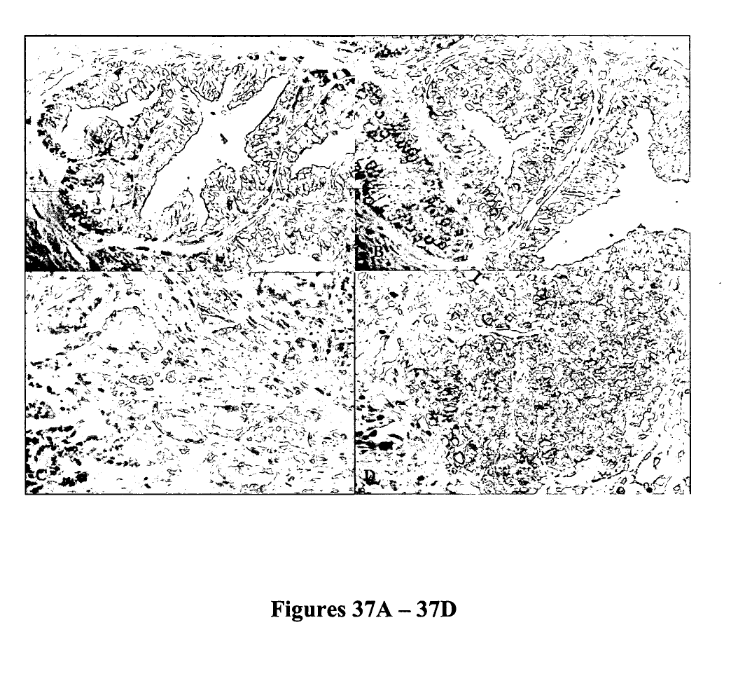

Anti-PCDH-PC antibodies were examined on frozen and on paraffin sections of normal and cancer prostate tissues (FIGS. 36-37). For paraffin-embedded prostatic tissue, sections were deparaffinized by three washes in xylene and rehydrated in increasing ethanol dilutions. To unmask antigens, slides were heated in a microwave oven twice for 5 min in 0.01 M citrate buffer, pH 6.0, at 600 W. After three washes in phosphate-buffered-saline (PBS), sections were immersed for 15 min in PBS containing 3% H₂O₂ to block endogenous peroxidases. After washing with PBS, deparaffinized sections were incubated with 5% milk in PBS for 30 min to block non-specific sites, and were then incubated for overnight at 4° C. hybridoma culture supernatants or purified antibody anti-PCDH-PC (diluted at 1 μg/ml in PBS containing 0.1% tween 20, 10% goat serum and 10% human serum). Sections were washed and incubated with biotinylated goat anti-mouse IgG or anti-IgM (diluted 1/200 in PBS containing 2.5% milk, Jackson Immuno Research Laboratories) for 1 hour at room temperature. Specific binding was revealed by using the ABC peroxidase kit (Vectastain ABC Elite kit, Vector Laboratories) and diaminobenzidine-HCl as chomogen. The sections were rinsed and lightly counterstained with Gill's hematoxylin.

Characterization of Anti-PCDH-PC Antibodies

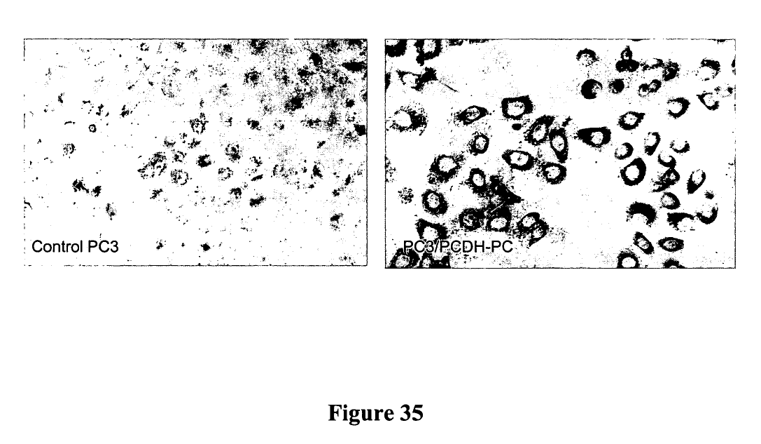

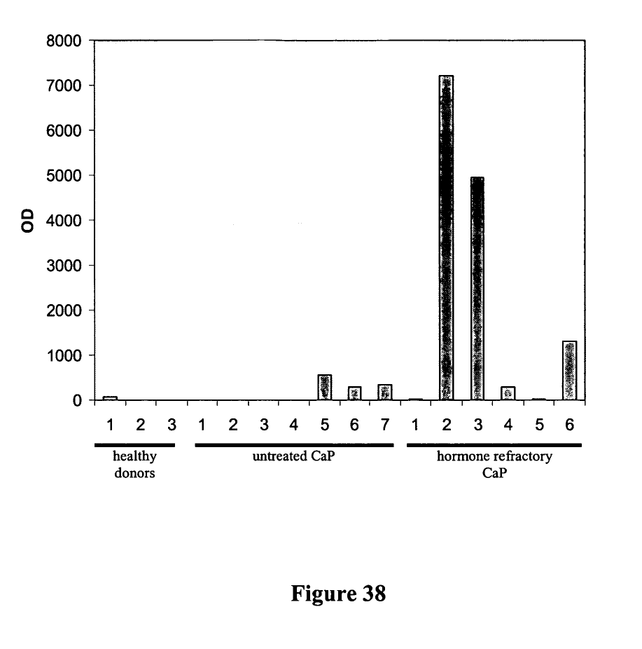

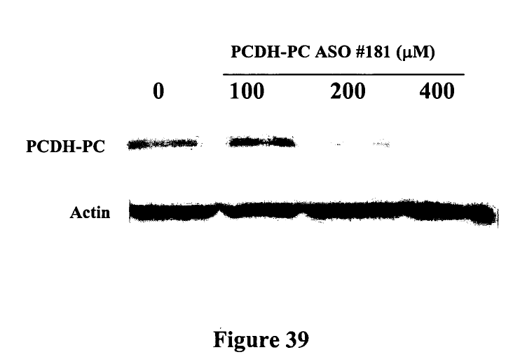

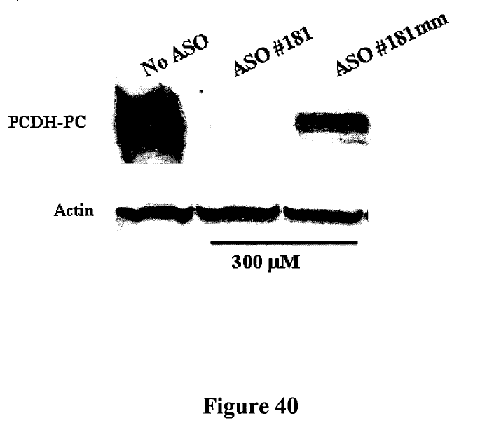

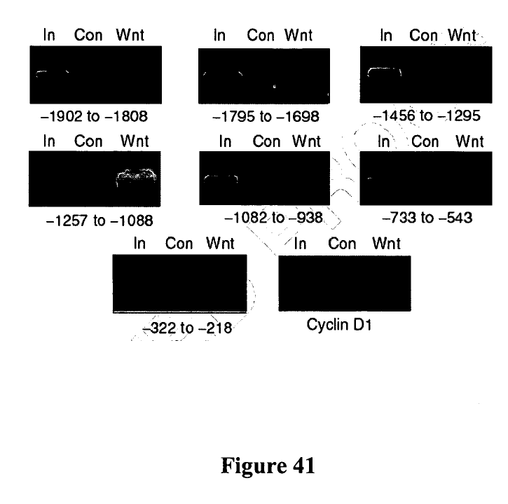

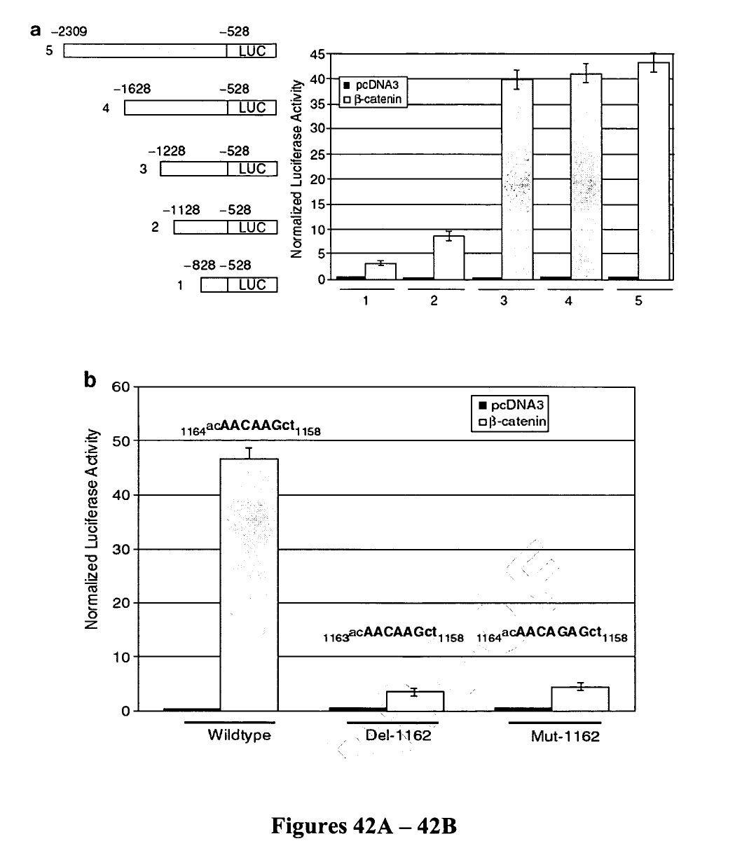

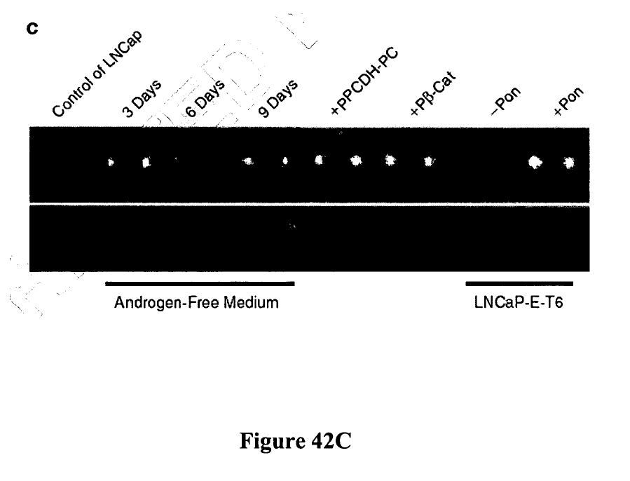

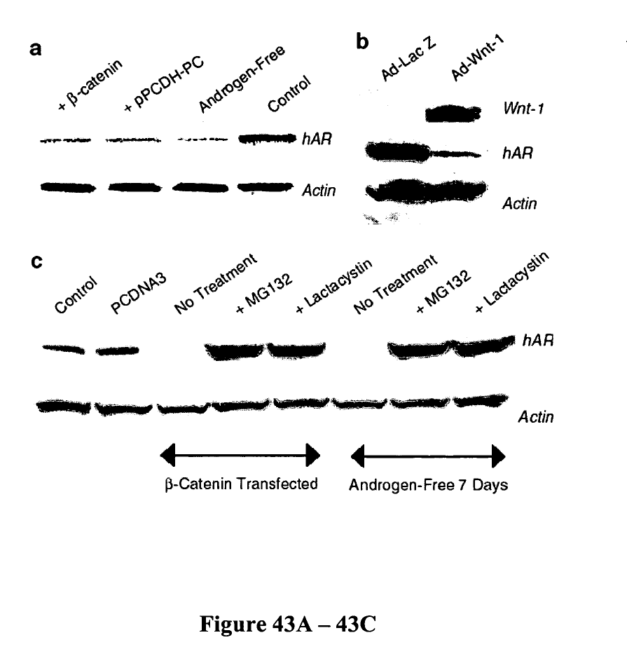

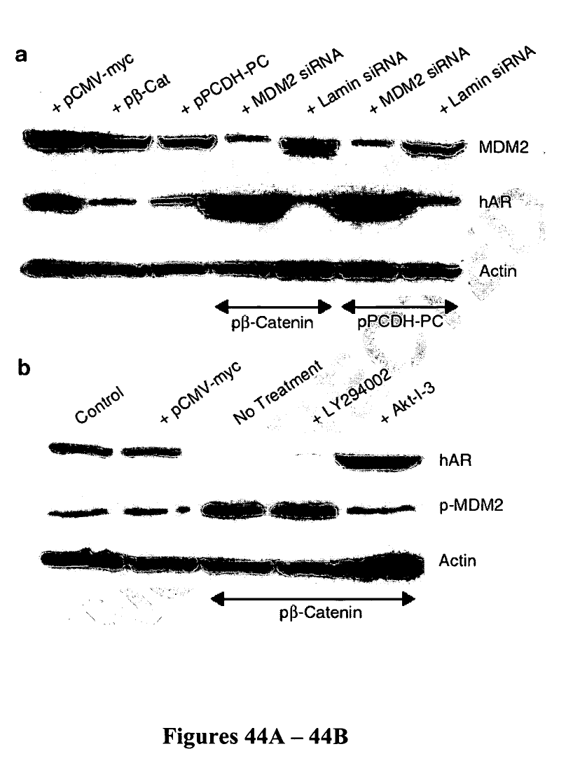

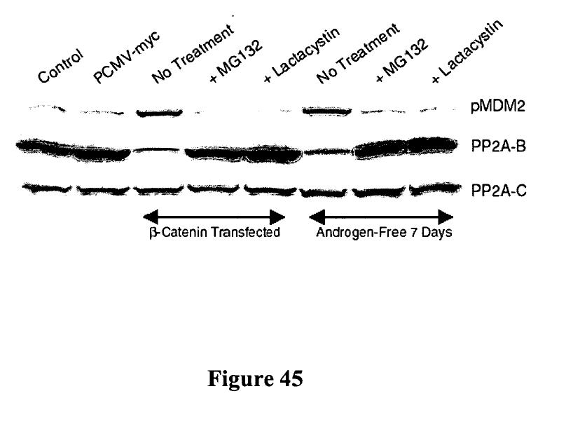

The specificities of antibodies to PCDH-PC protein were evaluated by techniques described above. The polyclonal and monoclonal antibodies produced are specifically recognized the protocadherin-PC and can be used in several and various methods: western-blotting, ELISA, immunohistochemistry. Particularly, the rabbit polyclonal anti-PCDH-PC detected the PCDH-PC protein on frozen prostate tissue sections. Monoclonal antibodies SSA and LIU are an IgM and IgG isotype respectively. These two antibodies bind specifically to PCDH-PC expressed in prostate cancer cell lines (See FIG. 35 for SSA results). The localization of PCDH-PC protein in prostate tissues was analyzed by using these 2 antibodies. Immunohistochemistry technique was performed on formalin fixed paraffin-embedded human tissues including normal and cancerous specimens of human prostate (FIGS. 36-37). In the normal prostate tissues, PCDH-PC expression was mainly found in the basal epithelium. For specimens containing BPH (benign prostatic hyperplasia) the staining was similar to that found in normal epithelium with labelling of normal cells. In specimens containing prostate tumors from untreated CaP patients, all tumor cells expressed PCDH-PC. However, more intense staining corresponding to PCDH-PC was observed in the cells of all tumors obtained from hormone refractory CaP patients (HRCaP). Monoclonal antibodies SSA and LIU were used to developed a sandwich ELISA for the determination of PCDH-PC in serum. This immunoassay allowed detecting a circulating form of PCDH-PC protein in serum of certain HRCaP patients (FIG. 38).

### Example 9

### Chemically-Synthesized Single-Stranded Antisense Oligonucleotides that Target PCDH-PC can Suppress Expression of the PCDH-PC Protein

A chemically-modified (phosphorothioate-modified) antisense deoxyribonucleic oligonucleotide (ASO) has been synthesized which corresponds to the antisense sequence of PCDH-PC (same sequences of PCDH-PC as targeted by the previously described siRNA #181 (SEQ ID NO:4)) and have tested this ASO for its ability to suppress PCDH-PC expression in cultured human prostate cancer cells (LNCaP) that were transiently transfected for 48 hrs with an expression vector designed to express a myc-tagged version of PCDH-PC. In the first experiment (FIG. 39), increasing concentrations (from 100 to 400 μM) of ASO #181 were tested for the ability to suppress PCDH-PC protein expression as measured in a Western blot assay of cell extracts (all transfected with equal amounts of PCDH-PC expression vector). Results show that ASO #181 in excess of 100 uM was able to suppress expression of PCDH-PC protein.

In a second experiment (FIG. 40), the activity of the ASO #181 was compared to a variant ASO (#181 mm) in which only 3 of the nucleotides of ASO #181 were rearranged to reduce the homology of the modified ASO to the PCDH-PC sequence. Using a similar experiment (co-transfection of an expression vector encoding a myc-tagged PCDH-PC protein with either ASO #181 or ASO #181 mm at 300 uM concentrations), Western blots of transfected cell extracts after 48 hrs were probed using an anti-myc antibody to detect expression of the PCDH-PC protein. The results show that ASO #181 at this concentration was able to completely suppress expression of the PCDH-PC protein (compared to control cells that were only transfected with the PCDH-PC expression vector), whereas the ASO #181 mm suppressed PCDH-PC protein levels by only 50%. These results indicate that suppression of PCDH-PC expression by ASO #181 was dependent upon the homology of the ASO to the antisense sequence of PCDH-PC mRNA.

### Example 10

### Complex Regulation of Human Androgen Receptor Expression by Wnt Signaling in Prostate Cancer Cells

β-Catenin, a component of the Wnt signaling pathway, is a coactivator of human androgen receptor (hAR) transcriptional activity. Here, Wnt signaling is also shown to influence androgen-mediated signaling through its ability to regulate hAR mRNA and protein in prostate cancer (PCa) cells. Three functional LEF-1/TCF binding sites lie within the promoter of the hAR gene as shown by CHIP assays that captured β-catenin-bound chromatin from Wnt-activated LNCaP cells. Chimeric reporter vectors that use the hAR gene promoter to drive luciferase expression confirmed that these LEF-1/TCF binding elements are able to confer robust upregulation of luciferase expression when stimulated by Wnt-1 or by transfection with β-catenin and that dominant-negative TCF or mutations within the dominant TCF-binding element abrogated the response. Semi-quantitative and real time RT-PCR assays confirmed that Wnt activation upregulates hAR mRNA in PCa cells. In contrast, hAR protein expression was strongly suppressed by Wnt activation. The reduction of hAR protein is consistent with evidence that Wnt signaling increased phosphorylation of Akt and its downstream target, MDM2 that promotes degradation of hAR protein through a proteasomal pathway. These data indicate that the hAR gene is a direct target of LEF-1/TCF transcriptional regulation in PCa cells but also show the expression of the hAR protein is suppressed by a degradation pathway regulated by cross-talk of Wnt to Akt that is likely mediated by Wnt-directed degradation of the B regulatory subunit of protein phosphatase, PP2A.

Prostate cancer (PCa) is a prevalent human tumor that develops and progresses under the influence of androgenic steroids. As in normal prostate cells, androgen action in PCa cells is mediated by a nuclear receptor protein, the human androgen receptor (hAR) that binds androgenic ligands, enters the nucleus and stimulates the transcription of genes having cis-acting androgen response elements within their promoter or regulatory regions (Chang et al., 1995). Androgen depletion, induced by hormonal therapies used to treat advanced PCa patients, transiently suppresses disease progression. However, the cancer inevitably recurs in a hormone refractory form that continues to grow despite the diminished androgen levels in a hormone-treated patient (Miyamoto et al., 2005). In the in vivo setting, hormone refractory PCa cells are known to maintain hAR protein expression and there is a consensus that androgen mediated gene expression is also sustained despite the deficit in circulating androgen levels (Grossmann et al., 2001). This conundrum has led to extensive research to determine mechanisms through which androgen signaling might be maintained in PCa cells in hormone-treated patients. Various studies reveal that there are likely multiple pathways leading to increased androgen signaling in a low androgen environment involving mechanisms as diverse as hAR gene amplification (Ford et al., 2003), mutations that alter the ligand specificity of the hAR (Tilley et al., 1996) or by association of the hAR protein with coactivators that cooperate to increase transcriptional activity of hAR (Rahman et al., 2004).

One coactivator that markedly influences the transcriptional activity of hAR is β-catenin, a key molecule in the canonical Wnt signaling pathway (Truica et al., 2000; Yang et al., 2002). β-Catenin binds to the activation function 2 region within the N-terminal domain of liganded hAR protein and augments ligand-dependent hAR transcriptional activity in PCa cells (Song et al., 2003). The coactivator function of β-catenin likely involves increased recruitment of p160 coactivator proteins (Li et al., 2004) as well as tertiary proteins, such as histone methyltransferase (Koh et al., 2002). β-Catenin also alters ligand specificity of hAR-mediated transcription, enhancing transcriptional activation by and rostenedione and estradiol and diminishing antagonism by bicalutamide (Truica et al., 2000). Cultured PCa cells in which Wnt signaling is activated by Wnt ligand also show increased hAR-mediated transcriptional effects even in the absence of androgenic ligands (Verras et al., 2004), which implies that the Wnt signaling pathway has additional effects on hAR mediated signaling aside from those involving interaction of β-catenin with liganded hAR. This Example evaluates the ability of Wnt signaling, mediated by β-catenin activated LEF-1/TCF transcription and MDM2-mediated protein degradation, to influence expression of the hAR mRNA and protein in PCa cells. Results show that the hAR gene is a primary target of LEF-1/TCF transcriptional control and that the Wnt signaling pathway has additional effects that modulate the levels of the hAR-encoded protein through an ubiquitin-mediated degradation process controlled by Akt/Protein kinase B signaling.

Validation of Functional LEF-1/TCF Binding Sites in the 5′ Promoter Region of the hAR Gene.

A computerized search of a 2000 bp region immediately 5′ to the transcriptional start site of the hAR gene revealed the presence of eight core (minimal) sequences containing potential LEF-1/TCF binding elements (FIG. 1a). A CHIP assay was used to determine whether any of these potential binding elements were occupied by a protein complex that contained β-catenin in control LNCaP cells (transfected with empty vector) or in LNCaP cells with Wnt signaling activated either by transfection with a mutated (stabilized) β-catenin or with protocadherin-PC (PCDH-PC), another gene product known to stimulate LEF-1/TCF-mediated transcription in these cells (Yang et al., 2005). Fixed, sheared chromatin was immunoprecipitated using anti β-catenin antibody and the immunoprecipitated chromatin was PCR-amplified using primer sets that distinguished the various potential binding sites as described in FIG. 1a. A sample of DNA extracted from unprecipitated input control LNCaP cells was amplified as a positive control to ensure that each primer set was able to amplify the appropriate sized fragment. Primer sets that amplify known LEF-1/TCF binding regions within the cyclin D1 and c-myc promoters were used as positive controls to ensure that the assay was capable of detecting LEF-1/TCF binding sites within other genes known to be transcriptionally regulated by Wnt signaling. The results of these amplifications (FIG. 1b) identified three of the eight potential LEF-1/TCF binding elements within the hAR proximal promoter region as occupied by a protein complex containing β-catenin in Wnt-activated cells. None of these potential LEF-1/TCF binding sites were immunoprecipitated from chromatin obtained from control cells without Wnt activation. This experiment was repeated using a defective recombinant adenovirus that expresses Wnt-1 protein (Ad-Wnt-1) to stimulate Wnt signaling in the LNCaP cells and the results of the CHIP analysis (compared to cells transduced with a Lac Z expressing recombinant adenovirus, Ad-LacZ) were equivalent to that shown by β-catenin or PCDH-PC transfected cells (FIG. 1c).

hAR promoter-luciferase reporter fusion vectors demonstrate increased luciferase expression in Wnt activated LNCaP cells. A series of hAR promoter-luciferase reporter vectors were constructed that contained increasing lengths of the hAR promoter region. These vectors were cotransfected into LNCaP cells along with empty vector (Wnt unstimulated control) or with the β-catenin expression vector (Wnt stimulated). Transfection efficiency was monitored by inclusion of a β-galactosidase (β-gal) reporter vector. Transfected cells were collected 48 h later and luciferase and β-gal activity was measured in the cell extracts. Expression of normalized luciferase was low in all cells co-transfected with empty vector, however, normalized luciferase activity was progressively increased as the length of the hAR promoter was increased in cells co transfected with the β-catenin expression vector (FIG. 2a). Our results indicate that the two more proximal LEF-1/TCF binding elements of the hAR promoter identified in the CHIP assay were weakly, but additively active in promoting luciferase activity in Wnt-stimulated LNCaP cells, whereas the more distal LEF-1/TCF binding element found in the CHIP assay was much more robust in promoting luciferase expression in Wnt-stimulated cells, with levels of luciferase almost 40 times greater than in cells cotransfected with empty vector. Increasing hAR promoter length beyond this did not further increase luciferase activity in Wnt-stimulated cells. Likewise, stimulation of Wnt signaling using the Ad-Wnt-1 adenovirus to transduce cells immediately prior to transfection with the largest hAR promoted luciferase vector (vector #5) showed that this induced luciferase activity more than 40-fold when compared to control, non-Wnt-induced LNCaP cells (Table 4).

TABLE 4

| | | (β-gal) normalized luciferase |
|---|---|---|
| Wnt stimulation | Co-transfection^{a} | activity |
| None | pCDNA3 | 0.14 ± 0.007 |
| Ad-Wnt-1^{b} | pCDNA3 | 45.32 ± 1.97 |
| Ad-Wnt-1^{b} | pDN-TCF | 1.8 ± 0.09 |
| pβ-Catenin | pCDNA3 | 40.24 ± 1.87 |
| pβ-Catenin | pDN-TCF | 0.19 ± 0.07 |

^{a}All transfections included the phAR/luciferase vector #5 and β-galactosidase expression vector at 1/10 concentration.
^{b}The Ad-Wnt-1 (20 PFU/cell) was adsorbed for 1 h prior to transfection.

The ability of Wnt signaling stimulation (by Ad-Wnt-1 or mutated β-catenin) to upregulate luciferase expression from the chimeric hAR reporter vector (#5) was abrogated by co-transfection with a dominant negative TCF (pDN-TCF) expression plasmid but not by empty vector (pcDNA3) (Table 4) or by introducing site-specific mutations into the dominant TCF-binding element (at −1158 to −1163) (FIG. 2b) within the hAR promoter, thus confirming that the actions of Wnt signaling in upregulating expression of the reporter from this chimeric vector was dependent upon the activity of TCF transcription factors.
Expression of hAR RNA is induced by Wnt signaling in PCa cells. RNAs extracted from Wnt-stimulated LNCaP cells (induced by transduction with Ad-Wnt-1 or by transfection with by b catenin or PCDH-PC expression vectors) were reverse transcribed and the expression of hAR and β-actin mRNAs were quantitatively measured using a relative PCR (real-time) assay and compared to control cells (transduced by Ad-lac Z or by an empty expression vector, pcDNA3). Comparison of the hAR/actin mRNA ratio of Ad-Wnt-1 transduced LNCaP cells (at 48 h) to Ad-lac Z transduced cells showed that the ratio was increased by 14.52-fold in the Wnt-1 stimulated cells. Likewise, β-catenin transfected LNCaP cells were compared to empty vector transfected cells and showed an increase of 12.55-fold in the hAR/actin mRNA ratio. Finally, comparison of the hAR/actin mRNA ratio in PCDH-PC-transfected LNCaP cells to control transfected cells revealed an increase of 11.70-fold. A similar assay was performed to assess relative hAR expression in LNCaP cells that were grown for one week in androgen-free medium that were previously shown to have upregulated Wnt signaling activity in conjunction with induced expression of PCDH-PC (Yang et al., 2005). The hAR/actin mRNA ratio of androgen-free cells was 16.45-fold higher than cells maintained in normal medium. This effect was also assessed by a semiquantitative RT-PCR based assay in which amplification products resulting from 32 thermocycles were visualized on an agarose gel (FIG. 3). These latter results confirmed the findings of Real Time RT-PCR demonstrating that all conditions associated with increased Wnt signaling (culture in androgen-free medium, transfection with β-catenin or PCDH-PC or upregulation of PCDH-PC from a conditional expression vector in stably transfected LNCAP cells (by ponasterone)) were associated with upregulation of hAR mRNA levels. Finally, a real time RT-PCR-based assessment of the hAR/actin mRNA ratio of β-catenin transfected CWR 22rv-1 cells (another human PCa cell line with endogenous expression of hAR) showed that the ratio was increased by 11.65-fold compared to control transfected cells, similar to levels in β-catenin or PCDH-PC transfected LNCaP cells. Assessment of the effects of β-catenin transfection on PC-3 or DU145 human PCa cell lines (that do not endogenously express hAR protein) using the real time RT PCR procedure showed that there was an upregulation of hAR mRNA to a level (more than 10-fold greater than control cells) similar to that of the hAR expressing LNCaP and CWR22rv-1 cells, however the extremely low basal expression of hAR mRNA in the unstimulated cells makes it difficult to determine the significance of the increase.
Expression of hAR protein is suppressed by Wnt signaling in LNCaP cells. In contrast to hAR mRNA, which was greatly increased by Wnt signaling in LNCaP cells, expression of hAR protein was reduced by at least 89% as assessed by densitometry of films from Western blot analysis of hAR expression in LNCaP cells transfected with β-catenin or PCDH-PC or in LNCaP cells maintained for 7 days in androgen-free medium (FIG. 4a). In a similar manner, LNCaP cells transduced with Ad-Wnt-1 expressed <50% the amount of hAR protein compared to cells transduced with Ad-lac Z at 48 hrs subsequent to transduction (FIG. 4b). The suppression of hAR protein levels in Wnt-activated LNCaP cells is likely associated with loss of the protein through a ubiquitin-mediated proteasomal degradation process since transient exposure to 2 different proteasome inhibitors, MG132 or lactacystin increases hAR protein in β-catenin transfected cells to levels at least 12.3-fold higher than control-transfected cells (FIG. 4c).
The role of Akt and its downstream targetMDM2 in hAR protein degradation under Wnt-stimulated conditions. Prior evidence that activated (phosphorylated) Akt mediates an MDM2 directed ubiquitinylation and degradation of hAR (Lin et al., 2002) led to an evaluation of the effects of Wnt signaling on Akt and MDM2 in LNCAP cells tested by assessing the effects of β-catenin or PCDH-PC transfection on phospho-Akt (ser 473) levels and, as shown in FIG. 5a, phospho-Akt levels are greater than 50-fold enhanced by transfection with either of these molecules. The activation of Akt signaling was consistent with a similar increase in the phosphorylation (at ser 166) of the Akt downstream target, MDM2 (Ashcroft et al., 2002). Further evidence that Wnt mediates activation of Akt signaling is shown in the results of FIG. 5b wherein siRNAs against PCDH-PC or β-catenin or dominant negative TCF-4 strongly suppressed Akt (and MDM2) phosphorylation in LNCaP cells maintained in androgen-free medium. The critical participation of the MDM2 protein in the AR degradation process was shown in an experiment in which MDM2 expression was suppressed by an siRNA revealing that hAR levels, again were upregulated to higher than control levels in β-catenin transfected cells when MDM2 expression was suppressed (FIG. 5c). Whereas a recent report suggested that Wnt signaling influences Akt signaling in PCa cells (Ohigashi et al., 2005), there was no prior evidence of the mechanism of this cross-talk. As is shown in FIG. 5d, an inhibitor of PI3-kinase, LY2294002, was not able to suppress upregulation of MDM2 phosphorylation when LNCaP cells were transfected by β-catenin nor did this affect the downregulation of hAR protein expression. However, a direct inhibitor of Akt action (compound 5233705) (26) was able to suppress downstream phosphorylation of MDM2 in β-catenin-transfected cells and this resulted in a significant elevation in the levels of hAR protein, similar to effects of proteasomal inhibitors or MDM2 knockout. These results suggest that the effects of Wnt on Akt signaling are not mediated by stimulation of PI3-kinase activity. One potential indication of the mechanistic link between increased Wnt signaling and increased phosphorylation of Akt was found when protein extracts of β-catenin transfected or androgen-free LNCaP cells were reanalyzed for phosphorylated MDM2 levels (FIG. 6). Proteasome inhibitors suppressed phosphorylation of MDM2, which implies that some activity associated with Wnt signaling may stimulate proteolytic degradation of an endogenous inhibitor of Akt or MDM2 activation. When these same protein extracts were analyzed for expression of the Akt signaling inhibitor, protein phosphatase-2A (PP2A) (Stack et al., 2004), the B catalytic subunit of this complex enzyme was found to be reduced by approximately 87% in Wnt-stimulated cells and this loss was blocked by the proteasome inhibitors (FIG. 6). There was no effect of Wnt-stimulation or proteasome inhibition on expression of the catalytic C subunit of PP2A. Since the PP2A B subunit is known to bind to the β-catenin degradation complex that controls the canonical Wnt signaling pathway (Ratcliffe et al., 2000), the results suggest that Wnt crosstalk to Akt is mediated, at least partially, by proteasome-mediated destruction of the PP2A B subunit when Wnt signaling is activated.

Although the Wnt signaling pathway is involved in normal embryonic development, tissue differentiation and morphogenetic processes, it also plays an important role in human oncogenesis (Barker and Clevers, 2000; Lustig and Behrens, 2003). Intestinal/colon, breast, skin (melanoma) and oral cancers all show evidence for upregulation of wnt signaling during the natural history of their development and progression. As is best described in colon cancer (Sancho et al., 2004), Wnt signaling becomes dysregulated in association with mutations in the APC gene whose product is required for ubiquitin-mediated degradation of the β-catenin protein before it can activate LEF-1/TCF transcription or by mutations in the β-catenin gene that makes the protein refractory to the degradation process. Increasing evidence also indicates that the Wnt signaling pathway plays a role in PCa, especially in progression to the most aggressive and therapeutic-resistant state (de la Taille et al., 2003; Chen et al., 2004b). Mutations in both APC (Watanabe et al., 1996) and β-catenin (Voeller et al., 1998; Chesire et al., 2000) have been described in human PCa specimens, however, their apparent occurrence is at too low a frequency to account for the evidence for more frequent activation of Wnt signaling in this tumor system. Example 1 provides that a novel member of the protocadherin gene family, protocadherin-PC (PCDH-PC) is upregulated in apoptosis- and hormone-resistant human PCa cells and that a major effect of this gene product is the upregulation of Wnt signaling (Yang et al., 2005). Wnt signaling mediated by PCDH-PC expression or by expression of mutated β-catenin was shown to confer neuroendocrine-like characteristics on PCa cells and this phenotype is often described in association with aggressive PCa cells in vivo. Evidence presented here shows that PCDH-PC, β-catenin or Wnt-1 drastically increases levels of hAR mRNA and phospho-Akt. Since elevated Akt phosphorylation is also associated with aggressive PCa (Ghosh et al., 2003) the phenotypic transformation of the PCa cell mediated by PCDH-PC expression and Wnt signaling appears to confer many characteristics associated with the most aggressive forms of the disease.

These findings add to the growing body of literature showing that the Wnt signaling pathway crosstalks with the androgen-signaling pathway. Previous work showing that β-catenin promotes androgen signaling through coactivation of liganded hAR identified a synergistic relationship between Wnt and androgen signaling in PCa cells. Here, it is shown that Wnt signaling is also able to significantly upregulate hAR mRNA expression through transcriptional promotion mediated by TCF binding elements within the promoter of the hAR gene and, if this resulted in similar upregulation of hAR protein, would imply that the upregulation of Wnt signaling alone would be sufficient to confer virtually all the characteristics of the most aggressive form of PCa. However, increased Wnt signaling appears to have an opposite effect on expression of the hAR protein. Observations suggest that this effect is likely mediated by the influence of Wnt on the Akt signaling pathway leading to increased phosphorylation of the Akt target, MDM2 and increased proteasomal degradation of hAR protein. Inhibitors of proteasomal activity (MG132 and lactacystin), Akt signaling (by compound 5233705) or MDM2 expression (with siRNA that targets this gene) resulted in hAR levels that were approximately eight to 12-foldhigher in β-catenin transfected cells than in control PCa cells and this increase was consistent with increased hAR mRNA levels in Wnt-stimulated cells. The inability of the PI3-kinase inhibitor LY29004 to suppress Akt phosphorylation subsequent to activation of Wnt signaling indicates that the mechanism of Wnt to Akt crosstalk likely does not involve an effect of Wnt on PI3-kinase activity. However, the evidence that Wnt activation leads to specific degradation of the B subunit of PP2A supports the concept that loss of PP2A activity is involved in this phenomenon since PP2A downregulates Akt signaling. With regards to the situation in hormone refractory PCa cells found in specimens obtained from patients, there is evidence that these cells have upregulated hAR mRNA (Gil-Diez de Medina et al., 1998; Latil et al., 2001) as well as hAR protein (Ford et al., 2003). Similar findings are also reported for human PCa cell xenografts (Chen et al., 2004) and cultured PCa cells that are chronically deprived of androgen (Shi et al., 2004). If Wnt signaling is a driving force involved in the generation of hormone refractory PCa, this would imply that there might be a two-step process; one in which the hAR gene is transcriptionally upregulated under conditions of increasing Wnt signaling immediately following androgen deprivation and a second step, which involves suppression of the hAR protein degradative process in the presence of highly active Akt signaling. This two-step progression pathway would be consistent with the natural biology of PCa in which hormonal ablation therapies transiently suppress disease progress for a limited period followed by a breakthrough in which the cancer cells acquire the ability to grow in the absence of androgens as well as with observations in animal models of hormone-dependent PCa (Craft et al., 1999).

This Example includes the observation that expression of PCDH-PC in human prostate cancer cells increases expression of the androgen receptor protein which is needed for the growth of these cells. Some current research in the development of improved prostate cancer therapies is focused developing gene-targeting reagents that will suppress androgen receptor protein expression in prostate cancer cells. Based on the findings presented in this Example, a therapeutic benefit of PCDH-PC targeting agents, such as the siRNAs, ASOs and antibodies provided by this invention, is that these agents will also likely down-regulate expression of the androgen receptor protein, therefore enhancing the therapeutic potential of these agents.

Cell lines, plasmids and siRNAs. LNCaP, CWR22rv-1, PC-3 and DU145 cells were obtained from ATCC and were passaged in normal (for LNCaP, RPMI 1640 with 10% fetal calf serum and supplements) or androgen-free maintained as previously described (Yang et al., 2005). A defective adenovirus that expresses Wnt-1 protein (Ad-Wnt-1) and control, Lac Z expressing adenovirus (Ad-lac Z) were previously described (Young et al., 1998). These viruses were applied at 20 particles/cell in low serum (2%) medium for 1 h. Expression plasmids containing mutated (stabilized) human β-catenin (Tetsu and McCormick, 1999), dominant negative TCF-4 (Chen et al., 2001) or PCDH-PC cDNA were transfected into cells as previously described (Example 1; Yang et al., 2005). Small interfering (si) RNAs targeting β-catenin or lamin were purchased from Dharmacon Inc. siRNA targeting human MDM2 was purchased from Qiagen Inc (Valencia, Calif.). siRNAs were transfected into cells. Proteasome inhibitors MG132 and lactacystin were purchased from Sigma Chemical Co. (St Louis, Mo.) and were used at 5 (MG132) or 10 (lactacystin) mM for 12 h prior to cell harvesting. PI3-kinase inhibitor LY294002 (Sigma Chemical Co.) and Akt Inhibitor IV (compound 5233705, EMD Biosciences Inc., San Diego, Calif.) (Kau et al., 2003) were used at 4 and 50 mM concentrations, respectively, for 12 h prior to harvesting cells.

Preparation of cell extracts and western blots. Cells were harvested and protein extracts prepared, quantified and used to prepare Western blots as previously described (Example 1; Yang et al., 2005). Western blots were probed with mouse monoclonal antibodies against human Akt protein, phospho-MDM2 (ser 166), hAR (Santa Cruz Biotechnology Inc., Santa Cruz, Calif.), actin (Sigma Chemical Co., St Louis, Mo.) or with rabbit polyclonal antibodies against phospho-Akt (ser 473) or human MDM2 (Cell Signaling Technology, Beverly, Mass.). Recombinant Wnt-1 protein (HA-tagged) was detected with anti-HA antibody (Clontech Inc., Mountain View, Calif.). Antibody binding to the Western blot was detected as previously described (Yang et al., 2005). Densitometry of films was carried out using a Kodak Image Station 420.

CHIP assay of regions of the hAR gene promoter bound to β-catenin protein. A 2000 bp region immediately upstream of the hAR gene (Genbank accession #L14435) was analyzed for core LEF-1/TCF binding sites (5′-CTTTG-3′ (SEQ ID NO:27)) using the TransFac computer analysis program. PCR primer sets were designed to amplify small regions within this promoter sequence: Primer set #1 (−322 to −218) forward 5′-TTAGATTGGGCTTTGGAACC-3′ (SEQ ID NO:28), reverse 5′-GCTTCCTGAATAGCTCCTGCT-3′ (SEQ ID NO:29); Primer set #2 (−733 to −543) forward 5′-CAAAATTGAGCGCCTATGTG-3′ (SEQ ID NO:30), reverse 5′-TTGCTCTAGGAACCCTCAGC-3′ (SEQ ID NO:31); Primer set #3 (−1082 to −938) forward 5′-GGCAAAAATCTCGGAATGAC-3′ (SEQ ID NO:32), reverse 5′-AAAGGTGGAGATGCAAGTGG-3′ (SEQ ID NO:33); Primer set #4 (−1257 to −1088) forward 5′-ATCCAGTCTTCCTTGCCTTT-3′ (SEQ ID NO:34), reverse 5′-TTCTGGGAGGCTCTCTGTTC-3′ (SEQ ID NO:35); Primer set #5 (−1456 to −1295) forward 5′-CAGGTGAAAGGGTCTTCAGG-3′ (SEQ ID NO:36), reverse 5′-AGGACATAATTTGTTCTATGTTCCAC-3′ (SEQ ID NO:37); Primer set #6 (−1795 to −1698) forward 5′-TTTTTCAGGCCTCTTTGTGTC-3′ (SEQ ID NO:38), reverse 5′-TGTGTCTACACACTAACAGTGAAGGA-3′ (SEQ ID NO:39); Primer set #7 (−1902 to −1808) forward 5′-TGGTGATGTGGAAGCAACATA-3′ (SEQ ID NO:40), reverse 5′-AAGGTGAGAAATAATGCTCTGAAGTT-3′ (SEQ ID NO:41). Two additional primer sets were designed to amplify regions within the promoters of the human c-myc (He et al., 1998) (forward 5′-GCTCTCCACTTGCCCCTTTTA-3′ (SEQ ID NO:42), reverse 5′-GTTCCCAATTTCTCAGCC-3′ (SEQ ID NO:43)) and cyclin D1 gene (Tetsu and McCormick, 1999) (forward 5′-GGGAGGAATTCACCCTGAAA-3′ (SEQ ID NO:44), reverse 5′-CCTGCCCCAAATTAAGAAAA-3′ (SEQ ID NO:45)) that contain known LEF-1/TCF binding sites. CHIP assays were then performed on LNCaP cells that were transfected by empty vector (pCMV-myc), β-catenin or PCDH-PC expression plasmids for 48 h using the CHIP-IT kit of Active Motif Inc. (Carlsbad, Calif.) using the manufacturer's protocol. A specimen of formalin-fixed sheared chromatin from empty vector transfected LNCaP cells was used as ‘input DNA’ for control amplifications. Fixed chromatin was immunoprecipitated using monoclonal mouse anti-β-catenin antibody (Santa Cruz Biotechnology Inc.) and DNA was extracted from the immunoprecipitate and amplified using the primer sets described above. Amplification products on 1.2% agarose gels were visualized under UV light after ethidium bromide staining and sized according to molecular weight markers in adjacent lanes. Control immunoprecipitations was carried out using nonimmune mouse IgG (Santa Cruz Biotechnology Inc.) from each of the specimens did not yield any reaction products for any of the primer sets.

Construction of hAR promoter-luciferase reporter vectors and test for Wnt-responsiveness. A series of PCR primers were designed to amplify increasing regions of the hAR promoter region, each anchored at the 3′ termini at base-528 upstream the transcription start site (reverse primer 5′-GCGAAGCTTGTGGCATTGTGCCATTTG-3′ (SEQ ID NO:46)). The various upstream (forward) primers utilized were: 5′ position-2129, 5′-GCGCTCGAGTCAAAATCCAAATAAAGTATATGGCC-3′ (SEQ ID NO:47); 5′ position-1628,5′-GCGCTCGAGAGCCCACTCAATTCCTATTGAG-3′ (SEQ ID NO:48); 5′ position-1228,5′-CTCGAGACCTTCTTTGGTCAAGGTAAGTAAA-3′ (SEQ ID NO:49); 5′ position-1128,5′-CTCGAGACCTTCTTTGGTCAAGGTAAGTAAA-3′ (SEQ ID NO:50) and; 5′ position-828, 5′-CTCGAGCCTTGGATAGTTCCAGTTGTAAAG-3′ (SEQ ID NO:51). Primers were utilized to amplify DNA extracted from human LNCaP cells using thermocycles of 94° C. for 20 s for one cycle, 94° C. for 3 min, 56° C. for 30 s and 72° C. for 30 s for 32 cycles and finished by a 10 min cycle at 72° C. DNA fragments from the various amplifications were inserted into the pGEM-T Easy vector (Promega Life Sciences Inc., Madison, Wis.). Inserted fragments were removed using HindIII and XhoI restriction endonucleases and were purified using the Nucleo Trap Nucleic Acid Purification Kit (BD Biological Science Inc., Palo Alto, Calif.) and ligated into HindIII, XhoI cleaved pGL3 vector (Promega) using the Rapid DNA Ligation Kit (Roche Applied Science, Indianapolis, Ind.). Reporter vectors (3 mg) were co-transfected with 3 mg of pCDNA3 (empty vector) or β-catenin along with 0.3 mg of a β-galactosidase vector (Promega). After 48 h, luciferase and β-gal activity was measured using the Luciferase Assay System and β-galactosidase Assay Systems of Promega Inc. Normalized luciferase activity is calculated as Light Units normalized to β-gal activity present in each specimen. Each assay was performed in triplicate.

Semiquantitative and real time RT-PCR analysis of AR mRNA expression. RNA was extracted from control or transfected cells using the Rneasy Kit from Qiagen Inc. and RNA was quantified by spectrophotometry at 260 nm. RNA (1 mg) was converted to cDNA using oligo-dT primer and reverse transcriptase (Superscript III, Invitrogen Life Technologies). For semi-quantitative evaluation of hAR and G3PDH mRNA expression, 1/50 reverse transcription reaction product was amplified with the hAR primer set (forward, 5′-GGACTTCACCGCACCTGATG-3′ (SEQ ID NO:52); reverse, 5′-CTGGCAGTCTCCAAACGCAT-3′ (SEQ ID NO:53)) or the G3PDH primer set (forward, 5′-GGATTTGGTCGTATTGGGCGC-3′ (SEQ ID NO:54); reverse, 5′-GTTCTCAGCCTTGACGGTGC-3′ (SEQ ID NO:55)) using Amplitaq GoldTaq polymerase (Invitrogen Life Sciences) for 5 min at 90° C. followed by 35 cycles of 92° C. for 1 min, 57° C. for 1 min and 72° C. for 1 min and finished by 10 min at 72° C. Ethidium bromide-stained amplification products were visualized after electrophoresis under UV light. For semi-quantitative (real time) RT-PCR, 1/50 reverse transcription reaction product was amplified using hAR (forward, 5′-CGGAAGCTGAAGAAACTTGG-3′ (SEQ ID NO:56); reverse 5′-CGTGTCCAGCACACACTACA-3′ (SEQ ID NO:57)) or actin (forward, 5′-ATGGATGATGATATCGCCGC-3′ (SEQ ID NO:58); reverse, 5′-AAGCATTTGCGGTGGACGAT-3′ (SEQ ID NO:59)) primer sets in triplicate for each specimen using the reagents of the Roche Applied Biosystems LightCycler® FastStart reaction mix that monitors amplification products based upon SYBR Green I fluorescence on a LightCycler 2.0 instrument (Roche Diagnostics Inc.). Data was analyzed using the LightCycler® software that calculates the crossing point of each sample on the quantification curve. The specificity of each reaction was demonstrated by conducting a melting curve analysis of the PCR product at the end of each run.

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention.

### REFERENCES

Aberle, H., Bauer, A., Stappert, J., Kispert, A., Kemler, R. (1997) Beta-catenin is a target for the ubiquitin-proteasome pathway. EMBO J., 16: 3797-3804.
Abrahamsson, P. A. (1999) Neuroendocrine differentiation in prostatic carcinoma. Prostate, 39: 135-148.
Abrahamsson, P. A., Dizeyi, N., Alm, P., di Sant'Agnese, P. A., Deftos, L. J., Aumuller, G. (2000) Calcitonin and calcitonin gene-related peptide in the human prostate gland. Prostate, 44: 181-186.
Abrahamsson, P. A. and Di Sant'Agnese, P. A. (1993) Neuroendocrine cells in the human prostate gland. J. Androl., 5: 307-309.
Adriani, F., Nan, B., Yu, J., Li, X., Weigel, N. L., McPaual, M. J., Kasper, S., et al. (2001) Use of the probasin promoter ARR2PB to express Bax in androgen receptor-positive prostate cancer cells. J. N. C. I. 93: 1314-1324.
Ahlegren G, Pedersen K, Lundberg S, Aus G, Hugosson J, Abrahamsson P. Neuroendocrine differentiation is not prognostic of failure after radical prostatectomy but correlates with tumor volume. Urol 2000; 56: 1011-1015.
Angst, B. D., Marcozzi, C. and Magee, A. L., The cadherin superfamily: diversity in form and function. J. Cell Sci., 114: 629-641.
Apakam, I., Robinson, M. C., Walter, N. M., Charlton, R. G., Royds, J. A., Fuller, C. E., Neal, D. E. and Hamdy, F. C. (1996) bcl-2 overexpression combined with p53 protein accumulation correlates with hormone-refractory prostate cancer. Br. J. Cancer, 74: 1258-1262.
Aprikian, A. G., Han, K., Guy, L., Landry, F., Begin, L. R., Chevalier, S. (1998) Neuroendocrine differentiation and the bombesin/gastrin-releasing peptide family of neuropeptides in the progression of prostate cancer. Prostate, 8 (S): 52-61.
Arnold, J. T. and Isaacs, J. T. (2002) Mechanisms involved in the progression of androgen-independent prostate cancers: it is not only the cancer cell's fault. Endocrine Related Cancer, 9: 61-73.
Ashcroft M, Ludwig R L, Woods D B, Copeland T D, Weber H O, MacRae E J et al. (2002). Oncogene 21: 1955-1962.
Assikis, V. J., Do, K. A., Wen, S., et al. (2004) Clinical and biomarker correlates of androgen-independent, locally aggressive prostate cancer with limited metastatic potential. Clin. Cancer Res., 10: 6770-6778.
Ayala, G., Thompson, T., Yang, G., et al. (2004) High levels of phorphorylated form of Akt-1 in prostate cancer and non-neoplastic prostate tissues are strong predictors of biochemical recurrence. Clin. Cancer Res., 10: 6572-6578.
Bang Y J, Prinia F, Fang W G, et al. Terminal neuroendocrine differentiation of human prostate carcinoma cells in response to increased intracellular cyclic AMP. Proc Natl Acad Sci USA. 1994; 91:5330-5334.
Barker N, Clevers H. (2000). Bicessays 22: 961-965.
Barth, A. I., Nathke, I. S., Nelson, W. J. (1997) Cadherins, catenins and APC protein: interplay between cytoskeletal complexes and signaling pathways. Curr. Op. Cell Biol., 9: 693-690.
Berma, N. K., Dey, C. S. (2004) RNA-mediated gene silencing: mechanisms and its therapeutic application. J. Clin. Pharm. Therap., 29: 395-404.
Berruti A, Dogliotti L, Mosca A, et al. Circulating neuroendocrine markers in patients with prostate carcinoma. Cancer 2000; 88:2590-2597.
Bierie, B., Nozawa, M., Renou, J. P., Shillingoford, J. M., Morgan, F., Oka, T., et al. (2003) Activation of beta-catenin in prostate epithelial induces hyperplasias and squamous transdifferentiation. Oncogene, 22: 3875-3887.
Blanco, P., Saargent, C. A., Boucher, C. A., Mitchell, M. and Affara, N. A. (2000) Conservation of PCDHX in mammals, expression of human X/Y genes predominantly in brain. Mammalian Genome, 11: 906-914
Blanco-Arias, P., Sargent, C. A. and Affara, N. A. (2004) Protocadherin X (PCDHX) and Y (PCDHY) genes; multiple mRNA isoforms encoding variant signal peptides and cytoplasmic domains. Mammalian Genome, 15: 41-52.
Bollito E, Berruti A, Bellina M, et al. Relationship between neuroendocrine features and prognostic parameters in human adenocarcinoma. Ann Oncol 2001; 12(S1): 159-164.
Bright-Thomas, R. M., Haargest, R. (2003) APC, beta-catenin and hTCF-4: an unholy trinity in the genesis of colorectal cancer. Eur. J. Surg. Oncol., 29: 107-117.
Brown, A. M. (2001) Wnt signaling in breast cancer: have we come full circle? Breast Cancer Res., 3:351-355.
Buchanan G, Irvine R A, Coetzee G A, Tilley W D. Contribution of the androgen receptor to prostate cancer predisposition and progression. Cancer Mets Rev 2001; 20:207-223.
Burchardt, M., Burchardt, T., Shabsigh, A., Ghafar, M., Chen, M.-W., Anastasiadis, A., de la Taille, A., Kiss, A. and Buttyan, R. (2001) Reduction of wild type p53 function confers a hormone resistant phenotype on LNCaP prostate cancer cells. Prostate, 48: 225-230.
Buttyan, R. and Slawin, K. (1993) Rodent models for targeted oncogenesis of the prostate gland. Cancer Met Rev., 12: 11-19.
Carter H B and Isaacs J T. Overview of hormonal therapy for prostate cancer. Prog Clin Biol Res 1990; 350: 129-40.
Catz, S. D. and Johnson, J. L. (2003) Bcl-2 in prostate cancer; a mini-review. Apoptosis, 8: 29-37.
Chang C, Saltzman A, Yeh S, Young W, Keller E, Lee H-J et al. (1995). Crit Rev Eukaryot Gene Exp 5: 97-125.
Chen T, Wang L H, Farrar W L. Interleukin 6 activates androgen receptor-mediated gene expression through a signal transducer and activator of transcription 3-dependent pathway in LNCaP prostate cancer cells. Cancer Res 2000; 60:2132-2135.
Chen, M.-W., Vacherot, F., de la Taille, A., Gil-Diez-de-Medina, S., Shen, R., Friedman, R., Burchardt, M., Chopin, D. K. and Buttyan, R. (2002) The emergence of protocadherin-PC expression during the acquisition of apoptosis-resistance by prostate cancer cells. Oncogene, 21: 7861-7871.
Chen, S. Guttridge, D. C., You, Z., Zhang, Z., Fribley, A., Mayo, M. W., Kitajewski, J. and Wang, C. Y. (2001) Wnt-1 signaling inhibits apoptosis by activating beta-catenin/T cell factor-mediated transcription. J. Cell Biol., 152: 87-96.
Chen G, Shukier N, Potti A, et al. Up-regulation of Wnt-1 and beta-catenin production in patients with advanced metastatic prostate cancer: potential pathogenetic and prognostic implications. Cancer 2004; 101:1345-1356.
Chen C D, Welsbie D S, Tran C, et al. Molecular determinants of resistance to antiandrogen therapy. Nat Med 2004; 10:33-9.
Cheshire D R and Isaacs W B. Beta-catenin signaling in prostate cancer: an early perspective. Endocr Relat Cancer 2003; 10:537-60.
Chesire D R and Isaacs W B. Ligand-dependent inhibition of beta-catenin/TCF signaling by androgen receptor. Oncogene 2002; 21: 8453-69.
Chesire D R, Ewing C M, Sauvageot J, Bova G S, Isaacs W B. Detection and analysis of beta-catenin mutations in prostate cancer. Prostate 2000; 45:323-334.
Chesire D R, Ewing C M, Gage W R, Isaacs W B. In vitro evidence for complex modes of nuclear beta-catenin signaling during prostate growth and tumorigenesis. Oncogene 2002; 21:2679-2694.
Chevalier, S., Defoy, I., Lacoste, J., Hamel, L., Guy, L., Begin, L. R. and Aprikian, A. G. (2002) Vascular endothelial growth factor and signaling in the prostate: more than angiogenesis. Mol. Cell. Endocrinol., 189: 169-179.
Chi, K. N., Gleave, M. E. (2004) Antisense approaches in prostate cancer. Exp. Op. Biol. Ther., 4: 927-936.
Chirgwin J M, Przybyla A E, MacDonald R J, and Rutter W J. Isolation of biologically active ribonucleic acid from sources enriched in ribonuclease. Biochemistry 1979; 18: 5294-9.
Cohen, R. J., Glezerson, G., Taylor, L. F., Grundle, H. A. J. and Naude, J. H. (1993) The neuroendocrine cell population of the human prostate gland. J. Urol., 150: 365-368.
Colombel M, Olsson C A, Ng P Y, and Buttyan R. Hormone-regulated apoptosis results from reentry of differentiated prostate cells onto a defective cell cycle. Cancer Res 1992; 52:4313-9.
Colombel, M., Symmans, F., Gil, S., O'Toole, K. M., Chopin, D., Benson, M., Olsson, C. A., Korsmeyer, S. and Buttyan, R. (1993) Detection of the apoptosis-suppressing oncoprotein bcl-2 in hormone refractory human prostate cancers. Am. J. Pathol., 143: 390-400.
Conacci-Sorrell, M., Zhurinsky, J. and Ben-Ze'ev, A. (2002) The cadherin-catenin adhesion system in signaling and cancer. J. Clin. Invest., 109: 987-991.
Cornauer M V, Schulz W A, Burchardt T, et al. The androgen receptor in hormone-refractory prostate cancer: relevance of different mechanisms of androgen receptor signaling. Int J Oncol 2003; 23:1095-1102.
Craft N, Sawyers C L. Mechanistic concepts in androgen-dependence of prostate cancer. Cancer Mets Rev 1999; 17:421-427.

Craft N, Chhor C, Tran C, et al. Evidence for clonal outgrowth of androgen-independent prostate cancer cells from androgen-dependent tumors through a two-step process. Cancer Res 1999; 59:5030-6.

Crawford, E. D. (2003) Epidemiology of prostate cancer. Urology, 62 (suppl): 3-12.
Crooke, S. T. (1993) Therapeutic potential of oligonucleotides. Curr. Opin. Invest. Drugs, 2: 1045-1048.
Crooke, S. T. (2003) Progress in Antisense Technology. Ann. Rev. Med., 55: 61-95.
Culig, Z. (2003) Role of the androgen receptor axis in prostate cancer. Urology, 62: 21-26, 2003.
Culig Z, Klocker H, Bartsch G, Steiner H, Hobish A. Androgen receptors in prostate cancer. J Urol 2003; 170:1363-1369.
Debruyne, F. (2002) Hormonal therapy of prostate cancer. Sem Urol. Oncol. 20: 4-9.
Deeble, P. D., Murphy, D. J., Parsons, S. J. and Cox, M. E. (2001) Interleukin-6 and cyclic AMP-mediated signaling potentiates neuroendocrine differentiation of LNCAP prostate tumor cells. Mol. Cell. Biol., 21: 8471-8482.
De la Taille, A., Rubin, M. A., Chen, M.-W., Vacherot, F., Gil-Diez-de-Medina, S., Burchardt, M., Buttyan, R. and Chopin, D. K. (2003) β-catenin-related anomalies in apoptosis-resistant and hormone-refractory prostate cancer cells. Clin. Cancer Res., 9: 1801-1807.
Denmeade S R, Lin X S, Isaacs J T. (1997) Role of programmed (apoptotic) cell death during the progression and therapy for prostate cancer. Prostate 28; 1996:251-265.
Denmeade, S. R. and Isaacs, J. T. (2002) A history of prostate cancer treatment. Nature Rev., 2: 389-396.
di Sant'Agnese P A. Neuroendocrine differentiation in human prostatic carcinoma. Hum Pathol 1992; 23:287-296.
di Sant'Agnese, P. A. and Cockett, A. T. (1996) Neuroendocrine differentiation in prostatic malignancy. Cancer, 78: 357-361.
di Saint'Agnese, P. A. (2001) Neuroendocrine differentiation in prostatic carcinoma: an update on recent developments. Ann. Oncol., 12: S135-149.
Dizeya, N., Bjartell, A., Nilsson, E., Hansson, J., Gadaleauneu V., Cross, N. Abrahamsson, P. A. (2004) Expression serotonin receptors and the role of serotonin in human prostate cancer tissues and cell lines. Prostate, 59: 328-336.
Drobnjak, M., Osman, I., Scher, H. I., Fazzari, M. and Cordon-Cardo, C. (2000) Overexpression of cyclin D1 is associated with metastatic prostate cancer to the bone. Clin. Cancer Res., 6: 1891-1895.
Evangelou, A. I., Winter, S. F., Huss, W. J., Bok, R. A., Greenberg, N. M. (2004) Steroid hormones, polypeptide growth factors, hormone refractory prostate cancer and the neuroendocrine phenotype. J. Cell. Biochem., 91: 671-683.
Feldman B J and Feldman D. The development of androgen-independent prostate cancer. Nat Rev Cancer 2001; 1:34-45.
Ford O H, Gregory C W, Kim D, Smitherman A B, Mohler J L. (2003). J Urol 170: 1817-1821.
Fournari, F., Signoretti, S., Bronson, R., Klein, L., Sellers, W. R., Kum, J., Siermann, A., et al. (2002) Stabilization of β-catenin induces lesions reminiscent of prostatic intraepithelial neoplasia, but terminal squamous transdifferentiation of other secretory epithelial. Oncogene, 21: 4099-4107.
Frank, M. and Kemler, R. (2002) Protocadherins. Curr. Op. Cell Biol., 14: 557-562.
Furumurthy S, Vasudevan K M, Rangnekar V M. Regulation of apoptosis in prostate cancer. Cancer Mets Rev 2001; 20:225-243.
Ghosh P M, Malik S, Bedolla R, Kreisberg J I. (2003). Curr Drug Metabol 4: 487-496.
Gil-Diez de Medina S, Salomon L, Colombel M, et al. Modulation of cytokeratin subtype, EGF receptor, and androgen receptor expression during progression of prostate cancer. Hum Pathol 1998; 29:1005-12.
Gittes R F. Carcinoma of the prostate. N Engl J Med 1991; 324: 236-45.
Gkonos, P. J., Krongrad, A. and Roos B. A. (1995) Neuroendocrine peptides in the prostate. Urol. Res., 23: 81-87.
Gleave, M., Miyaki, H., Zangemeister-Wittke, U., Jansen, B. (2002) Antisense therapy: current status in prostate cancer and other malignancies. Cancer Mets. Rev., 21: 79-92.
Gleave, M., Jansen, B. (2003) Clusterin and IGFBPs as antisense targets in prostate cancer. Ann. N.Y. Acad. Sci., 1002: 95-104.
Gottardi, C. J. and Gumbiner, B. M. (2001) Adhesion signaling: how beta-catenin interacts with its partners. Curr. Biol., 11: R792-794.
Grayhack J T, Keeler T C, and Kozlowski J M. Carcinoma of the prostate. Hormonal therapy. Cancer 1987; 60:589-601.
Greenberg, N. M., DeMayo, F., Finegold, M. J., Medina, D., Tilley, W. D., et al. (1995) Prostate cancer in a transgenic mouse. Proc. Natl. Acad. Sci., U.S.A., 92: 3439-3443.
Grossmann M E, Huang H, Tindall D J. (2001). J Natl Cancer Inst 93: 1687-1697.
Hajra, K. M. and Fearon, E. R. (2002) Cadherin and catenin alterations in human cancer. Genes, Chrom. & Cancer, 34: 255-268.
Hansson, J., Abrahamsson, P. A. (2001) Neuroendocrine pathogenesis in adenocarcinoma of the prostate. Ann. Oncol., 12(S-2): 145-152.
Harding, M. A. and Theodorescu, D. (1999) Prognostic markers in localized prostate cancer: from microscopes to molecules. Cancer Met. Reviews, 17: 429-437.
Hawley, P., Gibson, I. (1996) Interaction of oligodeoxynucleotides with mammalian cells. Antisense & Nucleic Drug Dev., 6: 185-195.
He T C, Sparks A B, Rago C, Hermeking H, Zawel L, da Costa L T et al. (1998). Science 281: 1509-1512.
Hecht, A., Kemler, R. (2000) Curbing the nuclear activities of beta-catenin. Control over wnt target gene expression. EMBO Rep., 1: 24-28.
Heidenberg H B, Sesterhenn I A, Gaddipati J P, et al. Alteration of the tumor suppressor gene p53 in a high fraction of hormone refractory prostate cancer. J Urol 1995; 154:414-21.
Hendriks B, Reichmann E. Wnt signaling: a complex issue. Biol Res 2002; 35:277-286.
Hilschmann, N., Barnikol, H. U., Barnikol-Watanabe, S., Gotz, H., Kratzin, H. and Thinnes, F. P. (2001) The immunoglobulin-like genetic predetermination of the brain: the protocadherins, blueprint of the neuronal network. Naturwissenschaften, 88: 2-12.
Hirano D, Okada Y, Minei S, Takimoto Y, Nemoto N. Neuroendocrine differentiation in hormone refractory prostate cancer following androgen deprivation therapy. Eur Urol 2004; 45:586-592.
Huss, W. J., Gregory, C. W. and Smith, G. J. (2004) Neuroendocrine cell differentiation in the CWR22 human prostate cancer xenograft: association with tumor cell proliferation prior to recurrence.
Hvamstad, T., Jordal, A., Hekmat, N., Paus, E. and Fossa, S. D. (2003) Neuroendocrine serum tumour markers in hormone-resistant prostate cancer. Eur. Urol., 44: 215-221.
Isaacs J T, Furuya Y, Berges R. The role of androgen in the regulation of programmed cell death/apoptosis in normal and malignant human prostate tissue. Sem Cancer Biol 1994; 5:391-400.
Isaacs J T, Schulze H, and Coffey D S. Development of androgen resistance in prostatic cancer. Prog Clin Biol Res 1987; 243:21-31.
Isaacs, W. B., Bova, G. S., Morton, R. A., Bussemakers, M. J., Brooks, J. D. and Ewing, C. M. (1994) Genetic alterations in prostate cancer. Cold Springs Harbor Symp. Quant. Biol., 59: 653-659.
Isaacs, J. T., Furuya, Y. and Berges, R. (1994) The role of androgen in the regulation of programmed cell death/apoptosis in normal and malignant prostate tissue. Sem. Cancer Biol., 5: 391-400.
Ismail A, Landry F, Aprikian A G, Chevalier S. Androgen ablation promotes neuroendocrine cell differentiation in dog and human prostate. Prostate 2002; 51:117-125.
Isshiki S, Akakura K, Komiya A, Suzuki H, Kamiya N, Ito H. Chromogranin A concentration as a serum marker to predict prognosis after endocrine therapy for prostate cancer. J Urol 2002; 167:512-515.
Ito, T., Yamamoto, S., Ohno, Y., Namika, K., Aizawai, T., Akyama, A. and Tachibana, M. (2001) Up-regulation of neuroendocrine differentiation in prostate cancer after androgen deprivation therapy, degree and androgen independence. Oncol. Rep., 8: 1221-1224.
Ivanov, D. B., Philippova, M. P. and Tkachuk, V. A. (2001) Structure and function of classical cadherins. Biochem. 66: 1174-1186.
Jimenez, N., Jongsma, J., Calvo, A., van der Kwast, T. H., Treston, A. M, et al. Peptidylglycine alpha-amidating monooxygenase- and proadrenomedullin-derived peptide associated neuroendocrine deprivation are induced by androgen deprivation in the neoplastic prostate. Int. J. Cancer, 94: 28-34.
Jin, R. J., Wang, Y., Masumori, N., Ishii, K., Tusukamoto, T., Shappell, S. B., et al. (2004) NE-10 neuroendocrine cancer promotes the LNCaP xenograft growth in castrated mice. Cancer Res., 64: 5489-5495.

Kadmon, D., Thompson, T., Lynch, G. and Scardino, P. T. (1991) Elevated plasma chromogranin-A concentration in prostatic carcinoma. J. Urol. 146: 358-361.
Kalota, A., Shetzline, S. E., Gewirtz, A. M. (2004) Progress in the development of nucleic acid therapeutics for cancer. Cancer Biol. & Therapy, 3: 4-12.
Kamiya, N., Akakura, K., Suzuki, H., Isshiki, S., Komiya, A., Ueda, T. and Ito, H. (2003) Pretreatment serum level of neuron specific enolase (NSE) as a prognostic factor in metastatic prostate cancer patients treated with endocrine therapy. Eur. Urol., 44: 309-314.
Kaplan-Lefko, P. J., Chen, T. M., Ittmann, M. M., Barrios, R. J., Ayala, G. E., Huss, W. J, et al. (2003) Pathobiology of autochthonous prostate cancer in a pre-clinical transgenic mouse model. Prostate, 55: 219-237.
Karan, D., Kelly, D. L., Rizzino, A., Lin, M. F. and Batra, S. K. (2002) Expression profile of differentially-regulated genes during progression of androgen-independent growth in human prostate cancers. Carcinogenesis, 23: 967-975.
Kasper, S., Sheppard, P. C., Yan, Y., Pettigrew, N., Borowsky, A. D., Prins, G. S., et al. (1998) Development, progression and androgen-dependence of prostate tumors in probasin-large T antigen transgenic mice: a model for prostate cancer. Lab. Invest., 78: 319-333.
Kau T R, Schroeder F, Ramaswamy S, Wojciechowski C L, Zhao J J, Roberts T M et al. (2003). Cancer Cell 4: 463-476.
Kikuchi, A. (2003) Tumor formation by genetic mutations in the components of the wnt signaling pathway. Cancer Sci., 94: 225-229.
Klotz L. Hormone therapy for patients with prostate carcinoma. Cancer 2000; 88:3009-3014. Sampson E R, Yeh S Y, Chang H C, et al. Identification and characterization of androgen receptor associated coregulators in prostate cancer cells. J Biol Reg Homeostatic Agents 2001; 15:123
Koh S S, Li H, Lee Y H, Widelitz R B, Chuong C M, Stallcup M R. (2002). J Biol Chem 277: 26031-26035.
Kozak, M. (1983) Comparison of initiation of protein synthesis in procaryotes, eucaryotes, and organelles. Microbiol. Rev, 47: 1-45.
Kreisberg, J. I., Malik, S. N., Prihoda, T. J., et al. (2004) Phosphorylation of Akt (Ser 473) is an excellent predictor of poor clinical outcome in prostate cancer. Cancer Res., 64: 5232-5236.
Krupski T, Petroni G R, Frierson H F Jr, Theodorescu J U (2000) Microvessel density, p53, retinoblastoma and chromogranin A immunohistochemistry as predictors of disease free survival following radical prostatectomy for carcinoma of the prostate. Urol 2000; 55:743-749.
Kyprianou N, English H F, and Isaacs J T. Programmed cell death during regression of PC-82 human prostate cancer following androgen ablation. Cancer Res 1990; 50:3748-53.
Landis S H, Murray T, Bolden S, and Wingo P A. Cancer statistics, 1999. CA Cancer J Clin 1999; 49: 8-31.
Latil A, Bieche I, Vidaud D, et al. Evaluation of androgen, estrogen (ER alpha and ER beta), and progesterone receptor expression in human prostate cancer by real-time quantitative reverse transcription-polymerase chain reaction assays. Cancer Res 2001; 61: 1919-26.
Leckband, D., Sivasankar, S. (2000) Mechanisms of homophilic cadherin adhesion. Curr. Op. Cell Biol., 12: 587-592.
Lessard L, Mes-Masson A M, Lamarre L, Wall L, Lattouf J B, Saad F. NF-kappa B nuclear localization and its prognostic significance in prostate cancer. Br J Urol Intl 2003; 91:417-420. 30.
Leung J Y, Kolligs F T, Wu R, et al. Activation of AXIN2 expression by beta-catenin-T cell factor: A feedback repressor pathway regulating wnt signaling. J Biol Chem 2002; 277:21657-21665.
Li, Y., Welm, B., Podsypanina, K., Huang, S., Chamorro, M., Zhang, X., et al. (2003) Evidence that transgenes encoding components of the Wnt signaling pathway preferentially induce mammary cancers from progenitor cells. Proc. Natl. Acad. Sci., U.S.A., 100: 15853-15858.
Li H, Kim J H, Koh S S, Stallcup M R. (2004). J Biol Chem 279: 4212-4220.
Lilleby, W., Paus, E., Kovlund, E. and Fossa, S. D. (2001) Prognostic value of neuroendocrine serum markers and PSA in irradiation patients with pN0 localized prostate cancer. Prostate, 46: 126-133.

Lin H K, Yeh S, Kang H Y, Chang C. Akt suppresses androgen-induced apoptosis by phosphorylating and inhibiting androgen receptor. Proc Natl Acad Sci, USA 2001; 98:7200-7205
Lin H-K, Wang L, Hu Y-C, Altuwaijri S, Chang C. (2002). EMBO J 21: 4037-4048.
Linja M J, Savinainen K J, Saramaki O R, Tammela T L, Vessella R L, and Visakorpi T. Amplification and overexpression of androgen receptor gene in hormone-refractory prostate cancer. Cancer Res 2001; 61:3550-5.
Longo, K. A., Kennell, J. A., Ochocinska, M. J., Ross, S. E., Wright, W. S. and MacDougal, O. A. (2002) Wnt signaling protects 3T3-L1 preadipocytes from apoptosis through induction of insulin-like growth factors. J. Biol. Chem., 277: 38239-38244.
Lo Muzio, L. (2001) A possible role for the wnt-1 pathway in oral carcinogenesis. Critical Rev in Oral Biol. & Med., 12: 152-165.
Lustig, B., Behrens, J. (2003) The wnt signaling pathway and its role in tumor development. J. Cancer Res. Clin. Oncol., 129: 199-221.
Malik S N, Brattain M, Ghosh P M, et al. Immunohistochemical demonstration of phospho-Akt in high Gleason grade prostate cancer. Clin Cancer Res 2002; 8:1168-1171.
Martin, B., Schneider, R., Janetzky, S., Waibler, Z., Pandur, P., Kuhl, M., Behrens, J., et al. (2002) The LIM-only protein FHL2 interacts with beta-catenin and promotes differentiation of mouse myoblasts. J. Cell Biol., 159: 113-122.
Masumori, N., Tsuchiya, K., Tu, W. H., Lee, C., Kasper, S., Tsukamoto, T., Shappell, S. B. and Matusik, R. (2004) An allograft model of androgen independent prostatic neuroendocrine carcinoma derived from a large probasin promotor-T-antigen transgenic mouse line. J. Urol., 171: 439-442.
Masumori, N., Thomas, T. Z., Chaurand, P., Case, T., Paul, M., Kasper, S., et al. (2001) A probasin-large T antigen transgenic mouse line develops prostate adenocarcinoma and neuroendocrine carcinoma with metastatic potential. Cancer Res., 61: 2239-2249.
McNeal J E. The zonal anatomy of the prostate. Prostate 1981; 2:35-49.
McWilliam L J, Manson C, George N J. Neuroendocrine differentiation and prognosis in prostatic adenocarcinoma. Br J Urol 1997; 80:287-290.
Meyer-Siegler, K. (2001) Cox-2 specific inhibitor, NS-398, increases macrophage migration inhibitory factor expression and induces neuroendocrine differentiation in c4-2b prostate cancer cells. Mol. Med., 7: 850-860.
Miyake H, Tolcher A, and Gleave M E. Antisense Bcl-2 oligodeoxynucleotides inhibit progression to androgen-independence after castration in the Shionogi tumor model. Cancer Res 1999; 59:4030-4.
Miyamoto H, Messing E M, Chang C. (2005). Prostate 61: 332-353.
Monia, B. P., Johnston, J. F., Sasmor, H., Cummins, L L, Geiger, T., et al. (1996) Nuclease resistance and antisense activity of modified oligonucleotides targeted to Ha-ras. J. Biol. Chem., 271: 14533-1440.
Montuenga, L. M., Guembe, L., Burrell, M. A., Bodgetas, M. E., Calvo, A., et al. (2003) The diffuse endocrine system: from embryogenesis to carcinogenesis. Prog. Histochem. Cytochem., 38: 155-272.
Moon R T, Bowerman B, Boutros M, Perrimon N. The promise and perils of Wnt signaling through beta-catenin. Science 2002; 296:1644-1646.
Morin, P. J. (2003) Wnt signaling in human cancer. Cancer Treat. & Res., 115: 169-187.
Morin, P. J. (1999) Beta-catenin signaling and cancer. Bioessays, 21: 1021-1030.
Morlon, A. and Sassone-Corsi, P., (2003) The LIM-only protein FHL2 is a serum inducible transcriptional coactivator of AP-1. Proc. Natl. Acad. Sci., U.S.A., 100: 3977-3982.
Mucci N R, Akdas G, Manely S, Rubin, M A. Neuroendocrine expression in metastatic prostate cancer: evaluation of high throughput tissue microarrays to detect heterogenous protein expression. Hum. Pathol 2000; 31 406-414.
Mulholland D J, Read J T, Rennie P S, Cox M E, and Nelson C C. Functional localization and competition between the androgen receptor and T-cell factor for nuclear beta-catenin: a means for inhibition of the Tcf signaling axis. Oncogene 2003; 22:5602-13.
Muller, J. M., Isele, U., Metzger, E., Rempel, A., Moser, M., Pscherer, A. Breyer, T., et al. (2000) FHL2, a novel tissue-specific coactivator of the androgen receptor. EMBO J., 19, 359-369.
Murillo, H., Huang, H., Schmidt, L. J., Smith, D. I. and Tindall, D. J. (2001) Role of PI3-K signaling in survival and progression of LNCaP cells to the androgen refractory state. Endocrin., 142: 4795-4805.
Navone N M, Troncoso P, Pisters L L, et al. p53 protein accumulation and gene mutation in the progression of human prostate carcinoma. J Natl Cancer Inst 1993; 85:1657-69.
Noordzij, M. A., Van Steenbrugge, G. J., Van der Kwast, T. H. and Schroeder, F. H. (1995) Neuroendocrine cells in the normal, hyperplastic and neoplastic prostate. Urol. Res., 22: 331-341.
Nupponen N N, Kakkola L, Koivisto P, and Visakorpi T. Genetic alterations in hormone-refractory recurrent prostate carcinomas. Am J Pathol 1998; 153:141-8.
Ohigashi T, Mizuno R, Nakashima J, Marumo K, Murai M. (2005). Prostate 62: 61-68.
Orford, K., Orford, C. C. and Byers, S. W. (1999) Exogenous expression of beta-catenin regulates contact inhibition, anchorage-independent growth, anoikis and radiation-induced cell cycle arrest. J. Cell Biol., 146: 855-868.
Orr, R., Dorr, M., Andrew, F. (2005) Clinical studies of antisense oligonucleotides for cancer therapy. Meth. Mol. Med., 106: 85-111.
Papandreaou, C. N., Daliani, D. D., Thall, P. F., Tu, S. M., Wang, X., Reyes, A., et al. Results of a phase II study with doxorubicin, etopsoside and cisplatin in patients will fully characterized small cell carcinoma of the prostate. J. Clin. Oncol., 20: 3072-3080.
Paweletz C P, Charboneau L, Bichsel V E, et al. Reverse phase protein microarrays which capture disease progression show activation of pro-survival pathways at the cancer invasion front. Oncogene 2001; 20:1981-9.
Pawlowski, J. E., Ertel, J. R., Allen, M. P., Xu., M., Butler, C., Wilson, E. M. and Weirman, M. E. (2002) Liganded androgen receptor interaction with beta-catenin: nuclear co-localization and modulation of transcriptional activity in neuronal cells. J. Biol. Chem., 277: 20702-20710.
Petrylak, D. P., Tangen, C. M., Hussain, M. H., Lara, P. N. Jr, Jones, J. A., Taplin, M. E., et al. (2004) Docetaxel and estramustine compared with mitoxantrone and prednisone for advanced refractory prostate cancer. N. Engl. J. Med., 351: 1513-1520.
Polakis, P., Hart, M., Rubinfeld, B. (1999) Defects in the regulation of beta-catenin in colorectal cancer. Adv. Exptl. Med. Biol., 470: 23-32.
Queires, L., Gil-Diez-de-Medina, S., Terry, S., Chen, M.-W., de la Taille, A., Allory, Y., et al. (2005) Overexpression of protocadherin-PC mRNA in hormone resistant human prostate cancer. Clin. Cancer Res., (In Press).
Raffo A, Perlman H, Chen M-W, Day M, Buttyan R. Overexpression of bcl-2 protects prostate cancer cells from apoptosis in vitro and confers resistance to androgen depletion in vivo. Cancer Res 1995; 55:4438-4445.
Rahman M, Miyamoto H, Chang C. (2004). Clin Cancer Res 10: 2208-2219.
Randolph, T. L., Amin, M. B., Ro, J. Y. and Ayala, A. G. (1997) Histologic variants of adenocarcinoma and other carcinomas of prostate: pathologic criteria and clinical significance. Modern Pathol., 10: 612-629.
Ratcliffe M J, Itoh K, Sokol S Y. (2000). J Biol Chem 275: 35680-35683.
Retter, A. S., Gulley, J. L., Dahut, W. L. (2004) Novel therapeutic strategies in prostate cancer. Cancer Biol. & Ther., 3: 371-376.
Roudier M P, True L D, Higano C S, et al. Phenotypic heterogeneity of end-stage prostate carcinoma to the bone. Hum Pathol 2003; 34:646-653.
Rye, P. D., Stigbrand, T. (2004) Interfering with cancer: a brief outline of advances in RNA interference in oncology. Tumour Biol., 25: 329-336.
Sancho E, Batlle E, Clevers H. (2004). Ann Rev Cell Develop Biol 20: 695-723.
Schulze H, Isaacs J T, and Coffey D S. A critical review of the concept of total androgen ablation in the treatment of prostate cancer. Prog Clin Biol Res 1987; 243:1-19.
Segawa, N., Mori, I., Utsunomiya, H., Nakamura, M., Nakamura, Y., Shan, L., et al. (2001) Prognostic significance of neuroendocrine differentiation, proliferation activity and androgen receptor expression in prostate cancer. Pathol. Intl., 51: 452-459.
Shah, G. V., Rayford, W., Noble, M. J., Austenfeld, M., Weigel, J., et al. (1994) Calcitonin stimulation growth of human prostate cancer cells through receptor-mediated increase in cyclic adenosome 3′,5′ monophosphates and cytoplasmics Ca2+ transients. Endocrinology, 134: 596-602.
Shappell, S. B., Thomas, G. V., Roberts, R. L., Herbert, R., Ittmann, M. M., Rubin, M. A., et al. (2004) Prostate pathology of genetically engineered mice: definitions and classification. The concensus report from the Bar Harbor meeting of the Mouse Models of Human Cancer Consortium Prostate Pathology Committee. Cancer Res., 64, 2270-1305.
Shen, R., Dorai, T., Szabolcs, M., Katz, A. E., Olsson, C. A. and Buttyan, R. (1997) Transdifferentiation of cultured prostate cancer cells to a neuroendocrine cell phenotpe in hormone depleted medium. Urol. Oncol., 3: 67-75.
Shi X B, Ma A H, Tepper C G, Xia L, Gregg J P, Gandour-Edwards R et al. (2004). Prostate 60: 257-271.
Shoji, Y., Nakashima, H. (2004) Current status of delivery systems to improve target efficacy of oligonucleotides. Curr. Pharm. Design, 10: 785-796.
Song, L. N., Herrell, R., Beyers, S., Shah, S., Wilson, E. M. and Gelmann, E. P. (3003) Beta-catenin binds to the activation function 2 region of the androgen receptor and modulates the effects of the N-terminal domain and TIF2 on ligand-dependent transcription. Mol. Cell. Biol., 23: 1674-1686.
Srivastava M, Bubendorf L, Srikantan V, et al. ANX7, a candidate tumor suppressor gene for prostate cancer. Proc Natl Acad Sci USA 2001; 98:4575-80.
Stack S, Cribbs J T, Gomez L. (2004). J Biol Chem 279: 47732-47739.
Stein, C. A., Cheng, Y.-C. (1993) Antisense oligonucleotides as therapeutic agents—Is the bullet really magical. Science, 261: 1004-10012.
Stappert, J. and Kemler, R. (1994) A short core region of E-cadherin is essential for catenin binding and is highly phosphorylated. Cell Adhes. & Commun., 2: 319-327.
Sun B., Halmost, G., Schally A V., Wang X. and Martinez, M. (2000) Presence of receptors for bombesin/gastrin-releasing peptide mRNA for three receptor subtypes in human prostate cancers. Prostate, 42: 295-303.
Stubbs A P, Abel P D, Golding M, et al. Differentially expressed genes in hormone refractory prostate cancer: association with chromosomal regions involved with genetic aberrations. Am J Pathol 1999; 154:1335-43.
Suzuki, S. T. (1996) Protocadherins and diversity of the cadherin superfamily. J. Cell. Sci., 109: 2609-2611.
Suzuki, S. T. (2000) Recent progress in protocadherin research. Exp. Cell Res., 261: 13-18.
Taguchi A, Blood D C, del Toro G, et al. Blockade of RAGE-amphoterin signalling suppresses tumour growth and metastases. Nature 2000; 405:354-60.
Taplin, M. E. and Balk, S. P. (2004) Androgen receptor: a key molecule in the progression of prostate cancer to hormone independence. J. Cell. Biochem. 91: 483-490.
Tarle M, Frkovic-Graio S, Kraljic I, Kovacic K. A more objective staging of advanced prostate cancer-routine recognition of malignant endocrine structures: The assessment of serum TPS, PSA and NSE values. Prostate 1994; 24:143-148.
Tarle, M. and Rados, N. (1991) Investigation of serum neuron-specific enolase in prostate cancer diagnosis and monitoring: Comparative study of a multiple tumor marker assay. Prostate, 19: 23-33.
Tarle M, Ahel M Z, Kovacic K. Acquired neuroendocrine-positivity during maximal androgen blockade in prostate cancer patients. Anticancer Res 2002; 22:2425-2529.
Tetsu O, McCormick F. Beta-catenin regulates expression of cyclin D1 in colon cancer cells. Nature 1999; 398:422-426.
Tilley W D, Buchanan G, Hickey T E, Bentel J M. (1996). Clin Cancer Res 2: 277-285.
Tovar-Sepulveda, V. A., Falzon, M. (2003) Prostate cancer cell type-specific regulation of the human PTHrP gene via a negative VDRE. Mol. Cell. Endocrinol., 204: 51-64.
True, L. (2004) why we must better understand neuroendocrine differentiation in prostate cancer. J. Urol., 171: 443-444.
Truica C I, Byers S, and Gelmann E P. Beta-catenin affects androgen receptor transcriptional activity and ligand specificity. Cancer Res 2000; 60:4709-13.
Ueda T, Bruchovsky N, Sadar M D. Activation of the androgen receptor N-terminal domain by interleukin-6 via MAPK and STAT3 signal transduction pathways. J Biol Chem 2002; 277:7076-7085.
Ueda, Y., Hijikata, M., Takagi, S., Takada, R., Takada, S., Chiba, T. and Shimotohno, K. (2002) Wnt/beta-catenin signaling suppresses apoptosis in low serum medium and induces morphological changes in rodent fibroblasts. Int. J. Cancer, 99: 681-688.
Vainio, S. J. (2003) Nephrogenesis regulated by wnt signaling. J. Nephrol., 16: 279-285.
van Es, J. H., Barker, N., Clevers, H. (2003) You wnt some, you lose some: oncogenes in the wnt signaling pathway. Curr. Op. Genet. Develop., 13: 28-33.
Van Noort, M., Clevers, H. (2002) TCF transcription factors, mediators of wnt-signaling in development and cancer. Dev. Biol., 244: 1-8.
Verras M, Brown J, Li X, Nusse R, Sun Z. (2004). Cancer Res 64: 8860-8866.
Visakorpi T, Hyytinen E, Koivisto P, et al. In vivo amplification of the androgen receptor gene and progression of human prostate cancer. Nat Genet 1995; 9: 401-6.
Voeller J J, Truica C I, Gelmann E P. Beta-catenin mutations in human prostate cancer. Cancer Res 1998; 58:2520-2523.
Wang, Q., Horiatis, D. and Pinski, J. (2004) Interleukin-6 inhibits the growth of prostate cancer xenografts in mice by the process of neuroendocrine differentiation. Int. J. Cancer, 111: 508-513.
Watanabe M, Kakiuchi H, Kato H, Shraishi T, Yatani R, Sugimura T et al. (1996). Jap J Clin Oncol 26: 77-81.
Wei, Y., Renard, C. A., Labalette, C., Wu, Y., Levy, L., Neuveut, C., Prieur, X., et al. (2003) Identification of the LIM protein FHL-2 as a coactivator of beta-catenin. J. Biol. Chem., 278, 5188-5194.
Weinstein M H, Partin A W, Veltri R W, Epstein J I. Neuroendocrine differentiation in prostate cancer: enhanced prediction of progression after radical prostatectomy. Hum Pathol 1996; 27:683-687.
Westin P, Stattin P, Damber J E, and Bergh A. Castration therapy rapidly induces apoptosis in a minority and decreases cell proliferation in a majority of human prostatic tumors. Am J Pathol 1995; 146:1368-75.
Yanfeng, W., Saint-Jeannet, J. P. and Klein, P. S. (2003) Wnt-frizzled signaling in the induction and differentiation of the neural crest. Bioessays, 25: 317-325.
Yang F, Li X, Sharma M, et al. Linking beta-catenin to androgen-signaling pathway. J Biol Chem 2002; 277:11336-44.
Yang, Y. (2003) Wnts and wing: Wnt signaling in vertebrate limb development and musculoskeletal morphogenesis. Birth Def. Res., 69: 305-317.
Yang, X., Chen, M.-W., Terry, S., Vacherot, F., Chopin, D. K., Bemis, D. L., Kitajewski, J., Benson, M. C., Guo, Y. and Buttyan, R. (2005) A human- and male-specific protocadherin that acts through the Wnt signaling pathway to induce neuroendocrine transdifferentiation of prostate cancer cells. Cancer Research, 65: 5263-5271.
Yeh S, Lin H K, Kang H Y, Thin T H, Lin M F, Chang C. From HER2/Neu signal cascade to androgen receptor and its coactivators: a novel pathway by induction of androgen target genes through MAP kinase in prostate cancer cells. Proc Natl Acad Sci, USA 1999; 96:5458-5463.
You, Z., Saims, D., Chen, S., Zhang, Z., Guttridge, D. C., Guan, K. l., MacDougald, O. A., et al. (2002) Wnt signaling promotes oncogenic transformation by inhibiting c-Myc induced apoptosis. J. Cell Biol. 157: 429-440.
Yoshida K and Sugano S. Identification of a novel protocadherin gene (PCDH11) on the human XY homology region in Xq21.3. Genomics 1999; 62:540-3.
Young C S, Kitamura M, Hardy S, Kitajewski J. (1998). Mol Cell Biol 18: 2474-2485.
Yu, D. S., Hsieh, D. S., Chen, H. I. and Chang, S. Y. (2001) The expression of neuropeptides in hyperplastic and malignant prostate tissue and its clinical implications. J. Urol., 166: 871-875.
Zelivianski, S., Vemi, M., Moore, C., Kondrikov, D., Taylor, R. and Lin, M. F. (2001) Biochim et Biophys. Acta, 1539: 28-43.
Zhang, X., Chen, M.-W., Ng, A., Ng, P.-Y., Lee, C., Rubin, M., Olsson, C. A. and Buttyan, R. (1997) Abnormal prostate development in C3(1)-bcl-2 transgenic mice. Prostate, 32: 16-26.
Zhang, X., Lee, C., Ng, P.-Y., Rubin, M., Shabsigh, A. and Buttyan, R. (2000) Prostatic neoplasia in transgenic mice with prostate-directed overexpression of the c-myc oncoprotein. Prostate, 43: 278-286.

What is claimed is:

1. A nucleic acid comprising from about 7 to about 30 nucleotides that specifically binds to a region from about nucleotide 3023 to about nucleotide 3727 of SEQ ID NO:1, wherein the nucleic acid is capable of inhibiting expression of protocadherin-PC.

2. The nucleic acid of claim 1, wherein the nucleic acid comprises RNA, antisense RNA, small interfering RNA (siRNA), double stranded RNA (dsRNA), short hairpin RNA (shRNA), cDNA, DNA, or any combination thereof.

3. The nucleic acid of claim 1, wherein the nucleic acid comprises a sequence within the region of from about nucleotide 3023 to about nucleotide 3727 of SEQ ID NO:1.

4. The nucleic acid of claim 1, wherein the nucleic acid comprises at least one of SEQ ID NOS:3, 4, 5, 6, or 7.

5. The nucleic acid of claim 1, wherein the nucleic acid comprises a UU overhang or a TT overhang.

6. The nucleic acid of claim 1, wherein the nucleic acid comprises at least one chemically modified nucleotide or at least one modified internucleotide linkage to render it resistant to enzymatic degradation.

7. The nucleic acid of claim 6, wherein the modified nucleotide comprises a 2′-O-methoxy-residue.

8. The nucleic of claim 6, wherein the modified nucleotide linkage is a phosphorothioate linkage.

9. A composition comprising the nucleic acid of claim 1 and a pharmaceutically acceptable carrier.

10. A nucleic acid comprising a nucleic acid expression vector encoding a short hairpin RNA (shRNA), wherein the shRNA comprises the small interfering RNA (siRNA) nucleotide sequence of SEQ ID NO:3, 4, 5, 6, or 7.

11. A composition comprising the nucleic acid of claim 1 or 10 and a pharmaceutically acceptable carrier.

12. A host organism comprising the nucleic acid of claim 1 or 10.

13. The host organism of claim 12, wherein the host is a prokaryote or a eukaryote.

14. A cell comprising the nucleic acid of claim 1 or 10.

15. A mammal comprising one or more cells of claim 14.

16. An antibody or antigen-binding fragment thereof, that specifically binds to the Y-chromosome-encoded homologue of protocadherin-PC, comprising the amino acid sequence of SEQ ID NO:2, and wherein the antibody or antigen-binding fragment thereof does not bind to the X-chromosome-encoded homologue of protocadherin-PC.

17. An antibody or antigen-binding fragment thereof that binds to the Y-chromosome encoded homologue of protocadherin-PC and binds to the X-chromosome encoded homologue of protocadherin-PC.

18. A method for treating cancer in a subject, the method comprising administering to the subject an effective amount of an inhibitor of protocadherin-PC.

19. The method of claim 18, wherein the cancer comprises at least one of prostate, breast, melanoma, oral, colon, ovarian, endometrial, hepatocellular carcinoma, or head and neck tumors.

20. A method for treating hormone-resistant prostate cancer in a subject, the method comprising administering to the subject an effective amount of an inhibitor of protocadherin-PC.

21. A method for treating prostate cancer in a subject, the method comprising administering to the subject a combination of one or more androgen-withdrawal therapies and an effective amount of an inhibitor of protocadherin-PC.

22. The method of claim 18, 19, 20, or 21, wherein the inhibitor comprises a small interfering RNA (siRNA), an antisense oligonucleotide, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof.

23. The method of claim 18, 19, 20, or 21, wherein the inhibitor comprises a protein interaction inhibitor that disrupts protocadherin-PC binding domains, FHL-2 binding domains, or β-catenin binding domains.

24. The method of claim 18, 19, 20, or 21, wherein the subject is a human, mouse, rabbit, monkey, rat, bovine, pig or dog.

25. The method of claim 18, 19, 20, or 21, wherein the administering comprises intralesional, intraperitoneal, intramuscular, intratumoral or intravenous injection; infusion; liposome- or vector-mediated delivery; or topical, nasal, oral, ocular, otic delivery, or any combination thereof.

26. The method of claim 18, 19, 20, or 21, wherein an effective amount comprises an amount effective to arrest, delay or reverse the progression of the cancer.

27. The method of claim 20, wherein the hormone-resistant prostate cancer is also resistant to chemotherapy and/or radiation therapy.

28. The method of claim 21, wherein the androgen-withdrawal therapy comprises surgical orchiectomy.

29. The method of claim 21, wherein the androgen-withdrawal therapy comprises medical hormone therapies including but not limited to anti-androgens and luteinizing hormone-releasing hormone agonists.

30. A method for treating prostate cancer in a subject, the method comprising administering to a subject an effective amount of a radiolabeled compound capable of specifically binding to protocadherin-PC.

31. The method of claim 30, wherein the compound comprises comprises a small interfering RNA (siRNA), an antisense oligonucleotide, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof.

32. The method of claim 30, wherein the compound comprises a nucleic acid that is capable of specifically binding to a nucleic acid encoding protocadherin-PC, or a fragment thereof.

33. A method for in vivo imaging of cancer in a subject, the method comprising
(a) administering to the subject a radiolabeled compound capable of specifically binding to protocadherin-PC or FHL-2; and
(b) detecting the presence of the radiolabeled compound in the subject, thereby imaging cancer in the subject.

34. The method of claim 33, wherein the cancer comprises prostate cancer or breast cancer.

35. The method of claim 33, wherein the compound comprises comprises a small interfering RNA (siRNA), an antisense oligonucleotide, a peptide nucleic acid (PNA) that specifically binds a nucleic acid encoding protocadherin-PC, a ribozyme that specifically cleaves a nucleic acid encoding protocadherin-PC, a small molecule, an antibody or antigen binding fragment thereof, a peptide, a peptidomimetics, or any combination thereof.

36. The method of claim 33, wherein the compound comprises a nucleic acid specific for a nucleic acid, or a fragment thereof encoding protocadherin-PC or FHL-2.

37. The method of claim 33, wherein the compound is detected by MRI, SPECT, CT, or ultrasound.

38. A method for identifying whether a test compound is capable of inhibiting protocadherin-PC protein activity, the method comprising
(a) contacting a protocadherin-PC protein with
(i) a test compound and
(ii) a β-catenin or an FHL-2 or both; and
(b) determining whether activity of the protocadherin-PC protein of step (a) is inhibited as compared to the activity of a protocadherin-PC protein in the absence of the test compound, so as to identify whether the test compound is capable of inhibiting protocadherin-PC protein activity.

39. The method of claim 38, wherein the determining comprises (a) determining binding of the protocadherin-PC protein to the β-catenin and/or to the FHL-2, (b) determining whether the protocadherin-PC is capable of translocating β-catenin to the cytoplasm, (c) determining whether protocadherin-PC is activating the wnt signaling pathway or increasing the expression of LEF-1/TCF target genes in the cancer cell, (d) determining whether protocadherin-PC is modulating the expression of the androgen receptor protein, or (e) any combination thereof.

40. The method of claim 38, wherein the contacting is achieved by applying the test compound to cells expressing the protocadherin-PC, the β-catenin, and the FHL-2.

41. A method for identifying whether a test compound is capable of inhibiting protocadherin-PC binding to β-catenin or FHL-2, the method comprising
(a) contacting a protocadherin-PC protein with
(i) a test compound and
(ii) a β-catenin or an FHL-2 or both; and
(b) determining whether binding of the protocadherin-PC protein to the β-catenin and/or the FHL-2 is inhibited compared to binding of the protocadherin-PC protein to the β-catenin and/or the FHL-2 in the absence of the test compound, so as to identify whether the test compound is capable of inhibiting the protocadherin-PC binding to the β-catenin or the FHL-2.

42. The method of claim 41, wherein the test compound comprises a nucleic acid, a small molecule, a peptide, a PNA, a peptidomimetic, or an antibody.

43. The method of claim 41, wherein the method is carried out for more than one hundred compounds.

44. The method of claim 41, wherein the method is carried out in a high-throughput manner.

45. A method for identifying whether a test compound is capable of inhibiting gene expression of protocadherin-PC, the method comprising:
(a) contacting a nucleic acid encoding a protocadherin-PC protein with a test compound; and
(b) determining whether the protocadherin-PC gene expression is inhibited compared to protocadherin-PC gene expression in the absence of the test compound.

46. The method of claim 45, wherein the determining comprises measuring transcription levels of the protocadherin-PC gene by detecting a gene product.

47. The method of claim 45, wherein the determining comprises measuring levels of protocadherin-PC mRNA.

48. The method of claim 45, wherein the determining comprises measuring levels of protocadherin-PC protein.

49. The method of claim 45, wherein the determining comprises measuring activity levels of protocadherin-PC protein.

50. A kit for determining whether or not a subject has or may develop prostate cancer, the kit comprising
(a) an antibody or an antigen-binding fragment thereof, that specifically binds to a protocadherin-PC or an FHL-2; and
(b) at least one negative control sample that does not contain a protocadherin-PC antigen or an FHL-2 antigen.

51. The kit of claim 50, further comprising a positive control sample that contains a protocadherin-PC antigen in an amount characteristic of a human prostate cancer cell.

52. The kit of claim 50, wherein the antibody or antigen-binding fragment is labeled with a detectable signal.

53. A transgenic non-human mammal whose genome comprises a transgene comprising a nucleic acid encoding a protocadherin-PC operably linked to a tissue-specific promoter.

54. The transgenic non-human mammal of claim 53, wherein the mammal is a mouse, a primate, a bovine, or a porcine.

55. The transgenic non-human mammal of claim 53, wherein the tissue-specific promoter is a prostate-specific probasin gene promoter element.

56. An F1 transgenic mouse produced from a cross between the mouse of claim 53 and a transgenic mouse of the TRAMP (strain: C57BU6-Tg(TRAMP)8247Ng/J; Jackson Lab No. 003135) or any other mouse that develops prostate cancer.

57. A method for determining whether a test compound is capable of treating prostate cancer, the method comprising:
(a) administering an effective amount of a test compound to a transgenic non-human mammal whose genome comprises a transgene comprising a nucleic acid encoding a protocadherin-PC operably linked to a tissue-specific promoter, wherein the transgenic non-human mammal has prostate cancer;
(b) measuring progression of prostate cancer in the transgenic non-human mammal of (a);
(c) comparing the measurement of progression of prostate cancer of step (b) to that of a sibling of the transgenic non-human mammal, wherein the sibling was not administered the test compound, and wherein an arrest, delay or reversal in progression of prostate cancer in the transgenic non-human mammal of (a) indicates that the test compound is capable of treating prostate cancer.

58. An isolated prostate cancer cell that does not express a protocadherin-PC gene, wherein the naturally occurring prostate cancer cell does express the protocadherin-PC gene.

59. A hybridoma cell line deposited with the CNCM under No. I-3560.

60. A hybridoma cell line deposited with the CNCM under No. I-3561.

61. A monoclonal antibody produced by hybridoma cells deposited with the CNCM under No. I-3560.

62. A monoclonal antibody produced by hybridoma cells deposited at the CNCM under No. I-3561.

63. A method for determining whether a subject has or may develop prostate cancer, the method comprising
(a) administering to the subject antibodies of claim 16, 17, 61, or 62; and
(b) detecting the presence of the labeled antibodies in the subject;
wherein detection of the labeled antibodies indicates that the subject has or may develop prostate cancer.

64. A method for determining whether a subject has or may develop prostate cancer, the method comprising
(a) removing a biological sample from the subject;
(b) contacting the sample with antibodies of claim 16, 17, 61, or 62; and
(c) detecting the presence of the antibodies in the sample;
wherein detection of the labeled antibodies indicates that the subject has or may develop prostate cancer.

65. The method of claim 63, wherein the antibodies comprise a detectable label.

66. The method of claim 64, wherein the antibodies comprise a detectable label.

67. The method of claim 63, wherein the antibodies are used as tumor markers for early detection of prostate cancer.

68. The method of claim 64, wherein the antibodies are used as tumor markers for early detection of prostate cancer.

69. The method of claim 63, wherein the method is used for pre-treatment staging of prostate cancer.

70. The method of claim 64, wherein the method is used for pre-treatment staging of prostate cancer.

71. The method of claim 63, wherein the method is used for post-treatment monitoring of prostate cancer.

72. The method of claim 64, wherein the method is used for post-treatment monitoring of prostate cancer.

73. The method of claim 63, wherein the method is used to distinguish between indolent prostate cancer and aggressive prostate cancer.

74. The method of claim 64, wherein the method is used to distinguish between indolent prostate cancer and aggressive prostate cancer.

75. The kit of claim 50, wherein the antibody comprises monoclonal antibodies produced by hybridoma cells deposited with the CNCM under No. I-3560.

76. The kit of claim 50, wherein the antibody comprises monoclonal antibodies produced by hybridoma cells deposited with the CNCM under No. I-3561.

77. A nucleic acid comprising the sequence of SEQ ID NO:3.

78. A nucleic acid comprising the sequence of SEQ ID NO:4.

79. A nucleic acid comprising the sequence of SEQ ID NO:5.

80. A nucleic acid comprising the sequence of SEQ ID NO:6.

81. A nucleic acid comprising the sequence of SEQ ID NO:7.
